SEQUENCE LISTING (24 sequences)

SEQ ID NO: 1 - protein, 6 aa, Homo sapiens
KQLATK

SEQ ID NO: 2 - protein, 10 aa, Homo sapiens
KSAPATGGVK

SEQ ID NO: 3 - protein, 9 aa, Homo sapiens
SAPATGGVK

SEQ ID NO: 4 - protein, 9 aa, Homo sapiens
YRPGTVALR

SEQ ID NO: 5 - protein, 7 aa, Homo sapiens
STELLIR

SEQ ID NO: 6 - protein, 6 aa, Homo sapiens
KLPFQR

SEQ ID NO: 7 - protein, 7 aa, Homo sapiens
EIAQDFK

SEQ ID NO: 8 - protein, 7 aa, Homo sapiens
RVTIMPK

SEQ ID NO: 9 - protein, 6 aa, Homo sapiens
VTIMPK

SEQ ID NO: 10 - protein, 9 aa, Homo sapiens
  MOD_RES (6)..(6): ACETYLATION
KSTGGKAPR

SEQ ID NO: 11 - protein, 9 aa, Homo sapiens
  MOD_RES (1)..(1): ACETYLATION
KSTGGKAPR

SEQ ID NO: 12 - protein, 8 aa, Homo sapiens
  MOD_RES (5)..(5): ACETYLATION
STGGKAPR

SEQ ID NO: 13 - protein, 6 aa, Homo sapiens
  MOD_RES (1)..(1): ACETYLATION
KQLATK

SEQ ID NO: 14 - protein, 9 aa, Homo sapiens
  MOD_RES (6)..(6): ACETYLATION
KQLATKAAR

SEQ ID NO: 15 - protein, 9 aa, Homo sapiens
  MOD_RES (1)..(1): ACETYLATION
KQLATKAAR

SEQ ID NO: 16 - protein, 8 aa, Homo sapiens
  MOD_RES (5)..(5): ACETYLATION
QLATKAAR

SEQ ID NO: 17 - protein, 10 aa, Homo sapiens
  MOD_RES (1)..(1): ACETYLATION
KSAPATGGVK

SEQ ID NO: 18 - protein, 10 aa, Homo sapiens
  MOD_RES (1)..(1): ACETYLATION
KSAPATGGVK

SEQ ID NO: 19 - protein, 14 aa, Homo sapiens
  MOD_RES (1)..(1): ACETYLATION
KSAPATGGVKKPHR

SEQ ID NO: 20 - protein, 14 aa, Homo sapiens
  MOD_RES (1)..(1): ACETYLATION
KSAPATGGVKKPHR

SEQ ID NO: 21 - protein, 11 aa, Homo sapiens
  MOD_RES (4)..(4): ACETYLATION
RYQKSTELLIR

SEQ ID NO: 22 - protein, 13 aa, Homo sapiens
  MOD_RES (7)..(7): ACETYLATION
RVTIMPKDIQLAR

SEQ ID NO: 23 - protein, 12 aa, Homo sapiens
  MOD_RES (6)..(6): ACETYLATION
VTIMPKDIQLAR

SEQ ID NO: 24 - protein, 20 aa, Homo sapiens
ARTKQTARKSTGGKAPRKQY

(19) United States
(12) Patent Application Publication
(10) Pub. No.: US 2010/0203647 A1
(43) Pub. Date: Aug. 12, 2010

(54) Chemical Reporters of Protein Acylation

(75) Inventors: HOWARD C. HANG, NEW YORK, NY (US); GUILLAUME CHARRON, NEW YORK, NY (US); JOHN P. WILSON, NEW YORK, NY (US); ANURADHA RAGHAVAN, NEW HAVEN, CT (US); MINGZI ZHANG, NEW YORK, NY (US); YU-YING YANG, STAMFORD, CT (US); KAVITA RANGAN, NEW YORK, NY (US); LUN K. TSOU, NEW YORK, NY (US); JACOB YOUNT, BROOKLYN, NY (US)

Correspondence Address: THOMPSON COBURN LLP, ONE US BANK PLAZA, SUITE 3500, ST LOUIS, MO, 63101, US

(73) Assignee: THE ROCKEFELLER UNIVERSITY, New York, NY (US)

(21) Appl. No.: 12/623,335

(22) Filed: Nov. 20, 2009

Related U.S. Application Data
(60) Provisional application No. 61/119,545, filed on Dec. 3, 2008.
(60) Provisional application No. 61/117,002, filed on Nov. 21, 2008.

(51) Int. Cl. G01N 33/00; C09B 29/00; C07C 245/24

(52) U.S. Cl. 436/86; 534/752; 534/551

(57) ABSTRACT

Methods and kits for detecting acylated proteins produced by cells that have been cultured or by cells within an organism are provided. Also provided are methods and kits for detecting acylated proteins produced by cells where affinity purification tags are that facilitate detection are used. Compounds useful for the detection of acylated proteins are also provided.

### CROSS-REFERENCE TO RELATED APPLICATIONS

This application claims the benefit of priority to U.S. Appl. No. 61/117,002, filed Nov. 21, 2008, and the benefit of priority to U.S. Appl. No. 61/119,545, filed Dec. 3, 2008, both of which are incorporated herein by reference in their entireties.

### STATEMENT REGARDING FEDERALLY SPONSORED RESEARCH OR DEVELOPMENT

Not Applicable.

### INCORPORATION OF SEQUENCE LISTING

An electronic computer readable (CRF) form of the sequence listing entitled “Seq_Lst_{—}49248_{—}86461_ST25.txt”, which is 5231 bytes (measured in MS-Windows), which contains 24 sequences, and which was created on Nov. 18, 2009, is filed herewith and herein incorporated by reference in its entirety.

### BACKGROUND OF THE INVENTION

Protein acylation, including fatty-acylation and acetylation, regulates diverse biological processes that include signal transduction, gene expression, cellular growth and differentiation, and cell-cell communication (Resh, M. D. (2006) Nat. Chem. Biol., 2: 584-590; Linder, M. E. (2007) Nat. Rev. Mol. Cell Biol., 8: 74-84; Yang X. J., Seto E. (2008) Mol. Cell 31(4): 449-461). Fatty-acylation of proteins in eukaryotes includes N-myristoylation and S-palmitoylation, characterized by the attachment of myristic acid (14:0) or palmitic acid (16:0) to proteins, respectively (FIG. 1). Myristoylation occurs predominantly on N-terminal glycine residues of nascent polypeptides, whereas S-palmitoylation or S-acylation of proteins takes place on the thiol side chain of cysteine residues. Fatty acid modifications target proteins to discrete membrane compartments thus enabling spatial and temporal regulation of complex signaling pathways. Fatty-acylation can also dramatically influence the extracellular signaling properties of secreted proteins in tissues (Miura, G. I. and Treisman, J. E. (2006) Cell Cycle, 5: 1184-1188). While many fatty-acylated proteins have been identified and are associated with signal transduction pathways, analysis of protein lipidation has remained difficult, hampering the understanding of mechanisms that regulate protein fatty-acylation (Resh, M. D. (2006) Methods, 40: 191-197).

The quantitative biochemical analysis of lipidated proteins has been traditionally performed with radiolabeled (3H or 14C) fatty acids used to visualize fatty-acylated proteins in cell lysates or after immunoprecipitation with specific antibodies (Resh, M. D. (2006) Methods, 40: 191-197). While effective, autoradiography often requires days to weeks to visualize lipidated proteins. Radioactive 125iodinated-fatty acids improve the detection of fatty-acylated proteins by autoradiography, but these reagents are hazardous, cumbersome and not readily available (Berthiaume, L. and Resh, M. D. (1995) J. Biol. Chem., 270: 22399-22405). To circumvent the limitations of radiolabeled fatty acids, the acyl-biotin exchange (ABE) protocol developed by Drisdel and Green affords a non-radioactive means to visualize S-palmitoylated proteins by streptavidin blot (Drisdel, R. C. and Green, W. N. (2004) Biotechniques, 36:276-285). Chemical reporters of protein fatty-acylation that enable rapid non-radioactive detection of N-myristoylated and S-palmitoylated proteins from mammalian cells using bioorthogonal labeling methods have also been reported (Hang, H. C. (2007) J. Am. Chem. Soc., 129: 2744-2745) (FIG. 2). Various bioorthogonal labeling methods for various biomolecules have been reviewed (Prescher and Bertozzi, Nature Chem. Biol, 2005, 1(1)13-21). This chemical approach involves metabolic labeling of cells with azido-fatty acid chemical reporters (FIG. 3A) followed by reaction of azide modified proteins with detection tags, such as phosphine-biotin via the Staudinger ligation (FIG. 3B) and visualization of biotinylated-polypeptides by streptavidin blot (Hang, H. C. (2007) J. Am. Chem. Soc., 129: 2744-2745). This method appears to be quite general, and has also been employed to visualize fatty-acylation of secreted proteins such as Wnt (Ching, W., et al., (2008) J. Biol. Chem., 283: 17092-17098). Other laboratories have also utilized this method (Heal, W. P., et al., (2008a) Chem. Commun. 480-482; Heal, W. P. et al., (2008b) Org. Biomol. Chem., 6: 2308-2315; Kostiuk, M. A., et al., (2008) FASEB J., 22: 721-732; Martin, D. D., et al., (2008) FASEB J. 22: 797-806). The selective biotinylation with ABE (Drisdel, R. C. and Green, W. N. (2004) Biotechniques, 36: 276-285) or azido-fatty acids/phosphine-biotin (Hang, H. C. (2007) J. Am. Chem. Soc., 129: 2744-2745) provides a convenient means to visualize lipidated proteins. The application of the ABE protocol in combination with streptavidin affinity chromatography and Multidimensional Protein Identification Technology (MudPIT) enabled the global analysis of S-palmitoylation in budding yeast, which identified several new S-palmitoylated proteins and highlighted the differential and overlapping substrate specificity of the DHHC-PATs (Roth, A. F., et al. (2006a) Cell, 125(5): 1003-1013; Roth, A. F., et al., (2006) Methods, 40(2): 135-142

Protein acetylation is characterized by the attachment of an acetyl group onto the □-amino group of lysine side chain on proteins (Kourzrides, T. (2007) Cell 128, 693-705). From a chemical perspective, the acetylation of lysines alters their charge state from a positive ammonium species at physiological pH to a neutral amide functional group. This subtle chemical modification not only changes the biochemical properties of proteins but have dramatic affects on cellular pathways (Kourzrides, T. (2007) Cell 128, 693-705) Protein acetylation on lysine residues is best characterized on histones, a set of conversed nuclear proteins that are involved regulating chromatin structure and gene expression (Shahbazian, M. D., and Grunstein, M. (2007) Annu. Rev. Biochem. 76, 75-100). Consequently, the enzymes that initiate protein acetylation on lysine residues were termed histone acetyltransferases (HATs), which utilize acetyl coenzyme A (acetyl-CoA) as their nucleotide substrate (Roth, S. Y., et al., (2001) Annu. Rev. Biochem 70, 81-120; Lee, K. K. et al., (2007) Nat. Rev. Mol. Cell Biol. 8, 284-95; Brownell, J. E. et al., (1996) Cell, 84, 843-51).

The acetylation of histones is critical for controlling many aspects of gene expression (Shahbazian, M. D., and Grunstein, M. (2007) Annu. Rev. Biochem. 76, 75-100). At the macromolecular level, histone acetylation on specific lysine residues regulates nucleosome assembly and chromatin folding (Shahbazian, M. D., and Grunstein, M. (2007) Annu. Rev. Biochem. 76, 75-100). In addition, acetylation of histones modulates DNA recombination, replication and repair pathways (Shahbazian, M. D., and Grunstein, M. (2007) Annu. Rev. Biochem. 76, 75-100). More specifically, lysine acetylation can recruit unique protein binding motifs such as chromodomains that assembly protein complexes responsible for the transcription of genes (Shahbazian, M. D., and Grunstein, M. (2007) Annu. Rev. Biochem. 76, 75-100). In general, acetylation of histones is correlated with active regions of transcription on chromosomes, whereas non-acetylated histones are associated with gene repression (Shahbazian, M. D., and Grunstein, M. (2007) Annu. Rev. Biochem. 76, 75-100). The protein acetylation is regulated by two families of enzymes, HATs and histone deacetylases (HDACs) that install and remove acetyl groups, respectively (Shahbazian, M. D., and Grunstein, M. (2007) Annu. Rev. Biochem. 76, 75-100). HATs can be divided into three major subclasses: GNATs (i.e. Gcn5), MYST (i.e. Mori) and ‘orphan class’ (i.e. p300/CBP and Taf1)¹. HDACs on the other hand are composed of four subclasses: class I (i.e. HDAC1), class IIa (i.e. HDAC4), class IIb (i.e. HDAC6), class III (i.e. SIRT1) and class IV (i.e. HDAC11) (Bolden, J. E., et al., (2006) Nat. Rev. Drug Discov. 5, 769-84). The activities of both enzyme families are regulated by other protein subunits, which determine their overall substrate specificities in cells (Bolden, J. E., et al., (2006) Nat. Rev. Drug Discov. 5, 769-84). Histone acetylation occurs on multiple lysines residues and is regulated by specific HATs and HDACs. For example, the MYST family SAS complex (Sas2-HAT, Sas4 and Sas5) acetylates histone 4 (H4) on lysine 16 (K16) and modulates gene silencing at telomeres. Moreover, HATs exhibit overlapping substrate specificity, as Esa1-HAT can also aceylate H4K16 in the context of INO1 transcription (Shahbazian, M. D., and Grunstein, M. (2007) Annu. Rev. Biochem. 76, 75-100). While a few selective substrates for HATs have been identified, there is no primary amino acid consensus sequence for acetylation.

Understanding the complex interplay between the various HATs and HDACs and the substrates they modify is a major challenge. To complicate matters even further, lysine residues are also modified with other PTMs, such as methylation and ubiquitinylation (Kouzarides, T., (2007) Cell 128, 693-705). In particular, ubiquitinylation often targets proteins for destruction by the proteasome, which would be antagonized by acetylation (Sadoul, K., et al., (2007) Biochimie). Acetylation of histones can be further attenuated by the modification of adjacent amino acid residues with other PTMs, such as phosphorylation (Kouzarides, T., (2007) Cell 128, 693-705). This dynamic regulation of histone modifications is at the core of controlling gene expression and fundamental to the growth and differentiation of cells. This complex regulation of gene expression that is not controlled at the level of DNA sequence has been term “epigenetics” (Goldberg, A. D., et al., (2007) Cell 128, 635-8)

Given the intimate role of protein acetylation on gene expression, it is not surprising in hindsight that the degree of histone acetylation is closely associated with cancer (Bolden, J. E., et al., (2006) Nat. Rev. Drug Discov. 5, 769-84; Jones. P. A. and Baylin, S. B. (2007) Cell 128, 683-92). Indeed, increased levels of non-acetylated histone correlates with condensed chromatin, transcription repression and tumorigenesis (Bolden, J. E., et al., (2006) Nat. Rev. Drug Discov. 5, 769-84; Jones. P. A. and Baylin, S. B. (2007) Cell 128, 683-92) Specifically, the loss of H4K16 acetylation is common hallmark of human cancer. (Bolden, J. E., et al., (2006) Nat. Rev. Drug Discov. 5, 769-84; Jones. P. A. and Baylin, S. B. (2007) Cell 128, 683-92). These observations have paved the way for the development of HDAC inhibitors (HDACi) as anticancer drugs (Bolden, J. E., et al., (2006) Nat. Rev. Drug Discov. 5, 769-84; Jones. P. A. and Baylin, S. B. (2007) Cell 128, 683-92). In fact, suberoylanilide hydroxamic acid (SAHA), a global mechanism-based inhibitor of HDACs, has just been approved by the FDA for treatment of T cell cutaneous lymphoma (Bolden, J. E., et al., (2006) Nat. Rev. Drug Discov. 5, 769-84; Jones. P. A. and Baylin, S. B. (2007) Cell 128, 683-92). While these advances are very exciting for cancer therapy, the contribution of histone acetylation to many areas of physiology that include immunity (Foster, S. L., et al., (2007) Nature 447, 972-8) and neurobiology (Fischer, A., et al., (2007) Nature 447, 178-82) demands a detailed understanding of protein acetylation. In addition to histones, a variety of non-histone proteins are also acetylated (Shahbazian, M. D., and Grunstein, M. (2007) Annu. Rev. Biochem. 76, 75-100; Lee. K. K. and Workman, J. L, (2007) Nat. Rev. Mol. Cell Biol. 8, 284-95; Glozak, M. A., et al., (2005) Gene 363, 15-23; Sadoul, K., et al., (2007) Biochimie; Kim, S. C., et al., (2006) Mol Cell 23, 607-18). One of the first non-histone acetylated proteins was the tumor suppressor p53 (Luo, J., et al., (2004) Proc. Natl. Acad. Sci. U.S.S. 101, 2259-64; Gu, W. and Roeder, R. G., (1997) Cell 90, 595-606). Acetylation of p53 has been shown to enhance sequence-specific DNA binding and is correlated with transcription activation of p53 target genes (Luo, J., et al., (2004) Proc. Natl. Acad. Sci. U.S.A. 101, 2259-64; Gu, W. and Roeder, R. G., (1997) Cell 90, 595-606). p53 acetylation is mediated by CREB-binding protein (CBP)/p300 HAT activity and can be reversed by HDAC1 and Sir2 deacetylase activities (Luo, J., et al., (2004) Proc. Natl. Acad. Sci. U.S.A. 101, 2259-64; Gu, W. and Roeder, R. G., (1997) Cell 90, 595-606). Moreover, p53 acetylation enhances the stability of the protein itself that is thought to occur by antagonizing ubiquitinylation and proteasome-mediated degradation (Luo, J., et al., (2004) Proc. Natl. Acad. Sci. U.S.A. 101, 2259-64). Other classes of non-histone acetylated proteins include transcription factors, RNA splicing and translation factors, chaperones, structural proteins, metabolic enzymes, signaling proteins as well as mitocondrial proteins (Shahbazian, M. D., and Grunstein, M. (2007) Annu. Rev. Biochem. 76, 75-100; Lee, K. K. and Workman, J. L, (2007) Nat. Rev. Mol. Cell Biol. 8, 284-95; Glozak, M. A., et al., (2005) Gene 363, 15-23; Sadoul, K., et al., (2007) Biochimie; Kim, S. C., et al., (2006) Mol Cell 23, 607-18). These findings suggest that protein acetylation plays a broader role in regulating cellular pathways beyond modulating histone function. For most of these acetylated proteins the specific HATs and HDACs that regulated their modification state is unknown. A more complete understanding of substrate specificity of HATs and HDACs is required to fully appreciate the roles of protein acetylation in normal physiological process and diseases. These fundamental studies are particularly important since HDACi are being developed to treat a variety of cancers (Bolden, J. E., et al., (2006) Nat. Rev. Drug Discov. 5, 769-84). Acetylation of proteins like many other PTMs is challenging to study due to heterogeneity (one protein can have multiple sites of modification), low abundance (only a fraction of a particular protein maybe chemically modified at a given time) and dynamic regulation by enzyme families (addition of PTMs by enzymes—HATs) is often counterbalance by enzymes that remove PTMs (HDACs) (Shahbazian, M. D., and Grunstein, M. (2007) Annu. Rev. Biochem. 76, 75-100). Consequently, those skilled in the art are only beginning to understand the role of PTMs in regulating complex biological pathways. To fully appreciate the role of PTMs on protein function, new methods are needed for their detection and identification in complex mixtures. Traditionally, PTMs have been visualized with radiolabeled substrates, such as 3H/14C-acetyl-CoA in the case of protein acetylation (Brownell, J. E., and Allis, C. D., (1995) Proc. Natl. Acad. Sci. U.S.A. 92, 6364-8). However, radiolabelled substrates suffer from low specific activity, are cumbersome to handle and do not provide a means for affinity enrichment from complex mixtures. Alternatively, mass spectrometry can be used to detect acetylation of proteins, but this usually requires purified materials for precise analysis (Garcia. B. A., et al., (2007) Curr. Opin. Chem. Biol. 11, 66-73). Antibodies present a powerful method for the detection of specific antigens and have afforded excellent reagents for the detection of histone acetylation (Garcia. B. A., et al., (2007) Curr. Opin. Chem. Biol. 11, 66-73). Unfortunately, the high specificity of antibodies often renders these reagents selective for the peptide antigen used to immunize animals and often do not provide general reagents for analysis of PTMs, the exception being anti-phosphoTyr antibodies. Furthermore, antibodies that can detect antigens by blotting methods are not necessarily effective for affinity enrichment. To address the limitation of anti-AcLys antibodies, Zhao and coworkers generated polyclonal sera to a mixture of acetylated-Lys containing peptides derived from bovine-serum albumin (BSA) and demonstrated this anti-sera could be used to affinity purify acetylated-peptides from cell lysates (Kim, S. C., et al., (2006) Mol. Cell 23, 607-18). While this approach has identified several new acetyled proteins, particularly mitochondrial proteins, the generality of this anti-AcLys polyclonal serum for affinity enrichment of acetylated proteins is unknown (Kim, S. C., et al., (2006) Mol. Cell 23, 607-18). Alternatively, chloroacetyl-CoA has been shown to be an effective substrate for Gcn5 with purified histones in vitro (Yu, M., et al., (2006) J. Am. Chem. Soc. 128, 15356-7). The resulting chloroacetamide group can be selectively reacted with thiol-containing probes for detection of modified lysines, however, the presence of mM concentrations of thiols in cell lysates precludes the application of this approach to complex mixtures (Yu, M., et al., (2006) J. Am. Chem. Soc. 128, 15356-7). General and robust methods are therefore still needed for the analysis of protein acylated proteins in complex mixtures.

### SUMMARY OF INVENTION

Certain embodiments of the present invention provide for methods for detecting one or more acylated protein(s) produced by a cell. Such methods comprise the steps of: (a) obtaining an protein lysate from a cell provided with one or more chemical reporter(s); (b) labeling one or more protein(s) in said lysate with one or more detection tag(s); and (c) detecting one or more acylated protein(s) labelled with said detection tag(s), thereby detecting one or more acylated protein(s) produced by a cell. In certain embodiments, the chemical reporter(s) is/are an alkynyl-chemical reporter and the detection tag is an azido detection tag. In certain embodiments, the chemical reporter(s) is/are an azido-chemical reporter and the detection tag is an alkynyl detection tag. In certain embodiments, detection of the one or more acylated protein(s) comprises quantitative detection of said one or more acylated protein(s). In certain embodiments, cells are provided with one or more alkynyl-chemical reporter(s) by incubating the cells with the alkynyl-chemical reporter(s). In certain embodiments, an alkynylated protein lysate is labeled with one or more azido detection tag(s) by performing Cu^{I}-catalyzed Huisgen [3+2] cycloaddition. In certain embodiments, one or more azido detection tag(s) used to label acylated-proteins comprises a fluorescent label or an epitope tag. In certain embodiments, the epitope tag is a biotin group, an immunoreactive peptide, or a polyhistidine group. In certain embodiments, the cell can be a prokaryotic cell or a eukaryotic cell. In certain embodiments, a eukaryotic cell is selected from the group consisting of an algal cell, a fungal cell, yeast cell, an insect cell, a fish cell, a plant cell, and a mammalian cell. In certain embodiments, a eukaryotic cell is a mammalian cell. In certain embodiments, a cell is provided with one or more chemical reporter(s) in step (a) above by in vivo administration of one or more of said chemical reporter(s) to an organism. In certain embodiments, the organism is a non-human organism such as an insect, a fish, or a mammal. In certain embodiments, in vivo administration is systemic. In certain embodiments, in vivo administration is localized. In certain embodiments, in vivo administration is by intraperitoneal injection or intravenous injection. In certain embodiments, the method of detecting an acylated protein produced by a cell can further comprise the step of separating one or more detection tag labeled protein(s) from step (b) above before detection in step (c) above. In certain embodiments, one or more detection tag labeled protein(s) from step (b) above are separated by gel electrophoresis, chromatography, or capillary electrophoresis. In certain embodiments, separation by chromatography includes size exclusion chromatography, ion exchange chromatography, affinity chromatography, or a combination thereof. In certain embodiments, one or more alkynyl-chemical reporter(s) comprise a C4 to C24 alkynyl-chemical reporter. In certain embodiments, one or more azido-chemical reporter(s) comprise a C4 to C24 azido-chemical reporter. In certain embodiments, one or more alkynyl chemical reporter(s) comprise at least one of hexa-5-ynoic acid, pent-4-ynoic acid, buta-3-ynoic acid, tetradec-13-ynoic acid, hexadec-15-ynoic acid, octadec-17-ynoic acid, or any combination thereof. In certain embodiments, the acylated protein comprises at least one amino acid residue selected from the group consisting of glycine, lysine, cysteine, serine, tyrosine, and threonine that is acylated. In certain embodiments, the one or more acylated proteins detected is a fatty-acylated protein. In embodiments where the one or more acylated proteins detected is a fatty-acylated protein, the one or more alkynyl-chemical reporter(s) comprise(s) a C7 to C24 alkynyl carbon chain. In certain embodiments where the one or more acylated proteins detected is a fatty-acylated protein, one or more alkynyl-chemical reporter(s) comprise at least one of tetradec-13-ynoic acid, hexadec-15-ynoic acid, octadec-17-ynoic acid, or any combination thereof. In certain embodiments, alkynyl-chemical reporter(s) comprise tetradec-13-ynoic acid and the N-myristoylated proteins produced by the cell are preferentially labeled in comparison to the S-palmitoylated proteins produced by the cell. In certain embodiments, one or more alkynyl-chemical reporter(s) comprise at least one of hexadec-15-ynoic acid, octadec-17-ynoic acid, or any combination thereof and the S-palmitoylated proteins produced by the cell are preferentially labeled in comparison to the N-myristoylated proteins produced by the cell. In certain embodiments, the chemical reporter(s) is/are: i) of the formula R—(CH₂)ₙ—CO—S—CoA, wherein R is an alkynyl or an azido group and n=4-24; or, ii) of the formula R—(CH₂)ₙ—COOH, wherein R is an alkynyl group or an azido group, and wherein n=4-24, or iii) a corresponding cationic salt of the chemical reporter of the formula R—(CH₂)ₙ—COOH, wherein R is an alkynyl group or an azido group, and wherein n=4-24. Such cationic salts include, but are not limited to, Na⁺, K⁺ or Li⁺ salts. In certain embodiments of the methods, the one or more acylated protein(s) detected is an acetylated protein. In certain embodiments where the one or more acylated proteins detected is an acetylated protein, one or more alkynyl-chemical reporter(s) can comprise a C4 to C6 alkynyl carbon chain. In certain embodiments where the one or more acylated proteins detected is an acetylated protein, one or more azido chemical reporter(s) can comprise a C4 to C6 carbon chain. In certain embodiments where the one or more acylated proteins detected is an acetylated protein, one or more alkynyl-chemical reporter(s) can comprise at least one of hexa-5-ynoic acid, pent-4-ynoic acid, buta-3-ynoic acid, or any combination thereof. In certain embodiments where the one or more acylated proteins detected is an acetylated protein, the acetylated protein is a histone. In certain embodiments where the one or more acylated proteins detected is an acetylated protein, one or more chemical reporter(s) can comprise at least one or more compounds of the formula R—(CH₂)ₙ—CO—S—CoA, wherein R is an alkynyl (C≡C) group and n=2 or 3 or wherein R is an azido (N₃) group and n=2 or 3. For cellular studies, chemical reporters may also be administered to cells as their corresponding cationic (Na⁺, K⁺ or Li⁺) salts to facilitate metabolic incorporation. In certain embodiments where the one or more acylated proteins detected is an acetylated protein, one or more alkynyl-chemical reporter(s) can comprise 4-pentynyl-CoA. In certain embodiments where the one or more acylated proteins detected is an acetylated protein, one or more chemical reporter(s) can comprise at least one or more compounds of the formula R—(CH₂)ₙ—COOH, wherein R is an alkynyl (C≡C) group and n=2 or 3 or wherein R is an azido (N₃) group and n=2 or 3. For cellular studies, such chemical reporters may also be administered to cells as their corresponding cationic salts to facilitate metabolic incorporation. Such cationic salts include, but are not limited to, Na⁺, K⁺ or Li⁺ salts. Corresponding cationic salts of the compounds of the formula R—(CH₂)ₙ—COOH, wherein R is an alkynyl (C≡C) group or wherein R is an azido (N₃) group, include, but are not limited to, chemical reporters as shown in FIG. 51.

Certain embodiments of the present invention provide methods for detecting one or more acylated protein(s) produced by a cell where an alkynylated protein is isolated before labeling with an azido tag. Such methods comprise the steps of: (a) obtaining an alkynylated protein lysate from a cell provided with one or more alkynyl-chemical reporter(s); (b) isolating one or more alkynylated protein(s) from said alkynylated protein lysate; (c) labeling one or more of said isolated alkynylated protein(s) with one or more azido detection tag(s); and (d) detecting one or more acylated protein(s) labelled with said detection tag(s), thereby detecting one or more acylated protein(s) produced by a cell. In certain embodiments, detection of one or more acylated protein(s) comprises quantitative detection of said one or more acylated protein(s). In certain embodiments, one or more alkynylated protein(s) isolated in step (b) above are isolated by immuno-precipitation or affinity chromatography. In certain embodiments, a cell is provided with one or more alkynyl-chemical reporter(s) by incubating the cell with said one or more alkynyl-chemical reporters. In certain embodiments, a cell is provided with one or more alkynyl-chemical reporter(s) in step (a) above by in vivo administration of one or more of said alkynyl-chemical reporter(s) to an organism. In certain embodiments, the organism is a non-human organism such as a insect, fish, or mammal. In certain embodiments, the methods further comprise the step of separating one or more azido detection tag labeled protein(s) from step (c) above before detection in step (d) above. In certain embodiments, the one or more azido detection tag(s) used to label acylated-proteins comprises a fluorescent label or an epitope tag. In certain embodiments, the epitope tag is a biotin group, an immunoreactive peptide, or a polyhistidine group. In certain embodiments of the methods for detecting one or more acylated protein(s) produced by a cell where an alkynylated protein is isolated before labeling with an azido tag, the acylated protein is an acetylated protein. In certain embodiments of the methods for detecting one or more acylated protein(s) produced by a cell where an alkynylated protein is isolated before labeling with an azido tag, the acylated protein is an fatty-acylated protein.

Certain embodiments of the present invention provide for kits comprising: (a) one or more alkynyl-chemical reporter(s); (b) one or more azido detection tag(s) for labeling said alkynylated protein lysate; and (c) containers for said chemical reporter(s) and detection tag(s). In certain embodiments, the one or more alkynyl-chemical reporters contained in a kit comprise(s) at least one of hexa-5-ynoic acid, pent-4-ynoic acid, buta-3-ynoic acid, tetradec-13-ynoic acid, hexadec-15-ynoic acid, octadec-17-ynoic acid or any combination thereof. In certain embodiments, kits further comprise instructions for detecting one or more acylated proteins produced by a cultured cell. In certain embodiments, kits further comprise instructions for detecting one or more acylated proteins produced by a cell in an organism. In certain embodiments, the instructions are for detecting one or more acylated proteins produced by a cell in a non-human organism. In certain embodiments, kits further include reagents for performing Cu^{I}-catalyzed Huisgen [3+2] cycloaddition. In certain embodiments, one or more azido detection tag(s) used to label acylated-proteins comprises a fluorescent label or an epitope tag. In certain embodiments, the epitope tag is a biotin group, an immunoreactive peptide, or a polyhistidine group. In certain embodiments, the chemical reporter(s) is/are: i) of the formula R—(CH₂)ₙ—CO—S—CoA, wherein R is an alkynyl or an azido group and n=4-24; or, ii) of the formula R—(CH₂)ₙ—COOH, wherein R is an alkynyl group or an azido group, and wherein n=4-24, or iii) a corresponding cationic salt of the chemical reporter of the formula R—(CH₂)ₙ—COOH, wherein R is an alkynyl group or an azido group, and wherein n=4-24. Such cationic salts include, but are not limited to, Na⁺, K⁺ or Li⁺ salts. In certain embodiments, the kits provide for detection of an acylated protein that is an acetylated protein or that is a fatty-acylated protein. In certain embodiments where the one or more acylated proteins detected is an acetylated protein, one or more chemical reporter(s) in the kit can comprise at least one or more compounds of the formula R—(CH₂)ₙ—CO—S—CoA, wherein R is an alkynyl (C≡C) group and n=2 or 3 or wherein R is an azido (N₃) group and n=2 or 3. For cellular studies, chemical reporters can be provided in the kit as their corresponding cationic salts to facilitate metabolic incorporation. In certain embodiments where the one or more acylated proteins detected is an acetylated protein, one or more alkynyl-chemical reporter(s) can comprise 4-pentynyl-CoA. In certain embodiments where the one or more acylated proteins detected is an acetylated protein, one or more chemical reporter(s) in the kit can comprise at least one or more compounds of the formula R—(CH₂)ₙ—COOH, wherein R is an alkynyl (C≡C) group and n=2 or 3 or wherein R is an azido (N₃) group and n=2 or 3. For cellular studies, such chemical reporters can be provided in the kit as their corresponding cationic salts to facilitate metabolic incorporation. Such cationic salts include, but are not limited to, Na⁺, K⁺ or Li⁺ salts. Corresponding cationic salts of the compounds of the formula R—(CH₂)ₙ—COOH, wherein R is an alkynyl (C≡C) group or wherein R is an azido (N₃) group, include, but are not limited to, chemical reporters as shown in FIG. 51.

Certain embodiments of the present invention provide for methods for detecting one or more acylated proteins produced by a cell, the method comprising the steps of: (a) obtaining a protein lysate comprising one or more protein(s) acylated by one or more chemical reporter(s) from a cell provided with one or more chemical reporter(s); (b) labeling acylated protein(s) in said protein lysate of step (a) with one or more detection tag(s) attached to an affinity purification tag by a cleavable linkage; (c) capturing one or more acylated protein(s) linked to said affinity purification tag in step (b) on a solid support comprising an agent that binds said affinity purification tag; (d) releasing from said solid support of step (c) one or more acylated protein(s) labelled with said detection tag by cleaving said cleavable linkage of said detection tag to said affinity purification tag; and (e) detecting one or more said acylated protein(s) released in step (d), thereby detecting one or more acylated protein(s) produced by a cell. In certain embodiments, the detection of one or more acylated protein(s) comprises quantitative detection of said one or more acylated protein(s). In certain embodiments, detection tags further comprises a detectable label that remains linked to the detection tag attached to an acylated protein, following cleavage of the cleavable linkage to an affinity purification tag in step (d) above. In certain embodiments, a detectable label is an isotope or a fluorophore. In certain embodiments, the detectable label is a halogen selected from the group consisting of chlorine, bromine, fluorine, and iodine. In certain embodiments, the chemical reporter(s) are alkynyl-acid chemical reporter(s) and the detection tag is an azido detection tag. In certain embodiments, alkynyl-chemical reporter(s) comprise at least one of hexa-5-ynoic acid, pent-4-ynoic acid, buta-3-ynoic acid, tetradec-13-ynoic acid, hexadec-15-ynoic acid, octadec-17-ynoic acid, or any combination thereof. In certain embodiments, the chemical reporter(s) are azido-chemical reporter(s) and the detection tag is an alkynyl detection tag. In certain embodiments, azido-chemical reporter(s) comprise at least one of 12-azido-dodecanoic acid, 15-azido-pentadecanoic acid, or the combination of both. In certain embodiments, the chemical reporter(s) is/are: i) of the formula R—(CH₂)ₙ—CO—S—CoA, wherein R is an alkynyl or an azido group and n=4-24; or, ii) of the formula R—(CH₂)ₙ—COOH, wherein R is an alkynyl group or an azido group, and wherein n=4-24, or iii) a corresponding cationic salt of the chemical reporter of the formula R—(CH₂)ₙ—COOH, wherein R is an alkynyl group or an azido group, and wherein n=4-24. Such cationic salts include, but are not limited to, Na⁺, K⁺ or Li⁺ salts. In certain embodiments, an affinity purification tag comprises a biotin group, an immunoreactive peptide, or a polyhistidine group. In certain embodiments, a cleavable linkage comprises an acid cleavable linker, a base cleavable linker, or a diazo linker. In certain embodiments, an affinity purification tag comprises a biotin group and a cleavable linkage is a diazo linkage. In certain embodiments, wherein capture of acylated proteins is effected in step (c) above with a streptavidin linked solid support, cleavage is effected in step (d) above with sodium dithionite. In any of the embodiments where an alkynyl chemical reporter is used, a detection tag attached to an affinity purification tag by a cleavable linkage can comprise (II) (E)-4-((5-(2-azidoethyl)-2-hydroxyphenyl)diazenyl)-N-(15-oxo-19-(2-oxohexahydro-1H thieno[3,4-d]imidazol-4-yl)-4,7,10-trioxa-14-azanonadecyl)benzamide or (III) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-2-azido-ethyl)-phenylazo]-benzamide. In any of the embodiments where an azido chemical reporter is used, a detection tag attached to an affinity purification tag by a cleavable linkage can comprise (I) (E)-4-((5-(2-hept-6-ynamidoethyl)-2-hydroxyphenyl)diazenyl)-N-(15-oxo-19-(2-oxohexa hydro-1H-thieno[3,4-a]imidazol-4-yl)-4,7,10-trioxa-14-azanonadecyl)benzamide or (IV) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-buta-4-ynl)-phenylazo]-benzamide. In certain embodiments, detection of one or more acylated protein(s) in step (e) above is by staining, immunolabelling, fluorescence, radiometry, or mass spectrometry. In certain embodiments, detection comprises identification of one or more of said acylated protein(s) by mass spectroscopy. In certain embodiments, any of these aforementioned methods can further comprise the step of washing said solid support of step (c) above comprising said captured acylated protein(s) prior to cleavage in step (d) above. In certain embodiments of the methods, the acylated protein is an acetylated protein or a fatty-acylated protein. In certain embodiments where the one or more acylated proteins detected is an acetylated protein, one or more chemical reporter(s) can comprise at least one or more compounds of the formula R—(CH₂)ₙ—CO—S—CoA, wherein R is an alkynyl (C≡C) group and n=2 or 3 or wherein R is an azido (N₃) group and n=2 or 3. For cellular studies, chemical reporters may also be administered to cells as their corresponding cationic salts to facilitate metabolic incorporation. In certain embodiments where the one or more acylated proteins detected is an acetylated protein, one or more alkynyl-chemical reporter(s) can comprise 4-pentynyl-CoA. In certain embodiments where the one or more acylated proteins detected is an acetylated protein, one or more chemical reporter(s) can comprise at least one or more compounds of the formula R—(CH₂)ₙ—COOH, wherein R is an alkynyl (C≡C) group and n=2 or 3 or wherein R is an azido (N₃) group and n=2 or 3. For cellular studies, such chemical reporters may also be administered to cells as their corresponding cationic salts to facilitate metabolic incorporation. Such cationic salts include, but are not limited to, Na⁺, K⁺ or Li⁺ salts. Corresponding cationic salts of the compounds of the formula R—(CH₂)ₙ—COOH, wherein R is an alkynyl (C≡C) group or wherein R is an azido (N₃) group, include, but are not limited to, chemical reporters as shown in FIG. 51.

Certain embodiments of the present invention provide for kits comprising: (a) one or more chemical reporter(s); (b) one or more detection tag(s) attached to an affinity purification tag by a cleavable linkage; and (c) containers for said chemical reporter(s) and said detection tag(s). In certain embodiments, the one or more chemical reporter(s) contained in a kit is an azido-chemical reporter or an alkynyl-chemical reporter. In certain embodiments, the chemical reporter(s) is/are: i) of the formula R—(CH₂)ₙ—CO—S—CoA, wherein R is an alkynyl or an azido group and n=4-24; or, ii) of the formula R—(CH₂)ₙ—COOH, wherein R is an alkynyl group or an azido group, and wherein n=4-24, or iii) a corresponding cationic salt of the chemical reporter of the formula R—(CH₂)ₙ—COOH, wherein R is an alkynyl group or an azido group, and wherein n=4-24. Such cationic salts include, but are not limited to, Na⁺, K⁺ or Li⁺ salts. In certain embodiments, the one or more chemical reporter(s) contained in a kit is an azido-chemical reporter that comprises at least one of azido-butanoic acid, 5-azido-pentanoic acid, 6-azido-hexanoic acid, 12-azido-dodecanoic acid, 15-azido-pentadecanoic acid, or any combination thereof. In certain embodiments, the one or more chemical reporter(s) is an alkynyl-chemical reporter that comprise at least one of hexa-5-ynoic acid, pent-4-ynoic acid, buta-3-ynoic acid, tetradec-13-ynoic acid, hexadec-15-ynoic acid, octadec-17-ynoic acid, or any combination thereof. In certain embodiments, kits further comprise instructions for detecting one or more acylated proteins produced by a cultured cell. In certain embodiments, kits further comprise instructions for detecting one or more acylated proteins produced by a cell in an organism. In certain embodiments, the organism is a non-human organism. In certain embodiments, kits further comprise reagents for performing Cu^{I}-catalyzed Huisgen [3+2] cycloaddition or strain-promoted Huisgen [3+2] cycloaddition. In certain embodiments, one or more detection tag(s) further comprise(s) a detectable label that remains linked to said detection tag attached to said acylated protein, following cleavage of said cleavable linkage to said affinity purification tag in step (d) above. In certain embodiments, one or more detection tag(s) comprising a detectable label is an azido detection tag or an alkenyl detection tag. In certain embodiments, kits further comprise a solid support comprising an agent that binds said affinity purification tag. In certain embodiments, kits further comprise at least one of a solid support, an agent that binds said affinity purification tag, or a combination of both. In certain embodiments, a cleavable linkage comprises an acid cleavable linker, a base cleavable linker, or a diazo linker. In certain embodiments, an affinity purification tag comprises a biotin group and a cleavable linkage is a diazo linkage. In certain embodiments, a solid support comprises a streptavidin linked solid support. In any of the embodiments where an alkynyl chemical reporter is used, a detection tag attached to an affinity purification tag by a cleavable linkage can comprise (II) (E)-4-((5-(2-azidoethyl)-2-hydroxyphenyl)diazenyl)-N-(15-oxo-19-(2-oxohexahydro-1H thieno[3,4-d]imidazol-4-yl)-4,7,10-trioxa-14-azanonadecyl)benzamide or (III) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-2-azido-ethyl)-phenylazo]-benzamide. In any of the embodiments where an azido chemical reporter is used, a detection tag attached to an affinity purification tag by a cleavable linkage can comprise (I) (E)-4-((5-(2-hept-6-ynamidoethyl)-2-hydroxyphenyl)diazenyl)-N-(15-oxo-19-(2-oxohexa hydro-1H-thieno[3,4-a]imidazol-4-yl)-4,7,10-trioxa-14-azanonadecyl)benzamide or (IV) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-buta-4-ynl)-phenylazo]-benzamide. In certain embodiments where the one or more acylated proteins detected is an acetylated protein, one or more chemical reporter(s) un the kit can comprise at least one or more compounds of the formula R—(CH₂)ₙ—CO—S—CoA, wherein R is an alkynyl (C≡C) group and n=2 or 3 or wherein R is an azido (N₃) group and n=2 or 3. For cellular studies, chemical reporters are provided in the kit as their corresponding cationic (Na⁺, K⁺ or Li⁺) salts to facilitate metabolic incorporation. In certain embodiments where the one or more acylated proteins detected is an acetylated protein, one or more alkynyl-chemical reporter(s) can comprise 4-pentynyl-CoA. In certain embodiments where the one or more acylated proteins detected is an acetylated protein, one or more chemical reporter(s) can comprise at least one or more compounds of the formula R—(CH₂)ₙ—COOH, wherein R is an alkynyl (C-E) group and n=2 or 3 or wherein R is an azido (N₃) group and n=2 or 3. Such cationic salts include, but are not limited to, Na⁺, K⁺ or Li⁺ salts. Corresponding cationic salts of the compounds of the formula R—(CH₂)ₙ—COOH, wherein R is an alkynyl (C≡C) group or wherein R is an azido (N₃) group, include, but are not limited to, chemical reporters as shown in FIG. 51.

Chemical compounds that comprise one or more detection tag(s) attached to an affinity purification tag by a cleavable linkage are also provided herein. In certain embodiments, the chemical compounds comprise the compound of the formula (A):

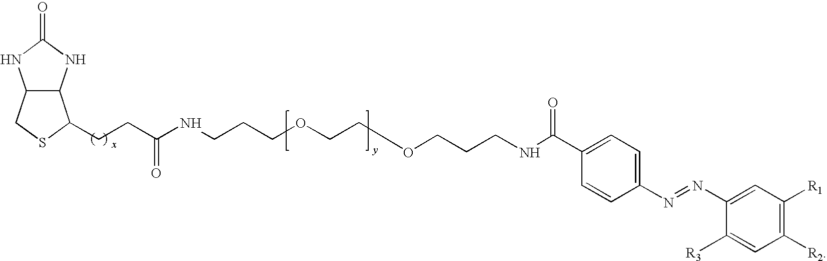

wherein: x is an integer 1 to 5 inclusive; y is an integer 1 to 10 inclusive; R₁ is hydrogen, —(CH₂)z—N═N═N, or —(CH₂)z—NH—CO—(CH₂)z-C≡CH, wherein z is an integer between 2 to 5 inclusive; R₂ is —H, —O—(CH₂)z-N═N═N, or —O—(CH₂)z-C≡CH, wherein z is an integer between 2 to 5 inclusive; and R₃ is H or OH. In certain embodiments, R₁ is —H, —(CH₂)2-N═N═N, or —(CH₂)₂—NH—CO—(CH₂)₄—C≡CH, R₂ is —H, —O—(CH₂)₂—N═N═N, or —O—(CH₂)₂—C≡CH, and R₃ is H or OH. In certain embodiments, the compound is selected from: (I) (E)-4-((5-(2-hept-6-ynamidoethyl)-2-hydroxyphenyl)diazenyl)-N-(15-oxo-19-(2-oxohexa hydro-1H-thieno[3,4-a]imidazol-4-yl)-4,7,10-trioxa-14-azanonadecyl)benzamide; (II) (E)-4-((5-(2-azidoethyl)-2-hydroxyphenyl)diazenyl)-N-(15-oxo-19-(2-oxohexahydro-1H thieno[3,4-d]imidazol-4-yl)-4,7,10-trioxa-14-azanonadecyl)benzamide; (III) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-2-azido-ethyl)-phenylazo]-benzamide; or (IV) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-buta-4-ynl)-phenylazo]-benzamide. In certain embodiments, the compound is (III) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-2-azido-ethyl)-phenylazo]-benzamide. In certain embodiments, the compound is (IV) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-buta-4-ynl)-phenylazo]-benzamide. In certain embodiments, the compound is (I) (E)-4-(5-(2-hept-6-ynamidoethyl)-2-hydroxyphenyl)diazenyl)-N-(15-oxo-19-(2-oxohexa hydro-1H-thieno[3,4-a]imidazol-4-yl)-4,7,10-trioxa-14-azanonadecyl)benzamide. In certain embodiments, the compound is (II) (E)-4-((5-(2-azidoethyl)-2-hydroxyphenyl)diazenyl)-N-(15-oxo-19-(2-oxohexahydro-1H thieno[3,4-d]imidazol-4-yl)-4,7,10-trioxa-14-azanonadecyl)benzamide.

### BRIEF DESCRIPTION OF THE SEVERAL VIEWS OF THE DRAWINGS

FIG. 1 illustrates N-Myristoylation (attachment of myristic acid (14:0) to proteins) and S-Palmitoylation (attachment of palmitic acid (16:0) to proteins), two types of fatty-acylation of proteins that occurs in eukaryotes.

FIG. 2 is a general schematic of the metabolic labeling of cells with chemical reporters and bioorthogonal labeling of proteins.

FIG. 3A shows a general structure of azido-fatty acid chemical reporters.

FIG. 3B is a schematic of the Staudinger ligation reaction.

FIG. 4A is a schematic of the Cu^{I}-catalyzed Huisgen [3+2] cycloaddition or “click chemistry” reaction that enables selective covalent attachment of detection tags to azide/alkyne-modified substrates.

FIG. 4B shows a general structure of alkynyl-fatty acid chemical reporters and non-limiting examples wherein the number of carbon atoms in the fatty acid chain varies.

FIG. 5 is a general synthetic scheme for the synthesis of alkynyl-fatty acid reporters and non-limiting examples of various length fatty acids. Reagents: i) Li, t-BuOK, 1,3-diaminopropane, 80%. ii) CrO₃, H₂SO₄, 75%.

FIG. 6 illustrates the chemical structures of the exemplary detection tags azido-biotin, azido-diazo-biotin, and azido-rhodamine.

FIG. 7A is a general synthetic scheme for the synthesis of an azido-biotin detection tag. Reagents: iv) 5-azidopentanoic acid, isobutylchloroformate, N-methylmorpholine, 45%.

FIG. 7B is a general synthetic scheme for the synthesis of an azido-rhodamine detection tag. Reagents: yl) 6-azidohexanoic acid, CDI, 70%.

FIG. 8 shows a streptavidin blot comparing the use of azido-fatty acid chemical reporters (az-12, az-15) with either the Staudinger ligation (phos-biotin) or click chemistry (alk-biotin).

FIG. 9 shows a comparison of azide/alkyne orientation by streptavidin blotting at long, medium, and short exposure times. Comparable levels of protein loading was demonstrated by Ponceau staining of the blot.

FIG. 10A shows a comparison of azide/alkyne orientation with click chemistry by in-gel fluorescence scanning. Experiments were performed with cell lysates from Jurkat T cells metabolically labeled with DMSO (−), ω-azido- or ω-alkynyl-fatty acids (20 μM az-12 and alk-12 or 200 μM az-15, alk-14, and alk-16).

FIG. 10B illustrates a quantitative comparative analysis of in-gel fluorescence with ImageJ software. Total lane pixel intensity values over respective background are reported. Relative fluorescence values were also normalized for protein loading based on signal intensity from Coomassie-stained gel (FIG. 10A). Experiments were performed with cell lysates from Jurkat T cells metabolically labeled with DMSO (−), ω-azido- or ω-alkynyl-fatty acids (20 μM az-12 and alk-12 or 200 μM az-15, alk-14, and alk-16).

FIG. 11 illustrates a schematic representation of lipidation sites for LAT (S-palmitoyl-Cys26, S-palmitoyl-Cys29), Lck (N-myristoyl-Gly2, S-palmitoyl-Cys3, S-palmitoyl-Cys5) and H-Ras (S-palmitoy-Cys181, S-palmitoyl-Cys184, S-prenyl-Cys186), exemplary examples of different classes of fatty-acylated proteins.

FIG. 12A shows a comparative analysis of click chemistry orientation with Lck, LAT and Ras by streptavidin blotting. Experiments were performed with cell lysates from Jurkat T cells metabolically labeled with DMSO (−), ω-azido- or ω-alkynyl-fatty acids (20 μM az-12 and alk-12 or 200 μM az-15, alk-14, and alk-16). Comparable protein load was demonstrated by blotting for the corresponding proteins.

FIG. 12B shows a comparative analysis of click chemistry orientation with Lck, LAT, and Ras by fluorescence scanning. Experiments were performed with cell lysates from Jurkat T cells metabolically labeled with DMSO (−), ω-azido- or ω-alkynyl-fatty acids (20 μM az-12 and alk-12 or 200 μM az-15, alk-14, and alk-16). Comparable protein load was demonstrated by blotting for the corresponding proteins.

FIG. 13 illustrates examples of detections tags linked to an affinity tag through a cleavable diazo linker.

FIG. 14A is a general schematic of an affinity purification method for the isolation of acylated proteins by labeling of alkynylated proteins with a detection tag linked to an affinity purification tag (shown here as biotin) through a cleavable linker (shown here as a diazo linker) that allows for the release of a protein from a solid substrate (shown here as streptavidin beads) through cleavage (shown here by sodium thionite) of the cleavable linker.

FIG. 14B shows the results of affinity enrichment of fatty-acylated proteins, as described in FIG. 14A, from mammalian cells using alk-12, alk-14, and alk-16 chemical reporters.

FIG. 15 shows the structure of exemplary alk- and az-detection tags.

FIG. 16A and FIG. 16B demonstrate the time and dose dependence of alkynyl-fatty acid labeling: A) labeling of Jurkat cells with alk-12 (20 μM) and alk-16 (200 μM) over time (hours); B) labeling of Jurkat cells with alk-12 and alk-16 at different concentrations over 4 hours. Upper panel, in-gel fluorescence. Lower panel, comparable levels of protein loading was demonstrated by Coomassie staining of the gel.

FIG. 17A and FIG. 17B show the profile of fatty-acylated proteomes in different cell lines and mouse tissues: A) mammalian cell lines (HeLa, 3T3, DC2.4, or Jurkat T cells) or B) splenocytes metabolically labeled with DMSO (−) or ω-alkynyl-fatty acids (20 μM alk-12 or 200 μM alk-16).

FIG. 18A is a general schematic of a method for the incorporation of alkynyl-chemical reporters in mice.

FIG. 18B shows the repertoire of fatty-acylated proteins from splenocytes, liver, and kidney detected one hour after intraperitoneal administration of alk-12 (50-250 mg/kg) or alk-16 (50-250 mg/kg) to mice. Comparable levels of protein loading were demonstrated by Coomassie staining of the gels (not shown).

FIG. 19. shows the stability of biotinylated-diazobenzene detection tags (alk-diazo-biotin, az-diazo-biotin) under click chemistry conditions and selective cleavage with sodium dithionite.

FIG. 20A, FIG. 20B, and FIG. 20C show the global analysis of fatty-acylated proteins in mammalian cells: A) in-gel fluorescence analysis of fatty-acylated proteins; B) selective retrieval of fatty-acylated proteins (* indicates streptavidin eluted from beads); C) validation of selectively retrieved fatty-acylated proteins using specific antibodies to Lck and Trf.

FIG. 21A shows the detection of acetylated proteins using alkynyl-chemical reporter labeling coupled with azido-detection tags and in-gel fluorescence detection. In particular, alkynyl-chemical reporters are shown to label histones.

FIG. 21B shows that the C6 alkynyl chemical reporter (i.e., alk-4) is more efficient than the shorter C5 (alk-3) and C4 (alk-2) alkynyl-chemical reporters for labeling acetylated proteins.

FIG. 22 shows exemplary compounds comprising an affinity purification tag, a cleavable linkage, and a detection tag. In these compounds, the affinity purification is a biotin group, the cleavable linkage is a diazo bond, and the detection tags comprise phenyl groups substituted with groups comprising azido or alkenyl groups as shown. The compounds shown are: (I) (E)-4-((5-(2-hept-6-ynamidoethyl)-2-hydroxyphenyl)diazenyl)-N-(15-oxo-19-(2-oxohexa hydro-1H-thieno[3,4-a]imidazol-4-yl)-4,7,10-trioxa-14-azanonadecyl)benzamide; (II) (E)-4-((5-(2-azidoethyl)-2-hydroxyphenyl)diazenyl)-N-(15-oxo-19-(2-oxohexahydro-1H thieno[3,4-d]imidazol-4-yl)-4,7,10-trioxa-14-azanonadecyl)benzamide; (III) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-2-azido-ethyl)-phenylazo]-benzamide; and (IV) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-buta-4-ynl)-phenylazo]-benzamide.

FIG. 23 shows a comparison of acylated-protein labeling by alkynyl-chemical reporters (Alk-analog) of various lengths. Use of the alk-4 (C6) chemical reporter was the most efficient of the shorter alkynyl-chemical reporters (i.e., alk-2 (C4), alk-3 (C5), or alk-4 (C6)) for labeling acylated proteins.

FIG. 24A shows that alk-4 (C6) chemical reporter labeling is dose dependant.

FIG. 24B shows that alk-4 (C6) chemical reporter labeling is time dependant. Incubation with 5 mM alk-4 for four hours provided the most efficient labeling.

FIG. 25A shows that labeling of acylated proteins with alkynyl-chemical reporters does not require de novo protein synthesis. Acylated proteins were still detected in the presence of cyclohexamide, an inhibitor of protein synthesis.

FIG. 25B shows that labeling of acylated proteins with alkynyl-chemical reporters does not require de novo fatty acid synthesis. Acylated proteins were still detected in the presence of cerulenin, an inhibitor of fatty acid synthesis.

FIG. 26A shows that labeling of acetylated proteins with the alkynyl-chemical reporter alk-4 is inhibited by the addition of butyric acid.

FIG. 26B shows that labeling of acetylated proteins with the alkynyl-chemical reporter alk-4 is not sensitive to addition of the histone deacetylase inhibitor suberoylanilide hydroxamic acid (SAHA).

FIG. 27 shows that the alkynyl-chemical reporter alk-4 (C6) labels distinct acetylated proteins in diverse cell types.

FIG. 28A is a general schematic of a method for the incorporation of alkynyl-chemical reporters in bacteria.

FIG. 28B shows that alkynyl-chemical reporters of various lengths can label distinct acylated proteins in bacterial cells.

FIG. 29A shows a general synthetic scheme for the synthesis of (I) (E)-4-((5-(2-hept-6-ynamidoethyl)-2-hydroxyphenyl)diazenyl)-N-(15-oxo-19-(2-oxohexa hydro-1H-thieno[3,4-a]imidazol-4-yl)-4,7,10-trioxa-14-azanonadecyl)benzamide (3) (alk-diazo-biotin).

FIG. 29B shows a general synthetic scheme for the synthesis of (II) (E)-4-((5-(2-azidoethyl)-2-hydroxyphenyl)diazenyl)-N-(15-oxo-19-(2-oxohexahydro-1H thieno[3,4-d]imidazol-4-yl)-4,7,10-trioxa-14-azanonadecyl)benzamide (6) (az-diazo-biotin).

FIG. 30A shows competition of alk-12, alk-14, and alk-16 (10 μM) labeling in Jurkat cells with different concentrations of myristic acid. Upper panels, in-gel fluorescence. Lower panels, comparable levels of protein loading was demonstrated by Coomassie staining of the gel.

FIG. 30B shows competition of alk-12, alk-14, and alk-16 (10 μM) labeling in Jurkat cells with different concentrations of palmitic acid. Upper panels, in-gel fluorescence. Lower panels, comparable levels of protein loading was demonstrated by Coomassie staining of the gel.

FIG. 31A shows inhibition of alk-12, alk-14, and alk-16 (20 μM) labeling in Jurkat cells with cycloheximide (CHX) (10 μM). Upper panels, in-gel fluorescence. Lower panels, comparable levels of protein loading was demonstrated by Coomassie staining of the gel.

FIG. 31B shows inhibition of alk-12, alk-14, and alk-16 (20 μM) labeling in Jurkat cells with 2-hydroxymyristic acid (HMA) (1 mM). Upper panels, in-gel fluorescence. Lower panels, comparable levels of protein loading was demonstrated by Coomassie staining of the gel.

FIG. 31C shows inhibition of alk-12, alk-14, and alk-16 (20 μM) labeling in Jurkat cells with 2-bromopalmitate (BPA) (50 μM). Upper panels, in-gel fluorescence. Lower panels, comparable levels of protein loading was demonstrated by Coomassie staining of the gel.

FIG. 32A shows hydroxylamine sensitivity of proteins in Jurkat cell lysates labeled with alkynyl-fatty acids. Experiments were performed with cell lysates from Jurkat T cells metabolically labeled with DMSO (−), azido- or alkynyl-fatty acids (20 μM az-12, az-15, alk-12, alk-14, or alk-16).

FIG. 32B shows hydroxylamine sensitivity of alkynyl-fatty acid Lck and LAT labeling. Experiments were performed with cell lysates from Jurkat T cells metabolically labeled with DMSO (−), azido- or alkynyl-fatty acids (20 μM az-12, az-15, alk-12, alk-14, or alk-16).

FIG. 33 shows comparative analysis of acylation state of wild type, G2A mutant and C3,6S mutant Fyn. HeLa cells were metabolically labeled with DMSO (−) or alkynyl-fatty acids (20 μM alk-12 or alk-16). Comparable protein load was demonstrated by blotting for the corresponding proteins.

FIG. 34 shows immunoprecipitation of Ras and Lck form Jurkat cell lysates labeled with alk-12 or alk-16 (20 μM) (left panels) and immunoprecipitation of LAT and p53 from Jurkat cell lysates labeled with alk-12 or alk-16 (20 μM) (right panels). Upper panels, in-gel fluorescence. Lower panels, comparable levels of protein loading was demonstrated by blotting for the corresponding protein.

FIG. 35. Synthesis of clickable CyFur dyes.

FIG. 36. Spectral properties of clickable CyFur dyes. (A) Emission spectra of clickable CyFur dyes. Emission spectra were acquired at excitation of 470 nm and 580 nm for az-CyFur-1 (solid line) and alk-CyFur (dash line), respectively. (B) Emission spectra of alk-CyFur (x), az-CyFur-1 (Δ) and fluorescent adduct 3 (□), excitation at 410 nm.

FIG. 37. In-gel fluorescence imaging of azido-fatty acid (az-FA)-modified proteins from metabolically labeled Jurkat T cells after bioorthogonal ligation with clickable CyFur dyes.

FIG. 38. Fluorescence microscopy of azido (az-12)- and alkynyl (alk-12)-fatty acid labeled HeLa cells after bioorthogonal ligation with clickable dyes. (A) Imaging with 488 nm excitation and 560 nm emission long-pass filter. Top panel: az-CyFur-1-labeled proteins. Bottom panel: alk-CyFur-labeled proteins. (B) Imaging with 633 nm excitation and 646-753 nm emission filter. Top panel: az-CyFur-1-labeled proteins. Bottom panel: alk-CyFur-labeled proteins. (C) Imaging of az-Rho-labeled proteins. 543 nm excitation and 560-615 nm emission filter. DAPI imaging in all panels was performed by 405 nm excitation and 420-480 nm emission filter.

FIG. 39. (A) S-Palmitoylation is a reversible and dynamic protein modification. (B) Following metabolic labeling with two chemical reporters, immunopurification and sequential click chemistry reactions with orthogonal detection tags allow simultaneous visualization of the fatty-acylation and protein synthesis. (C) Fatty acid chemical reporters for N-myristoylation and S-palmitoylation. (D) Amino acid chemical reporter for protein synthesis. (E) Clickable fluorescent detection tags.

FIG. 40. (A) In-gel fluorescence scanning allows tandem detection of az-12- and alk on Lck using alk-Cyfur and az-Rho respectively. Anti-Lck blot reflects total protein levels. (*) refers to non-specific bands. (B) Pulse-chase analysis of Lck. (C) Data from multiple pulse-chase experiments (n=10). Data points from the same chase times, after normalizing alk-16 to az-12 signals, are compiled and displayed as average values±s.e.m.

FIG. 41. (A) Anti-phosphotyrosine, anti-Lck blots and (B) alk-16 labeling of unstimulated and PV-treated Jurkat T cells. (C) Anti-phosphotyrosine, anti-Lck blots and alk-16 labeling of immunopurified Lck from unstimulated and PV-treated Jurkat T cells. Mobility shift of immunopurified Lck with PV stimulation. (D) Pulse-chase analysis of Lck in the presence of 0.1 mM PV. (E) PV activation data from multiple pulse-chase experiments (n=7). Data points from the same chase times, after normalizing alk-16 to az-12 signals, are compiled and displayed as average values±s.e.m. (inset).

FIG. 42. (A) Pulse-chase analysis of Lck in the presence of chemical inhibitors. (B) Data from multiple pulse-chase experiments (n=2). Data points from the same chase times, after normalizing alk-16 to az-12 signals, are compiled and displayed as average values±s.e.m.

FIG. 43. (A) Pulse chase analysis of H-Ras^{G12V} with labeling of AHA and alk-16. (B) Data from multiple pulse-chase experiments (n=5). Data points from the same chase limes, after normalizing alk-16 to AHA signals, are compiled and displayed as average values±s.e.m.

FIG. 44. (A) In-gel fluorescence scanning of az-12 and alk-16 modified Lck after hydroxylamine treatment. (B) Pulse-chase experiment in the presence of excess myristate. In-gel fluorescence scanning in the alk-CyFur channel shows no significant turnover of az-12 on Lck. (*) refers to non-specific bands.

FIG. 45. (A) In-gel fluorescence scanning allows tandem detection of az-12 and alk-16 on Fyn using alk-Cyfur and az-Rho respectively. (B) In-gel fluorescence scanning of Fyn pre- and post-hydroxylamine treatment. (C) Pulse-chase analysis of Fyn. (D) Analysis of palmitate t_{1/2} on Fyn relative to that of Lck. (*) refers to non-specific bands.

FIG. 46. In-gel fluorescence, coomassie blue (CB) and western blot analyses of cellular lysates and immunopurification of dual labeling of H-Ras^{G12V} with AHA and alk-16. (*) refers to non-specific bands.

FIG. 47. (A) Two-step labeling strategy for detection of protein acetylation using bioorthogonal chemical reporters and CuAAC method. (B) In-gel fluorescence detection of p300-catalyzed acylation of histone H3. Comparable levels of protein loading are demonstrated by coomassie blue (CB) staining.

FIG. 48. (A) Selective metabolic labeling of core histones. (B) Selective metabolic labeling of histone H3. (C) In-gel fluorescent detection of acetylated proteins in total Jurkat T cell lysates. (D) Functional distribution of acetylated proteins enriched from 4-pentynoate-metabolically labeled Jurkat T cells.

FIG. 49. Chemical synthesis and characterization of alkynyl-acetyl-CoA analogs. (a) Synthetic scheme of alkynyl-acetyl-CoA analogs. (b) MALDI-TOF mass spectra of synthetic alkynyl-acetyl-CoA analogs. Measured in negative mode.

FIG. 50. Characterization of p300-catalyzed in vitro acylation on histone H3 peptide and histone H3. (a) Analysis of the crude in vitro acylation products of histone H3 peptide by MALDI-TOF mass spectrometry. 25 μmol of H3 peptide [aa 2-21 (L21Y): ARTKQTARKSTGGKAPRKQY] (SEQ ID NO:24) and 20 μM of acetyl-CoA or alkynyl-acetyl-CoA analogs were subjected to in vitro acetylation condition. The peptide products were extracted with ZipTip (Millipore) for MS analysis. (b) Crude in vitro acylation products of histone H3 (˜1.7 μg) resolved on 15% SDS-PAGE. The gel slices containing histone H3 were later excised from the gels and subjected to in-gel trypsin digestion followed by MS analysis to determine the in vitro modification sites. (c) Selected MS/MS spectra of peptides derived from in-gel trypsin digestion of in vitro acylated histone H3 products. (d) In-gel fluorescent detection of in vitro time-dependent acylation of histone H3 and p300. Histone H3 (˜1.7 μg) were co-incubated with p300 (50 or 100 ng) and 4-pentynyl-CoA (50 μM or 160 μM) at 30° C. The reaction was stopped by adding ⅔ reaction volume of 4% SDS buffer (4% SDS, 150 mM NaCl, 50 mM triethanolamine, pH 7.4), followed by reacting with az-Rho via CuAAC and in-gel fluorescence scanning1. Comparable levels of protein loading are demonstrated by coomassie blue staining.

FIG. 51. 1H NMR spectra of sodium 3-butynoate (4), sodium 4-pentynoate (5) and sodium 5-hexynoate (6).

FIG. 52. Characterization of chemical reporters, 3-butynoate, 4-pentynoate and 5-hexynoate, in metabolic labeling of mammalian cells. (a) Dose-dependent labeling of Jurkat T cells. Jurkat T cells were labeled with different concentrations of 3-butynoate, 4-pentynoate or 5-hexynoate, and harvested after 6 hr incubation. The lysates were reacted with az-Rho and analyzed by in-gel fluorescence scanning. (b) Time-dependent labeling of Jurkat T cells. 32 mL (4 mL×8) of Jurkat T cells were labeled with 5 mM of 3-butynoate, 4-pentynoate or 5-hexynoate for different time length. At each time point, 4 mL of labeling cells were collected, spun down and frozen in liquid nitrogen. Negative control (dd H₂O) was carried out in a separate flask. The lysates were reacted with az-Rho and analyzed by in-gel fluorescence scanning.

FIG. 53. Further characterization of chemical reporters, 3-butynoate (4), 4-pentynoate (5) and 5-hexynoate (6), in metabolic labeling of mammalian cells. (a) Inhibition of cellular protein biosynthesis by cycloheximide (CHX) in the presence of alkynyl-acetate analogs (10 mM), alkynyl-fatty-acid analogs (20 μM)1 and AHA (azidohomoalanine, 4 mM)2. In this experiment, Jurkat T cells were either labeled with the chemical reporter alone for 1 hr, or pre-treated with 100 μM CHX for 0.5 hr and then labeled with the chemical reporter for additional 1 hr. Cells labeled with AHA were cultured in HEPES-buffered saline (10 mM HEPES, 119 mM NaCl, 2 mM CaCl₂, 2 mM MgCl₂, 5 mM KCl and 30 mM glucose)2. In the presence of 100 μM CHX, the incorporation of methionine surrogate, AHA, into proteins was completely blocked. As demonstrated in fatty-acylation by alk-12 (mainly target N-myristoylation) and alk-16 (mainly target cysteine palmitoylation), CHX diminished N-acylation, therefore, CHX can reveal the difference of labeling patterns between lysine acylation and N-terminal acylation labeled by alkynyl-acetate analogs in the leftmost figure. All the lysates were reacted with az-Rho and analyzed by in-gel fluorescence scanning. (b) Comparison of protein labeling profiles and labeling efficiencies among alkynyl-acetate analogs and alkynyl-fatty-acid analogs in Jurkat T cells. In this experiment, cells were labeled with 5 mM of 3-butynoate, 4-pentynoate and 5-hexynoate and 20 μM of alk-12 and alk-16 for 6 hrs. The lysates were reacted with az-Rho and analyzed by in-gel fluorescence scanning. (c) Inhibition of HDACs by SAHA. SAHA induced changes in protein labeling profiles of total cell lysates and extracted core histones. Jurkat T cells were either labeled with 10 mM of alkynyl-acetate analog alone for 8 hr, or co-incubated with 10 mM of alkynyl-acetate analog as well as 10 μM SAHA for 8 hrs. The cell lysates and core histones were reacted with az-Rho and analyzed by in-gel fluorescence scanning. SAHA was chemically synthesized by following the reported synthetic procedures 3. The Western blot stained with anti-acetyl-Lys antibody showed the elevated acetylation level of histones in the presence of SAHA.

FIG. 54. In-gel fluorescent detection of acetylated proteins in different mammalian cell lines using alkynyl-acetate analogs. ˜50 μg of total cell lysates were reacted with az-Rho via CuAAC. All the different cell lines were labeled with 5 mM alkynyl-acetate analogs for 6 hr.

FIG. 55. Proteomic analysis of 3-butynoate (4)-, 4-pentynoate (5)- and 5-hexynoate (6)-metabolically labeled proteins of Jurkat T cells. (a) Schematic of proteomic profiling of metabolically labeled proteins by employing the cleavable linker (azido-diazo-biotin). (b) Profiles of enriched labeled proteins from two independent experiments. The in-gel fluorescence images (top and bottom panels) showed the metabolic labeling profiles in Jurkat T cells, in which ˜50 μg of total cell lysate were reacted with az-Rho via CuAAC. The coomassie blue-stained gels (top and bottom panels) showed the profiles of the eluted labeled proteins, in which the total cell lysates (12-20 mg) were reacted with azido-diazo-biotin via CuAAC followed by streptavidin-enrichment and Na₂S₂O₄-elution. (c) Confirming MS/MS-identified protein hits by Western blot analysis. An aliquot of eluent from each sample was separated on SDS-PAGE and transferred onto PVDF membrane. The proteins of interest were probed with their corresponding antibodies. (d) The Venn diagram shows the protein counts of unique and overlapped proteins labeled by different alkynyl-acetate analogs.

FIG. 56. Proteomic analysis of 4-pentynoate (3)-metabolically labeled proteins (Jurkat T cells). (a) Profiles of enriched labeled proteins. The in-gel fluorescence image showed the metabolic labeling profiles in Jurkat T cells, in which ˜50 μg of total cell lysate were reacted with az-Rho via CuAAC. The coomassie blue-stained gels showed the profiles of the eluted labeled proteins, in which the total cell lysates (25 mg) were reacted with azido-diazo-biotin via CuAAC followed by streptavidin-enrichment and Na₂S₂O₄-elution. (b) Confirming MS/MS-identified protein hits by Western blot analysis. An aliquot of eluent from each sample was separated on SDS-PAGE and transferred onto PVDF membrane. The proteins of interest were probed with their corresponding antibodies. (c) The pie charts show the sublocation distribution and functional classification of 4-pentynoate-metabolically labeled proteins.

FIG. 57. Preliminary results of mapping 4-pentynoate-modification sites in Jurkat T cells. (a) MS/MS-identified 4-pentynoate-modification sites in histones. (b)-(c) Selected MS/MS spectra of 4-pentynoate-modified histone peptides.

### DETAILED DESCRIPTION OF THE INVENTION

Methods and kits for the rapid and robust detection of acylated proteins produced by a cell with chemical reporters are provided herein. These methods and kits enable quantitative detection and analysis of protein acylation that occurs in a cell. Acylated proteins produced by cultured cells or by cells located within a whole organism can be detected and quantitated by the methods provided herein. In certain embodiments, the use of alkynyl-chemical reporters in combination with azido-detections tags with detectable labels is shown herein to provide improvements in detection of acylated proteins produced by cells. Other embodiments of the methods and kits provided herein for the detection of acylated proteins produced by a cell comprise the use of either alkynyl-chemical reporters or azido-chemical reporters in conjunction with detection tags that are cleavably linked to affinity purification tags.

### DEFINITIONS

Unless otherwise defined, all technical and scientific terms used herein have the same meaning as commonly understood by one of ordinary skill in the art to which this invention pertains. To the extent to which any of the following definitions is inconsistent with definitions provided in any patent or non-patent reference incorporated herein or in any reference found elsewhere, it is understood that the following definition will be used herein.

As used herein, the terms “quantitative” detection or detected “quantitatively”, when used in reference to acylated proteins, refer to determining either an absolute numeric value or relative numeric value for the amount of a acylated protein in a sample.

As used herein, the term “qualitative” detection, when used in reference to acylated proteins, refers to a visual or other representation of proteins that detects the presence of a protein without providing an either an absolute numeric value or relative numeric value for the amount of protein present.

As used herein, the term “acyl group” refers to a chemical group with the formula —C(O)—R. One of skill in the art will recognize that R can be chosen from numerous chemical groups.

As used herein, the term “acylated protein” refers to a protein that has been post-translationally modified by the covalent attachment of an acyl group C(O)—R, where R comprises a hydrocarbon chain of 1 to 50 carbons. Non-limiting examples of acylated proteins include acetylated proteins, acylated hormone peptides, and fatty-acylated proteins.

As used herein, the term “acylated amino acid residue” refers to an amino acid residue that has been modified by the covalent attachment of an acyl group.

As used herein, the term “acetylated protein” refers to a protein that has been post-translationally modified by the covalent attachment of an acetyl group (—COCH₃). As used herein, acetylated proteins are one example of acylated proteins.

As used herein, the terms “protein,” “polypeptide,” or “peptide,” are used interchangeably to refer to a polymer comprising at least two amino acids.

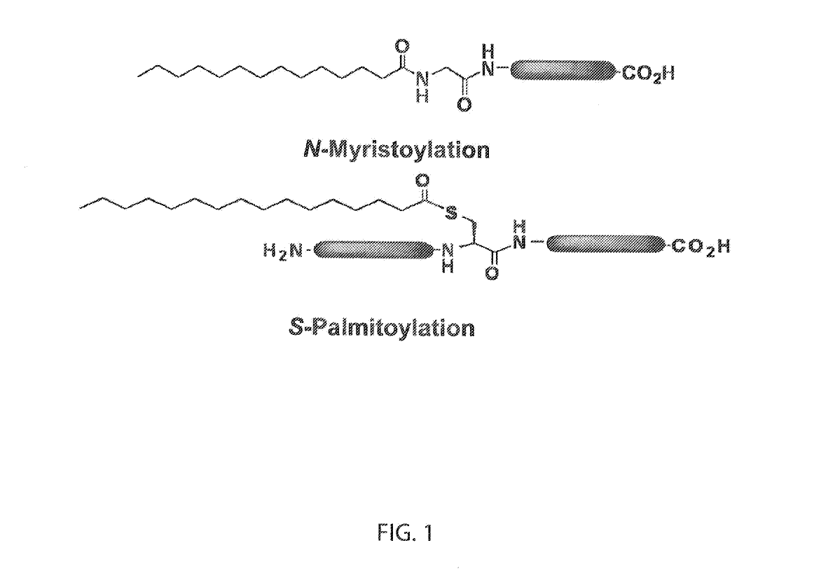

As used herein, the term “fatty-acylated protein” refers to a protein that has been post-translationally modified by the covalent attachment of one or more fatty acid chain(s) via an acyl group. Examples of fatty-acylation of proteins include, but are not limited to, N-myristoylation, which is characterized by the covalent attachment of myristic acid (14:0) to the N-terminal glycine residues of proteins and S-palmitoylation, which is characterized by the covalent attachment of palmitic acid (16:0) to cysteine residues of proteins in the form of a thioester (FIG. 1).

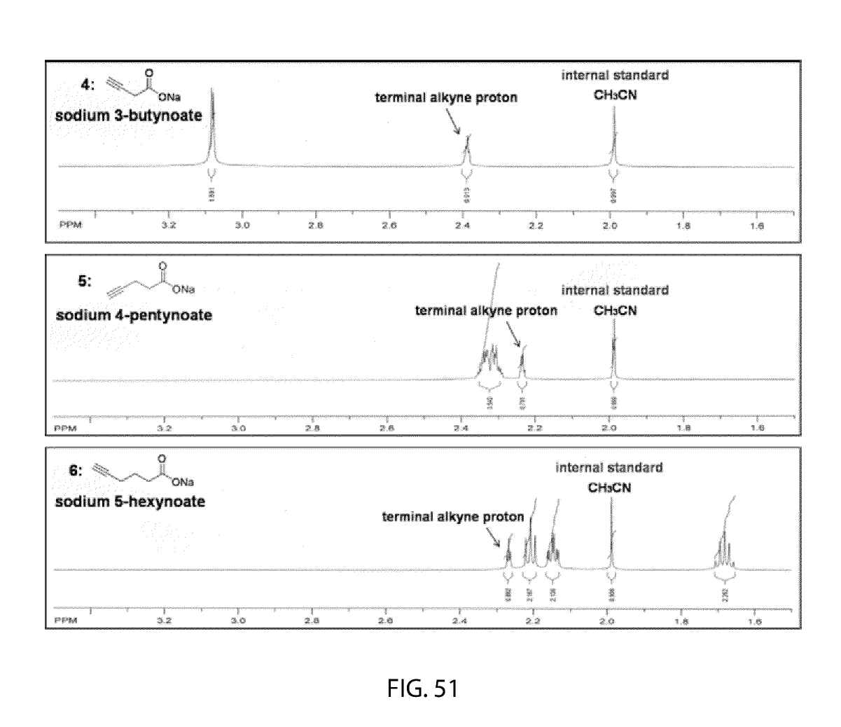

As used herein, the term “chemical reporter,” refers to a chemical agent that when provided to a cell can be linked by the cell to certain acylated proteins. As used herein, chemical reporters comprise in certain embodiments the formula R—(CH₂)ₙ—COOH, wherein R is an alkynyl (C≡C) or an azido (N₃) group. When R is an alkynyl group, n is an integer of 1 or more. When R is an azido group, n is an integer of 3 or more. As used herein, chemical reporters are sometimes referred to according to the total number of carbon atoms in the molecule. For example, a chemical reporter with six total carbon atoms is referred to as a C6 chemical reporter, twelve total carbon atoms would be C12, eighteen total carbon atoms would be C18, etc. When the chemical reporter is an alkynyl-chemical reporter comprising an alkynyl group (C≡C), the total number of carbon atoms in the chemical reporter molecule includes the two carbon atoms of the alkynyl group. In some instances, chemical reporters are referred to according to the type of group, either alkynyl or azido, and the number of carbon atoms in addition to the group. For instance, an alkynyl-chemical reporter referred to as alk-12, comprises 12 carbon atoms in addition to the two carbon atoms of the alkynyl group. Thus, the chemical reporter alk-12 is also referred to as a C14 alkynyl-chemical reporter. For instance, an azido-chemical reporter referred to as az-12, comprises 12 carbon atoms, but the azido group (N₃) does not comprise any additional carbon atoms. Thus, the chemical reporter az-12 is also referred to as a C12 azido-chemical reporter. As used herein the chemical reporter(s) can also comprise in certain embodiments compounds: i) of the formula R—(CH₂)ₙ—CO—S—CoA, wherein R is an alkynyl or an azido group and n=4-24; or, ii) of the formula R—(CH₂)ₙ—COOH, wherein R is an alkynyl group or an azido group, and wherein n=4-24, or iii) a corresponding cationic salt of the chemical reporter of the formula R—(CH₂)ₙ—COOH, wherein R is an alkynyl group or an azido group, and wherein n=4-24. Such cationic salts include, but are not limited to, Na⁺, K⁺ or Li⁺ salts. As used herein, chemical reporters can also comprise in certain embodiments the formula R—(CH₂)ₙ—CO—S—CoA, wherein R is an alkynyl (C≡C) or an azido (N₃) group. In certain embodiments the chemical reporters are of the formula R—(CH₂)ₙ—CO—S—CoA, R is an alkynyl (C≡C) group and n=1, 2, or 3 or wherein R is an azido (N₃) group and n=1, 2 or 3. In certain embodiments, chemical reporter(s) can comprise at least one or more compounds of the formula R—(CH₂)ₙ—COOH, wherein R is an alkynyl (C≡C) group and n=2 or 3 or wherein R is an azido (N₃) group and n=2 or 3. For cellular studies, such chemical reporters may also be administered to cells as their corresponding cationic salts to facilitate metabolic incorporation. Such cationic salts include, but are not limited to, Na⁺, K⁺ or Li⁺ salts. In certain embodiments, chemical reporters can comprise corresponding cationic salts of the compounds of the formula R—(CH₂)ₙ—COOH, wherein R is an alkynyl (C≡C) group or wherein R is an azido (N₃) group, and include, but are not limited to, chemical reporters as shown in FIG. 51.

As used herein, the term “alkynyl-chemical reporter” refers to a chemical reporter comprising an alkynyl group (C≡C).

As used herein, the term “azido-chemical reporter” refers to a chemical reporter comprising an azido group (N₃).

As used herein, the term “detection tag” refers to compounds with selective reactivity with a given chemical reporter. Detection tags can comprise a functional group for detection or visualization, such as a biotin group, an epitope tag, or fluorescent label.

As used herein, the term “azido detection tag” refers to a detection tag comprising an azido group that selectively recognizes alkynyl-chemical reporters. Non-limiting examples of selective reactions between azido detection tags and alkynyl-chemical reporters include Staudinger ligation and click chemistry. Azido detection tags can comprise a functional group for detection or visualization, such as a biotin group, an epitope tag, or fluorescent label.

As used herein, the term “alkynyl detection tag” refers to a detection tag comprising an alkynyl group that selectively recognizes azido-chemical reporters. A non-limiting example of a selective reaction between alkynyl detection tags and azido-chemical reporters is click chemistry. Alkynyl detection tags can comprise a functional group for detection or visualization, such as a biotin group, an epitope tag, or fluorescent label.

As used herein, the term “click chemistry” refers to reactions comprising Cu^{I}-catalyzed Huisgen [3+2] cycloaddition.

As used herein, the phrase “in vivo administration” refers to the provision of an agent to one or more cells and or tissues in a living organism by introducing the agent into the living organism.

As used herein, the term “separating,” “separation” or the like of proteins refers to any methods, techniques, protocols, or technologies that can be used to effect partial or complete isolation of proteins in a sample. Examples of such methods, techniques, protocols, or technologies include, but are not limited to, gel electrophoresis (for example SDS-PAGE), two dimensional gel electrophoresis, capillary electrophoresis, chromatography including size exclusion chromatography, ion exchange chromatography, and affinity chromatography, immunoprecipitation, and combinations thereof.

As used herein, the phrase “combination thereof” “combination of both,” or the like refers to the use of multiple elements from a group of elements. For example, for the group of elements A, B, and C: “A, B, C, or any combination thereof” refers to element A, element B, element C, elements A and B, elements A and C, elements B and C, and elements A and B and C. In such context, a “combination” does not refer to the chemical joining of two or more substances to make a single substance.

As used herein, the term “preferentially labels” or “preferentially labeled”, when used in reference to the acylation of proteins, refers to the characteristic of shorter carbon chain chemical reporters (for example similar in length to myristic acid) to be incorporated into N-myristoylated proteins at a much higher frequency than they are incorporated into S-palmitoylated proteins, and the characteristic of longer carbon chain chemical reporters (for example similar in length to palmitic acid and longer) to be incorporated into S-palymitoylated proteins at a much higher frequency than they are incorporated into N-myristoylated proteins.

### Methods for Detecting Lipidated Proteins

### I. Bioorthogonal Labeling of Acylated Proteins Produced by Cells

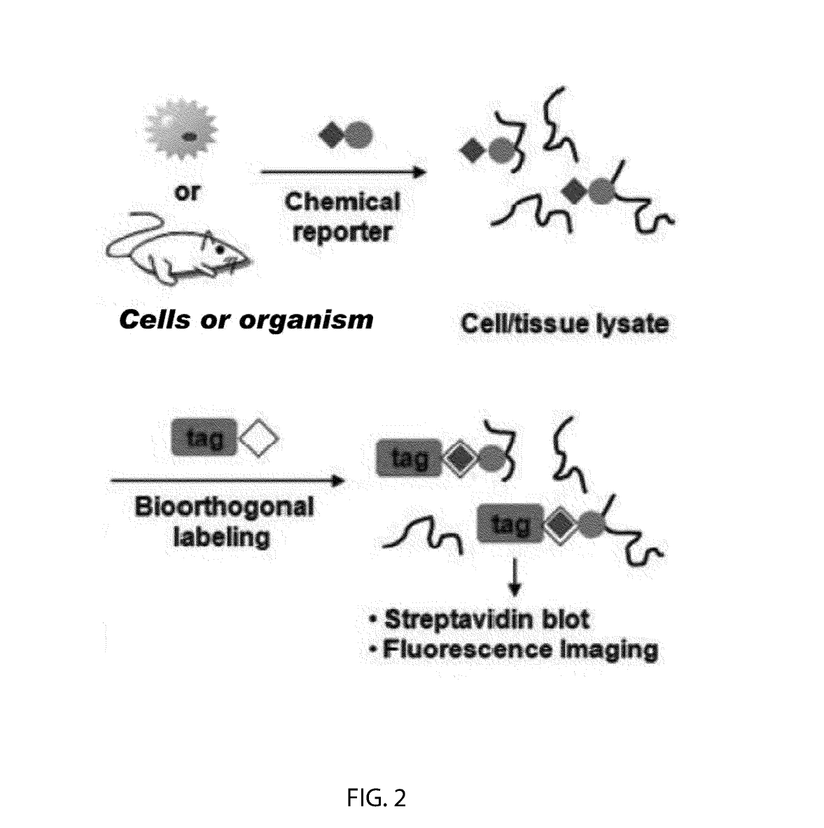

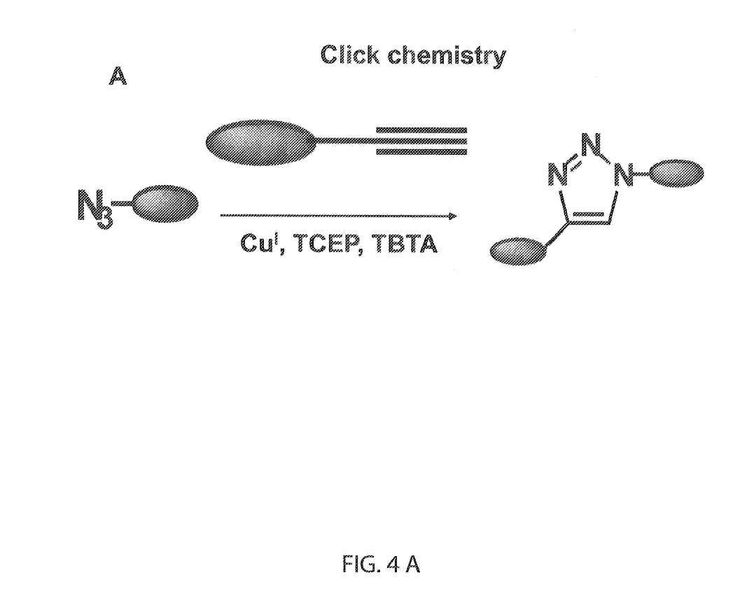

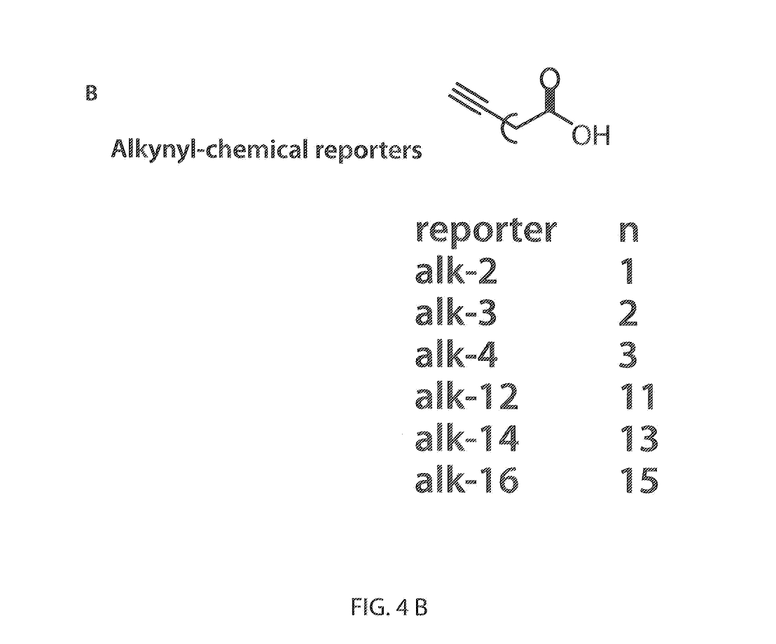

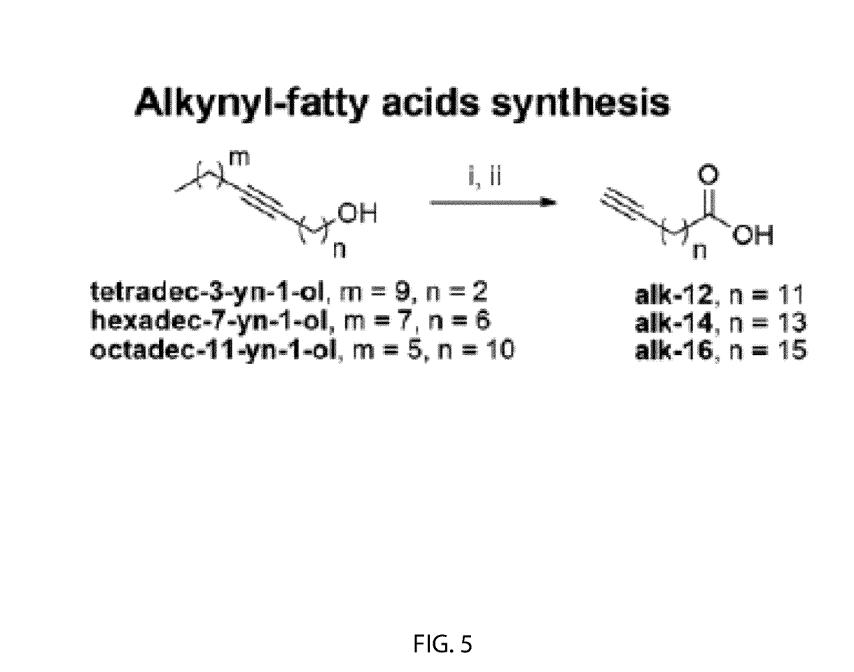

Certain embodiments of the present invention are drawn to methods for detecting one or more acylated proteins produced by a cell. Such methods utilize alkynyl-chemical reporters (FIG. 4B) that are provided to cells for labeling of acylated proteins (FIG. 2). Non-limiting examples of alkynyl-chemicals reporters of protein acylation include hexa-5-ynoic acid (alk-4), pent-4-ynoic acid (alk-3), buta-3-ynoic acid (alk-2), tetradec-13-ynoic acid (alk-12), hexadec-15-ynoic acid (alk-14), and octadec-17-ynoic acid (alk-16). An exemplary synthetic scheme for alkynyl-chemical reporter synthesis is shown in FIG. 5.

After labeling with alkynyl-chemical reporters, cultured cells or cells obtained from an organism that comprise proteins that have incorporated one or more chemical reporters, are collected, harvested, or the like. These cell are then disrupted to obtain a cell or tissue lysate (collectively “cell/tissue lysate” or just “cell lysate”) comprising alkynyl-chemical reporter labeled proteins (i.e., “alkynylated proteins”) (FIG. 2). This cell lysate is thus an “alkynylated protein lysate.”

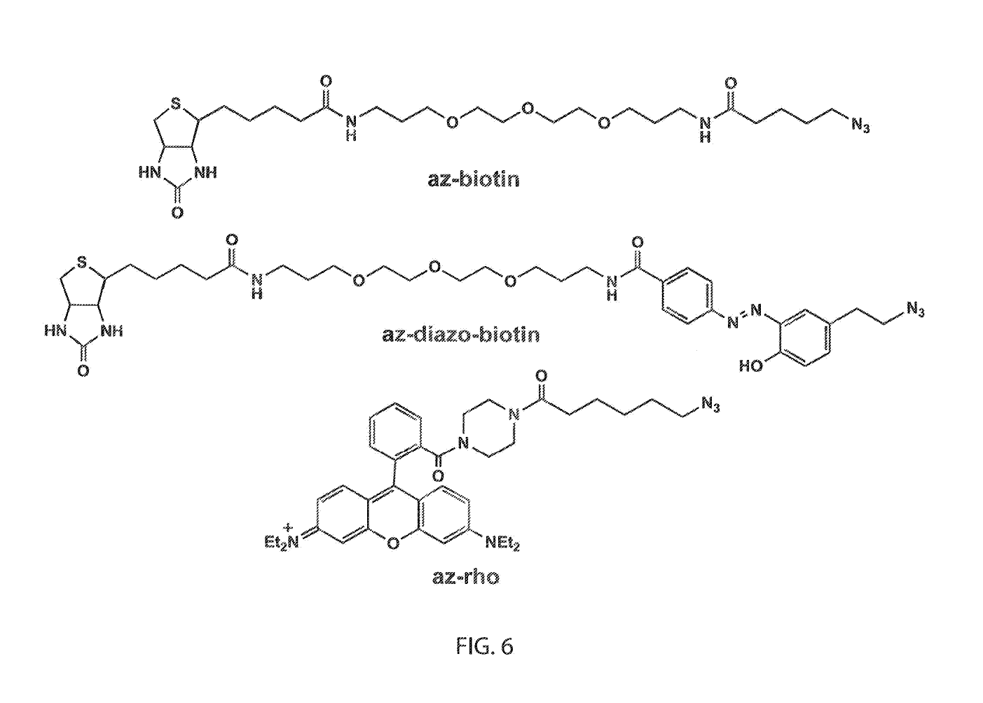

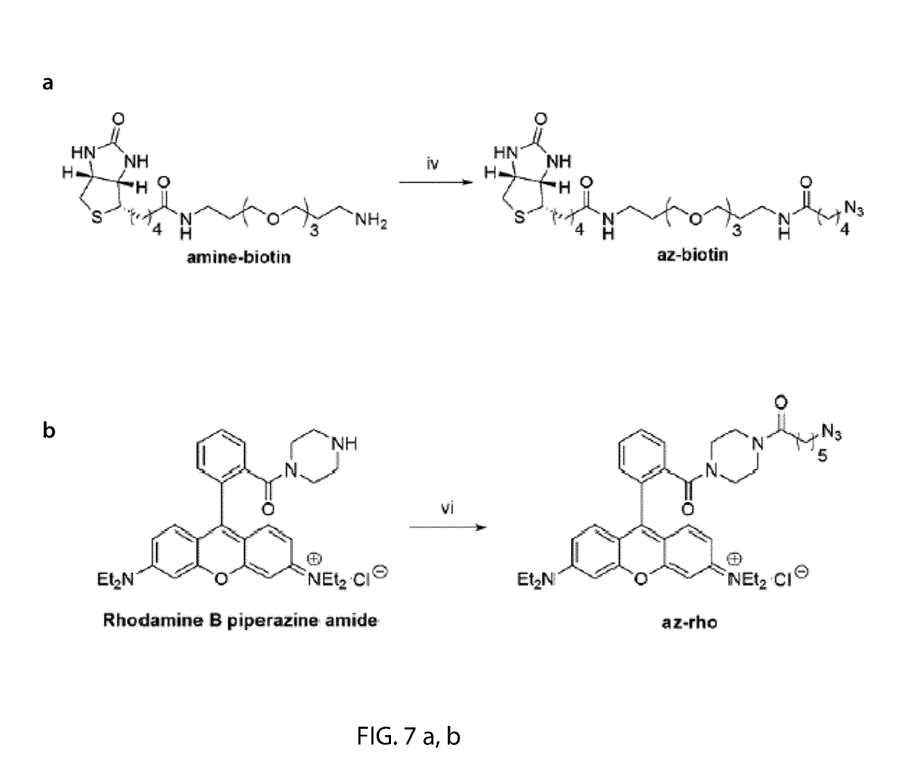

The next step is a bioorthogonal labeling step wherein alkynylated proteins are further labeled with an azido detection tag that selectively reacts with the alkynyl-chemical reporter (FIG. 2). In certain embodiments, the detection tag comprises a detectable label or tag. Detectable labels include, but are not limited to, fluorescent labels. Tags include epitope tags. The key feature of the detectable label or tag is that it allows for, for example, visualization, isolation, quantitation, or other detection or identification of the protein. One of skill in the art will appreciate that numerous fluorescent labels, epitope tags, affinity tags, and the like are well characterized and widely applied in the art of cellular and molecular biology, biochemistry, and other related and relevant fields and could be used in the detectable tags described herein. Common non-limiting examples of fluorescent labels include rhodamine, fluorescein, and the Alexa fluor family. Non-limiting examples of epitope tags include a biotin group, an immunoreactive peptide, and a polyhistidine group (His-tag). Examples of immunoreactive peptides include, but are not limited to, a myc-tag (EQKLISEEDL), a gfp (green fluorescent protein)-tag, a FLAG™-tag (DYKDDDDK), an HA-tag (YPYDVPDYA), a VSV-G-tag (YTDIEMNRLGK), an HSV-tag (QPELAPEDPED), a V5-tag (GKPIPNPLLGLDST), and any immunoreactive variants thereof. Non-limiting examples of azido detection tags for labeling of alkynyl-chemical reporters or alkynylated acylated proteins labelled with such reporters include azido-biotin, azido-diazo-biotin, and azido-rhodamine (FIG. 6). An exemplary synthetic scheme for azido-biotin detection tag synthesis is shown in FIG. 7A and an exemplary synthetic scheme for azido-rhodamine detection tag synthesis is shown in FIG. 7B.

The detection tags used to label an alkynylated protein lysate can all comprise the same detectable label. Alternatively, a mixture of detection tags comprising different detectable labels may be used to label an alkynylated protein lysate.

Following labeling with azido detection tags, the acylated proteins produced by a cell can then be detected by the appropriate method such as, but not limited to, steptavidin blotting or in-gel fluorescence scanning (FIG. 2).

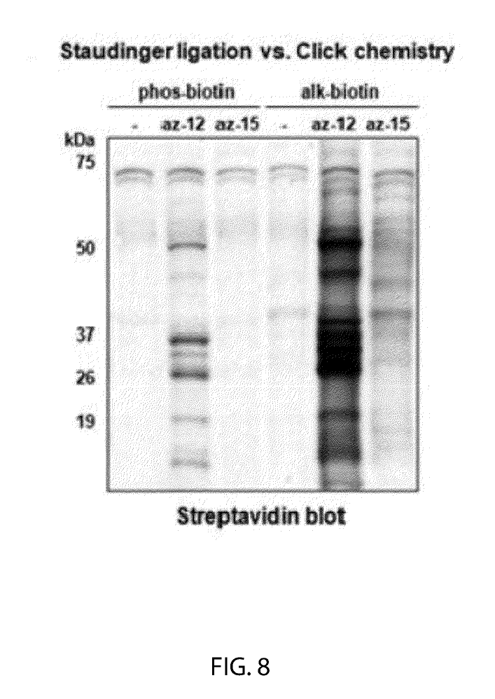

In the methods of the present invention, alkynyl-chemical reporters are complementary to azido-detection tags, and alkynylated proteins can be labeled by azido detection tags. Comparative analysis of the Staudinger ligation and Cu^{I}-catalyzed Huisgen [3+2] cycloaddition (“click chemistry”) (FIG. 4A) with azido-chemical reporter-labeled cell lysates and biotinylated detection probes revealed significantly improved detection of acylated proteins by streptavidin blotting with click chemistry (FIG. 8; phos-biotin for Staudinger ligation and alk-biotin for click chemistry). Thus, in certain embodiments, labeling of alkynylated proteins is done by performing click chemistry.

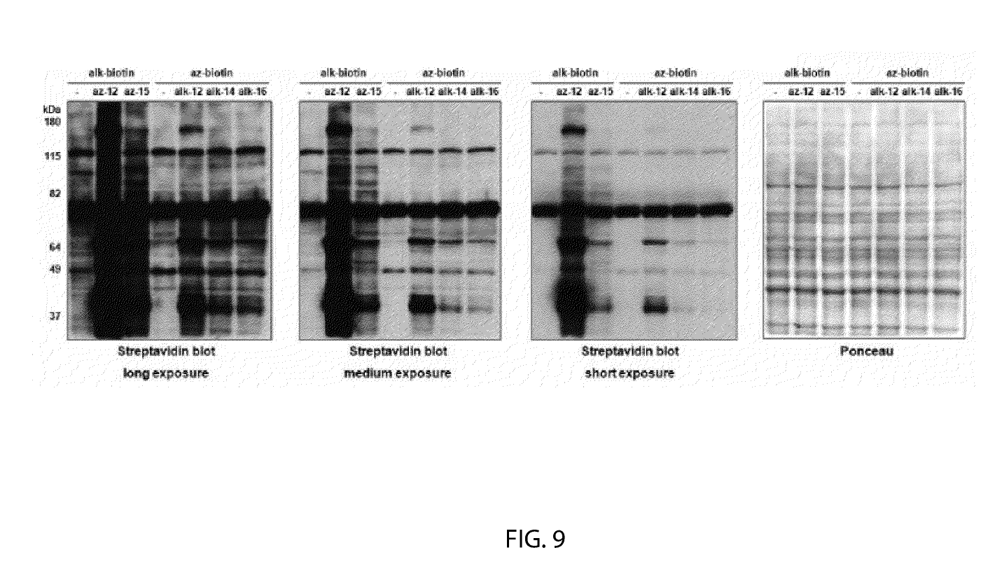

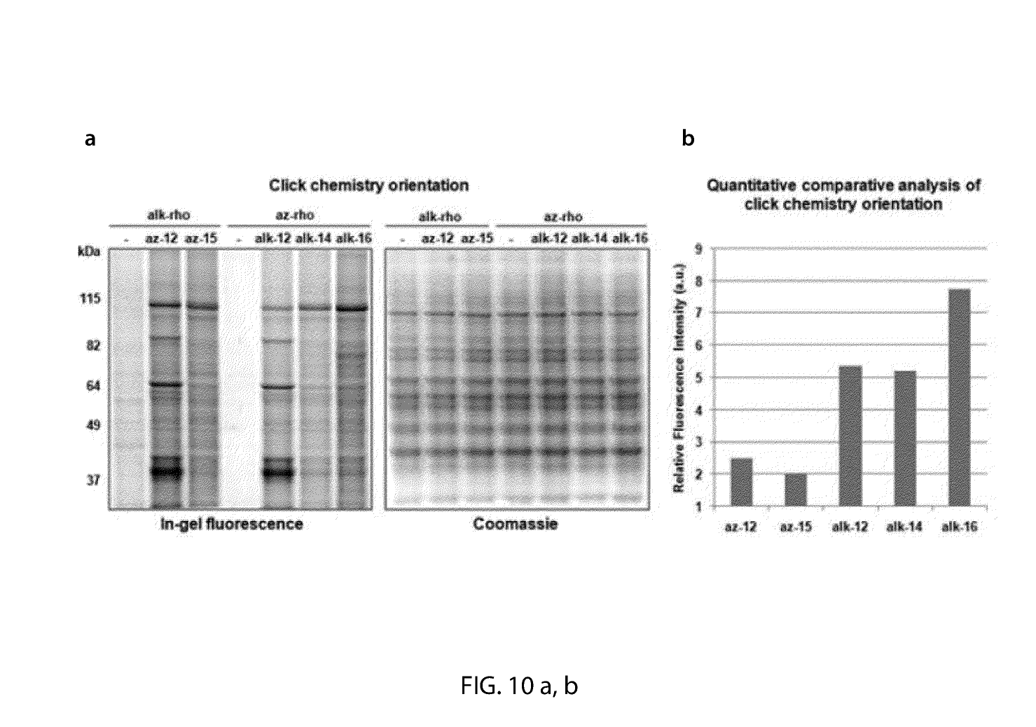

The in-gel fluorescence detection of acylated proteins circumvents the need to transfer proteins onto membranes for immunoblotting, which can be problematic for hydrophobic polypeptides, and thus provides a more direct and sensitive means to analyze proteins in a quantitative fashion. In experiments performed to date, profiles of acylated proteins visualized by in-gel fluorescence scanning revealed substantially more proteins compared to streptavidin blotting. It is further demonstrated herein that in comparison to azido-chemical reporters in combination with alkynyl detection tags, the opposite configuration, that is, alkynyl-chemical reporters in combination with azido detection tags, provides the optimal signal-to-noise for the visualization of acylated proteins (FIG. 9, FIG. 10A, and FIG. 10B). Quantitative comparative analysis of acylated proteins visualized by in-gel fluorescence scanning demonstrated that alkynyl-chemical reporters, in combination with the az-rho detection tag, afforded ˜3 to 6 fold better labeling efficiency and a 2 to 4 fold improvement in specificity of labeling compared to the reverse click chemistry orientation owing to lower background signal (FIGS. 10A and 10B).

In certain embodiments, proteins of the alkynylated protein lysate are separated before detection of the acylated proteins. Thus, acylated proteins are separated from one another and/or from other proteins before the detection step. One of skill in the art will appreciate the wide variety of protein separation techniques and protocols that are available. Exemplary methods of protein separation techniques include, but are not limited to, protein precipitation, gel electrophoresis, chromatography, and capillary electrophoresis. Methods of chromatography include, but are not limited to, size exclusion chromatography, ion exchange chromatography, and affinity chromatography. In one embodiment, proteins of the alkynylated protein lysate are separated by SDS-PAGE. In certain embodiments, multiple separation steps can be employed such as, but not limited to, multi-dimensional gel electrophoresis, a combination of protein precipitation and chromatography, or multiple chromatography steps.

Acylation of proteins is known to occur in prokaryotic and eukaryotic cells. Thus, it is contemplated that embodiments of the present invention can be used for labeling and detecting acylated proteins in prokaryotic and eukaryotic cells. In certain embodiments, the cell is a prokaryotic cell such as a bacterial cell. In certain embodiments, the cell is a eukaryotic cell such as an algal cell, a fungal cell, a yeast cell, an insect cell, a fish cell, a bird cell, a reptilian cell, an amphibian cell, a plant cell, or a mammalian cell. In certain embodiments, the cell is a mammalian cell. Exemplary mammalian cells that can be used include, but are not limited to, Jurkat cells (human T lymphoma), HeLa cells, 3T3 cells, DC2.4 cells, or HEK cells.

Labeling of acylated proteins with alkynyl-chemical reporters requires that the chemical reporters be provided to the cells producing such acylated proteins. Cells grown in culture can be incubated with chemical reporters. For example, chemical reporters can be added into media in which the cells are growing, or cells can be collected and resuspended in media or a solution containing the desired chemical reporters for an amount of time and under conditions sufficient to allow metabolic labeling of acylated proteins with alkynyl-chemical reporters. In certain embodiments, cells are incubated with chemical reporters for about 4 to about 6 hours. However, in other embodiments, different incubation regimes can be also used. For example, cells can be “pulse labelled” by incubation the cells with the chemical reporter(s) for a given time interval and then culturing the same cells in the absence of the chemical reporter(s). Such “pulse labeling” experiments are useful in analyzing the fate of acylated proteins produced by a cell.

Detection of acylated proteins produced by cells within living animals is also provided herein. Such detection of acylated proteins produced by cells within living animals provides new opportunities to address protein acylation in physiology and disease. Thus, in certain embodiments, alkynyl-chemical reporters are provided to a cell by in vivo administration to an organism. In certain embodiments, the organism is a non-human organism. Non-limiting examples of non-human organisms include plants, reptiles, amphibians, insects, worms, fish, birds, and non-human mammals. In certain embodiments, the non-human organism is a mammal, such as a rodent, canine, swine, or primate. In certain embodiments, the non-human mammal is a mouse (see Example 4).

In certain embodiments, alkynyl-chemical reporters may be administered in vivo systemically, such as by intraperitoneal injection or intravenous injection. Systemic administration is used in certain applications because it allows for chemical reporters to distribute throughout an organism to cells located in a variety of tissues and organs. In certain other embodiments, chemical reporters can be administered in vivo by localized administration, such as by direct injection into a target cell population or tissue. Localized administration can be advantageous, for example, when labeling of a specific organ or tissue is desired to allow a greater concentration of an agent to be administered to a target area or to directly label cells within an organism that are less susceptible to labeling though systemic administration. Localized in vivo administration can also be used in instances where it is desirable to avoid any potential side-effects associated with systemic administration. In certain embodiments, an vivo labeling period of about 1 to about 3 hours following administration is sufficient for detection of proteins.

The chemical reporters used in the methods of the present invention can be of various lengths. In certain embodiments, chemical reporters comprise from four to twenty-four carbon atoms (i.e. C4 to C24). In certain embodiments, the alkynyl-chemical reporters are hexa-5-ynoic acid (C6), pent-4-ynoic acid (C5), buta-3-ynoic acid (C4), tetradec-13-ynoic acid (C14), hexadec-15-ynoic acid (C16), and octadec-17-ynoic acid (C18). In other embodiments, the alkynyl-chemical reporters are hexa-5-ynoic acid (C6), pent-4-ynoic acid (C5), and buta-3-ynoic acid (C4). In other embodiments, the alkynyl-chemical reporters are tetradec-13-ynoic acid (C14), hexadec-15-ynoic acid (C16), and octadec-17-ynoic acid (C18). Alkynyl-chemical reporters of different lengths or with other differences in compositional properties may be used individually to label acylated proteins produced by a cell, or they may be used in combination. For purposes of illustration, cells can be provided with just tetradec-13-ynoic acid (C14), just hexadec-15-ynoic acid (C16), or just octadec-17-ynoic acid (C18), or with a mixture of equal or unequal parts of tetradec-13-ynoic acid (C14) and hexadec-15-ynoic acid (C16), tetradec-13-ynoic acid (C14) and octadec-17-ynoic acid (C18), hexadec-15-ynoic acid (C16) and octadec-17-ynoic acid (C18), or tetradec-13-ynoic acid (C14) and hexadec-15-ynoic acid (C16) and octadec-17-ynoic acid (C18).

Very short chain alkynyl-chemical reporters, such as hexa-5-ynoic acid, pent-4-ynoic acid, and buta-3-ynoic acid, can be used to label acetylated proteins. Alkynyl-chemical reporters of around about 12 to 14 carbons, such as 12-azido-dodecanoic acid and tetradec-13-ynoic acid, can be used to preferentially label N-myristoylated proteins produced by a cell in comparison to S-palmitoylated proteins produced by a cell. Longer chain alkynyl-chemical reporters of around about 15 to 18 carbons, such as 15-azido-pentadecanoic acid, hexadec-15-ynoic acid, and octadec-17-ynoic acid, can be used to preferentially label S-palmitoylated proteins produced by a cell in comparison to N-myristoylated proteins produced by a cell. Thus, in certain embodiments of the methods of the present invention, the length of the alkynyl-chemical reporter utilized is selected according to the type of protein acylation that is of interest.

### II. Isolation of Acylated Proteins

Certain embodiments of the present invention are drawn to methods for detecting one or more acylated proteins produced by a cell that include a step of isolating alkynylated proteins labeled within the cell from non-alkynylated proteins. In certain embodiments, this step can occur before labeling with azido detection tags.

The first step of the method is to provide a cell with alkynyl-chemical reporters for labeling of acylated proteins, and obtaining a cell lysate (“alkynylated protein lysate”) comprising alkynylated proteins. Alkynylated proteins of the alkynylated protein lysate are then isolated from non-alkynylated proteins. In certain embodiments, specific alkynylated proteins can also be isolated from other alkynylated proteins.

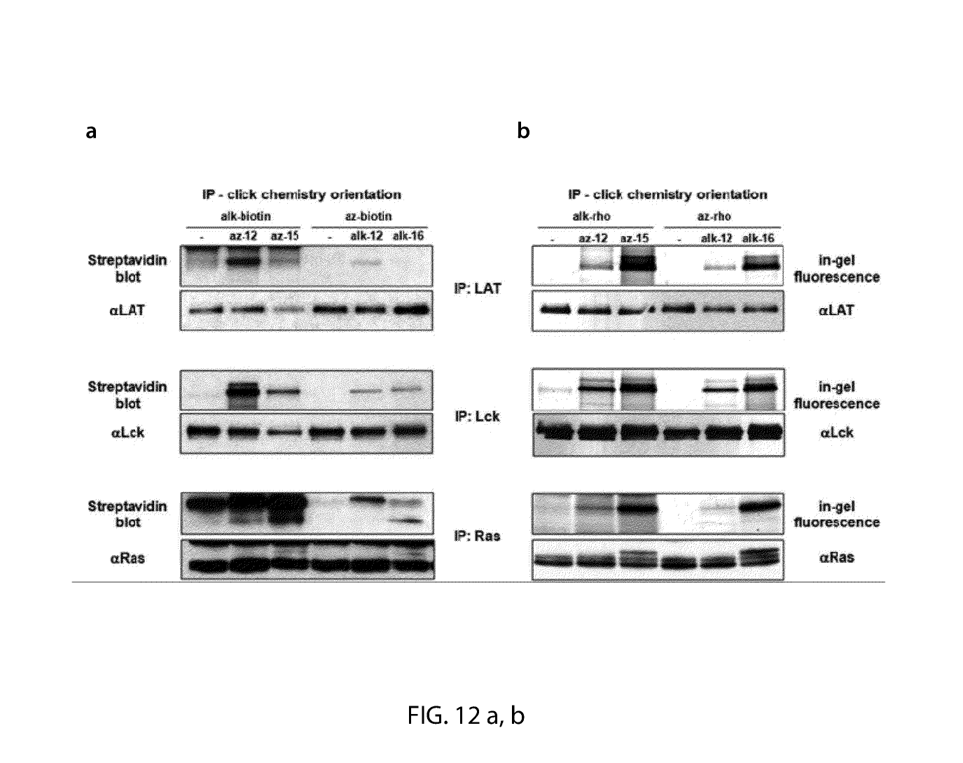

Following isolation, alkynylated proteins are further labeled with azido detection tags, and can then be detected by the appropriate methods or protocols as previously described. For example, in-gel fluorescence detection of immunopurified fatty-acylated proteins (FIG. 12B) was markedly improved compared to streptavidin blotting (FIG. 12A). Thus, in certain embodiments, fatty-acylated proteins are detected by in-gel fluorescence. In certain embodiments, isolated alkynylated proteins that have been labeled with azido detection tags are further separated, as described previously, before detection.

Isolation of alkynylated proteins can be achieved by various techniques, methods, and protocols well known to those skilled in the art. Non-limiting examples include immuno-precipitation or affinity chromatography. An exemplary and non-limiting embodiment where immunoprecipitation from alkynylated protein lysates of the N-myristoylated and S-palmitoylated protein Lck, the S-palmitoylated only protein Linker for Activation of T Cells (LAT), and the S-palmitoylated and S-prenylated protein Ras is provided in Example 3.

### III. Affinity Purification of Acylated Proteins

Also provided herein are methods and kits for detecting acylated proteins produced by a cell where certain chemical reporters can be coupled with detection tags attached to affinity purification tags to allow for affinity purification of acylated proteins from a cell lysate. In certain embodiments, the detection tag is coupled to the affinity purification tag by a cleavable linkage to facilitate recovery of the acylated protein by affinity purification and cleavage of that linkage. Certain embodiments of the present invention also provide for identification of the acylated proteins isolated by these methods to be identified with mass spectrometry. In embodiments where all of the acylated proteins are labelled with the same affinity purification tag and purified with the same affinity tag binding reagent, a global analysis of the different types of acylated proteins produced by a cell is obtained.

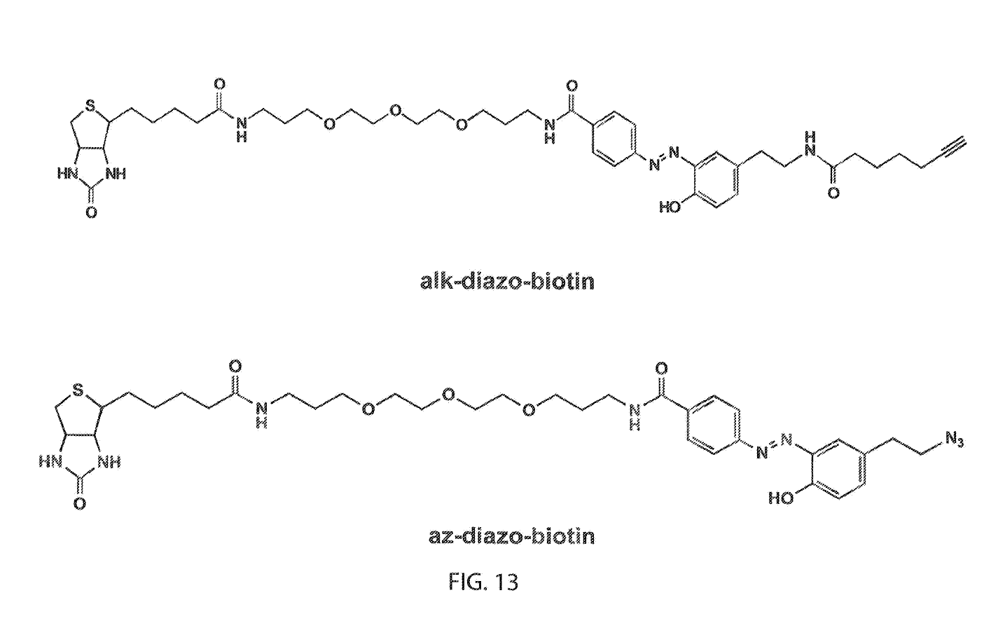

Certain embodiments of the present invention are thus drawn to methods for detecting acylated proteins produced by a cell, the methods including the step of isolating proteins labeled with a detection tag attached to an affinity purification tag. Unless otherwise specified, such methods are consistent with the methods previously described. Cells are first provided with chemical reporters for labeling of acylated proteins. The chemical reporters are not limited to alkynyl-chemical reporters. Chemical reporters can be either alkynyl- or azido-chemical reporters (FIG. 13). Exemplary, non-limiting examples of chemical reporters that can be used in these methods include 12-azido-dodecanoic acid (az-12), 15-aziod-pentadecanoic acid (az-15), hexa-5-ynoic acid (alk-4), pent-4-ynoic acid (alk-3), buta-3-ynoic acid (alk-2), tetradec-13-ynoic acid (alk-12), hexadec-15-ynoic acid (alk-14), and octadec-17-ynoic acid (alk-16).

After labeling with chemical reporters, cultured cells or cells obtained from an organism that comprise proteins that have incorporated a chemical reporter, are collected, harvested, or the like. These cells are then disrupted to obtain a cell or tissue lysate comprising chemical reporter labeled proteins. This cell lysate is also referred to herein as a “protein lysate.”

The next step is a bioorthogonal labeling step wherein chemical reporter labeled proteins are further labeled with detection tags that selectively react with the chemical reporter. When the chemical reporter comprises an azido group, labeling with a detection tag comprising an alkynyl group can be accomplished by either Cu^{I}-catalyzed Huisgen [3+2] cycloaddition or strain-promoted Huisgen [3+2] cycloaddition. In certain embodiments, strain-promoted Huisgen [3+2] cycloaddition can be performed with cyclooctynes or with difluorinated cyclooctyne (Agard et al., J Am Chem Soc. (2004) 126(46):15046-15047; Baskin et al., (2007) Proc. Natl. Acad. Sci. USA, 104(43)16793-16797). The detection tag is attached to an affinity purification tag that allows for selective capture of the detection tag (that is linked to a chemical reporter incorporated onto to an acylated protein) on a solid support comprising an agent the binds the affinity purification tag. Thus, the acylated protein is captured on the solid support. Numerous affinity purification tags and binding agents are well known to those of skill in the art. In certain embodiments of the methods and kits provided herein, the affinity purification tag is a biotin group, an immunoreactive peptide, or a polyhistidine group (His-tag). Examples of immunoreactive peptides include, but are not limited to, a myc-tag (EQKLISEEDL), a gfp (green fluorescent protein)-tag, a FLAG™-tag (DYKDDDDK), an HA-tag (YPYDVPDYA), a VSV-G-tag (YTDIEMNRLGK), an HSV-tag (QPELAPEDPED), a V5-tag (GKPIPNPLLGLDST), and any immunoreactive variants thereof. In certain embodiments, the affinity purification tag comprises a biotin group and the solid support comprises streptavidin as a binding agent.

Following capture of the acylated proteins on the solid support, the proteins can be released. One of skill in the art will recognize that the method used to release the captured proteins will be dependent on the type of purification tag, binding agent, and solid support used, and can include, for example but not limited to, varying the ionic concentration, varying the pH, varying detergent concentration, and/or by adding a competitive binding agent.

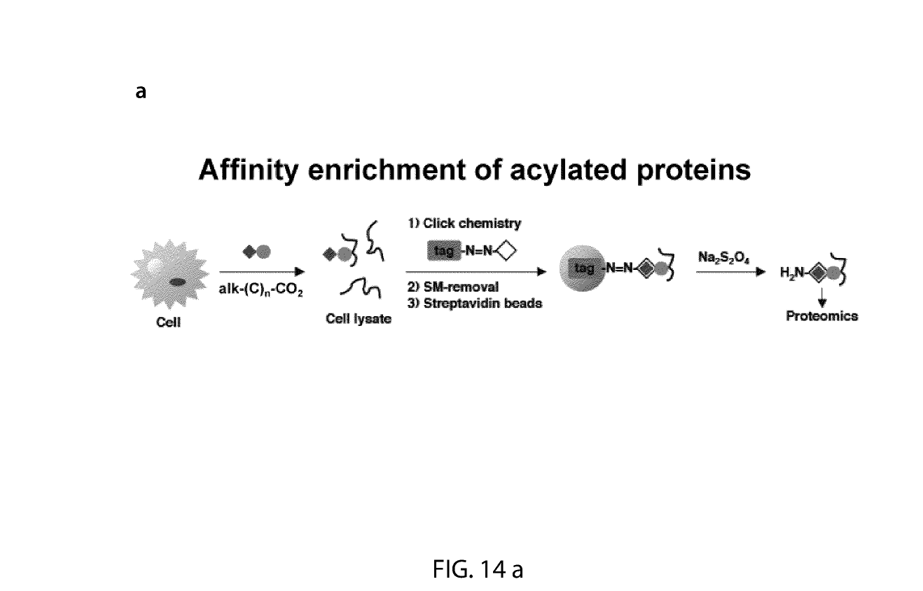

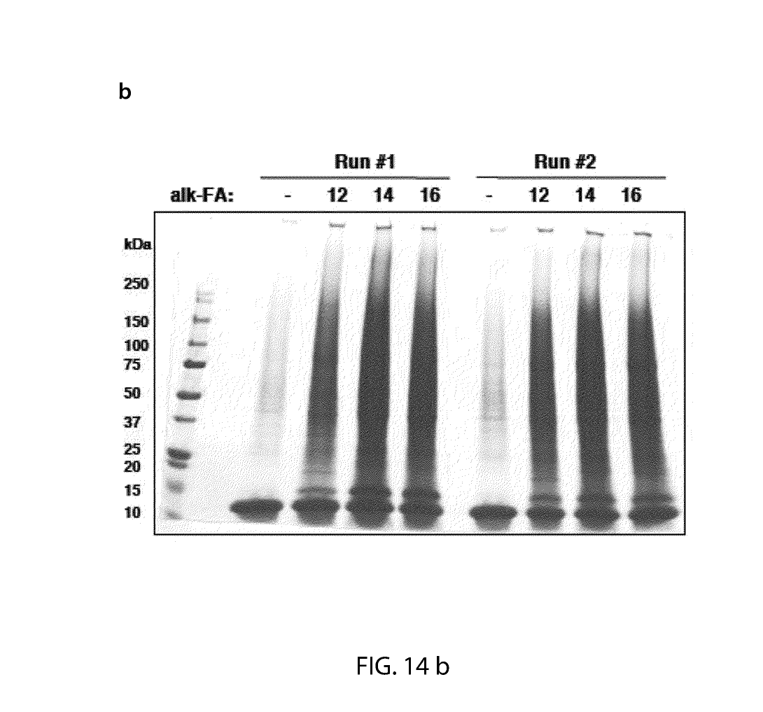

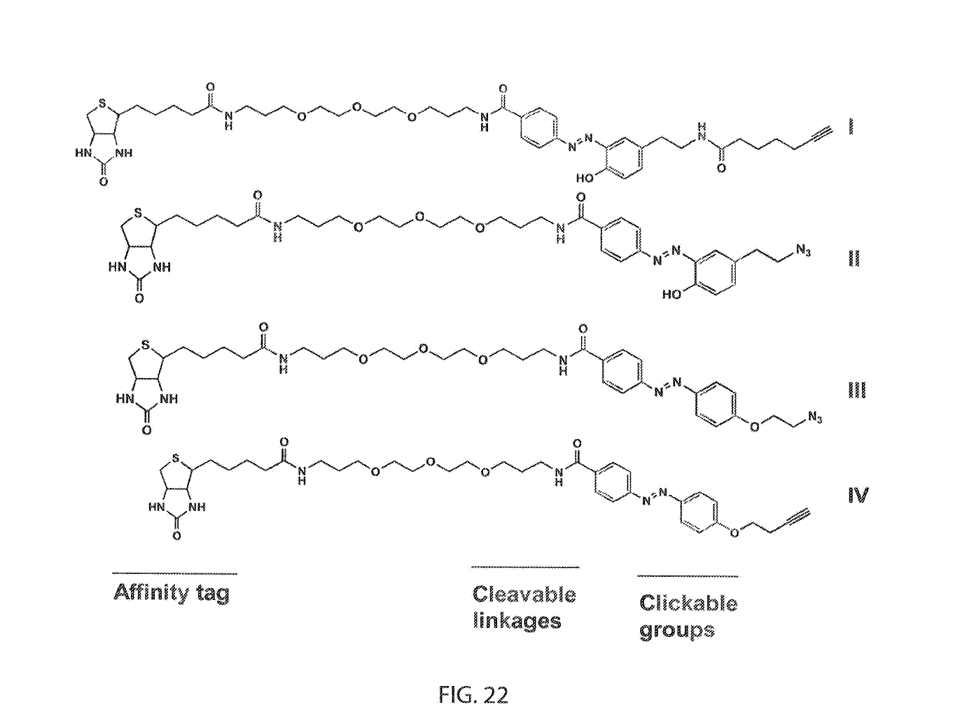

In certain embodiments, the detection tag is attached to an affinity purification tag by a cleavable linkage that allows the captured acylated proteins to be released from the solid support by cleaving the attachment between the detection tag and the affinity purification tag. Non-limiting examples of such cleavage strategies known in the art include disulfide, acid- and base-sensitive functional groups, and protease-sensitive peptides. In other embodiments, the cleavable linkage can comprise an acid cleavable linker, a base-cleavable linker, or a diazo linker. Diazo linkers can be efficiently cleaved by reduction with sodium dithionite (Na₂S₂O₄) and can be readily incorporated as a linker between the detection tag and affinity purification tag. Thus, in certain embodiments, the affinity purification tag comprises a biotin group and the cleavable linkage is a diazo linkage. Non-limiting examples of detection tags linked to an affinity purification tags by a cleavable diazobenzene linkage include the following compounds shown in FIG. 22: (I) (E)-4-((5-(2-hept-6-ynamidoethyl)-2-hydroxyphenyl)diazenyl)-N-(15-oxo-19-(2-oxohexa hydro-1H-thieno[3,4-a]imidazol-4-yl)-4,7,10-trioxa-14-azanonadecyl)benzamide and (II) (E)-4-((5-(2-azidoethyl)-2-hydroxyphenyl)diazenyl)-N-(15-oxo-19-(2-oxohexahydro-1H thieno[3,4-d]imidazol-4-yl)-4,7,10-trioxa-14-azanonadecyl)benzamide. Further, in certain embodiments, the affinity purification tag comprises a biotin group, the solid support comprises streptavidin as a binding agent, the cleavable linkage is a diazobenzene linkage, and cleavage of the diazobenzene linkage is effected with sodium dithionite (FIG. 14).

One of skill in the art will recognize that to increase the purity of affinity purification products, it may be beneficial to wash the solid support comprising captured acylated proteins to remove proteins associated with the solid support through non-specific interactions before releasing the affinity purified proteins from the solid support. Illustrative wash regimens include, but are not limited to, buffers of low, moderate, or high ionic strength that can optionally comprise one or more detergents and/or urea. The wash solution used is such that it will provide for removal of proteins or other impurities from the solid support while permitting acylated proteins captured by the affinity purification tag binding agent to be retained.

Following release of the acylated proteins from the solid support, the acylated proteins can be detected by any applicable method or protocol. Such various methods include, but are not limited to, staining, immunolabelling, fluorescence, radiometry, and mass spectrometry.

Detection of acylated proteins that have been released from an affinity purification solid support can be attained by assaying for the presence of detectable label that remains linked to the detection tag that is attached through the chemical reporter to the acylated protein following cleavage of the protein from an affinity purification tag. It is contemplated herein that examples of detectable labels include, but are not limited to, a fluorophore or a halogen. Further, the detectable label may also be an isotope. In certain embodiments, the detectable label is a halogen such as chlorine, bromine, fluorine, and iodine. It is contemplated that any acylated protein that is labeled with a chemical reporter and a detection tag so as to yield an acylated protein with a different mass than the naturally occurring form of the acylated protein that is not so labelled can be detected and/or identified by mass spectrometry.

Also provided herein are methods and kits where more than one chemical reporters are provided to the cell and more than one detectable tags attached to an affinity purification tag by a cleavable linkage are used to label the acylated proteins produced by a cell. In one embodiment, the use of both a chemical reporter that preferentially labels N-myristoylated proteins and a chemical reporter that preferentially labels S-palmitoylated proteins is provided. Shorter chain alkynyl- or azido-chemical reporters, such as 12-azido-dodecanoic acid and tetradec-13-ynoic acid, can be used to preferentially label N-myristoylated proteins produced by a cell, whereas longer chain alkynyl- or azido-chemical reporters, such as 15-azido-pentadecanoic acid, hexadec-15-ynoic acid, and octadec-17-ynoic acid, can be used to preferentially label S-palmitoylated proteins produced by a cell. In some embodiments, very short chain alkynyl- or azido-chemical reporters, such as hexa-5-ynoic acid (C6), pent-4-ynoic acid (C5), and buta-3-ynoic acid (C4), can be used to label acetylated proteins. A detection tag that is then specific for each distinct chemical reporter can then be used to specifically identify each type of acylated protein produced. For example, an azido-detection tag can be used to specifically identify those acylated proteins labelled with one of the chemical reporters that comprises an alkynyl group while an alkynyl-detection tag can be used to detect those acylated proteins with one of the chemical reporters that comprises an azido group. In one embodiment of these methods, the detection tags can be cleavably linked to the same affinity purification tag but would contain different detectable labels that would permit the acylated proteins to be distinguished following release from the solid support by the cleaving the cleavable linkage. For example, one of the detection tags could comprise a fluorophore that is spectrally distinct from the fluorophore linked to the other detection tag to provide for differential detection of the proteins. In other embodiments, the detection tags can be cleavably linked to the distinct affinity purification tags to provide for separation of the proteins labelled with distinct chemical reporters and detection tags.

### IV. Kits

The current invention provides for kits, for example for research or commercial use that provide some or all of the reagents necessary to perform methods of the invention to detect, isolate, and/or identify acylated proteins produced by a cell.

### Kits for the Detection of Acylated Proteins

Certain embodiments contemplated herein provided for kits comprising one or more chemical reporter(s) and one or more detection tag(s). As previously described, detection tags are able to selectively label proteins incorporating one or more chemical reporters and thus label an acylated protein lysate. Certain embodiments contemplated herein provided for kits comprising one or more alkynyl-chemical reporter(s) and one or more azido detection tag(s). As previously described, azido detection tags are able to selectively label proteins incorporating one or more alkynyl-chemical reporters and thus label an alkynylated protein lysate. The kits further comprise containers for the chemical reporters and detection tags. In certain embodiments, kits also comprise reagents for performing the click chemistry reaction that allows for selective covalent attachment of detection tags to alkyne-modified substrates, as well as containers for said reagents.

In certain embodiments, the one or more alkynyl-chemical reporter(s) contained in the kit is at least one of hexa-5-ynoic acid, pent-4-ynoic acid, buta-3-ynoic acid, tetradec-13-ynoic acid, hexadec-15-ynoic acid, or octadec-17-ynoic acid. Each chemical reporter can be provided in its own separate container, or multiple chemical reporters can be provided as a mixture in a common container.

As previously described, in certain embodiments, the azido detection tag comprises a detectable label, such as a fluorescent label or epitope tag. In certain embodiments, the detectable label is biotin. In certain embodiments, a mixture of azido detection tags can be provided.

In certain embodiments, kits comprise instructions for detecting one or more acylated proteins produced by a cultured cell. In certain embodiments, the kit comprise instructions for detecting one or more acylated proteins produced by a cell in an organism. In certain embodiments, the organism is a non-human organism. The instructions can be printed instructions. The instructions can also take advantage of a variety of electronic formats such as providing information on a diskette, or providing an internet location or address for viewing, printing, or downloading online instructions.

### Kits for Affinity Purification of Acylated Proteins

Certain embodiments contemplated herein provide for kits comprising one or more chemical reporter(s) and one or more detection tag(s) attached to an affinity purification tag. In certain embodiments, the detection tag is attached to the affinity purification tag via a cleavable linkage. Such kits further comprise containers for the chemical reporters and detection tags. In certain embodiments, kits also comprises reagents for performing the click chemistry reaction that allows for selective covalent attachment of detection tags to azide/alkyne-modified substrates, as well as containers for said reagents.

As previously described, the affinity purification tag binds to a solid support comprising a binding agent and immobilizes proteins labeled with the affinity purification tag on the solid support. In certain embodiments, the affinity purification tag comprises a biotin group. Immobilized proteins can subsequently be eluted from the solid support for detection, identification, analysis, and the like. A cleavable linkage such as an acid cleavable linker, a base cleavable linker, or a diazo linker further allows for the immobilized proteins to be efficiently eluted. In certain embodiments, the cleavable linkage is a diazo linker. In certain embodiments, the affinity purification tag comprises a biotin group and the cleavable linkage is a diazo linker.

In certain embodiments, kits also include a solid support or an agent that binds an affinity purification tag. In certain embodiments, the kits include a solid support comprising an agent that binds an affinity purification tag. In certain embodiments, the solid support comprises streptavidin as a binding agent.

In certain embodiments, the chemical reporters can be alkynyl-chemical reporters and/or azido-chemical reporters. In certain embodiments, the chemical reporter is an azido-chemical reporter such as 12-azido-dodecanoic acid, 15-azido-pentadecanoic acid, or a combination of the two. In certain embodiments, the chemical reporter is an alkynyl-chemical reporter such as hexa-5-ynoic acid, pent-4-ynoic acid, buta-3-ynoic acid, tetradec-13-ynoic acid, hexadec-15-ynoic acid, octadec-17-ynoic acid, or any combination thereof. Depending on whether the chemical reporter is an alkynyl- or azido-chemical reporter, the corresponding detection tag is an azido- or alkynyl-detection tag respectively. In certain embodiments, the detection tag comprises a detectable label. As previously described, such detectable label remains attached to the detection tag following cleavage of the cleavable linkage between a detection tag and an affinity purification tag.

In certain embodiments, kits comprise instructions for detecting one or more acylated proteins produced by a cultured cell. In certain embodiments, the kit comprises instructions for detecting one or more acylated proteins produced by a cell in an organism. In certain embodiments, the organism is a non-human organism. The instructions can be printed instructions. The instructions can also take advantage of a variety of electronic means such as providing information on a diskette, or providing an interne location or address for viewing, printing, or downloading online instructions.

### EXAMPLES

The following disclosed embodiments are merely representative of the invention, which may be embodied in various forms. Thus, specific structural and functional details disclosed herein are not to be interpreted as limiting.

All reagents and chemicals are either commercially available or can be prepared by standard procedures found in the literature or are known to those of skill in the arts of cell or molecular biology, organic chemistry, biochemistry, and the like.

### Example 1

### Experimental Methods

### Metabolic Labeling.

Jurkat cells (human T lymphoma) were cultured in RPMI medium 1640 supplemented with 10% fetal bovine serum (FBS), 100 U/mL penicillin, and 100 μg/mL streptomycin and maintained in a humidified 37° C. incubator with 5% CO₂. Trypan blue exclusion was used to determine cell viability. For labeling of N-myristoylated or S-palmitoylated proteins, cells were pelleted and resuspended in either az-12 or alk-12 (20 μM, 5 mM stock solution in DMSO) or az-15, alk-14 or alk-16 (200 μM, 50 mM stock solution in DMSO) respectively in RPMI medium 1640 supplemented with 2% FBS, 100 U/mL penicillin, and 100 μg/mL streptomycin. For labeling of acetylated proteins, cells were pelleted and resuspended in alk-2, alk-3, or alk-4 (stock solution in DMSO) in RPMI medium 1640 supplemented with 2% FBS, 100 U/mL penicillin, and 100 μg/mL streptomycin. The same volume of DMSO was used as a negative control. After 4-6 hours of labeling at 37° C., the cells were pelleted at 1,000 g for 5 minutes and washed once with ice-cold PBS, directly lysed or flash frozen in liquid nitrogen and stored at −80° C. prior to lysis. No significant loss of signal was observed for frozen cell pellets.

HeLa, 3T3, Jurkat and DC2.4 cells were cultured in DMEM, supplemented with 10% fetal bovine serum (FBS), 100 U/mL penicillin, and 100 μg/mL streptomycin and maintained in a humidified 37° C. incubator with 5% CO₂. Trypan blue exclusion was used to determine cell viability. Cells were treated with either alk-12 (20 μM, 5 mM stock solution in DMSO) or alk-16 (200 μM, 50 mM stock solution in DMSO) in DMEM supplemented with 2% FBS, 100 U/mL penicillin, and 100 μg/mL streptomycin. The same volume of DMSO was used as a negative control. After 4-6 hours of labeling at 37° C., the cells were washed once with ice-cold PBS, harvested with a cell scraper and pelleted at 1,000 g for 5 minutes. Jurkat, HeLa, DC 2.4, COST, 3T3, and Raw 264.7 were similarly treated with alk-4.

Spleens were harvested from 6 week-old female C57/13L6 mice. Splenocytes were prepared by manual disruption of spleens using forceps. Red blood cells were eliminated using ACK lysis buffer. Splenocytes were pelleted and resuspended in either alk-12 (20 μM, 5 mM stock solution in DMSO) or alk-16 (200 μM, 50 mM stock solution in DMSO) in RPMI medium 1640 supplemented with 2% FBS, 100 U/mL penicillin, and 100 μg/mL streptomycin using one spleen per labeling condition. The same volume of DMSO was used as a negative control. After 4-6 hours of labeling at 37° C., the cells were pelleted at 1,000 g for 5 minutes, washed once with ice-cold PBS, and directly lysed.

### In Vivo Labeling.

PBS containing 10% fatty acid free BSA (Sigma, St. Louis, Mo., USA) was added to alk-12 and alk-16 (5 mg/mL), followed by brief sonication, warming to 37° C., and IP injection of 200 μL into 6 week-old female C57/BL6 mice for 1 or 3 hours. Livers were harvested and incubated with Liberase 3 Blendzyme™ (Roche, Mannheim, Germany) at 37° C. for 30 minutes+ and homogenized prior to filtration with 0.4 μm cell strainers. Splenocytes were prepared by manual disruption of spleens using forceps. Liver and splenocyte preparations were subjected to red blood cell lysis using ACK lysis buffer. Cells were pelleted at 1,000 g for 5 minutes, washed once with ice-cold PBS, and directly lysed.

### Preparation of Cell Lysates.

Cell pellets obtained from 10×10⁶ Jurkat cells or 1 confluent well of a 6-well plate of HeLa, 3T3 or DC2.4 cells were lysed with 100 μL of ice-cold modified RIPA lysis buffer (1% Nonidet P 40, 1% sodium deoxycholate, 0.1% SDS, 50 mM triethanolamine pH 7.4, 150 mM NaCl, 5×EDTAfree Roche protease inhibitor cocktail, 10 mM phenylmethylsulfonyl fluoride (PMSF)) by first disrupting the pellet by sonication, and then vortexing 3×10 seconds, cooling the lysate on ice between pulses. Cell lysates were collected after centrifuging at 1,000 g for 5 minutes at 4° C. to remove cell debris. Protein concentration was determined by the BCA assay. Typical lysate protein concentrations obtained: Jurkat 2 to 3 mg/mL, HeLa 1 to 2 mg/mL, 3T3 0.5 to 1 mg/mL and DC2.4 1 to 2 mg/mL. Cell lysates were diluted with modified RIPA lysis buffer to achieve final protein concentration of ˜1 mg/mL for labeling reactions. Cell pellets obtained from a spleen or a liver were lysed with 400 μL of ice-cold Brij lysis buffer (1% Brij 97, 50 mM triethanolamine pH 7.4, 150 mM NaCl, 5×EDTA-free Roche protease inhibitor cocktail) as mentioned above. Protein concentration was determined by the BCA assay. Typical lysate protein concentrations obtained: spleen 10 mg/mL, liver 10 mg/mL. Cell lysates were diluted with Brij lysis buffer to achieve final protein concentration of ˜1 mg/mL for labeling reactions.

### Staudinger Ligation.

Cell lysates (50 μg) in 46.5 μL modified RIPA lysis buffer were reacted with 1 μL phosphinebiotin (200 μM, 10 mM stock solution in DMSO) and 2.5 μL DTT (5 mM, 100 mM stock solution in deionized water) for a total reaction of volume of 50 μL for 1 hour at room temperature (Vocadlo et al., (2003) Proc. Nat. Acad. Sci. U.S.A. 100, 9116-9121). DTT prevents non-specific oxidation of phosphine-biotin, which can increase levels of background labeling. The reactions were terminated by the addition of −20° C. methanol (1 mL) and placed at −20° C. for at least 1 hour, centrifuged at 18,000 g for 10 minutes at 0° C. to precipitate proteins. The supernatant from the samples was discarded. The protein pellets were allowed to air dry for 10 minutes, resuspended in 354 of resuspension buffer (4% SDS, 50 mM triethanolamine pH 7.4, 150 mM NaCl), diluted with 12.5 μL 4× reducing SDS-loading buffer (40% glycerol, 200 mM Tris-HCl pH 6.8, 8% SDS, 0.4% bromophenol blue) and 2.5 μL 2-mercaptoethanol, heated for 5 minutes at 95° C. and ˜20 μg of protein was loaded per gel lane for separation by SDS-PAGE (10% or 4-20% Bio-Rad Criterion Tris-HCl gel).

Cu^{I}-catalyzed Huisgen [3+2] Cycloaddition.

Cell lysates (50 μg) in 47 μL modified RIPA lysis buffer were reacted with 3 μL freshly premixed click chemistry reaction cocktail [azido- or alkynyl-detection tag (100 μM, 10 mM stock solution in DMSO), tris(2-carboxyethyl)phosphine hydrochloride (TCEP) (1 mM, 50 mM freshly prepared stock solution in deionized water), tris[(1-benzyl-1H-1,2,3-triazol-4-yl)methyl]amine (TBTA) (100 μM, 10 mM stock solution in DMSO), and CuSO₄.5H₂O (1 mM, 50 mM freshly prepared stock solution in deionized water)] for a total reaction volume of 50 μL for 1 hour at room temperature. The reactions were terminated by the addition of ice-cold methanol (1 mL) and placed at −80° C. overnight, centrifuged at 18,000 g for 10 minutes at 4° C. to precipitate proteins. The supernatant from the samples was discarded. The protein pellets were allowed to air dry for 10 minutes, resuspended in 35 μL of resuspension buffer (4% SDS, 50 mM triethanolamine pH 7.4, 150 mM NaCl), diluted with 12.5 μL 4× reducing SDS-loading buffer (40% glycerol, 200 mM Tris-HCl pH 6.8, 8% SDS, 0.4% bromophenol blue) and 2.5 μL 2-mercaptoethanol, heated for 5 minutes at 95° C. and ˜20 μg of protein was loaded per gel lane for separation by SDS-PAGE (10% or 4-20% Bio-Rad Criterion™ Tris-HCl gel).

### Immunoprecipitation.

Cell pellets obtained from 15×10⁶ Jurkat cells were lysed with 50 μL of ice-cold Brij lysis buffer (1% Brij 97, 50 mM triethanolamine pH 7.4, 150 mM NaCl, 5×EDTA-free Roche protease inhibitor cocktail, 10 mM PMSF) by first disrupting the pellet by sonication, and then vortexing 3×10 seconds, cooling the lysate on ice between pulses. Cell lysates were collected after centrifuging at 1,000 g for 5 minutes at 4° C. to remove cell debris. Protein concentration was determined by the BCA assay. Typical lysate protein concentration obtained: 6-8 mg/mL. LAT, Lck and Ras proteins were immunoprecipitated from 200 μg Jurkat cell lysate using the following antibodies at recommended concentrations: mouse anti-Lck (p56lck) monoclonal (Clone 3A5, Thermo Scientific, Waltham, Mass., USA), rat anti-v-H-ras (Ab-1) monoclonal (Y13-259 agarose conjugate, Calbiochem, San Diego, Calif., USA), and rabbit anti-LAT polyclonal (Millipore, Billerica, Mass., USA). 25 μL of packed protein A-agarose beads (Roche, Mannheim, Germany) was used per sample. Upon incubation at 4° C. for an hour with an end-over-end rotator (Barnstead/Thermolyne, Waltham, Mass., USA), the beads were washed three times with ice cold modified RIPA lysis buffer (1% Triton X-100, 1% sodium deoxycholate, 0.1% SDS, 10 mM Tris pH 7.4, 150 mM NaCl). The beads were resuspended in 20 μL of resuspension buffer (4% SDS, 50 mM triethanolamine pH 7.4, 150 mM NaCl) and freshly premixed click chemistry reagents (same as above) were added. After 1 hour at room temperature, the reaction mixture was diluted with 6.7 μL 4× reducing SDS-loading buffer (40% glycerol, 200 mM Tris-HCl pH 6.8, 8% SDS, 0.4% bromophenol blue) and 1.3 μL 2-mercaptoethanol, heated for 5 minutes at 95° C. and 20 μL of the supernatant was loaded per gel lane for separation by SDS-PAGE (10% or 4-20% Bio-Rad Criterion Tris-HCl gel).

### In-Gel Fluorescence Scanning.

Proteins separated by SDS-PAGE were visualized by directly scanning the gel on an Amersham Biosciences Typhoon 9400 variable mode imager (excitation 532 nm, 580 nm filter, 30 nm band-pass) (Piscataway, N.J., USA).

### Immuno-Blotting.

Proteins separated by SDS-PAGE were transferred (50 mM Tris, 40 mM glycine, 0.0375% SDS, 20% MeOH in deionized water, Bio-Rad Trans-Blot Semi-Dry Cell, 20 V, 1 hour) onto a PVDF membrane which was blocked (5% non-fat dried milk, 1% BSA and 0.1% Tween-20 in PBS) for 1 hour at 25° C. or overnight at 4° C. The membrane was washed thrice with PBST (0.1% Tween-20 in PBS), incubated with streptavidin-horseradish peroxidase (1 mg/mL diluted 1:25,000 in PBST, Pierce, Waltham, Mass., USA), and subsequently developed with ECL Western blotting detection reagents (Amersham, Piscataway, N.J., USA). Alternatively, Lck, LAT and Ras protein levels were visualized by incubating the blots at recommended concentrations in 5% casein, 1% BSA in PBST with mouse anti-Lck (p56lck) monoclonal (Clone 3A5, Thermo Scientific, Waltham, Mass., USA), mouse anti-LAT monoclonal (2E9, Millipore, Billerica, Mass., USA)) or mouse anti-Ras monoclonal (RAS10, Millipore, Billerica, Mass., USA)), respectively, followed by a goat anti-mouse-HRP conjugated secondary antibody (Millipore, Billerica, Mass., USA) in the blocking buffer mentioned above.

### Example 2

### Synthesis of Chemical Reports and Detection Tags

### General Procedures:

All chemicals were obtained either from Sigma-Aldrich (Saint Louis, Mo., USA), MP Biomedicals (Solon, Ohio, USA), Alfa Aesar (Ward Hill, Mass., USA), TCI America (Portland, Ore, USA), Fluka (Division of Sigma-Aldrich) or Acros Organics USA (Morris Plains, N.J., USA) and were used as received unless otherwise noted. The silica gel used in flash column chromatography was Fisher 5704 (60-200 Mesh, Chromatographic Grade). Analytical thin layer chromatography (TLC) was conducted on Merck silica gel plates with fluorescent indicator on glass (5-20 μm, 60 Å) with detection by ceric ammonium molybdate, basic KMnO₄ or UV light. The ¹H and ¹³C NMR spectra were obtained on a Bruker DPX-400 spectrometer or a Bruker AVANCE-600 spectrometer equipped with a cryoprobe. Chemical shifts are reported in δ ppm values downfield from tetramethylsilane and J values are reported in Hz. MALDI-TOF mass spectra were obtained on an Applied Biosystems Voyager-DE. LC/MS were obtained on a Waters 500E pump and controller equipped with a Waters XBridge C18 5 μm 4.6×150 mm column, Waters 996 photodiode array detector and Waters Micromass ZQ mass spectrometer and the samples were single peak purity. 4-(2-azidoethyl)phenol (Battersby, A. R., Chrystal, E. J., Staunton, J. (1980) J. Chem. Soc. [Perkin 1], 1: 31-42); 6-heptynoic acid-NHS ester (Luo, Y., Knuckley, B., Lee, Y. H., Stallcup, M. R., Thompson, P. R. (2006) J. Am. Chem. Soc., 128: 1092-1093); and biotin-PEG-NH₂ (Wilbur, d. S., Hamlin, D. K., Vessella, R. L., Stray, J. E., Buhler, K. R., Stayton, P. S., Klumb, L. A., Pathare, P. M., Weerawarna, S. A. (1996) Bioconjug. Chem., 7: 689-702) were synthesized as previously described.

### Alkynyl-Fatty Acids Synthesis:

Alkynyl-fatty acids were synthesized according to reported procedures and were identical by ¹H NMR analysis. (alk-12 and alk-14: Hebert et al., (1992) J. Org. Chem. 57, 1777-1783 and alk-16: Augustin and Schaefer (1991) Liebigs Ann. Chem. (1991) 1991, 1037-1040).

Tetradec-13-ynoic acid (alk-12): ¹H NMR (400 MHz, CDCl₃): δ 1.22-1.30 (s, 14H), 1.29-1.70 (m, 4H), 1.96 (t, 1H, J=4), 2.22 (dt, 2H, J=4, 7), 2.36 (t, 2H, J=7).

Hexadec-15-ynoic acid (alk-14): ¹H NMR (400 MHz, CDCl₃): δ 1.22-1.30 (s, 18H), 1.29-1.70 (m, 4H), 1.96 (t, 1H, J=4), 2.22 (dt, 2H, J=4, 7), 2.36 (t, 2H, J=7).

Octadec-17-ynoic acid (alk-16): ¹H NMR (600 MHz, CDCl₃): δ 1.22-1.30 (m, 18H), 1.29-1.35 (m, 2H), 1.35-1.42 (m, 2H), 1.52 (qu, 2H, J=7.1), 1.63 (qu, 2H, J=7.5), 1.93 (t, 1H, J=2.6), 2.18 (dt, 2H, J=2.6, 7.1), 2.35 (t, 2H, J=7.5).

### Biotin Detection Tag Synthesis:

N-(15-oxo-19-((3aS,4S,6aR)-2-oxohexahydro-1H-thieno[3,4-d]imidazol-4-yl)-4,7,10-trioxa-14-azanonadecyl)hept-6-ynamide (alk-biotin): In a round-bottom flask equipped with a magnetic stir bar was dissolved 6-heptynoic acid (819 mg, 6.5 mmol) in CH₂Cl₂ (10 mL). N-methylmorpholine (656 mg, 6.5 mmol) and isobutyl-chloroformate (890.5 mg, 6.5 mmol) were added, and this reaction mixture was stirred for 30 minutes at 0° C. Then, this solution of activated acid was transferred via a syringe to a solution of amine-biotin (538 mg, 1.3 mmol) in DMF (10 mL) in another round-bottom flask equipped with a magnetic stir bar and stirred at room temperature for 3 hours. (Wilbur et al., (1996) Bioconjugate Chem. 7, 689-702) The solvent was evaporated under reduced pressure and the crude mixture was purified by flash chromatography on silica gel (60% EtOAc/30% MeOH/10% H₂O) to afford 332 mg of alk-biotin as a white solid (62%). ¹HNMR (400 MHz, CDCl₃): δ 1.5 (m, 2H), 1.6 (m, 2H), 1.6-1.8 (m, 10H), 1.9 (t, 1H, J=2.5), 2.2 (m, 6H), 2.7 (d, 1H, J=12.7), 2.9 (dd, 1H, J=4.8, 12.7), 3.1 (ddd, 1H, J=4.8, 7.4, 7.4), 3.2-3.4 (m, 4H), 3.5-3.7 (m, 12H), 4.3 (m, 1H), 4.5 (m, 1H), 5.2 (br, 1H), 5.9 (br, 1H), 6.4 (br, 1H), 6.6 (br, 1H). ¹³C NMR (100 MHz, CDCl₃): δ 18.6, 25.2, 26.1, 28.4, 28.5, 28.6, 29.4, 36.4, 38.0, 40.9, 56.1, 60.6, 62.2, 69.0, 70.3, 70.8, 84.6, 164.3, 173.2, 173.6. MALDI-TOF: 555.4 [M+H]⁺.

5-azido-N-(15-oxo-19-((3aS,4S,6aR)-2-oxohexahydro-1H-thieno[3,4-d]imidazol-4-yl)-4,7,10-trioxa-14-azanonadecyl)pentanamide (az-biotin): In a round-bottom flask equipped with a magnetic stir bar was dissolved 5-azidopentanoic acid (140 mg, 1 mmol) in CH₂Cl₂ (10 mL). N-methylmorpholine (121 mg, 1.2 mmol) and isobutyl-chloroformate (163.8 mg, 1.2 mmol) were added, and this reaction mixture was stirred for 30 minutes at 0° C. Then, this solution of activated acid was transferred via a syringe to a solution of amine-biotin (150 mg, 0.3 mmol) in DMF (10 mL) in another round-bottom flask equipped with a magnetic stir bar and stirred at room temperature for 3 hours. (Wilbur et al., (1996) Bioconjugate Chem. 7, 689-702) The solvent was evaporated under reduced pressure and the crude mixture was purified by flash chromatography on silica gel (70% EtOAc/20% MeOH/10% H₂O) to afford 76 mg of az-biotin as a white solid (45%). ¹HNMR (400 MHz, CDCl₃): δ 1.5 (m, 2H), 1.6-1.8 (m, 12H), 2.2 (m, 4H), 2.7 (d, 1H, J=12.7), 2.9 (dd, 1H, J=4.8, 12.7), 3.2 (ddd, 1H, J=4.8, 7.4, 7.4), 3.3-3.4 (m, 6H), 3.5-3.7 (m, 12H), 4.3 (m, 1H), 4.5 (m, 1H), 5.2 (br, 1H), 5.8 (br, 1H), 6.4 (br, 1H), 6.6 (br, 1H). ¹³C NMR (100 MHz, CDCl₃): δ 19.5, 22.9, 23.2, 26.4, 26.6, 28.4, 28.7, 28.9, 36.2, 37.9, 51.5, 66.2, 68.5, 70.1, 168.03, 168.04, 173.1. MALDI-TOF: 572.5 [M+H]⁺.

Synthesis of Detection Tags with Cleavable Biotin Affinity Purification Tags

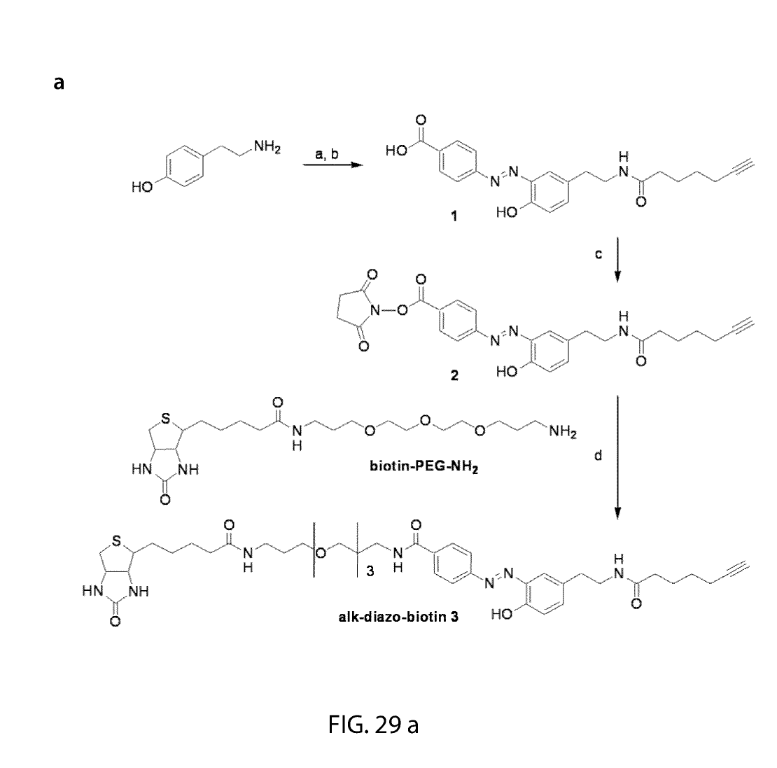

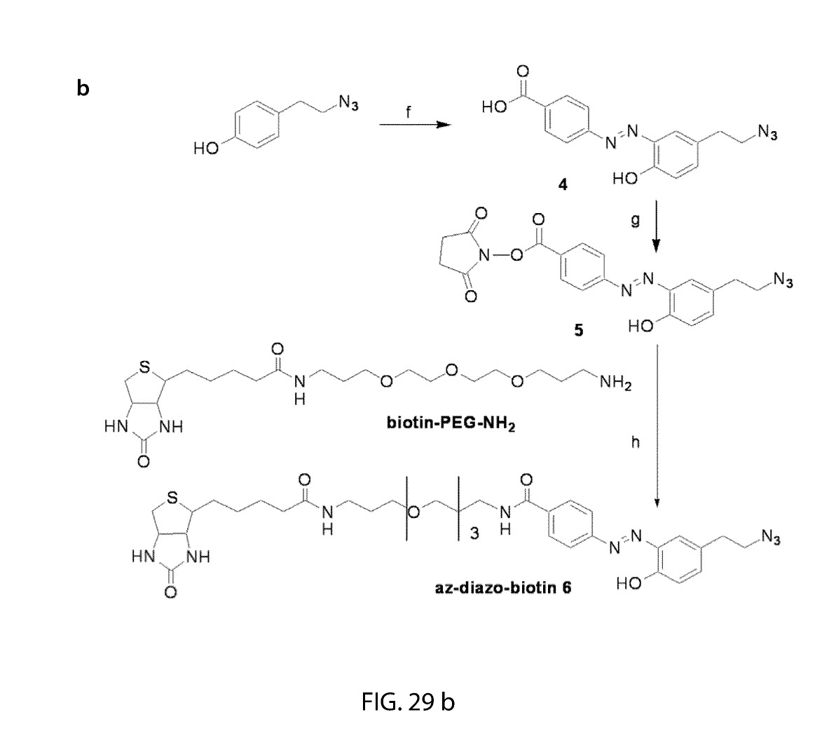

A general synthesis scheme for compounds (1), (2), and (3) is shown in FIG. 29A.

(E)-4-((5-(2-hept-6-ynamidoethyl)-2-hydroxyphenol)diazenyl)benzoic acid (1).

Solid NaNO₂ (345 mg, 5 mmol) was added to a cooled suspension of 4-aminobenzoic acid (275 mg, 2 mmol) in 6 M HCl (4 mL) in a 100 mL round-bottomed flask. The resulting mixture was stirred at 0° C. and turned into a slightly yellowish-brown solution. After 30 minutes the diazonium salt solution was slowly added to a solution of tyramine (137 mg, 1 mmol) in THF (10 mL) and a slight excess of triethylamine to keep the solution basic. The solution was slowly allowed to warm to rt overnight. Next, 6-heptynoic acid-NHS ester2 (280 mg, 1.1 mmol) was added to the reaction mixture and stirred 1 hour. The reaction was acidified with 10% aqueous HCl, diluted with water (25 mL) and extracted with EtOAc (3×15 mL). The combined organic layers were then washed with water, saturated NaHCO₃, dried over Na₂SO₄ and concentrated under reduced pressure. The crude solid was purified by silica gel flash chromatography, eluting MeOH:CH₂Cl₂ (1:9). The fractions containing the desired compound were concentrated under reduced pressure to yield 270 mg of a reddish orange solid (70% yield). ¹H-NMR (DMSO-d₆): ^{TM} 1.3-1.5 (m, 2H), 1.5-1.6 (m, 2H), 2.0-2.1 (t, 2H, J=7.7), 2.1-2.2 (t, 2H, J=7.4), 2.7 (m, 3H), 3.2-3.3 (m, 2H), 7.0 (d, 1H, J=7.9), 7.3 (d, 1H, J=7.9), 7.6 (s, 1H), 7.7 (m, 1H), 8.0-8.1 (d, 2H), 8.1-8.2 (d, 2H, J=7.6); ¹³C-NMR (DMSO-d₆): ^{TM} 18.2, 25.2, 28.3, 34.9, 35.6, 72.0, 75.6, 85.1, 119.1, 122.3, 123.4, 131.3, 131.8, 133.2, 135.8, 139.2, 154.6, 154.9, 157.7, 167.6, 172.6. MALDI-TOF calculated for C₂₂H₂₄N₃O₄ [M+H]⁺394.1, found 394.4.

(E)-2,5-dioxopyrrolidin-1-yl-4-((5-(2-hept-6-ynamidoethyl)-2-hydroxyphenyl)diazenyl)benzoate (2). Compound (1) (50 mg, 0.13 mmol) was added to a solution of N-hydroxysuccinimide (14.9 mg, 0.13 mmol) in EtOAc (10 mL) in a 100 mL round-bottomed flask. A solution of dicyclohexylcarbodiimide (16.4 mg, 0.13 mmol) in EtOAc (5 mL) was then added and the reaction was stirred over night at rt. The reaction mixture filtered and concentrated under reduced pressure to yield white crystals. The crude material was purified by silica gel flash chromatography, eluting with hexanes:EtOAc (7:3). The fractions containing the product were combined, concentrated under vacuum to yield 30 mg of a white solid (50% yield). ¹H-NMR (CDCl₃): ^{TM} 1.5-1.6 (m, 2H), 1.7-1.8 (m, 2H), 1.9 (s, 1H), 2.2 (m, 4H), 2.9 (m, 2H), 2.9-3.0 (m, 4H), 3.6 (m, 2H), 7.0 (d, 1H, J=7.0), 7.3 (t, 1H, J=7.4), 7.8 (s, 1H), 8.0 (d, 2H, J=8.0), 8.3 (d, 2H, J=8.0). ¹³C-NMR (DMSO-d₆): ^{TM} 20.6, 24.2, 25.1, 27.7, 35.6, 35.7, 41.1, 68.7, 83.3, 116.2, 122.9, 125.1, 129.8, 131.4, 132.4, 149.4, 157.9, 164.7, 169.7, 171.5. MALDI-TOF calculated for C₂₆H₂₆N₄O₆Na [M+Na]⁺513.2, found 513.2.

(I) (E)-4-((5-(2-hept-6-ynamidoethyl)-2-hydroxyphenyl)diazenyl)-N-(15-oxo-19-(2-oxohexa hydro-1H-thieno[3,4-a]imidazol-4-yl)-4,7,10-trioxa-14-azanonadecyl)benzamide (3). To a 100 mL flask was added compound (2) (30 mg, 0.07 mmol) in DMF (2 mL), biotin-PEG-NH₂³ (N-(13-Amino-4,7,10-trioxamidecanyl)biotinamide, 30.3 mg, 0.07 mmol) in DMF (10 mL). The reaction mixture was stirred at rt for 4 hours and concentrated down under vacuum. The crude material was purified by silica gel flash chromatography, eluting with EtOAc:MeOH:water (30:2:1 to 10:2:1). The fractions containing the product were combined, concentrated under vacuum to yield 14 mg of a white solid (24% yield). ₁H-NMR (DMSO-d₆): ^{TM} 1.3-1.4 (m, 3H), 1.4-1.7 (m, 10H), 1.7-1.8 (m, 2H), 2.0-2.1 (m, 4H), 2.1 (m, 2H), 2.6-2.7 (m, 2H), 2.75 (s, 1H), 2.8 (m, 1H), 3.0-3.1 (m, 4H), 3.4-3.5 (m, 14H), 4.1-4.2 (s, 1H), 4.3 (m, 1H), 6.3 (s, 1H), 6.4 (s, 1H), 7.0 (d, 1H, J=6.6), 7.1-7.2 (m, 2H), 7.2-7.3 (m, 2H), 7.6 (s, 1H), 7.7-7.8 (t, 1H, J=6.8), 7.8-7.9 (t, 1H), 8.6 (m, 1H). ¹³C-NMR (CD₃OD): δ 17.3, 24.8, 25.1, 27.7, 27.8, 28.3, 29.9, 31.0, 34.1, 35.0, 35.2, 36.5, 37.3, 39.9, 41.1, 55.5, 61.7, 63.4, 64.2, 67.8, 70.5, 123.1, 125.1, 127.8, 131.4, 132.4, 136.4, 149.4, 164.7, 167.7. MS (ESI) calcd. for C₄₂H₆₀N₇O₈S 822.4 [M+H]⁺, found 822.2; MALDI-TOF calcd. for C₄₂H₅₉N₇O₈SNa [M+Na]⁺844.4, found 844.5.

A general synthesis scheme for compounds (4), (5), and (6) is shown in FIG. 29B.

(E)-4-((5-(2-azidoethyl)-2-hydroxyphenyl)diazenyl)benzoic acid (4). Solid NaNO₂ (3.30 g, 6.55 mmol) was added to an ice-cooled suspension of 4-aminobenzoic acid in 6M HCl (40 mL). The resulting mixture was stirred at 0° C. and turned into a yellow-colored solution. In the meantime, 4-(2-azidoethyl)phenol1 (1.19 g, 7.30 mmol) was dissolved in THF (15 mL) and cooled to 0° C., followed by the addition of K₂CO₃ to adjust the reaction mixture to pH 8. After 40 minutes, the diazonium salt solution was slowly added to reaction mixture containing compound (2) at 0° C. The pH of the reaction mixture was kept around pH 8 by adding more K₂CO₃. The reaction mixture was stirred for 4 hours at rt, concentrated under vacuum, dissolved in EtOAc (100 mL), washed with 10% aqueous HCl (3×50 mL), dried over MgSO₄, filtered and concentrated. The crude product was purified by silica gel flash column chromatography, eluting with EtOAc:hexanes (1:2) followed by MeOH:CH₂Cl₂ (1:9) to afford 1.27 g of yellow-colored compound (4) (56% yield). ¹H-NMR (CD₃OD, 600 MHz): ^{TM} 8.24 (d, 2H, J=8.5 Hz), 8.05 (d, 2H, J=8.5 Hz), 7.89 (d, 1H, J=1.9 Hz), 7.40 (dd, 1H, J=8.3, 2.0 Hz), 7.04 (d, 1H, J=8.4 Hz), 3.61 (t, 2H, J=7.0 Hz), 2.97 (t, 2H, J=7.0 Hz); ¹³C-NMR (CD₃OD, 150 MHz): ^{TM} 176.6, 163.9, 163.8, 148.3, 144.9, 140.4, 139.6, 132.5, 131.3, 128.2, 61.4, 43.3. MS (ESI) calcd for C₁₅H₁₃N₅O₃ [M−H]⁻: 310.0940. Found 310.2.

(E)-2,5-dioxopyrrolidin-1-yl-4-((5-(2-azidoethyl)-2-hydroxyphenyl)diazenyl)benzoate (5). To compound (4) (164 mg, 0.53 mmol) dissolved in anhydrous THF (20 mL) was added dicyclohexyl carbodiimide (119 mg, 0.58 mmol) and N-hydroxy-succinimide (66.7 mg, 0.58 mmol) under Ar. The reaction was stirred at room temperature for 4 hours, concentrated, redissolved in EtOAc, filtered and concentrated under vacuo. The crude product was purified by silica gel flash column chromatography, eluting with EtOAc:hexanes (1:2) to give 170 mg of yellow-colored compound 5 (79% yield). ¹H-NMR (CDCl₃, 600 MHz): ^{TM} 8.31 (d, 2H, J=8.3 Hz), 8.00 (d, 2H, J=8.3 Hz), 7.87 (d, 1H, J=1.9 Hz), 7.31 (dd, 1H, J=8.5, 2.0 Hz), 7.05 (d, 1H, J=8.5 Hz), 3.60 (t, 2H, J=7.0 Hz), 2.95-2.98 (m, 6H); ¹³C-NMR (CDCl₃, 150 MHz): ^{TM} 169.1, 161.2, 154.3, 151.7, 137.4, 135.3, 133.6, 131.9, 130.0, 126.6, 122.4, 118.7, 52.4, 34.2, 25.7. MS (ESI) [M+H]⁺: 409.1260. Found: 409.3.

(II) (E)-4-((5-(2-azidoethyl)-2-hydroxyphenyl)diazenyl)-N-(15-oxo-19-(2-oxohexahydro-1H thieno[3,4-d]imidazol-4-yl)-4,7,10-trioxa-14-azanonadecyl)benzamide (6): To compound (5) (13 mg, 0.03 mmol) dissolved in anhydrous DMF (3 mL) was added biotin-PEG-NH₂³ (27.0 mg, 0.06 mmol). The reaction stirred at room temperature for 10 hours and concentrated under vacuum. The crude product was re-dissolved in CH₃CN:H2O (1:1) and purified by reversed preparative HPLC column (CH₃CN: 5% to 40% in 10 minutes, then 40% to 100% in 40 minutes, compound 6 was eluted at 33 minutes) to give yellow-colored product (18.0 mg, 80%). ¹H-NMR (CD₃OD, 400 MHz): ^{TM} 8.44 (brs, 1H), 8.00 (brs, 4H), 7.83 (d, 1H, J=2.0 Hz), 7.33 (dd, 1H, J=8.5, 2.0 Hz), 6.99 (d, 1H, J=8.4 Hz), 4.46 (dd, 1H, J=7.7, 5.0 Hz), 4.27 (dd, 1H, J=7.8, 4.4 Hz), 3.66-3.47 (m, 18H), 3.23 (t, 2H, J=6.7 Hz), 3.16 (td, 1H, J=4.6, 9.2 Hz), 2.92 (t, 2H, J=7.0 Hz), 2.68 (d, 1H, J=12.7 Hz), 2.16 (t, 2H, J=7.3 Hz), 1.93 (t, 1H, J=6.3 Hz), 1.89 (t, 1H, J=6.3 Hz), 1.76-1.52 (m, 6H), 1.42 (t, 1H, J=7.6 Hz), 1.38 (t, 1H, J=7.7 Hz), 1.28 (brs, 2H), 0.89 (m, 1H); ¹³C-NMR (CD₃OD, 100 MHz): ^{TM} 175.9, 169.1, 166.1, 154.3, 153.3, 139.0, 137.8, 135.8, 131.8, 131.3, 129.6, 123.4, 119.3, 71.5, 71.3, 71.2, 70.3, 63.3, 61.6, 57.0, 53.5, 41.0, 38.9, 37.8, 36.8, 35.1, 30.7, 30.4, 29.8, 29.4, 26.8. MALDI-TOF calculated for C₃₅H₄₉N₉O₇S [M+Na]⁺: 762.8744. Found: 762.26.

The alkynyl detection tag with cleavable biotin affinity purification tag (IV) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-buta-4-ynl)-phenylazo]-benzamide and the azido detection tag with cleavable biotin affinity purification tag (III) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-2-azido-ethyl)-phenylazo]-benzamide were synthesized in a manner similar to the synthesis of compound (1) and (II) respectively.

### Rhodamine Detection Tags Synthesis:

N-(6-(diethylamino)-9-(2-(4-hept-6-ynoylpiperazine-1-carbonyl)phenyl)-3H-xanthen-3-ylidene)-N-ethylethanaminium (alk-rho): In a previously flame-dried under an argon atmosphere round-bottom flask equipped with a magnetic stir bar, was dissolved 6-heptynoic acid (6 mL, 0.050 mmol) in dry DMF (0.5 mL). 1-1′-Carbonyl diimidazole (8 mg, 0.050 mmol) was added in one portion, and the reaction mixture was stirred at room temperature for one hour. Rhodamine B piperazine amide (24 mg, 0.044 mmol) was then added in one portion and the reaction mixture was stirred at room temperature overnight. (Nguyen and Francis (2003) Org. Lett. 5, 3245-3248) The solvent was evaporated under reduced pressure and the crude mixture was purified by flash chromatography on silica gel (80% EtOAc/13% MeOH/7% H₂O) to afford 22 mg of alk-rho as a purple solid (76%). ¹H NMR (400 MHz, CD₃OD): δ 1.31 (t, 12H, J=7.1), 1.46-1.57 (m, 2H), 1.61-1.71 (m, 2H), 2.15-2.25 (m, 3H), 2.38 (t, 2H, J=7.3), 3.4 (br, 8H), 3.70 (quartet, 8H, J=7.1), 6.98 (d, 2H, J=2.4), 7.08 (dd, 2H, J=2.4, 9.5), 7.29 (d, 2H, J=9.5), 7.50-7.55 (m, 1H), 7.68-7.73 (m, 1H), 7.76-7.80 (m, 2H). ¹³C NMR (100 MHz, CD₃OD): δ 12.8, 18.7, 25.3, 29.1, 33.4, 42.7, 46.0, 46.9, 69.9, 84.7, 97.4, 114.9, 115.4, 128.9, 131.2, 131.3, 131.8, 132.3, 133.2, 136.5, 157.0, 157.2, 159.3, 169.6, 174.0. LCMS: 619.55 [M]⁺.

N-(9-(2-(4-(6-azidohexanoyl)piperazine-1-carbonyl)phenyl)-6-(diethylamino)-3H-xanthen-3-ylidene)-N-ethylethanaminium (az-rho): In a previously flame-dried under an argon atmosphere round-bottom flask equipped with a magnetic stir bar, was dissolved 6-azidohexanoic acid (8 mg, 0.050 mmol) in dry DMF (0.5 mL). 1-1′-Carbonyl diimidazole (8 mg, 0.050 mmol) was added in one portion, and the reaction mixture was stirred at room temperature for one hour. Rhodamine B piperazine amide (25 mg, 0.046 mmol) was then added in one portion and the reaction mixture was stirred at room temperature overnight. (Nguyen and Francis (2003) Org. Lett. 5, 3245-3248) The solvent was evaporated under reduced pressure and the crude mixture was purified by flash chromatography on silica gel (80% EtOAc/13% MeOH/7% H₂O) to afford 22 mg of az-rho as a purple solid (70%). ¹H NMR (400 MHz, CD₃OD): 1.31 (t, 12H, J=7.1), 1.34-1.48 (m, 2H), 1.53-1.68 (m, 4H), 2.36 (t, 2H, J=7.4), 3.3 (m, 2H), 3.40 (br, 8H), 3.69 (quartet, 8H, J=7.1), 6.97 (d, 2H, J=2.4), 7.08 (dd, 2H, J=2.4, 9.5), 7.28 (d, 2H, J=9.5), 7.50-7.54 (m, 1H), 7.68-7.72 (m, 1H), 7.75-7.80 (m, 2H). ¹³C NMR (100 MHz, CD₃OD): δ 12.8, 25.8, 27.4, 29.7, 33.6, 42.7, 46.0, 46.9, 52.3, 97.4, 114.9, 115.4, 128.9, 131.2, 131.3, 131.8, 132.3, 133.2, 136.5, 157.0, 157.2, 159.3, 170.6, 174.0. LCMS: 650.57 [M]⁺.

### Example 3

### Robust Fluorescent Detection of Acylated Proteins with Chemical Reporters

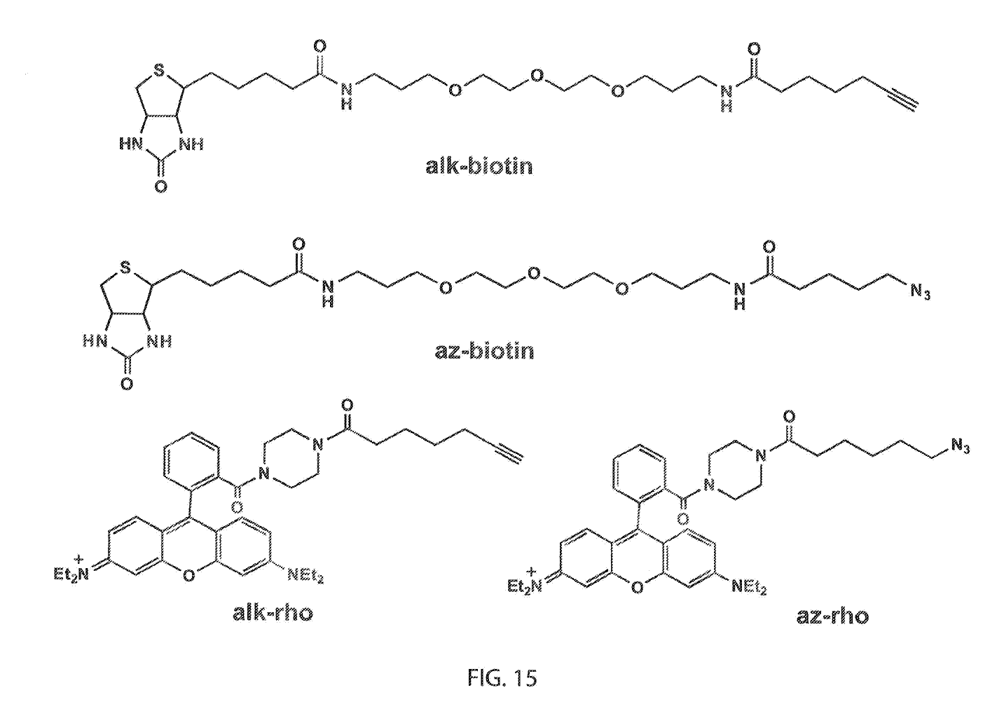

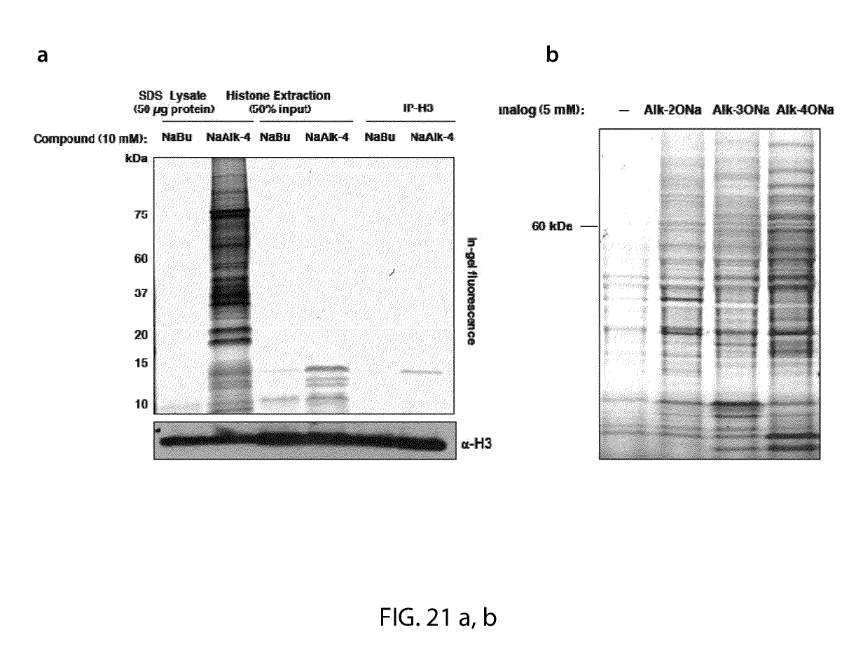

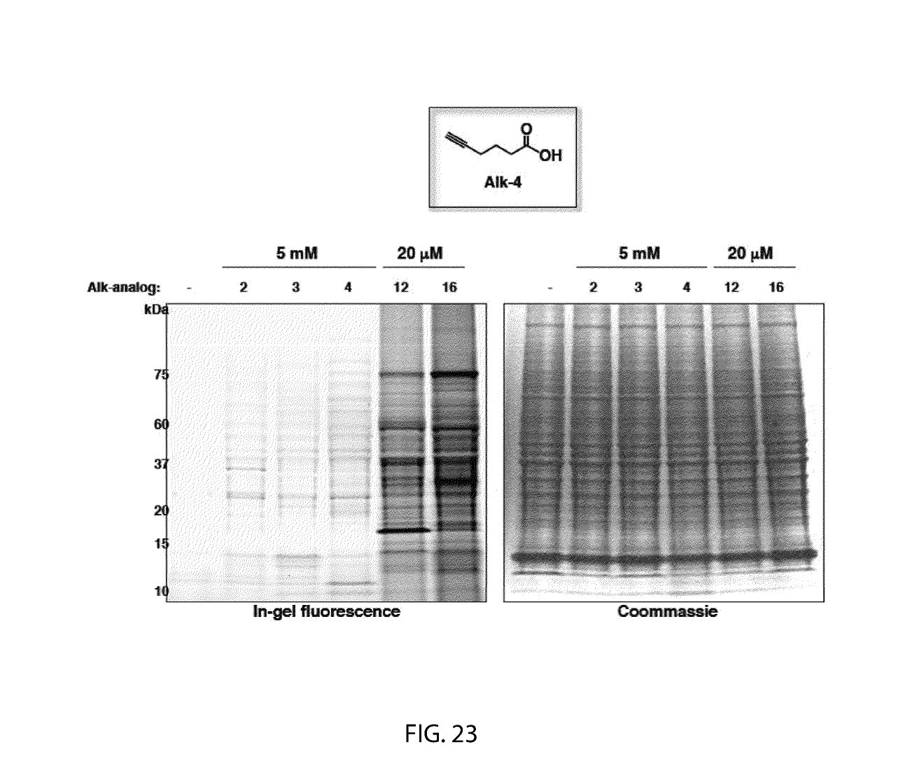

Advances in bioorthogonal labeling methods employing the Cu^{I}-catalyzed Huisgen [3+2] cycloaddition or “click chemistry” reaction between alkyl azides and alkynes (Prescher and Bertozzi (2005) Nat. Chem. Biol. 1, 13-21) (FIG. 4A), suggested an opportunity to improve the analysis of acylated proteins with chemical reporters. We therefore synthesized a series of potential alkynyl-chemical reporters as well as a panel of biotinylated (alk-biotin, az-biotin) and fluorescent (alk-rho, az-rho) detection tags to explore the detection of acylated proteins with click chemistry (FIG. 15). Comparative analysis of the Staudinger ligation and click chemistry reaction with azido-labeled cell lysates and biotinylated detection probes (phos-biotin and alk-biotin, respectively) revealed significantly improved detection of acylated proteins by streptavidin blotting with the Cu^{I}-catalyzed Huisgen [3+2] cycloaddition (FIG. 8). We then investigated whether the orientation of alkyne and azide functional groups would influence the overall sensitivity of acylated protein analysis using click chemistry. Cells were metabolically labeled with azido-(az-12, az-15) or alkynyl-(alk-12, alk-14 & alk-16) chemical reporters and assayed for the specific detection of fatty-acylated proteins in cell lysates using biotin (alk-biotin, az-biotin) or fluorescence (alk-rho, az-rho) detection tags and streptavidin blotting (FIG. 9) or in-gel fluorescence scanning (FIG. 10A), respectively. Profiles of fatty-acylated proteins visualized by in-gel fluorescence scanning revealed substantially more proteins compared to streptavidin blotting, particularly with the palmitic acid analogs (az-15, alk-14 & alk-16). Like their azide counterparts, the alkynyl-chemical reporters (alk-12, alk-14 & alk-16) functioned as efficient chemical reporters of protein acylation and exhibited chain length-dependent protein labeling (FIG. 10A). The shorter chain chemical reporters (az-12 & alk-12) were designed to preferentially label N-myristoylated proteins, whereas the longer chain chemical reporters (az-15, alk-14 & alk-16) were targeted for S-palmitoylated proteins. The nearly identical profile of fatty-acylated proteins visualized by azido- or alkynyl-chemical reporter metabolic labeling and click chemistry in-gel fluorescence analysis reinforces the concept that the small azide and alkyne chemical tags accurately report protein fatty-acylation states with minimal perturbation (FIG. 10A). In addition, very short chain alkynyl-chemical reporters (alk-2, alk-3, and alk-4) were found to label acetylated proteins such as histones (FIG. 21A). In experiments to date, the alk-4 chemical reporter provided the most efficient labeling of acetylated proteins (FIG. 21B and FIG. 23).

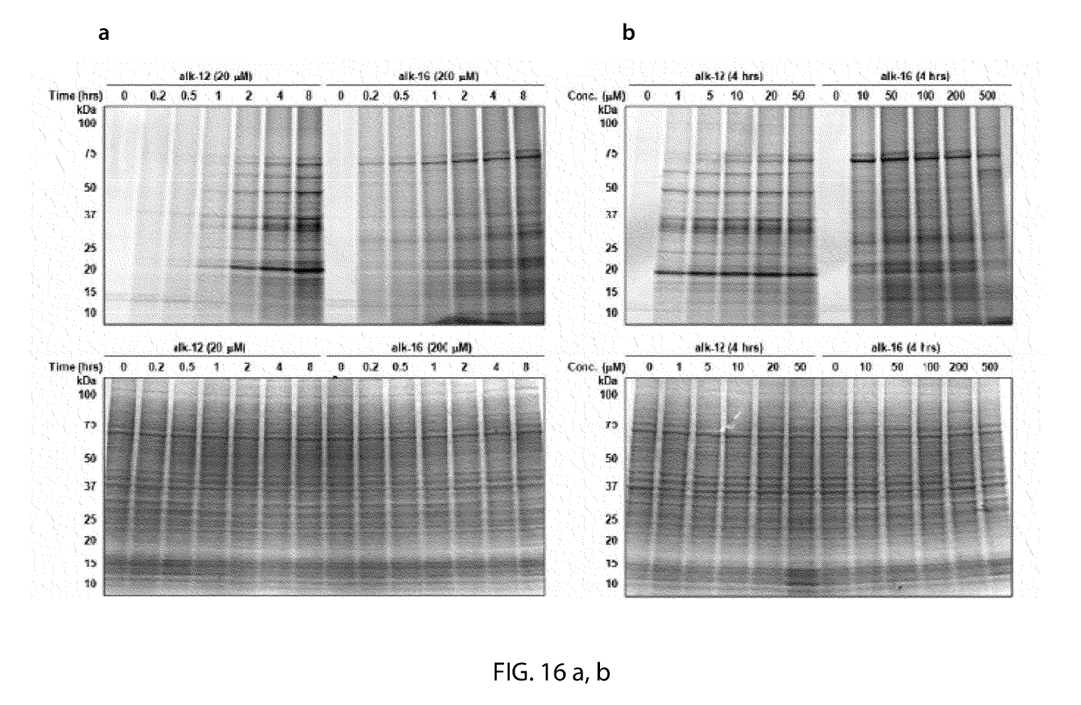

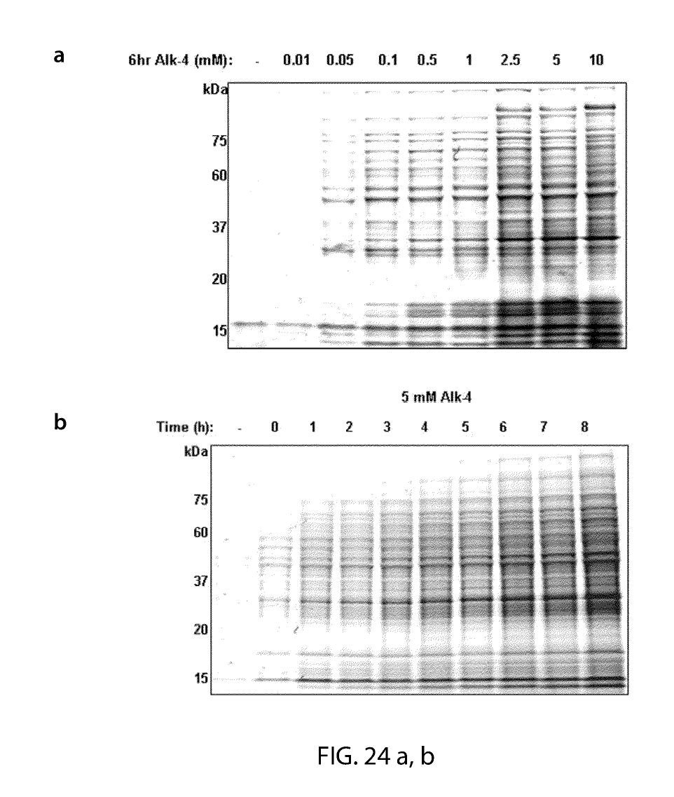

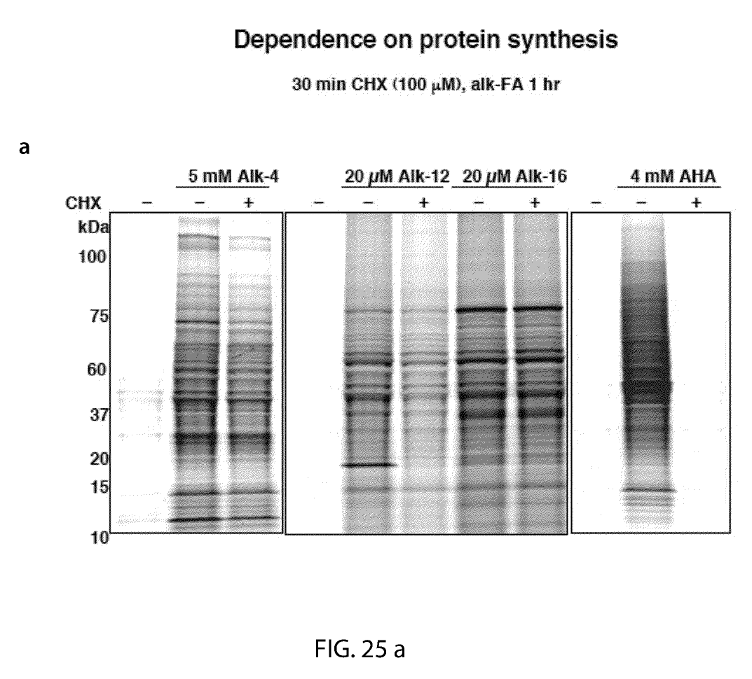

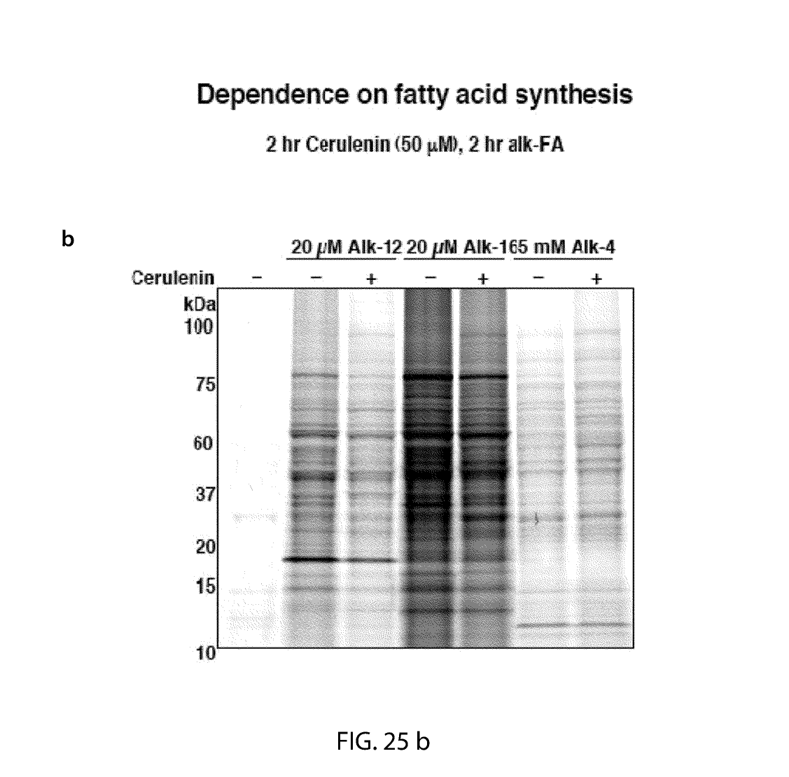

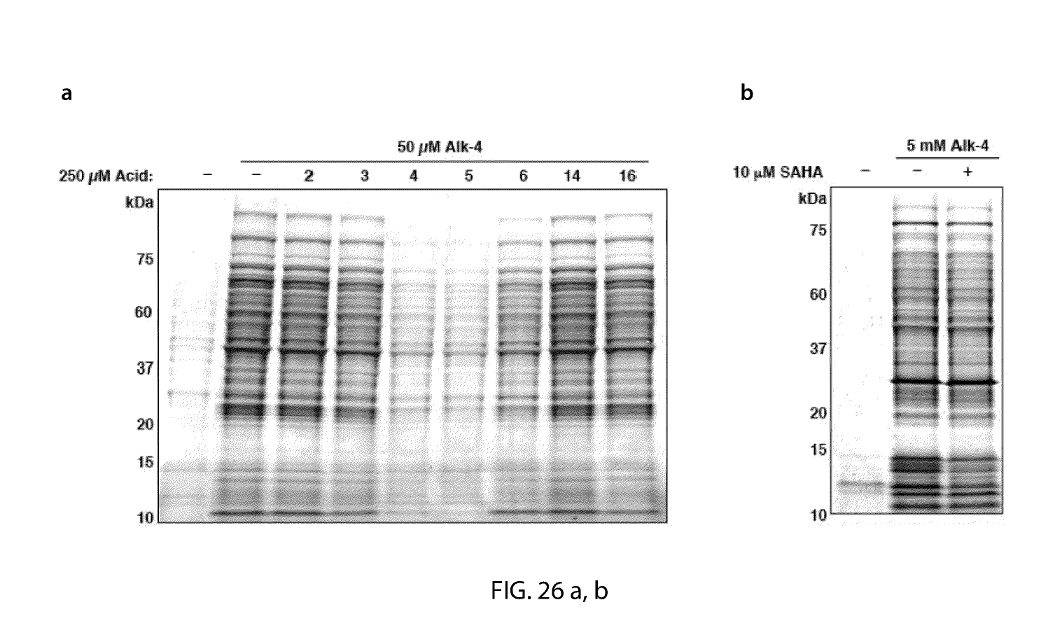

Quantitative comparative analysis of acylated proteins visualized by in-gel fluorescence scanning demonstrated that alkynyl-chemical reporters, in combination with the az-rho detection tag, afforded 2 to 4 fold improvement in specificity of labeling compared to the reverse click chemistry orientation owing to lower background signal (FIG. 10B). These observations are consistent with other studies using alkyne- or azide-functionalized activity-based probes (Speers and Cravatt (2004) Chem. Biol. 11, 535-546). Time- and dose-dependent analyses of metabolic labeling with the alkynyl-chemical reporters revealed that the click chemistry and in-gel fluorescence imaging protocol required shorter labeling time (minutes) and lower concentrations than previous methods (Hang et al., (2007) J. Am. Chem. Soc. 129, 2744-2745) to detect acylated proteins with alkynyl-chemical reporters. FIG. 16 shows the time and dose dependence of labeling with the alk-12 and alk-16 chemical reporters of protein fatty-acylation. FIGS. 24A and 24B show the time and does dependence of labeling with the alk-4 chemical reporter of protein acetylation. To test whether labeling with alkynyl-chemical reporters was dependent on de novo protein synthesis, cells were incubated with the protein synthesis inhibitor cyclohexamide (CHX). Treatment with CHX did not inhibit the metabolic incorporation of chemical reporters of various lengths (i.e, alk-4, alk-12, alk-16; FIG. 25A). Labeling with alkynyl-chemical reporters is also not dependent on de novo fatty acid synthesis. Incubation of cells with the fatty acid synthesis inhibitor cerulenin did not inhibit metabolic incorporation of alk-4, alk-12, or alk-16 (FIG. 25B). Further labeling of acetylated proteins with the short chemical reporter alk-4 was shown to be insensitive to addition of the histone deacetylase inhibitor suberoylanilide hydroxamic acid (SAHA) (FIG. 26B). Labeling of acetylated proteins with alk-4 however was inhibited by the addition of butyric acid (FIG. 26A).

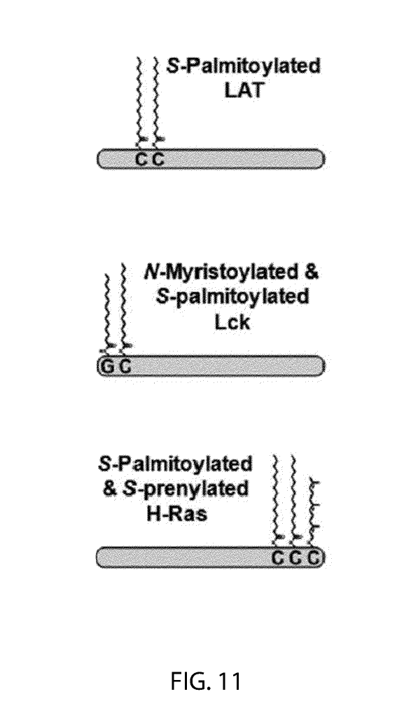

We further evaluated the efficiency and specificity of our chemical reporters in the detection of different classes of fatty-acylated proteins (Resh, M. D. (2006) Nat. Chem. Biol., 2: 584-590): N-myristoylated & S-palmitoylated—Lck, S-palmitoylated only—Linker for Activation of T Cells (LAT), and S-palmitoylated & S-prenylated Ras (FIG. 11). For these studies, Jurkat cells were metabolically labeled with azido-(az-12, az-15) or alkynyl-(alk-12, alk-16) chemical reporters. Proteins of interest were immunoprecipitated from cell lysates, subjected to click chemistry with biotinylated (alk-biotin, az-biotin) or fluorescent (alk-rho, az-rho) detection tags prior to visualization by streptavidin blot (FIG. 12A) or in-gel fluorescence scanning (FIG. 12B), respectively. In-gel fluorescence detection of the immunopurified fatty-acylated proteins (Lck, LAT & Ras) was markedly improved compared to streptavidin blotting. For example, S-palmitoylation of LAT using palmitic acid analogs (az-15, alk-16) was nearly undetectable by streptavidin blotting (FIG. 12A top panels), but was robustly visualized by in-gel fluorescence scanning (FIG. 12B top panels). Similar observations were observed with Lck and Ras (FIGS. 12A and 12B middle and bottom panels). The fatty-acylation of proteins without N-terminal Gly residues (LAT & Ras) with the myristic acid analogs (az-12, alk-12) was not unexpected, as S-palmitoylation or S-acylation is known to involve a heterogeneous composition of fatty acids (Liang et al., (2004) J. Biol. Chem. 279, 8133-8139).

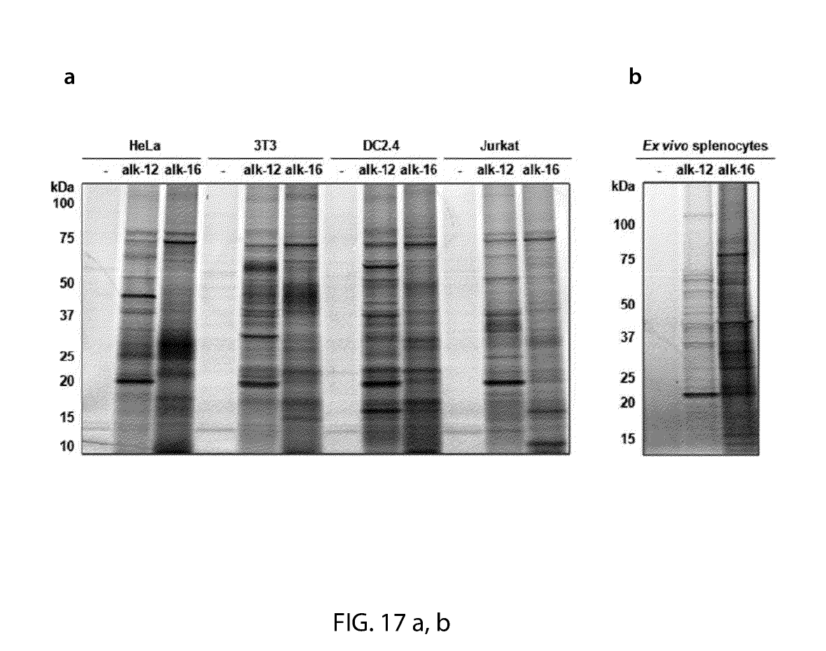

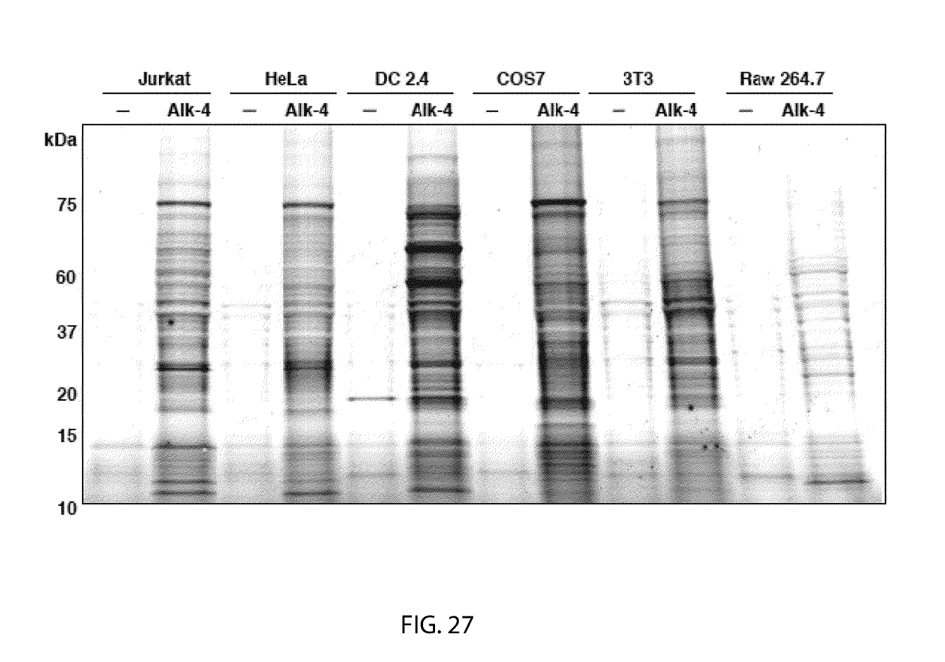

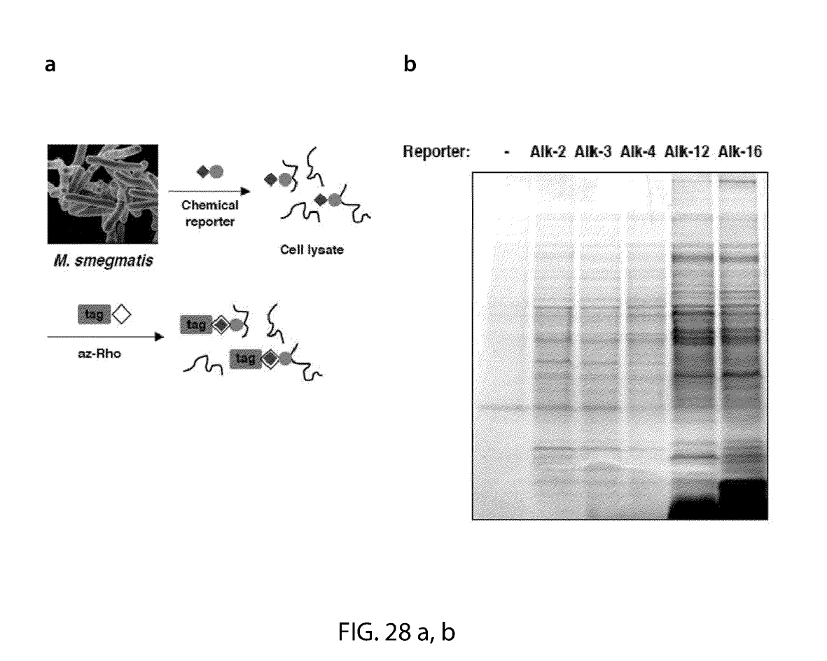

The generality of our method was evaluated by labeling different mammalian cell types (HeLa, 3T3, DC2.4, Jurkat and primary splenocytes) with alkynyl-chemical reporters (alk-12 or alk-16) (FIG. 17). Labeling of the different mammalian cells types (Jurkat, HeLa, DC2.4, COST, 3T3, and Raw 264.7) was also demonstrated using alkynyl-chemical reporters of protein acetylation (alk-4) (FIG. 27). These comparative analysis revealed remarkably diverse profiles of acylated proteins amongst different cell types. While some acylated polypeptide bands are common among the different cell types, distinct N-myristoylation and S-palmitoylation patterns are apparent. Indeed, the complete repertoire of fatty-acylated proteins varies dramatically between epithelial cell lines (HeLa and NIH 3T3 fibroblasts), a monocyte-derived cell line (DC2.4), T cells (Jurkat) and splenocytes (FIG. 17). Use of any methods provided herein is not limited to mammalian cells. Alkynyl-chemical reporters of various lengths also label acylated-proteins in bacterial cells using the essentially the same method with media appropriate for bacterial cell culture (FIG. 28A). FIG. 28B shows that the alkynyl-chemical reporters alk-2, alk-3, alk-4, alk-12, and alk-16 all labeled distinct proteins in cells of the mycobacteria M. smegmatis.

These experiments highlight the utility of our chemical reporters and improved detection conditions, which demonstrate unique profiles of acylated proteins in a variety of organisms, discrete cell types, and primary tissues that undoubtedly contribute to specific cellular properties.

### Example 4

### In Vivo Labeling of Acylated Proteins

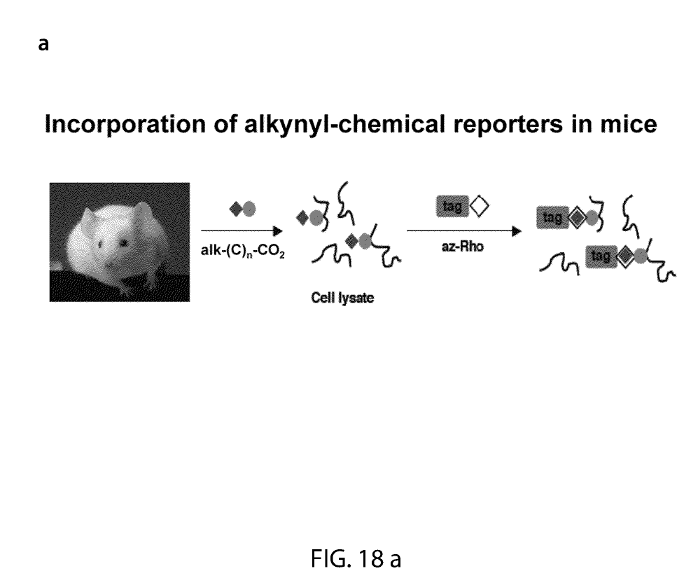

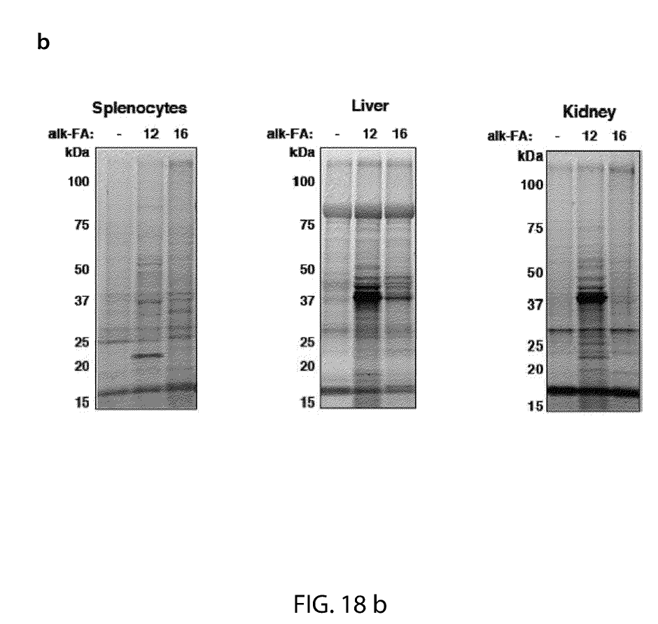

The visualization of acylated proteins from living animals would afford new opportunities to address protein acylation in physiology and disease. To explore the utility of our alkynyl-chemical reporters in vivo, mice were intraperitoneally injected with alkynyl-chemical reporters (alk-12 or alk-16) and analyzed for protein acylation in various tissues. Following one hour of metabolic labeling with our alkynyl-chemical reporters in vivo, protein acylation could be visualized in cell lysates prepared from splenocytes, liver, and kidney (FIG. 18). In particular, in vivo labeling with the alk-12 afforded a discrete profile of acylated proteins from splenocytes similar to ex vivo labeling (FIG. 17 and FIG. 18B). These experiments suggest that protein acylation is quite dynamic in vivo. Indeed, in vivo labeling with alk-16 did not reveal discrete profiles of acylated proteins in splenocytes or liver under these conditions compared to alk-12 (FIG. 18B). It is however contemplated that given the dynamic nature of protein S-palmitoylation, optimization in the dose, time and route of administration in vivo should enable specific visualization of acylated proteins with alk-16. These in vivo labeling experiments demonstrate for the first time that our chemical reporters (alk-12) can function in living animals and enable the specific detection of acylated proteins in primary tissues. It should also be noted that our chemical reporters were well tolerated by the mice, as no overt toxicity was observed following in vivo administration, even after several days.

### Example 5

### Global Analysis of Acylated Proteins in Mammalian Cells

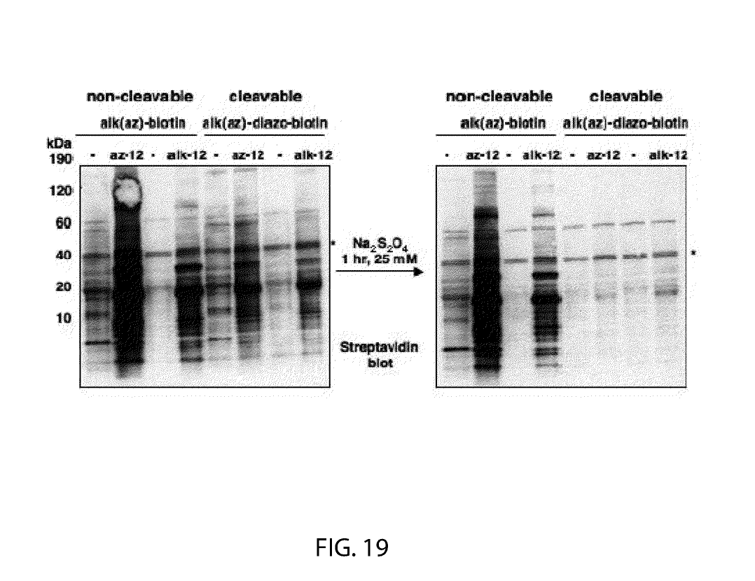

To identify acylated proteins targeted by our chemical reporters, we synthesized cleavable detection tags (alk-diazo-biotin, az-diazo-biotin) (FIG. 13) to affinity purify and selectively elute labeled polypeptides for proteomic analysis (FIG. 14). While the biotin-avidin interaction provides an excellent system for selective detection and retrieval of biomolecules under variety of conditions (high salt and detergent with extensive washing), the high affinity binding (˜KD 10-15 M) of this interaction makes quantitative elution of bound materials from streptavidin beads challenging. Of the various selective elution strategies that have been described in the literature (disulfide, acid- and base-sensitive functional groups, protease-sensitive peptides), we employed the diazobenzene linker since this functional group can be efficiently cleaved by reduction with sodium thionite (Na₂S₂O₄) and readily incorporated into our detection tags by chemical synthesis (Verhelst et al., (2007) Angew Chem Int Ed Engl, 46(9): 1284-1286; Fonovic et al., (2007) Mol Cell Proteomics). In comparison with non-cleavable biotinylated detection tags (alk-biotin, az-biotin), the diazobenzene-modified detection tags (alk-diazo-biotin, az-diazo-biotin) labeled a similar profile of polypeptides from azido- and alkynyl-chemical reporter labeled Jurkat T cell lysates (FIG. 19). Treatment of alk/az-diazo-biotin labeled cell lysates with sodium thionite efficiently cleaved biotin from targeted proteins, whereas no effect was observed on acylated proteins labeled with noncleavable detection tags (alk/az-biotin) (FIG. 19). HPLC analysis of the diazobenzene-modified detection tags confirmed their stability in the presence of 1 mM TCEP (optimal conditions for click chemistry) and the selective cleavage with 25 mM Na₂S₂O₄ (data not shown). These experiments demonstrate that the diazobenzene functionality survived the slightly reducing conditions of click chemistry and can be efficiently cleaved with sodium thionite.

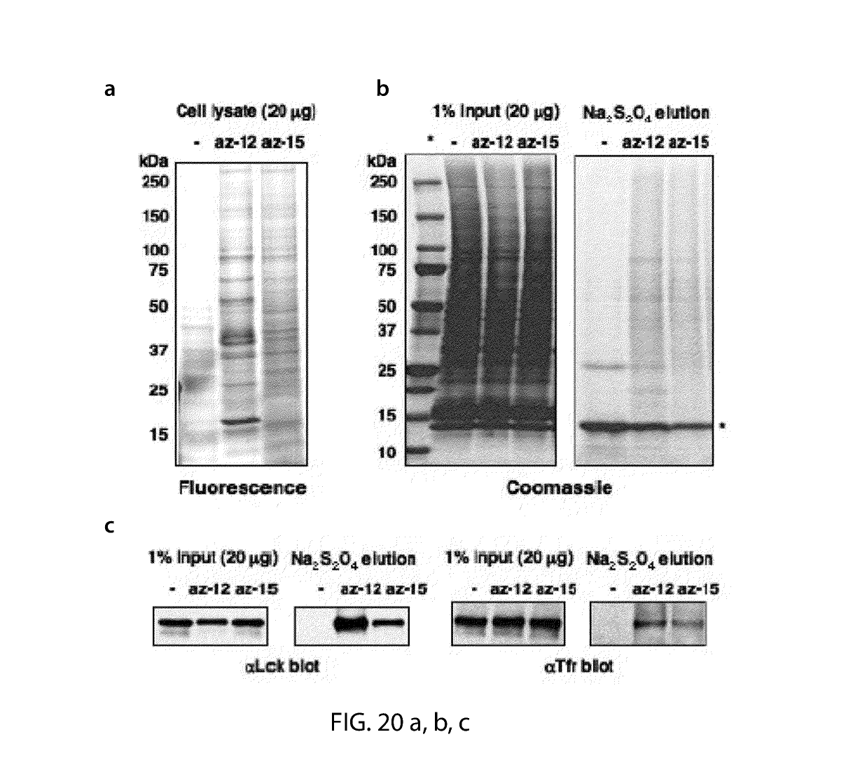

We then sought to selectively recover fatty-acylated proteins from cells. Azido- and alkynyl-chemical reporter labeled Jurkat T cell lysates were reacted with the diazobenzene-modified detection tags (alk-diazo-biotin shown as compound I in FIG. 22 and azdiazo-biotin shown as compound II in FIG. 22, respectively), subjected to affinity enrichment with streptavidin beads, Na₂S₂O₄ elution and proteomic analysis by mass spectrometry as well as immunoblotting with specific antibodies (FIG. 20A, 20B, 20C). The Src-family kinase Lck, an N-myristoylated and S-palmitoylated protein in Jurkat T cell lysates (Hang et al., (2007) J Am Chem Soc, 129(10): 2744-2745), was initially used to optimize conditions for the selective affinity enrichment and recovery of acylated proteins (data not shown). Using our optimized protocol, ˜2 mg of azido-chemical reporter labeled cell lysates were reacted with alk-diazo-biotin, proteins were precipitated and washed with ice-cold methanol several times to remove excess alk-diazo-biotin, resolubulized and subjected to streptavidin affinity enrichment. The streptavidin beads were then extensively washed with buffers containing high salt, detergent and urea to remove non-specifically bound proteins. Biotinylated polypeptides that remained bound to streptavidin beads were eluted with sodium thionite (Na₂S₂O₄), separated by SDS-PAGE and visualized by coomassie staining or by immunoblot for specific acylated proteins. Coomassie staining of the polypeptides selectively eluted from streptavidin beads revealed significantly greater amounts of proteins recovered from az-12- and az-15-labeled cell lysates compared to control (−) (FIG. 20A). A fraction of the cell lysates analyzed in parallel demonstrates equal levels of input material. The profile of selectively recovered polypeptides mirror the acylated proteins visualized by click chemistry and in-gel fluorescence scanning, which confirms the specificity and efficiency of affinity enrichment and elution protocols. Western blot analysis of Lck, a known fatty-acylated protein in Jurkat T cells (FIG. 12A and FIG. 12B), in these samples reinforces the selectivity of retrieval (FIG. 20C). Consequently, each lane (−, az-12, az-15) of the coomassie-stained gel was processed using standard protocols for protein extraction, reductive alkylation, protease digestion and submitted for peptide sequencing using our Thermo-LTQ-Orbitrap mass spectrometer (Scigelova and Makarov (2006) Proteomics, 6 Suppl 2: 16-21; Makarov et al., (2006) J. Am. Soc. Mass Spectrom., 17(7): 977-982) (jointly purchased with The Rockefeller Proteomics Facility). High-resolution MS/MS analysis of tryptic peptides followed by database searches using Mascot and Sequest/Bioworks, using the two peptide cut-off rule and subtractive analysis of the peptides recovered in the negative control (−) revealed known acylated proteins and many candidate acylated proteins in Jurkat T cells (Table 1). Acylated proteins that were selectively recovered and identified represented major classes of N-myristoylated and S-palmitoylated proteins reported in the literature, once again confirming the selectivity and efficiency of our methods. Several peptides were identified for Lck as well as the transferrin receptor (Tfr), which is known to be S-palmitoylated on Cys62 and Cys67 (Alvarez et al., (1990) J Biol Chem, 265(27): 16644-16655; Omary and Trowbridge (1981) J Biol Chem, 256(10): 4715-4718). Indeed, western blot analysis for Tfr in these samples confirmed selective recovery from both az-12 and az-15 chemical reporter-labeled cell lysates (FIG. 20C). Even though Tfr is a type II membrane protein that does not contain an N-terminal Gly residue, the recovery of Tfr from both az-12 and az-15-labeled cell lysates in not surprising since S-palmitoylation sites are known to contain fatty acids of heterogeneous chain lengths (Liang et al., (2004) J Biol Chem, 279(9): 8133-8139), which is consistent with our results for LAT and Ras (FIG. 12A and FIG. 12B). Thus, a fraction of the proteins labeled with our myristic acid chemical reporter analogs also target S-palmitoylated proteins.

Table 1. Known acylated proteins that were selectively recovered from az-12 and az-15 labeled Jurkat T cells lysates (http://www.ebi.ac.uk/). 70 additional proteins not previously reported to be acylated were also selectively recovered (data not shown).

Transferrin receptor protein 1 (CD71 antigen)
Proto-oncogene tyrosine-protein kinase Fyn
Proto-oncogene tyrosine-protein kinase Yes
Proto-oncogene tyrosine-protein kinase Src
40S ribosomal protein S2 (S4) (LLRep3 protein)
Ubiquinol-cytochrome c reductase complex ubiquinone-binding
protein QP-C
ADP-ribosylation factor 6 - Homo sapiens
Elongation factor 2 (EF-2)
CD82 antigen (Inducible membrane protein R2)
NADH-cytochrome b5 reductase
Guanine nucleotide-binding protein G(o) subunit alpha 2
Guanine nucleotide-binding protein alpha-13 subunit (G alpha-13)
Guanine nucleotide-binding protein alpha-12 subunit (G alpha-12)
ADP-ribosylation factor 3
26S protease regulatory subunit 4 (P26s4)
Guanine nucleotide-binding protein G(s) subunit alpha
Guanine nucleotide-binding protein G(t), alpha-1 subunit
Guanine nucleotide-binding protein G(t), alpha-2 subunit
Guanine nucleotide-binding protein G(olf) subunit alpha
ADP-ribosylation factor 4
Guanine nucleotide-binding protein G(k) subunit alpha (G(i) alpha-3)
Guanine nucleotide-binding protein G(o) subunit alpha 1
Guanine nucleotide-binding protein G(i), alpha-1 subunit (Adenylate
cyclase-inhibiting G alpha protein)
ADP-ribosylation factor 5
ADP-ribosylation factor 1
Tubulin alpha-1 chain (Alpha-tubulin 1)
Tubulin alpha-2 chain (Alpha-tubulin 2)
Calnexin precursor (IP90)
Proto-oncogene tyrosine-protein kinase LCK
MARCKS-related protein (MARCKS-like protein 1)
Tubulin alpha-6 chain (Alpha-tubulin 6)
Guanine nucleotide-binding protein G(i), alpha-2 subunit

### Example 6

### Experimental Methods

Example 6 describes the detailed experimental methods used in Examples 7 and 8.

### Metabolic Labeling

Jurkat cells (human T lymphoma) were cultured in RPMI medium 1640 supplemented with 10% fetal bovine serum (FBS), 100 U/mL penicillin, and 100 μg/mL streptomycin and maintained in a humidified 37° C. incubator with 5% CO₂. Trypan blue exclusion was used to determine cell viability. Cells were pelleted and resuspended in either az-12, az-15, alk-12, alk-14 or alk-16 (20 μM, 50 mM stock solution in DMSO) in RPMI medium 1640 supplemented with 2% FBS, 100 U/mL penicillin, and 100 μg/mL streptomycin. The same volume of DMSO was used as a negative control. After 2 hours of labeling at 37° C., the cells were pelleted at 1,000 g for 5 minutes and washed once with ice-cold PBS, directly lysed or flash frozen in liquid nitrogen and stored at −80° C. prior to lysis. No significant loss of signal was observed for frozen cell pellets.

HeLa, 3T3 and DC2.4 cells were cultured in DMEM, supplemented with 10% fetal bovine serum (FBS), 100 U/mL penicillin, and 100 μg/mL streptomycin and maintained in a humidified 37° C. incubator with 5% CO₂. Trypan blue exclusion was used to determine cell viability. Cells were treated with either alk-12 (20 μM, 5 mM stock solution in DMSO) or alk-16 (200 μM, 50 mM stock solution in DMSO) in DMEM supplemented with 2% FBS, 100 U/mL penicillin, and 100 μg/mL streptomycin. The same volume of DMSO was used as a negative control. After 4-6 hours of labeling at 37° C., the cells were washed once with ice-cold PBS, harvested with a cell scraper and pelleted at 1,000 g for 5 minutes.

Spleens were harvested from 6 week-old female C57/BL6 mice. Splenocytes were prepared by manual disruption of spleens using forceps. Red blood cells were eliminated using ACK lysis buffer. Splenocytes were pelleted and resuspended in either alk-12 or alk-16 (20 μM, 50 mM stock solution in DMSO) in RPMI medium 1640 supplemented with 2% FBS, 100 U/mL penicillin, and 100 μg/mL streptomycin using one spleen per labeling condition. The same volume of DMSO was used as a negative control. After 4-6 hours of labeling at 37° C., the cells were pelleted at 1,000 g for 5 minutes, washed once with ice-cold PBS, and directly lysed.

Competition of Metabolic Labeling with Naturally Occurring Fatty Acids

Jurkat cells were pelleted and resuspended in either alk-12, alk-14 or alk-16 (10 μM, 50 mM stock solution in DMSO) and either myristic acid (0, 10 or 100 μM, 100 mM stock solution in DMSO) or palmitic acid (0, 100 or 200 μM, 100 mM stock solution in DMSO) in RPMI medium 1640 supplemented with 2% FBS, 100 U/mL penicillin, and 100 μg/mL streptomycin. The same volume of DMSO was used as a negative control. After 2 hours of labeling at 37° C., the cells were pelleted at 1,000 g for 5 minutes and washed once with ice-cold PBS, and directly lysed.

Metabolic Labeling with Inhibitors

Jurkat cells were pelleted and resuspended in either cycloheximide (CHX) (10 μM, 100 mM stock solution in DMSO), 2-hydroxymyristic acid (HMA) (1 mM, 100 mM stock solution in DMSO) or 2-bromopalmitate (2-BP) (50 μM, 30 mM stock solution in DMSO) in RPMI medium 1640 supplemented with 2% FBS, 100 U/mL penicillin, and 100 μg/mL streptomycin. 1% fatty acid free BSA (Sigma, St. Louis, Mo., USA) was added to the medium in the case of HMA treatment. The same volume of DMSO was used as a negative control. After 30 minutes of pre-incubation at 37° C., either alk-12, alk-14 or alk-16 (20 μM, 50 mM stock solution in DMSO) was added to the medium. The same volume of DMSO was used as a negative control. After 2 hours of labeling at 37° C., the cells were pelleted at 1,000 g for 5 minutes and washed once with ice-cold PBS, and directly lysed.

In Vivo Labeling

PBS containing 10% fatty acid free BSA (Sigma, St. Louis, Mo., USA) was added to alk-12 and alk-16 (25 mg/mL), followed by brief sonication, warming to 37° C., and IP injection of 200 μL into 6 week-old female C57/BL6 mice for 1 hour. Livers and kidney were harvested and incubated with Liberase 3 Blendzyme (Roche, Mannheim, Germany) at 37° C. for 30 minutes and homogenized prior to filtration with 0.4 μm cell strainers. Splenocytes were prepared by manual disruption of spleens using forceps. Liver, kidney and splenocyte preparations were subjected to red blood cell lysis using ACK lysis buffer. Cells were pelleted at 1,000 g for 5 minutes, washed once with ice-cold PBS, and directly lysed.

Preparation of Cell Lysates

Cell pellets obtained from 10×10⁶ Jurkat cells or 1 confluent well of a 6-well plate of HeLa, 3T3 or DC2.4 cells were lysed with 100 μL of ice-cold modified RIPA lysis buffer (1% Nonidet P 40, 1% sodium deoxycholate, 0.1% SDS, 50 mM triethanolamine pH 7.4, 150 mM NaCl, 5×EDTAfree Roche protease inhibitor cocktail, 10 mM phenylmethylsulfonyl fluoride (PMSF)) by first disrupting the pellet by sonication, and then vortexing 3×10 seconds, cooling the lysate on ice between pulses. Cell lysates were collected after centrifuging at 1,000 g for 5 minutes at 4° C. to remove cell debris. Protein concentration was determined by the BCA assay. Typical lysate protein concentrations obtained: Jurkat 2-3 mg/mL, HeLa 1-2 mg/mL, 3T3 0.5-1 mg/mL and DC2.4 1-2 mg/mL. Cell lysates were diluted with modified RIPA lysis buffer to achieve final protein concentration of ˜1 mg/mL for labeling reactions.

Cell pellets obtained from a spleen, liver or kidney were lysed with 400 μL of ice-cold Brij lysis buffer (1% Brij 97, 50 mM triethanolamine pH 7.4, 150 mM NaCl, 5×EDTA-free Roche protease inhibitor cocktail) as mentioned above. Protein concentration was determined by the BCA assay (Pierce). Typical lysate protein concentrations obtained: spleen 10 mg/mL, liver 10 mg/mL. Cell lysates were diluted with Brij lysis buffer to achieve final protein concentration of ˜1 mg/mL for labeling reactions.

### Staudinger Ligation

Cell lysates (50 μg) in 46.5 μL modified RIPA lysis buffer were reacted with 1 μL phosphinebiotin (200 μM, 10 mM stock solution in DMSO) and 2.5 μL DTT (5 mM, 100 mM stock solution in deionized water) for a total reaction of volume of 50 μL for 1 hour at room temperature (Vocadlo et al., (2003) Proc. Nat. Acad. Sci. U.S.A. 100, 9116-9121). DTT prevents non-specific oxidation of phosphine-biotin, which can increase levels of background labeling. The reactions were terminated by the addition of −20° C. methanol (1 mL) and placed at −20° C. for at least 1 hr, centrifuged at 18,000 g for 10 minutes at 0° C. to precipitate proteins. The supernatant from the samples were discarded. The protein pellets were allowed to air dry for 10 min, resuspended in 35 μL of resuspension buffer (4% SDS, 50 mM triethanolamine pH 7.4, 150 mM NaCl), diluted with 12.5 μL 4× reducing SDS-loading buffer (40% glycerol, 200 mM Tris-HCl pH 6.8, 8% SDS, 0.4% bromophenol blue) and 2.5 μL 2-mercaptoethanol, heated for 5 minutes at 95° C. and ˜20 μg of protein was loaded per gel lane for separation by SDS-PAGE (10% or 4-20% Bio-Rad Criterion™ Tris-HCl gel) (Bio-Rad, Hercules, Calif., USA).

### Click Chemistry

Cell lysates (50 μg) in 47 μL modified RIPA lysis buffer were reacted with 3 μL freshly premixed click chemistry reaction cocktail [azido- or alkynyl-detection tag (100 μM, 10 mM stock solution in DMSO), tris(2-carboxyethyl)phosphine hydrochloride (TCEP) (1 mM, 50 mM freshly prepared stock solution in deionized water), tris[(1-benzyl-1H-1,2,3-triazol-4-yl)methyl]amine (TBTA) (100 μM, 10 mM stock solution in DMSO), and CuSO₄.5H₂O (1 mM, 50 mM freshly prepared stock solution in deionized water)] for a total reaction volume of 50 μL for 1 hour at room temperature. The reactions were terminated by the addition of ice-cold methanol (1 mL) and placed at −80° C. overnight, centrifuged at 18,000 g for 10 minutes at 4° C. to precipitate proteins. The supernatant from the samples were discarded. The protein pellets were allowed to air dry for 10 minutes, resuspended in 35 μL of resuspension buffer (4% SDS, 50 mM triethanolamine pH 7.4, 150 mM NaCl), diluted with 12.5 μL 4× reducing SDS-loading buffer (40% glycerol, 200 mM Tris-HCl pH 6.8, 8% SDS, 0.4% bromophenol blue) and 2.5 μL 2-mercaptoethanol, heated for 5 min at 95° C. and ˜20 μg of protein was loaded per gel lane for separation by SDS-PAGE (10% or 4-20% Bio-Rad Criterion™ Tris-HCl gel) (Bio-Rad, Hercules, Calif., USA).

Transfection of HeLa Cells

HeLa cells were grown in 10 cm dishes to approximately 90% confluence in DMEM, supplemented with 10% FBS, and transfected with wildtype, G2A or C3,6S Fyn constructs using Lipofectamine 2000 (Invitrogen). The human Fyn constructs, wild type and mutant Fyn cDNAs cloned into eukaryotic expression vector pCMV5, were gifts from Dr. Marilyn Resh, Memorial Sloan-Kettering Cancer Center. The following day, cells were metabolically labeled with alkynyl-fatty acid analogs as described above.

Immunoprecipitation

Cell pellets obtained from 15×10⁶ Jurkat cells or transfected HeLa cells were lysed with 50 μL of ice-cold Brij lysis buffer (1% Brij 97, 50 mM triethanolamine pH 7.4, 150 mM NaCl, 5×EDTAfree Roche protease inhibitor cocktail, 10 mM PMSF) by first disrupting the pellet by sonication, and then vortexing 3×10 seconds, cooling the lysate on ice between pulses. Cell lysates were collected after centrifuging at 1,000 g for 5 minutes at 4° C. to remove cell debris. Protein concentration was determined by the BCA assay. Typical lysate protein concentration obtained: 6-8 mg/mL. LAT, Lck and Ras proteins were immunoprecipitated from 200 μg Jurkat cell lysate using the following antibodies at recommended concentrations: mouse anti-Lck (p56lck) monoclonal (Clone 3A5, Thermo Scientific, Waltham, Mass., USA), rat anti-v-H-ras (Ab-1) monoclonal (Y13-259 agarose conjugate, Calbiochem, San Diego, Calif., USA), and rabbit anti-LAT polyclonal (Millipore, Billerica, Mass., USA). A rabbit anti-Fyn polyclonal (Millipore, Billerica, Mass., USA) was used to immunoprecipitate wild type and mutant Fyn proteins from transfected and metabolically labeled HeLa cells. 25 μL of packed protein A-agarose beads (Roche, Mannheim, Germany) was used per sample. Upon incubation at 4° C. for one hour with an end-over-end rotator (Barnstead/Thermolyne, Waltham, Mass., USA), the beads were washed thrice with ice-cold modified RIPA lysis buffer (1% Triton X-100, 1% sodium deoxycholate, 0.1% SDS, 10 mM Tris pH 7.4, 150 mM NaCl). The beads were resuspended in 20 μL of resuspension buffer (4% SDS, 50 mM triethanolamine pH 7.4, 150 mM NaCl) and freshly premixed click chemistry reagents (same as above) were added. After 1 hour at room temperature, the reaction mixture was diluted with 6.7 μL 4× reducing SDS-loading buffer (40% glycerol, 200 mM Tris-HCl pH 6.8, 8% SDS, 0.4% bromophenol blue) and 1.3 μL 2-mercaptoethanol, heated for 5 minutes at 95° C. and 20 L of the supernatant was loaded per gel lane for separation by SDS-PAGE (4-20% Bio-Rad Criterion Tris-HCl gel).

Hydroxylamine Cleavage of S-Acylated Proteins

After the proteins were separated by SDS-PAGE, the gel was soaked in 40% MeOH, 10% acetic acid, shaking overnight at room temperature, washed with deionized water (2×5 minutes) and scanned for the pre-hydroxylamine treatment fluorescence. The gel was then soaked in PBS, shaking 1 hour at room temperature, followed by soaking in 1 M NH₂OH (pH=7.4), shaking 8 hours at room temperature, washing with deionized water (2×5 minutes), and soaking in 40% MeOH, 10% acetic acid, shaking overnight at room temperature. The gel was finally washed with deionized water (2×5 minutes) and scanned for the post-hydroxylamine treatment fluorescence.

In-Gel Fluorescence Scanning

Proteins separated by SDS-PAGE were visualized by first soaking the gel in 40% MeOH, 10% acetic acid with shaking for 5 minutes, followed by soaking in deionized water with shaking for 5 min and directly scanning the gel on an Amersham Biosciences Typhoon 9400 variable mode imager (excitation 532 nm, 580 nm filter, 30 nm band-pass) (Piscataway, N.J., USA).

Immuno-Blotting

Proteins separated by SDS-PAGE were transferred (50 mM Tris, 40 mM glycine, 0.0375% SDS, 20% MeOH in deionized water, Bio-Rad Trans-Blot Semi-Dry Cell, 20 V, 1 hr) onto a PVDF membrane which was blocked (5% non-fat dried milk, 1% BSA and 0.1% Tween-20 in PBS) for 1 hour at 25° C. or overnight at 4° C. The membrane was washed thrice with PBST (0.1% Tween-20 in PBS), incubated with streptavidin-horseradish peroxidase (1 mg/mL diluted 1:25,000 in PBST, Pierce, Waltham, Mass., USA), and subsequently developed with ECL Western blotting detection reagents (Amersham, Piscataway, N.J., USA). Alternatively, Lck, LAT, Ras and Fyn protein levels were visualized by incubating the blots at recommended concentrations in 5% casein, 1% BSA in PBST with mouse anti-Lck (p56lck) monoclonal (Clone 3A5, Thermo Scientific, Waltham, Mass., USA), mouse anti-LAT monoclonal (2E9, Millipore, Billerica, Mass., USA), mouse anti-Ras monoclonal (RAS10, Millipore, Billerica, Mass., USA) or mouse anti-Fyn monoclonal (S1, Millipore, Billerica, Mass., USA), respectively, followed by a goat anti-mouse-HRP conjugated secondary antibody (Millipore, Billerica, Mass., USA) in the blocking buffer mentioned above.

### Example 7

### Competition and Inhibition Studies

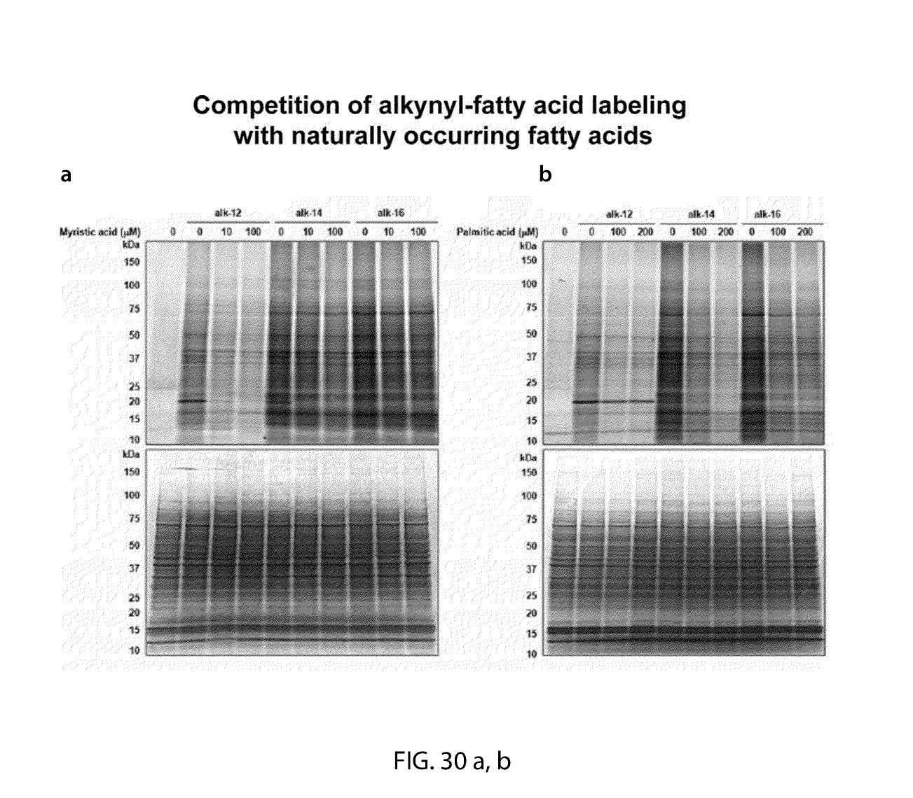

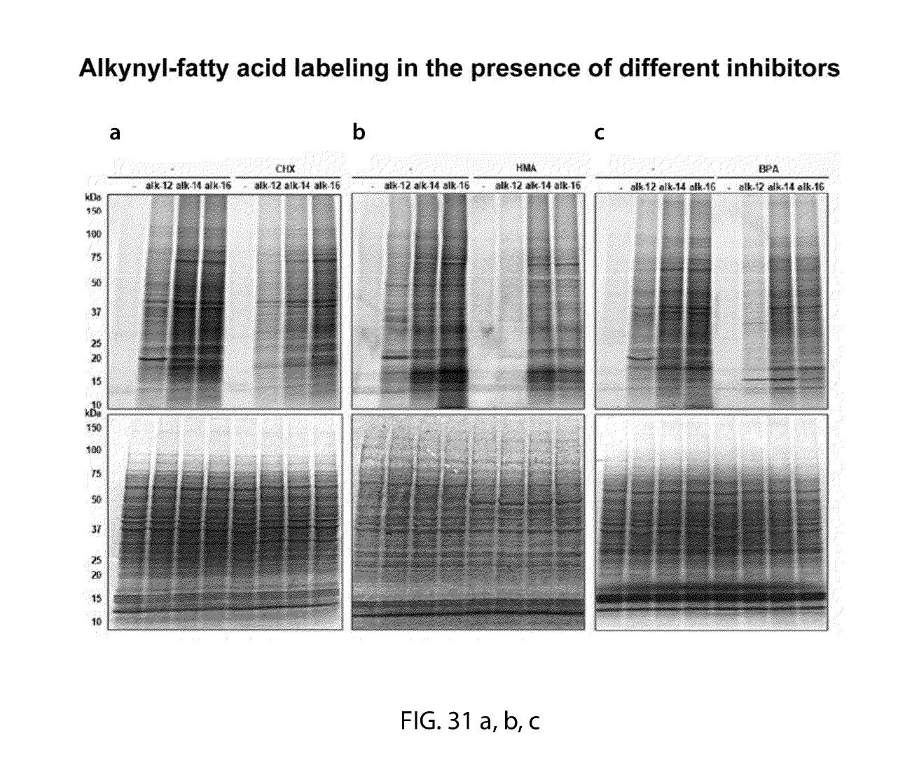

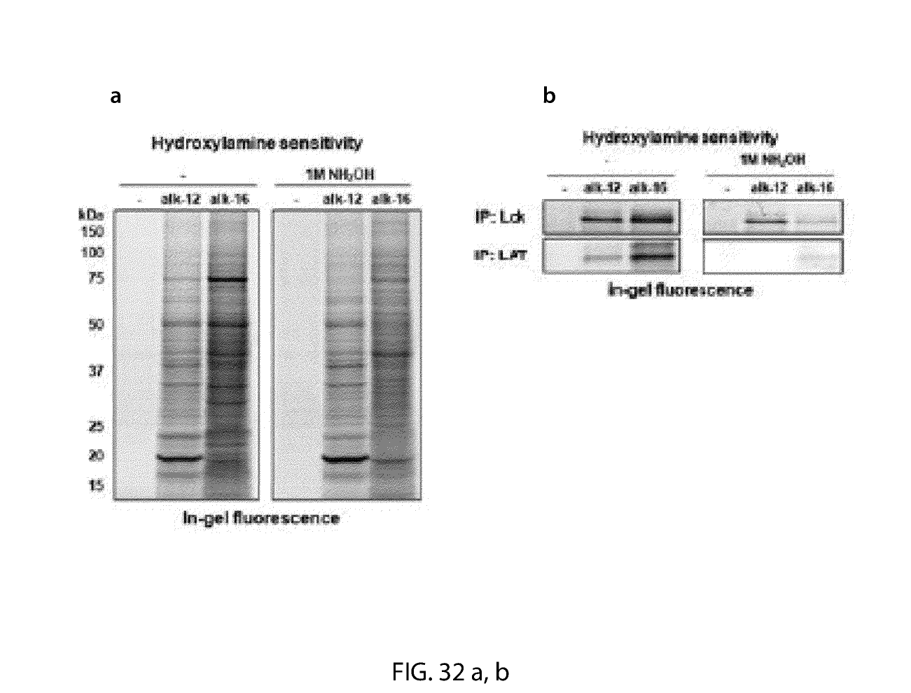

Having established robust fluorescence detection of proteins metabolically labeled with-alkynyl fatty acid chemical reporters, we determined the kinetics and specificity of our approach. Time- and dose-dependent analyses of metabolic labeling with the alkynyl-fatty acids revealed that the click chemistry and in-gel fluorescence imaging protocol required shorter labeling time (minutes) and lower concentrations of fatty acid chemical reporters than previous methods (Hang, H. C., et al., (2007) J. Am. Chem. Soc., 129: 2744-2745) to robustly detect fatty-acylated proteins (FIG. 16A and FIG. 16B). Dose-dependent competition of alkynyl-fatty acid protein labeling (alk-12, alk-14 & alk-16) with naturally occurring fatty acids revealed that alk-12 protein labeling is selectively blocked by myristic acid, whereas alk-14 and alk-16 protein labeling is most effectively reduced by palmitic acid (FIG. 30A and FIG. 30B). Inhibition of protein synthesis with cycloheximide (CHX) abrogated the metabolic labeling of several prominent polypeptides by alk-12, however, most proteins targeted by alk-12, alk-14 and alk-16 appear to occur post-translationally (FIG. 31A). Coincubation of the alkynyl-fatty acids with 2-hydroxymyristic acid (HMA), a reported N-myristoylation inhibitor, selectively blocked alk-12 protein labeling compared to alk-14 and alk-16 (FIG. 31B). In contrast, addition of 2-bromopalmitic acid (2-BP; BPA), a non-specific S-palmitoylation inhibitor, at concentrations that did not induce cell death reduced protein labeling with alk-12, alk-14 and alk-16 (FIG. 31C). To differentiate between N-myristoylated and S-acylated proteins, az-rho-modified alkynyl-fatty acid labeled cell lysates were subjected to in-gel treatment with hydroxylamine (NH₂OH), which preferentially cleaves thioesters at neutral pH. The fluorescent signal of alkynyl-fatty acid labeled proteins were reduced after in-gel exposure to NH₂OH, however, the CHX-sensitive proteins labeled by alk-12 were resistant to NH₂OH cleavage (FIG. 32A). These experiments suggest that alk-12 cotranslationally targets N-myristoylated proteins (CHX-sensitive and NH₂OH-resistant) as well as S-acylated proteins (CHX resistant and NH₂OH-sensitive), whereas alk-16 preferentially labels S-acylated proteins in cell lysates. Protein labeling with alk-14 appears to represent a combination of alk-12 and alk-16 labeling, which is consistent with previous observations with az-14 labeling (Hang, H. C., et al., (2007) J. Am. Chem. Soc., 129: 2744-2745).

### Example 8

### Fatty Acid Chemical Reporters Combined with Fluorescence Detection Enables Specific Detection of N-Myristoylated and S-Palmitoylated Proteins

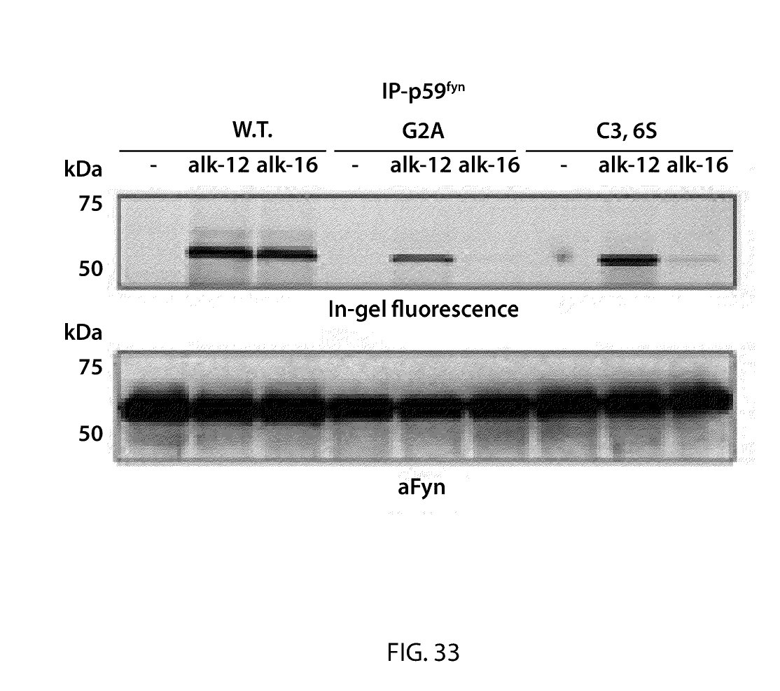

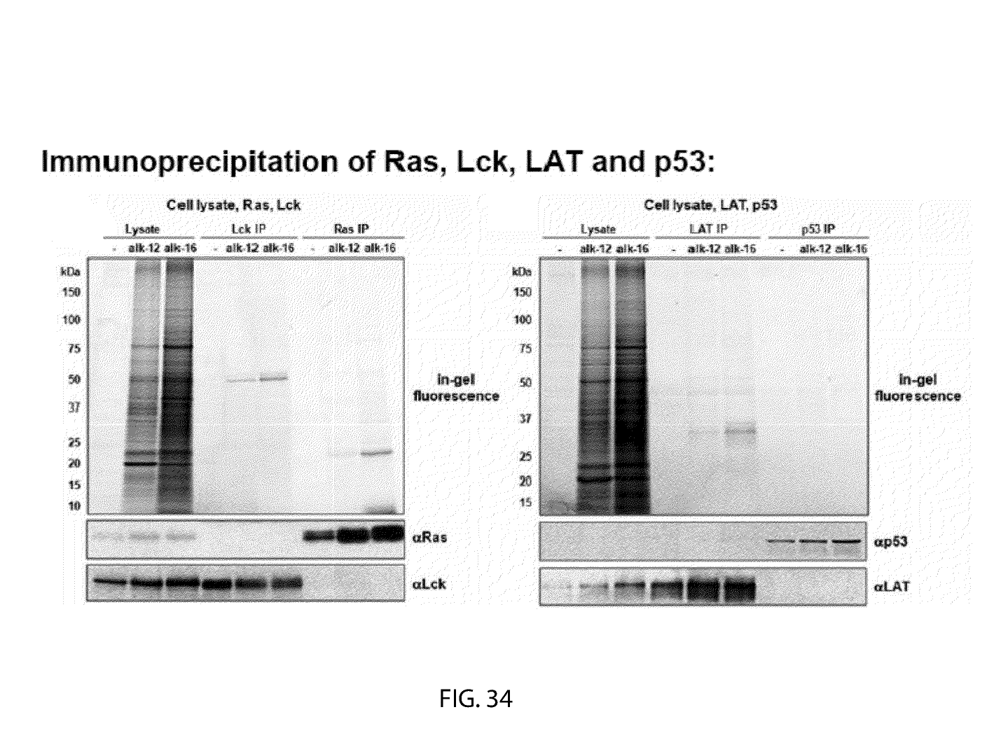

In-gel NH₂OH treatment of alkynyl-fatty acid labeled Lck and LAT reduced the fluorescent signal derived from alk-16 on both proteins, but did not alter the alk-12 labeling of Lck (FIG. 32B). We also analyzed the specificity of our fatty acid chemical reporters with wild-type and mutant constructs of p59 Fyn14, a well characterized N-myristoylated and S-palmitoylated Src-family kinase, by overexpression in HeLa cells, metabolic labeling and immunoprecipitation (FIG. 33). Fatty-acylation of wild-type Fyn is readily detected with alk-12 and alk-16 labeling, whereas the N-myristoylation G2A-mutant exhibited significantly reduced alk-12 labeling and was undetectable with alk-16 (FIG. 33). The dual S-palmitoylation-deficient C3,6S mutant Fyn was efficiently labeled with alk-12 and not with alk-16 (FIG. 33). These results are quantitatively identical to previously described experiments using radiolabeled fatty acids, which also demonstrated residual labeling of G2A-mutant Fyn with a ¹²⁵I-myristic acid analog and no labeling with ¹²⁵I-palmitic acid analog (Alland, L., et al., (1994) J. Biol. Chem., 269: 16701-16705). Our experiments therefore also support the model that N-myristoylation precedes S-palmitoylation and highlight the possibility of fatty-acylation at N-terminal alanine residues. In contrast to LAT, Lck, Ras and Fyn, no alkynyl-fatty acid labeling was observed for p53, a prominent acetylated protein for which fatty-acylation has not been reported (Tang, Y., et al., (2008) Cell, 133: 612-626), when analyzed in parallel with LAT, Lck and Ras (FIG. 34). Collectively, our experiments with cell lysates and specific proteins demonstrate that alk-12 and alk-14 label N-myristoylated and S-acylated proteins, whereas longer chain fatty chemical reporters such as alk-16 preferentially target S-acylated proteins.

### Example 9

### Additional Fluorophores and Dyes to Label N-Myristoylated and S-Palmitoylated Proteins

Development of modular fluorescent dyes compatible with bioorthogonal ligation methods will expand the repertoire of reagents for diverse imaging applications using mechanism-based probes or chemical reporters. Herein, we report a concise synthesis of clickable fluorescent dyes based on 2-dicyanomethylene-3-cyano-2,5-dihydrofuran (DCDHF) fluorophores for multimodal imaging applications using Cu(I)-catalyzed azide-alkyne cycloaddition (CuAAC) (E. M. Sletten and C. R. Bertozzi, Angew Chem Int Ed Engl, 2009, 48, 6974).

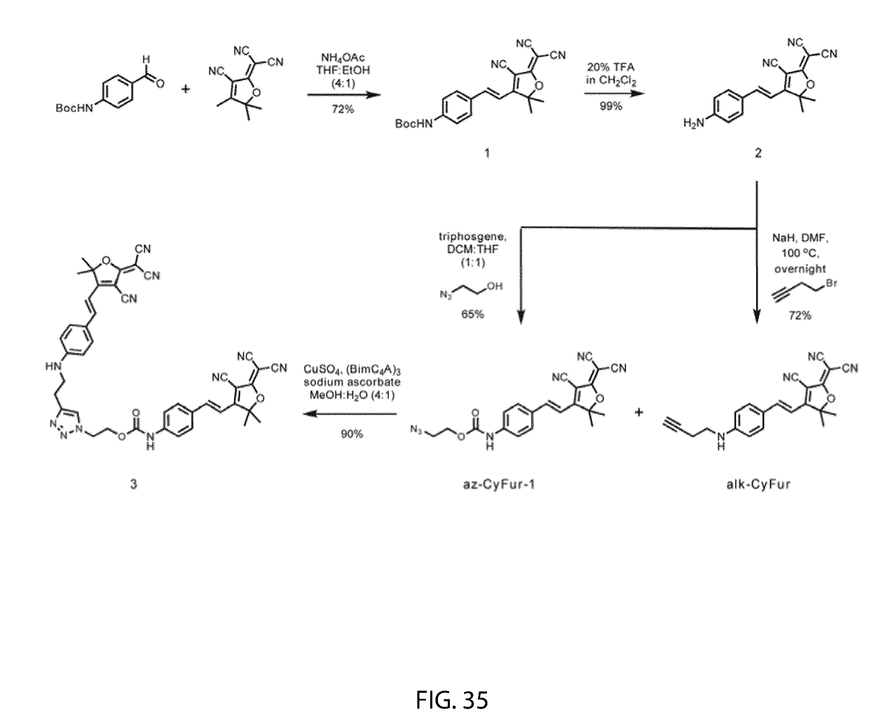

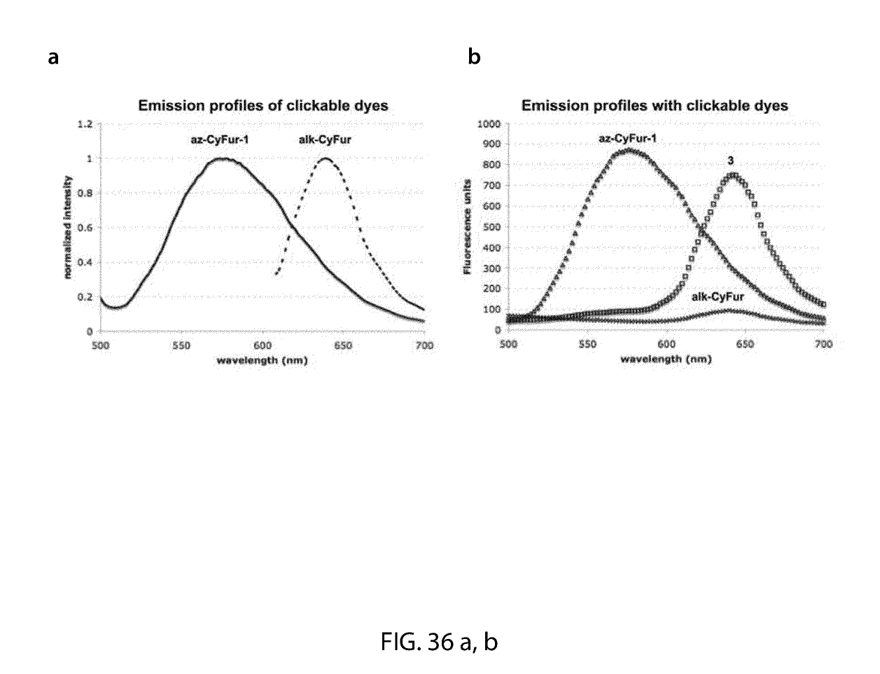

For fluorescent detection of alkyne- and azide-labeled proteins, we synthesized a new set of clickable fluorescent dyes based on DCDHF fluorophores, given their photostability for single-molecule imaging and tunable red-shifted fluorescent emission properties ((S. J. Lord, N. R. Conley, H. L. Lee, S. Y. Nishimura, A. K. Pomerantz, K. A. Willets, Z. Lu, H. Wang, N. Liu, R. Samuel, R. Weber, A. Semyonov, M. He, R. J. Twieg, and W. E. Moerner, Chemphyschem, 2009, 10, 55); (S. J. Lord, N. R. Conley, H. L. Lee, R. Samuel, N. Liu, R. J. Twieg, and W. E. Moerner, J Am Chem Soc, 2008, 130, 9204); (J. Bouffard, Y. Kim, T. M. Swager, R. Weissleder, and S. A. Hilderbrand, Org Lett, 2008, 10, 37). Condensation of tert-butyl 4-formylphenylcarbamate (J. H. Byun, H. Kim, Y. Kim, I. Mook-Jung, D. J. Kim, W. K. Lee, and K. H. Yoo, Bioorg Med Chem Lett, 2008, 18, 5591) and 3-cyano-2-dicyanomethylene-4,5,5-trimethyl-2,5-dihydrofuran (K. G. T. Zhang, Ling Qiu, Yuquan Shen, Synthetic Communications, 2006, 36, 1367)) afforded compound 1 in 72% yield (FIG. 35). Removal of t-Boc group and alkylation of compound 2 with 1-bromobutyne gave alkynyl-DCDHF derivative (alk-CyFur) in 72% yield (FIG. 35). Acylation of DCDHF fluorophore 2 with alkyl-azide substrates afforded az-CyFur-1 and az-CyFur-2 in 65% and 57% yield, respectively (FIG. 35). While alk-CyFur exhibited absorption (abs)/emission (em) maxima at 580 nm/640 nm, acylated-DCDHF derivatives (az-CyFur-1 and az-CyFur-2) yielded abs/em maxima at 470 nm/580 nm (FIG. 36A). The differential fluorescence properties of N-acylated compared to N-alkylated DCDHF derivatives are consistent with previous studies demonstrating that capping of the aniline functionality quenches the red-shifted emission of DCDHF fluorophore 2 ((S. J. Lord, N. R. Conley, H. L. Lee, S. Y. Nishimura, A. K. Pomerantz, K. A. Willets, Z. Lu, H. Wang, N. Liu, R. Samuel, R. Weber, A. Semyonov, M. He, R. J. Twieg, and W. E. Moerner, Chemphyschem, 2009, 10, 55); (S. J. Lord, N. R. Conley, H. L. Lee, R. Samuel, N. Liu, R. J. Twieg, and W. E. Moerner, J Am Chem Soc, 2008, 130, 9204); (J. Bouffard, Y. Kim, T. M. Swager, R. Weissleder, and S. A. Hilderbrand, Org Lett, 2008, 10, 37). Condensation of tert-butyl 4-formylphenylcarbamate (J. H. Byun, H. Kim, Y. Kim, I. Mook-Jung, D. J. Kim, W. K. Lee, and K. H. Yoo, Bioorg Med Chem Lett, 2008, 18, 5591) and 3-cyano-2-dicyanomethylene-4,5,5-trimethyl-2,5-dihydrofuran (K. G. T. Zhang, Ling Qiu, Yuquan Shen, Synthetic Communications, 2006, 36, 1367)). The quantum yields of all three clickable CyFur dyes are similar to previously described N-alkylated and quenched DCDHF derivatives (Table 1) ((S. J. Lord, N. R. Conley, H. L. Lee, S. Y. Nishimura, A. K. Pomerantz, K. A. Willets, Z. Lu, H. Wang, N. Liu, R. Samuel, R. Weber, A. Semyonov, M. He, R. J. Twieg, and W. E. Moerner, Chemphyschem, 2009, 10, 55); (S. J. Lord, N. R. Conley, H. L. Lee, R. Samuel, N. Liu, R. J. Twieg, and W. E. Moerner, J Am Chem Soc, 2008, 130, 9204); (J. Bouffard, Y. Kim, T. M. Swager, R. Weissleder, and S. A. Hilderbrand, Org Lett, 2008, 10, 37). Condensation of tert-butyl 4-formylphenylcarbamate (J. H. Byun, H. Kim, Y. Kim, I. Mook-Jung, D. J. Kim, W. K. Lee, and K. H. Yoo, Bioorg Med Chem Lett, 2008, 18, 5591) and 3-cyano-2-dicyanomethylene-4,5,5-trimethyl-2,5-dihydrofuran (K. G. T. Zhang, Ling Qiu, Yuquan Shen, Synthetic Communications, 2006, 36, 1367)). In accordance with other reported DCDHF-based fluorophores, the fluorescence emission of CyFtur dyes increases significantly in a more viscous or rigid environment such as glycerol. Based on the observed absorption and emission spectra of az-CyFur-1 and alk-CyFur, dimerization of these two fluorophores was predicted to undergo Förster resonance energy transfer (FRET). Indeed, coupling of az-CyFur-1 and alk-CyFur via CuAAC afforded clicked fluorophore 3, which exhibited FRET between the acylated- and alkylated-DCDHF derivatives. Excitation of fluorophore 3 at 410 nm resulted in strong fluorescence emission at 640 nm, whereas an unreacted 1:1 mixture of az-CyFur-1:alk-CyFur afforded similar spectral properties to the dyes alone (FIG. 36). These results demonstrate that differential modification of DCDHR fluorophores provides clickable red-shifted fluorescent dyes with tunable spectral properties that can also function as donor and acceptor FRET pairs.

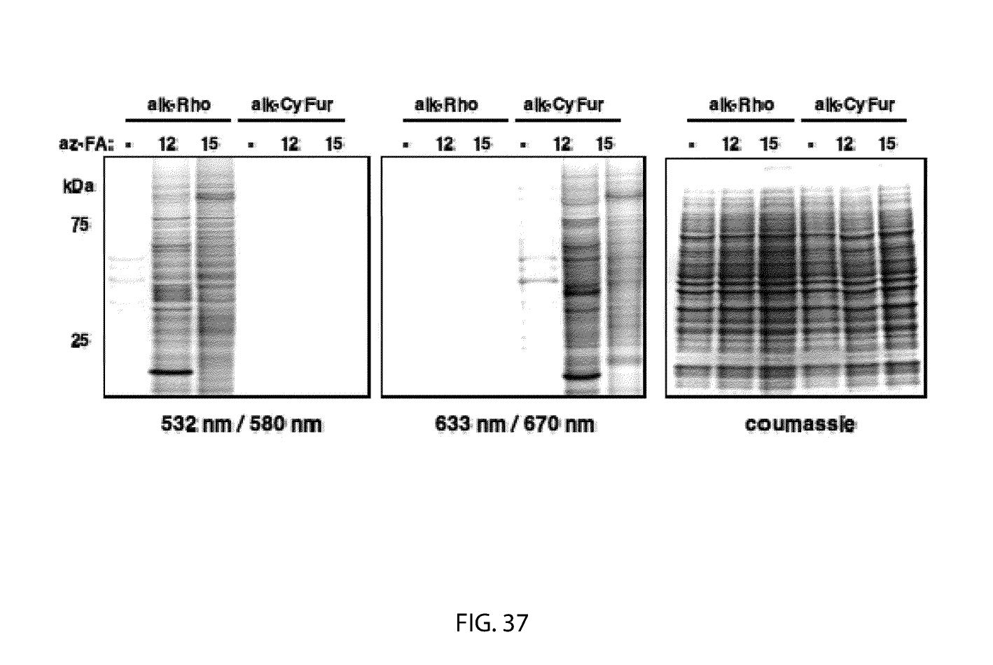

To explore the utility of these clickable CyFur dyes for imaging azide- and alkyne-modified proteins in vitro and in cells, we employed azido- and alkynyl-fatty acids chemical reporters to metabolically label N-myristoylated and S-palmitoylated proteins ((G. Charron, M. M. Zhang, J. S. Yount, J. Wilson, A. S. Raghavan, E. Shamir, and H. C. Hang, J Am Chem Soc, 2009, 131, 4967); (H. C. Hang, E. J. Geutjes, G. Grotenbreg, A. M. Pollington, M. J. Bijlmakers, and H. L. Ploegh, J Am Chem Soc, 2007, 129, 2744)). Jurkat T cell lysates labeled with azido-fatty acids (az-12, az-15) were subsequently reacted with alk-CyFur or alk-Rho via the CuAAC and separated by gel-electrophoresis (FIG. 37). Labeled proteins were visualized by in-gel fluorescence scanning at various excitation/emission channels to detect CyFur- and Rho-modified proteins. Fluorescence imaging at 633 nm excitation and 670 nm emission allowed selective detection of alk-CyFur-labeled proteins analogous to the profile of fatty-acylated proteins visualized with alk-Rho (excitation 532 nm/emission 580 nm) (FIG. 37) (Charron, M. M. Zhang, J. S. Yount, J. Wilson, A. S. Raghavan, E. Shamir, and H. C. Hang, J Am Chem Soc, 2009, 131, 4967). Similarly, Az-CyFur-1 allows selective fluorescent imaging of alk-12-labeled cell lysates. The near-infrared fluorescent properties of alk-CyFur enabled profiling of azide-modified proteins in gels with minimal spectral overlap to acylated-CyFur and Rho fluorophores (FIG. 37).

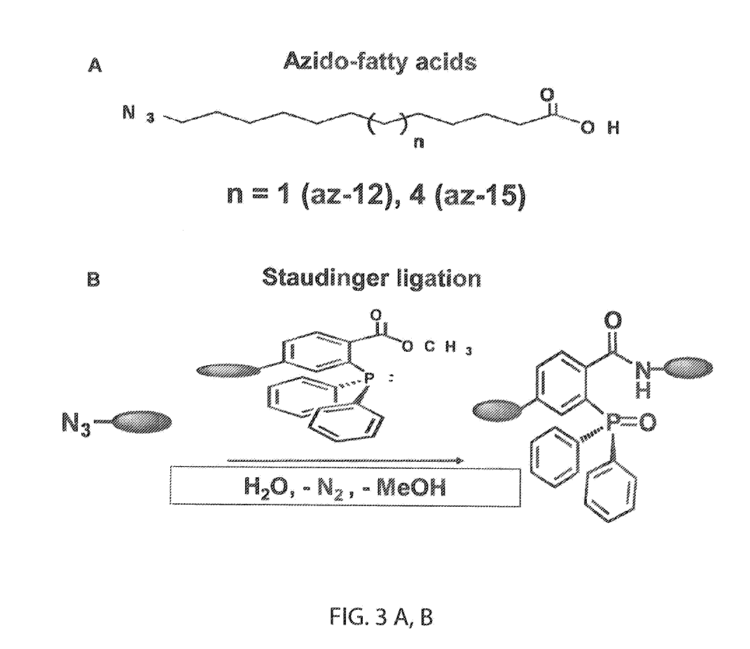

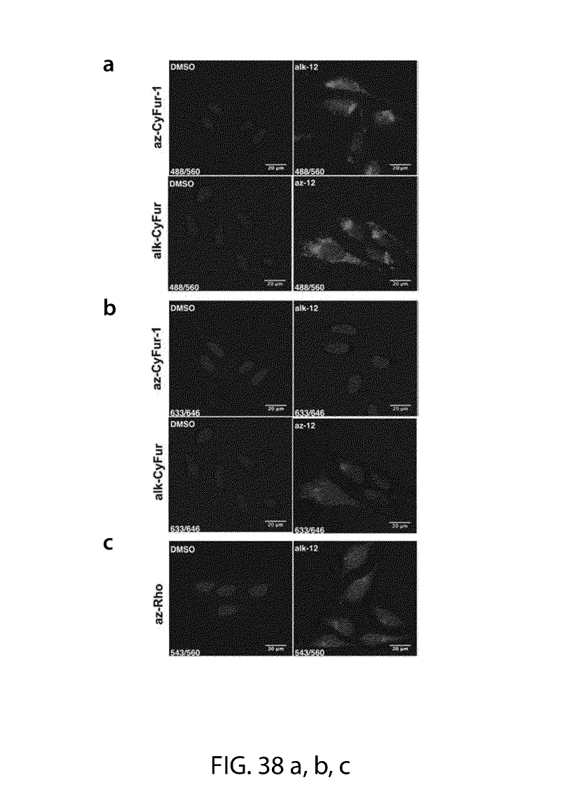

The clickable and environmentally-sensitive CyFur dyes also allow robust fluorescent imaging of azide- and alkyne-labeled proteins in cells. HeLa cells were metabolically labeled with az-12 or alk-12, fixed/permeabilized, reacted with alk-CyFur, az-CyFur-1 or az-Rho and imaged as previously described, (G. Charron, M. M. Zhang, J. S. Yount, J. Wilson, A. S. Raghavan, E. Shamir, and H. C. Hang, J Am Chem Soc, 2009, 131, 4967). Az-12 and alk-12-labeled HeLa cells yielded significantly higher levels of fluorescent labeling with alk-CyFur and az-CyFur-1, respectively, compared to DMSO control using settings for fluorescein dyes (excitation 488 nm/emission 560 nm) (FIG. 3A). In contrast, image settings for red-emitting Cy5 dyes (excitation 630 nm/emission 650 nm) enables visualization of alk-CyFur-labeled cells with no crosstalk to az-CyFur-labeled cells (FIG. 38B). CyFur-labeled proteins were concentrated within intracellular membranes and excluded from the nuclei, which is in accordance with previous imaging studies of fatty-acylated proteins ((G. Charron, M. M. Zhang, J. S. Yount, J. Wilson, A. S. Raghavan, E. Shamir, and H. C. Hang, J Am Chem Soc, 2009, 131, 4967); (R. N. Hannoush and N. Arenas-Ramirez, ACS Chem Biol, 2009, 4, 581)), and similar to that observed for cellular labeling with az-Rho done in parallel (FIG. 38C). Interestingly, fatty acid chemical reporter labeled cells visualized with clickable CyFur dyes yielded more punctate intracellular membrane structures compared to Rho dyes, which may be due to solvatochromism for hydrophobic environments reported for DCDHF fluorophores ((S. J. Lord, N. R. Conley, H. L. Lee, S. Y. Nishimura, A. K. Pomerantz, K. A. Willets, Z. Lu, H. Wang, N. Liu, R. Samuel, R. Weber, A. Semyonov, M. He, R. J. Twieg, and W. E. Moerner, Chemphyschem, 2009, 10, 55); (J. Bouffard, Y. Kim, T. M. Swager, R. Weissleder, and S. A. Hilderbrand, Org Lett, 2008, 10, 37)).

Synthesis of these clickable CyFur dyes is efficient, modular and scalable, which enables facile access to azide- or alkyne-modified fluorophores with different spectral properties by alkylation or acylation of single common DCDHF fluorescent precursor. The in-gel fluorescence scanning and cellular imaging studies of azide- and alkyne-modified fatty-acylated proteins showcase the utility of the clickable CyFur dyes for imaging endogenously expressed proteins. The fluorescent property of alk-CyFur complements previously reported clickable near-infrared dyes ((F. Shao, R. Weissleder, and S. A. Hilderbrand, Bioconjug Chem, 2008, 19, 2487); (P. Kele, X. Li, M. Link, K. Nagy, A. Herner, K. Lorincz, S. Beni, and O, S. Wolfbeis, Org Biomol Chem, 2009, 7, 3486); (P. Kele, G. Mezo, D. Achatz, and O, S. Wolfbeis, Angew Chem Int Ed Engl, 2009, 48, 344)). and should facilitate dual imaging of chemical reporters as well as in vivo imaging applications in the future. Additionally, the spectral overlap of the acylated- and alkylated-CyFur dyes yields useful donor and acceptor pairs for further FRET studies. The clickable CyFur dyes reported here provide alternative and readily accessible reagents for multimodal fluorescence imaging applications using bioorthogonal chemical probes/reporters to study cellular pathways.

TABLE 1
Optical properties of clickable CyFur dyes.

| | λₘₐₓ^{a} (nm) | λₘₐₓ^{a} (nm) | εₘₐₓ^{b} (M⁻¹cm⁻¹) | Φ_{F}^{c} |
|---|---|---|---|---|
| alk-CyFur | 580 | 640 | 33,933 | 0.0147 |
| az-CyFur-1 | 470 | 580 | 20,533 | 0.0067 |
| az-CyFur-2 | 470 | 580 | 12,100 | 0.0027 |

^{a}Spectra were obtained in DMSO.
^{b}Measurements were done in MeOH. Extinction coefficients at 470 nm for az-CyFur-1, az-CyFur-2 and at 580 nm for alk-CyFur are averaged over three independent experiments.
^{c}Quantum yields referenced against cresyl violet (ΦF = 0.54 in MeOH). Alk-CyFur was excited at 580 nm. Both az-CyFur-1 and az-CyFur-2 were excited at 470 nm.

Metabolic Labeling and Preparation of Cell Lysates.

Jurkat T cells and HeLa cells were cultured and metabolically labeled with DMSO, azido-fatty acids (az-12 and az-15) or alkynyl-fatty acids (alk-12) as previously described. Jurkat T cell lysates used for protein labeling studies were prepared as previously described (G. Charron, M. M. Zhang, J. S. Yount, J. Wilson, A. S. Raghavan, E. Shamir and H. C. Hang, J Am Chem Soc, 2009, 131, 4967-4975).

CuI-Catalyzed Huisgen [3+2] Cycloaddition/Click Chemistry.

Cell lysates (50 μg) in 44.5 μL of buffer (150 mM NaCl, 50 mM triethanolamine pH 7.4, 4% SDS) were reacted with freshly prepared click chemistry reaction cocktail: [azido- or alkynyl-CyFurs or az-rho (100 μM, 5 mM stock solution in DMSO), tris(2-carboxyethyl)phosphine hydrochloride (TCEP) (1 mM, 50 mM freshly prepared stock solution in deionized water), tris[(1-benzyl-1H-1,2,3-triazol-4-yl)methyl]amine (TBTA) (100 μM, 2 mM stock solution in DMSO) and CuSO₄.5H₂O (1 mM, 50 mM freshly prepared stock solution in deionized water)] for a total reaction volume of 50 μL for 1 h at room temperature. Following methanol-chloroform precipitation, the protein pellet was redissolved in 18 L of buffer (4% SDS, 50 mM triethanolamine pH 7.4, 150 mM NaCl) and separated by SDS-PAGE (G. Charron, M. M. Zhang, J. S. Yount, J. Wilson, A. S. Raghavan, E. Shamir and H. C. Hang, J Am Chem Soc, 2009, 131, 4967-4975).

In-gel Fluorescence Scanning. Proteins separated by SDS-PAGE were visualized by first soaking the gel in 40% MeOH, 10% acetic acid in water with shaking for 20 mins and directly scanning the gel on an Amersham Biosciences Typhoon 9400 variable mode imager. Proteins labeled by az-CyFur-1 were visualized with excitation at 488 nm and 555 nm emission filter with 30 nm band-pass. Proteins labeled by az-Rho were visualized with excitation at 532 nm and 580 nm emission filter with 30 nm band-pass. Proteins labeled by alk-CyFur were visualized with excitation at 633 nm and 670 nm emission filter with 30 nm band-pass.

Fluorescence imaging. Cells for fluorescence microscopy were prepared as previously reported (G. Charron, M. M. Zhang, J. S. Yount, J. Wilson, A. S. Raghavan, E. Shamir and H. C. Hang, J Am Chem Soc, 2009, 131, 4967-4975). Slides were mounted with Prolong Gold with DAPI from Invitrogen. Confocal images were collected using a Zeiss LSM 510 META laser scanning confocal microscope equipped with a C-Apochromat 40×/1.20 water objective. DAPI was excited at 405 nm with a Diode laser and emission was measured through a band-pass 420-480 nm filters. Az-CyFur-1 was excited with an argon laser at 488 nm and emission was collected at a LP560 nm filter. Az-rho was excited with a HeNe laser at 543 nm and emission was collected through a band-pass 560-615 nm filter. Alk-CyFur was excited with a HeNe laser at 633 nm, and emission was collected through a band-pass 646-753 nm filter.

Absorbance and fluorescence studies. Absorption spectra and fluorescence data were collected on SpectraMax M2 multi-detection reader (Molecular Devices). The spectra in solution were obtained at 25° C. using a quartz cuvette with a path length of 1 cm. Fluorescence quantum yields (ΦF) of CyFur dyes were determined against cresyl violet (ΦF=0.54 in methanol).

Chemical synthesis. All chemicals were obtained either from Sigma-Aldrich, MP Biomedicals, Alfa Aesar, TCI, Fluka or Acros and were used as received unless otherwise noted. The silica gel used in flash column chromatography was Fisher 5704 (60-200 Mesh, Chromatographic Grade). Analytical thin layer chromatography (TLC) was conducted on Merck silica gel plates with fluorescent indicator on glass (5-20 μm, 60 Å) with detection by ceric ammonium molybdate, basic KMnO4 or UV light. The 1H and 13C NMR spectra were obtained on a Bruker AVANCE-600 spectrometer equipped with a cryoprobe. Chemical shifts were reported in δ ppm values downfield from tetramethylsilane and J values were reported in Hz. MALDI-TOF mass spectra were obtained on an Applied Biosystems Voyager-DE. Literature procedures were followed for synthesis of the precursors tert-butyl-4-formylphenylcarbamate2 and 3-cyano-2-dicyanomethylene-4,5,5-trimethyl-2,5-dihydrofuran (T. Zhang, K P. Guo, L. Qiu, Yuquan Shen, Synthetic Communications, 2006, 36, 1367-1372). tert-butyl-4-formylphenylcarbamate was isolated in 82% yield over 2 steps from commercially available 4-aminobenzylalcohol. 3-cyano-2-dicyanomethylene-4,5,5-trimethyl-2,5-dihydrofuran was isolated in 68% yield. (BimC4A)₃ was obtained following reported synthetic procedure (V. O. Rodionov, S. I. Presolski, S. Gardinier, Y. H. Lim and M. G. Finn, J Am Chem Soc, 2007, 129, 12696-12704).

(E)-tert-butyl 4-(2-(4-cyano-5-(dicyanomethylene)-2,2-dimethyl-2,5-dihydrofuran-3-yl)vinyl)phenylcarbamate (1): 3-cyano-2-dicyanomethylene-4,5,5-trimethyl-2,5-dihydrofuran (500 mg, 2.5 mmol), tent-butyl-4-formylphenylcarbamate (555 mg, 2.5 mmol) and ammonium acetate (193 mg, 2.5 mmol) were dissolved in a mixture of THF (10 mL) and anhydrous EtOH (2.5 mL). The mixture was stirred overnight under argon in the dark at room temperature, turning from pale yellow to orange over the course of the reaction. The solution was diluted in water and extracted two times with 100 mL of ethyl acetate followed by 200 mL of brine wash and then dried over anhydrous Na₂SO₄ and filtered. Evaporation of the solvents under reduced pressure afforded crude product that was purified by silica column chromatography using 2:1 hexanes:ethyl acetate (Rf=0.25) as eluant to yield the final product as reddish-orange solid (726 mg, 72%). ¹H NMR (600 MHz, CD₂Cl₂): δ=7.64 (d, 2H, J=8.4 Hz), 7.60 (d, 1H, J=16.3 Hz), 7.52 (d, 2H, J=8.6 Hz), 6.97 (d, 1H, J=16.4 Hz), 1.78 (s, 6H), 1.51 (s, 9H); ¹³C-NMR (125 MHz, CD₂Cl₂): δ=176.3, 174.9, 152.4, 147.5, 143.6, 131.1, 128.7, 118.7, 113.6, 112.5, 111.9, 111.1, 99.1, 98.4, 81.8, 57.1, 28.4, 26.8; MALDI-TOF: calcd. for C₂₃H₂₂N₄NaO₃ [M+Na]⁺425.16, found 425.47.

(E)-2-(4-(4-aminostyryl)-3-cyano-5,5-dimethylfuran-2(5H)-ylidene)malononitrile (2): To a 25 mL round bottom flask loaded with 1 (200 mg, 0.5 mmol), 10 ml of 20% TFA in dry CH₂Cl₂ was added. The mixture was stirred under argon at room temperature for 2 hrs. The solvent was removed under reduced pressure and dried on high vacuum overnight to give product as a purple solid (150 mg recovered, 99%). The product was used for subsequent reactions without further purification. ¹H NMR (600 MHz, CD₂Cl₂): δ=7.60 (d, 1H, J=16.0 Hz), 7.53 (d, 2H, J=8.3 Hz), 6.83 (d, 1H, J=16.0 Hz), 6.72 (d, 2H, J=8.4 Hz), 1.76 (s, 6H); ¹³C-NMR (125 MHz, CD₂Cl₂): δ=176.8, 175.4, 152.6, 148.9, 132.7, 124.5, 115.4, 112.9, 112.4, 111.7, 110.7, 98.0, 96.6, 55.8, 26.9; MALDI-TOF: calcd. for C₁₈H₁₄N₄NaO [M+Na]⁺325.11, found 325.12.

(E)-2-(4-(4-(but-3-ynylamino)styryl)-3-cyano-5,5-dimethylfuran-2(5H)-ylidene)malononitrile (alk-CyFur): To a 10 mL round bottom flask equipped with a condenser, 2 (30 mg, 0.099 mmol) and 98% NaH (9 mg, 0.4 mmol) were dissolved in dry DMF and stirred at 70° C. under argon for 1 hr. 1-bromobutyne (0.131 g, 0.99 mmol, 10 equiv.) was then added to the mixture and the temperature was increased to 100° C. for overnight stirring. Another 2 equivalence of NaH and 5 equivalence of 1-bromobutyne were added to the reaction mixture and allowed to stir for 1 hr at room temperature. The mixture was cooled to room temperature and quenched with 1 mL of MeOH. The reaction mixture was diluted with 200 mL of water and extracted twice with ethyl acetate (100 mL each time). The resulting organic layer was washed with 10% HCl, brine, dried with anhydrous Na₂SO₄ and concentrated to yield purple crude product. Silica gel chromatography was used to purify the title compound, eluting with 1:1 ethyl acetate:hexanes (Rf=0.5) as the mobile phase to give product as purple solid (25 mg, 72%). ¹H NMR (600 MHz, CD₂Cl₂): δ=7.62 (d, 1H, J=16.0 Hz), 7.56 (d, 2H, J=8.7 Hz), 6.81 (d, 1H, J=16.0 Hz), 6.69 (d, 2H, J=8.7 Hz), 3.44 (t, 2H, J=6.6 Hz), 2.55 (dt, 2H, J=2.6 Hz, J=6.6 Hz), 2.12 (t, 1H, J=2.6 Hz), 1.76 (s, 6H). ¹³C-NMR (125 MHz, CD₂Cl₂): δ=176.9, 175.3, 152.9, 148.9, 132.8, 124.0, 113.6, 113.0, 112.5, 111.9, 110.2, 97.8, 81.4, 70.9, 55.4, 42.3, 30.2, 27.0, 19.5; MALDI-TOF: calcd. for C₂₂H₁₈N₄NaO [M+H]⁺355.15, found 355.42.

(E)-2-azidoethyl 4-(2-(4-cyano-5-(dicyanomethylene)-2,2-dimethyl-2,5-dihydrofuran-3-yl)vinylkthenylcarbamate (az-CyFur-1): To a stirred solution on ice of 1:1 mixture of dry CH₂Cl₂:THF (10 ml) containing 2 (5 mg, 0.017 mmol), triphosgene (14 mg, 0.05 mmol), anhydrous pyridine (12 μL, 0.15 mmol) was added. The mixture was stirred under argon for 2 hrs and the volume was reduced under argon to half to get rid of excess phosgene (Caution: perform in the properly working hood when working with larger scale). Azidoethanol (50 μL, 0.57 mmol, 35 equiv.) with 10 equiv. of triethylamine (20 μL) were then added to the solution, which then turned from yellow to reddish-orange. After stirring for another 2 hr, the solvent was diluted with 100 mL of water, extracted twice with 50 mL of CH₂Cl₂, washed with 1% HCl and then brine. The organic layer was dried over anhydrous Na₂SO₄, filtered, and concentrated under pressure. The crude product was purified by column chromatography on silica gel using 3:2 hexane:acetone (Rf=0.4) as the mobile phase to give product as orange solid (4.6 mg, 65%). ¹H NMR (600 MHz, CD₂Cl₂): δ=7.67 (d, 2H, J=8.7 Hz), 7.62 (d, 1H, J=16.4 Hz), 7.56 (d, 2H, J=8.6 Hz), 7.08 (br, 1H), 6.99 (d, 1H, J=16.4 Hz), 4.35 (t, 2H, J=5.0 Hz), 3.56 (t, 2H, J=5.0 Hz), 1.78 (s, 6H). ¹³C-NMR (125 MHz, CD₂Cl₂): δ=176.3, 174.9, 152.9, 147.3, 142.7, 131.1, 129.7, 119.3, 114.3, 112.4, 111.9, 111.1, 99.8, 98.5, 64.7, 50.7, 26.8, 25.8; MALDI-TOF: calcd. for C₂₁H₁₇N₇NaO₃ [M+Na]⁺438.13, found 438.35.

(E)-6-azido-N-(4-(2-(4-cyano-5-(dicyanomethylene)-2,2-dimethyl-2,5-dihydrofuran-3-yl)vinyl)phenyl)hexanamide (az-CyFur-2): One drop of DMF (catalytic) was added to a stirred solution at room temperature of 6-azidohexanoic acid (10 mg, 0.063 mmol) and 20 equiv. of oxalyl chloride in dry CH₂Cl₂. Reaction mixture was then concentrated under pressure and placed on high vacuum for 30 minutes. A solution of dry CH₂Cl₂ (5 mL) containing 2 (5 mg, 0.017 mmol) and 10 equiv. of triethylamine (20 μL) was then added to the flask containing the activated 6-azidohexanoyl chloride. After 1 hr reaction time, the solvent was diluted with 100 mL of water, extracted twice with washed with 1% HCl (50 mL) and then brine (50 mL). The organic layer was dried over anhydrous Na₂SO₄, filtered, and concentrated under pressure. The crude product was purified by silica gel with 3:2 hexanes:acetone (Rf=0.4) as eluant to provide product as orange solid (4.2 mg, 57%). ¹H NMR (600 MHz, CD₂Cl₂): S=7.71 (q, 4H, J=9 Hz, J=6.2 Hz), 7.62 (d, 1H, J=16.4 Hz), 7.44 (br, 1H), 7.03 (d, 1H, J=16.4 Hz), 3.33 (t, 2H, J=6.8 Hz), 2.43 (t, 2H, J=7.4 Hz), 1.82 (s, 6H), 1.80-1.75 (m, 2H), 1.69-1.65 (m, 2H), 1.51-1.48 (m, 2H). ¹³C-NMR (125 MHz, CD₂Cl₂): δ=191.3, 176.2, 174.8, 147.2, 142.9, 130.9, 120.1, 114.3, 112.4, 112.0, 111.8, 111.0, 99.7, 98.4, 51.8, 37.9, 29.1, 26.8, 25.8, 25.3, 25.2; MALDI-TOF: calcd. for C₂₄H₂₃N₇NaO₂ [M+Na]⁺464.18, found 464.33.

2-(4-(2-(4-((E)-2-(4-cyano-5-(dicyanomethylene)-2,2-dimethyl-2,5-dihydrofuran-3-yl)vinyl)phenylamino)ethyl)-1H-1,2,3-triazol-1-yl)ethyl 4-((E)-2-(4-cyano-5-(dicyanomethylene)-2,2-dimethyl-2,5-dihydrofuran-3-yl)vinyl)phenylcarbamate (3): Az-CyFur-1 (1.3 mg, 3.13 μmol) and alk-CyFur (1.2 mg, 3.38 μmol) were dissolved in 1 mL of MeOH and stirred at room temperature under argon in a 5 mL round bottom flask. A mixture of 1 mg of CuSO₄ (1.3 equiv.), 1 mg of sodium ascorbate (1.6 equiv.) and 3 mg of (BimC₄A)₃ (1.4 equiv.) in 250 μL of water was added to the stirred solution. After 1 hr of reaction time, the mixture was diluted with 30 mL of water, extracted twice with ethyl acetate (15 mL each time), washed with 1% HCl, brine, dried over anhydrous Na₂SO₄ and filtered. The concentrated crude product was purified by silica gel chromatography with 20:1 ethyl acetate: MeOH(Rf=0.3) as the mobile phase to give product as purple solid (2.1 mg, 90%). ¹H NMR (600 MHz, CD₂Cl₂): δ=7.68 (d, 2H, J=8.6 Hz), 7.64 (dd, 2H, J=16.4 Hz, J=16.0 Hz), 7.56 (m, 4H), 7.14 (s, 1H), 7.02 (d, 1H, J=16.4 Hz), 6.81 (d, 1H, J=16.0 Hz), 6.70 (d, 2H, J=8.6 Hz), 4.70 (t, 1H, J=5.0 Hz), 4.61 (t, 1H, J=4.9 Hz), 3.81 (t, 1H, J=5.0 Hz), 3.78 (t, 1H, J=5.4 Hz), 3.63 (t, 1H), 3.56 (t, 1H, J=5.0 Hz), 3.51 (t, 1H, J=5.4 Hz), 3.10 (t, 1H), 1.82 (s, 6H), 1.78 (s, 6H). MALDI-TOF: calcd. for C₄₃H₃₅N₁₁O₄ [M+Na]⁺770.29, found 770.79.

### Example 10

### Simultaneous Fluorescence Imaging of S-Acylation Dynamics and Protein Turnover

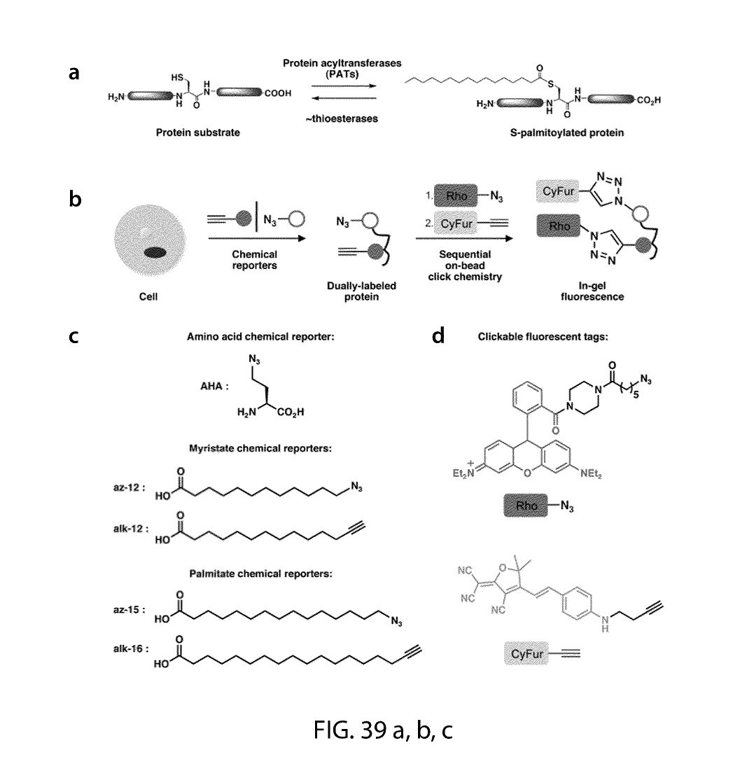

To simultaneously monitor palmitate and protein turnover, we envisioned a pulse-chase experiment employing distinct chemical reporters with orthogonal readouts (FIG. 39B). Protein immunopurification enables sequential on-bead click chemistry reactions, which allows removal of excess reagents that could interfere with the second CuAAC reaction. As for choice of chemical reporters, we exploited chain-length specificity between different fatty acid chemical reporters: the shorter analogs (az-12 and alk-12) preferentially label N-myristoylated proteins while the longer analogs (az-15 and alk-16) get incorporated onto S-palmitoylated proteins ((Hang H C, et al. (2007) Chemical probes for the rapid detection of Fatty-acylated proteins in Mammalian cells. J Am Chem Soc 129(10):2744-2745); Charron G, et al. (2009) Robust fluorescent detection of protein fatty-acylation with chemical reporters. J Am Chem Soc 131(13):4967-4975)) (FIG. 39B). Since N-myristoylation, unlike S-palmitoylation, is a cotranslational and constitutive modification (Johnson D R, Bhatnagar R S, Knoll L J, & Gordon J I (1994) Genetic and biochemical studies of protein N-myristoylation. Annu Rev Biochem 63:869-914), a myristate analog should function as a protein synthesis reporter for N-myristoylated proteins. For orthogonal imaging of azide- and alkyne-modified proteins, we developed clickable fluorescent detection tags (az/alk-CyFur) based on 2-dicyanomethylene-3-cyano-2,5-dihydrofuran fluorophores, which possesses near-IR photophysical properties with negligible crosstalk to rhodamine detection tags (az-Rho/alk-Rho) (Tsou L K, Zhang M M, & Hang H C (2009) Clickable fluorescent dyes for multimodal bioorthogonal imaging. Org. Biomol. Chem. DOI: 10.1039/b917119n). Alkyne- and azide-labeled proteins were visualized with az-Rho and alk-CyFur fluorescence respectively, following sequential on-bead click chemistry reactions (FIG. 39B). This aforementioned sequential click chemistry approach with orthogonal detection tags allows sensitive fluorescent detection of dual-modified proteins and is complementary to tandem copper-mediated (Beatty K E & Tirrell D A (2008) Two-color labeling of temporally defined protein populations in mammalian cells. Bioorg Med Chem Lett 18(22):5995-5999) and copper-free click chemistry strategies (Kele P, Mezo G, Achatz D, & Wolfbeis O S (2009) Dual labeling of biomolecules by using click chemistry: a sequential approach. Angew Chem Int Ed Engl 48(2):344-347); (Baskin J M, et al. (2007) Copper-free click chemistry for dynamic in vivo imaging. Proc Natl Acad Sci USA 104(43):16793-16797); (Laughlin S T, Baskin J M, Amacher S L, & Bertozzi C R (2008) In vivo imaging of membrane-associated glycans in developing zebrafish. Science 320(5876):664-667)).

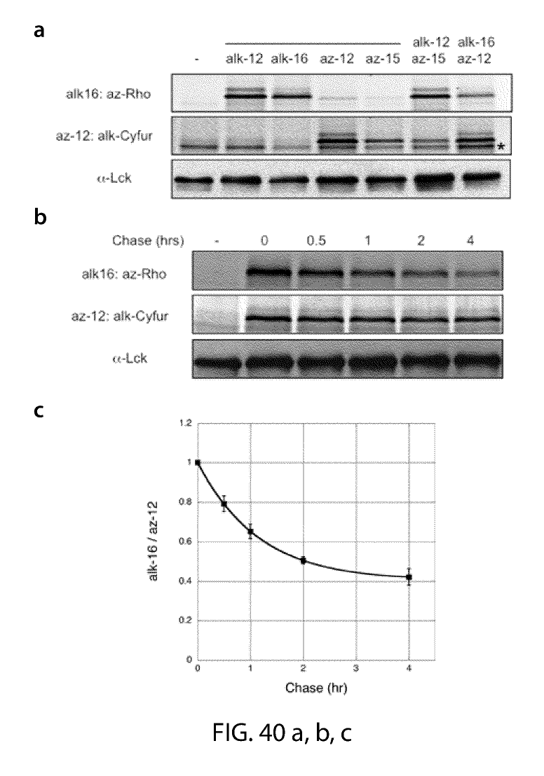

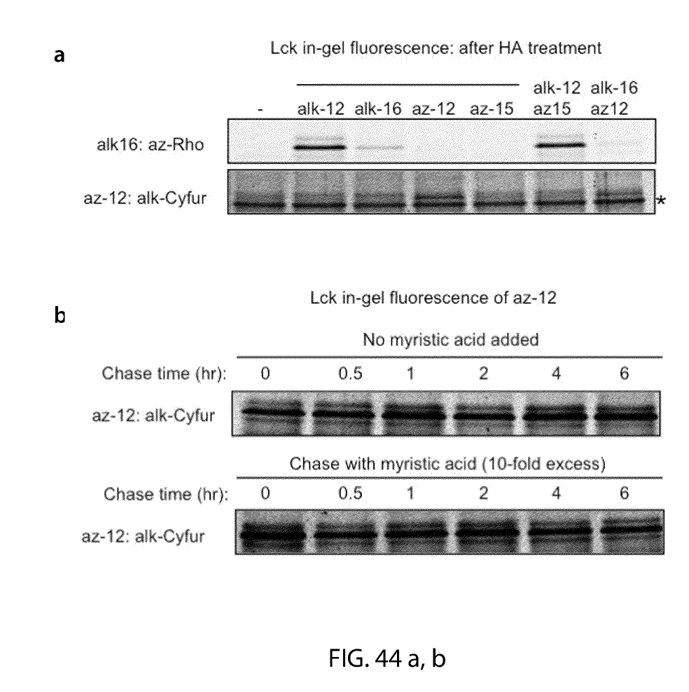

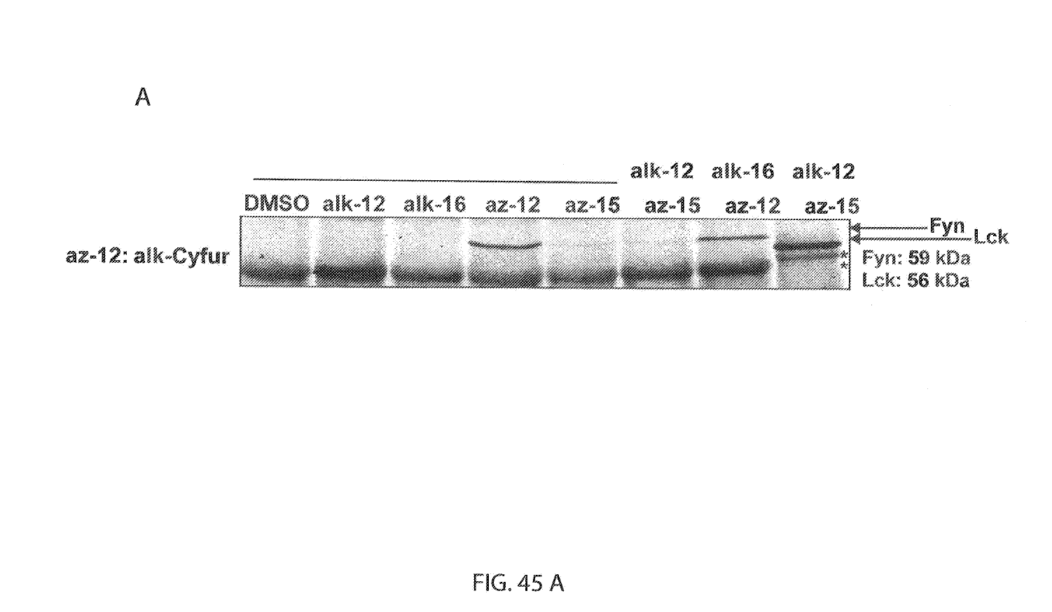

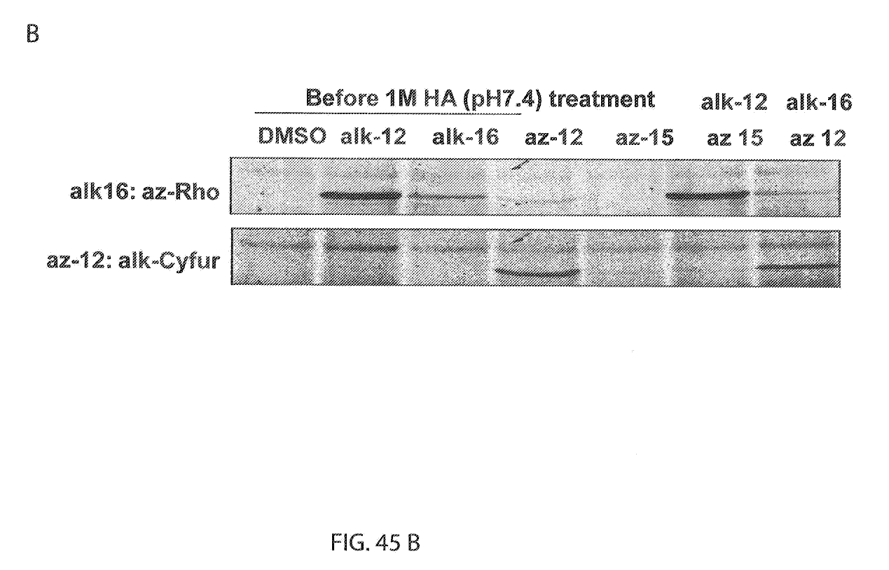

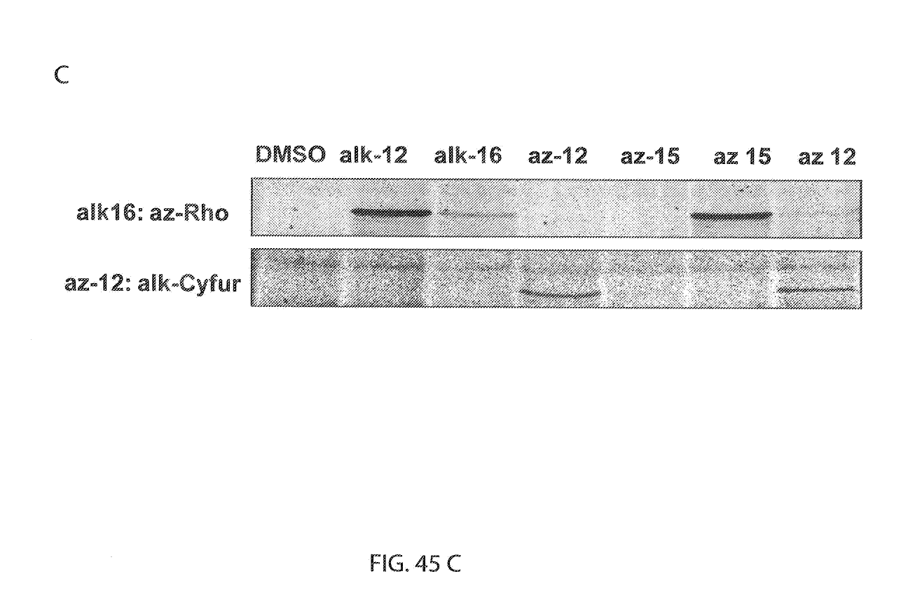

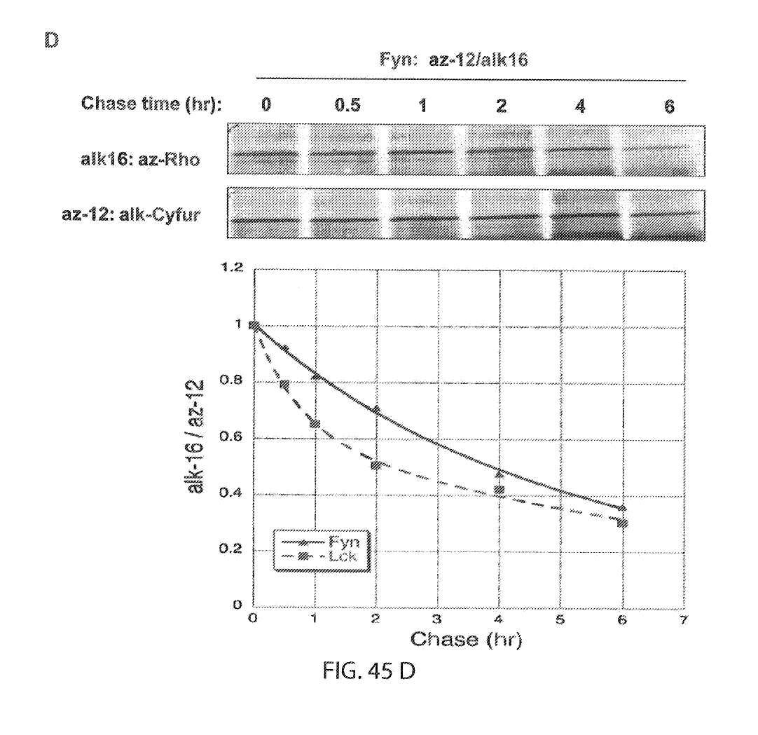

To establish conditions for dual imaging of S-acylation and protein turnover using chemical reporters, we focused on Lck, an N-myristroylated and S-palmitoylated Src-family protein kinase involved in T cell activation. Lck was immunopurified from Jurkat T cells metabolically labeled with either one or both of the myristate (az-12/alk-12) and palmitate (az-15/alk-16) analogs and subjected to sequential on-bead CuAAC with az-Rho and alk-CyFur. In-gel fluorescence scanning demonstrated orthogonal visualization of the two chemical reporters (FIG. 40). At 532 nm excitation/580 nm emission, az-Rho signal was observed for samples labeled with alkynyl-fatty acid reporters (alk-12 and alk-16), while alk-Cyfur fluorescence at 633 nm excitation/670 nm emission only correlated with samples exposed to azido-fatty acid reporters (az-12 and az-15). In-gel hydroxylamine treatment selectively reduced fluorescence associated with thioester-linked palmitate analogs over amide-linked myristate analogs (FIG. 44A), confirming specificity of the fatty acid chemical reporters and the dual detection strategy. To determine the rate of palmitate turnover on Lck, Jurkat cells were pulsed labeled with az-12 and alk-16 followed by a 10-fold excess palmitate chase for different lengths of time. If S-palmitoylation on Lck is dynamic, we expect faster decay of alk-16 signal compared to that of az-12 (FIG. 40B). The calculated palmitate t_{1/2} on Lck is ˜50 minutes, which is much shorter than the protein half-life determined by az-12 labeling (FIG. 40C). No significant decay of alk-16 or az-12 signal was observed with excess myristate as the chase additive over 6 hours (FIG. 44B), confirming the specific visualization of S-acylation/deacylation cycle in our experiments. Analysis of another fatty-acylated kinase, Fyn (Alland L, Peseckis S M, Atherton R E, Berthiaume L, & Resh M D (1994) Dual myristylation and palmitylation of Src family member p59fyn affects subcellular localization. J Biol Chem 269(24):16701-16705), from the same samples yielded a palmitate t_{1/2} of >200 minutes (FIG. 45). Notably, these values correlated with previously reported ³H-palmitate and ³⁵S-Met pulse-chase studies for both Lck and Fyn) ((Wolven A, Okamura H, Rosenblatt Y, & Resh M D (1997) Palmitoylation of p59fyn is reversible and sufficient for plasma membrane association. Mol Biol Cell 8(6):1159-1173); (PaigeLA, Nadler M J, Harrison M L, Cassady J M, & Geahlen R L (1993) Reversible palmitoylation of the protein-tyrosine kinase p56lck. J Biol Chem 268(12):8669-8674), demonstrating this tandem imaging method can be used to determine S-acylation turnover rates on proteins. These results showcase the utility of dual metabolic labeling and sequential on-bead click chemistry to efficiently visualize relative turnover rates of two orthogonal chemical reporters on endogenously expressed proteins.

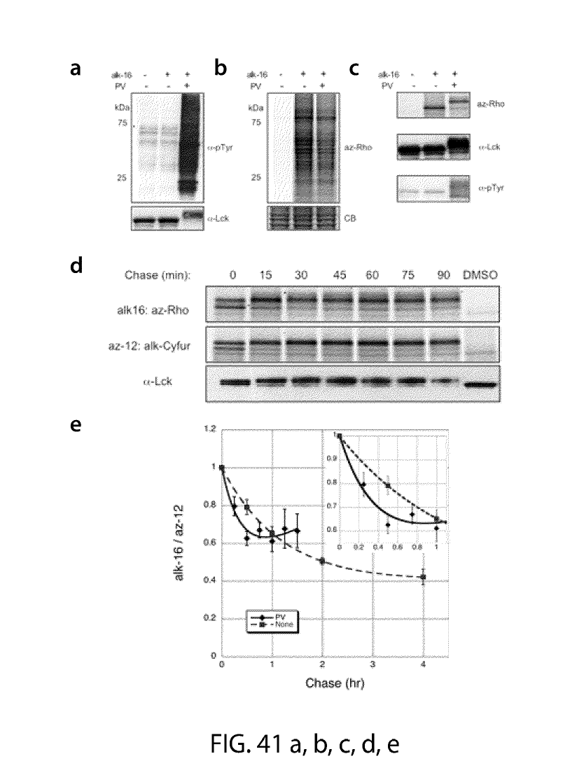

T cell activation accelerates palmitate cycling on Lck. Receptor stimulation has been shown to increase palmitate turnover various proteins (El-Husseini Ael D, et al. (2002) Synaptic strength regulated by palmitate cycling on PSD-95. Cell 108(6):849-863); Bouvier M, et al. (1995) Dynamic palmitoylation of G-protein-coupled receptors in eukaryotic cells. Methods Enzymol 250:300-314)). Since Lck is recruited to immunological synapses and its S-acylation is crucial for T cell activation (Holdorf A D, Lee K H, Burack W R, Allen P M, & Shaw A S (2002) Regulation of Lck activity by CD4 and CD28 in the immunological synapse. Nat Immunol 3(3):259-264), we sought to determine if palmitate cycling on Lck is modulated by T cell receptor (TCR) activity. We utilized pervandate (PV), a phosphatase inhibitor, to activate Jurkat T cells since it triggers an activation response similar to that of TCR cross-linking (Secrist J P, Burns L A, Karnitz L, Koretzky G A, & Abraham R T (1993) Stimulatory effects of the protein tyrosine phosphatase inhibitor, pervanadate, on T-cell activation events. J Biol Chem 268(8):5886-5893). Anti-phosphotyrosine immunoblots revealed substantial increase in protein phosphorylation upon PV-treatment (FIG. 41A) and mobility shift of Lck due to phosphorylation was also evident from anti-Lck blots and in-gel fluorescence scans in PV-treated samples (FIGS. 41B and 41C). PV-induced T cell activation resulted in 2-3 fold acceleration of palmitate cycling on Lck (t_{1/2}˜15 min) (FIGS. 41D and 41E). The activation-induced depalmitoylation of Lck measured by our tandem imaging method was reproduced over several experiments (n=7) (FIG. 41E).

Accelerated depalmitoylation of Lck upon T cell activation raises interesting questions with regards to the function of dynamic S-acylation. While S-palmitoylation of enzymes (Lck) and adaptor proteins (LAT) are critical for TCR signaling (Kabouridis P S, Magee A I, & Ley S C (1997) S-acylation of LCK protein tyrosine kinase is essential for its signaling function in T lymphocytes. EMBO J 16(16):4983-4998); (Zhang W, Trible R P, & Samelson L E (1998) LAT palmitoylation: its essential role in membrane microdomain targeting and tyrosine phosphorylation during T cell activation. Immunity 9(2):239-246), the dynamics of S-acylation on these proteins during cellular stimulation is unclear. Mutagenesis of key Cys residues show that non-acylated Lck is not targeted to the plasma membrane (Kosugi A, et al. (2001) A pivotal role of cysteine 3 of Lck tyrosine kinase for localization to glycolipid-enriched microdomains and T cell activation. Immunol Lett 76(2):133-138). Nonetheless, evidence suggests that S-acylation of Lck is not solely a membrane targeting mechanism. An Lck chimera fused to the transmembrane domain of CD4, which targets it to the plasma membrane, shows reduced association with lipid rafts and decreased T cell signaling activity (Kabouridis P S, Magee A I, & Ley S C (1997) S-acylation of LCK protein tyrosine kinase is essential for its signaling function in T lymphocytes. EMBO J 16(16):4983-4998). Live cell imaging studies of Lck-GFP during T cell activation suggest that Lck is dynamically recruited to the periphery of immunological synapses (Li Q J, et al. (2004) CD4 enhances T cell sensitivity to antigen by coordinating Lck accumulation at the immunological synapse. Nat Immunol 5(8):791-799). It is therefore possible that increased palmitate turnover upon T cell activation may serve to limit the proportion of raft-associated Lck and thus modulate the strength of TCR signaling. A possible explanation is that protein thioesterase activity is stimulated by downstream effects of TCR signaling such as release of calcium from intracellular stores in the endoplasmic reticulum. Alternatively, activated Lck may assume a conformational change favorable towards spontaneous or enzymatic deacylation.

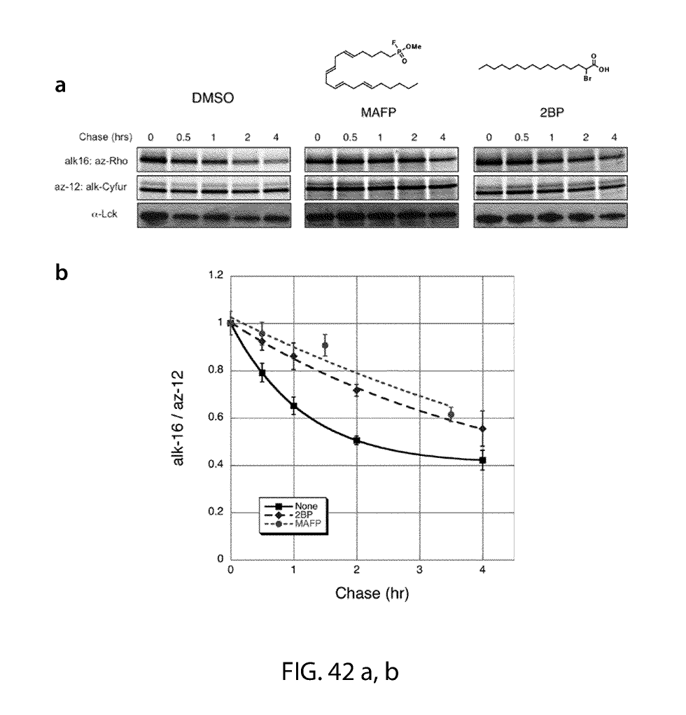

Pharmacological analysis of palmitate cycling on Lck. Efforts to identify enzymes that can deacylate proteins have suggested a cytosolic acyl protein thioesterase-1 (APT 1) and a lysosomal palmitoyl-protein thioesterase-1 (PPT1) as candidate depalmitoylation enzymes (Duncan J A & Gilman A G (1998) A cytoplasmic acyl-protein thioesterase that removes palmitate from G protein alpha subunits and p21(RAS). J Biol Chem 273(25):15830-15837); (Duncan J A & Gilman A G (2002) Characterization of Saccharomyces cerevisiae acyl-protein thioesterase 1, the enzyme responsible for G protein alpha subunit deacylation in vivo. J Biol Chem 277(35):31740-31752). Since both enzymes are predicted to be serine hydrolases based on sequence homology and structure studies, we investigated the effect of a broad-spectrum serine hydrolase inhibitor on Lck depalmitoylation. Addition of methyl arachidonyl fluorophosphonate (MAFP) during the chase significantly retarded palmitate turnover on Lck (FIG. 42), suggesting that serine hydrolases sensitive towards the reactive fluorophosphonate group of MAFP may contribute to the deacylation of Lck in T cells. In contrast, incubation with another broad-spectrum serine hydrolase inhibitor, phenylmethylsulfonyl fluoride (PMSF) had no apparent effect on the initial rate of palmitate removal. Structural studies suggest that the bulky aromatic group of PMSF sterically hinders its binding to the active site of lipid serine hydrolases such as PPT1 (Das A K, et al. (2000) Structural basis for the insensitivity of a serine enzyme (palmitoyl-protein thioesterase) to phenylmethylsulfonyl fluoride. J Biol Chem 275(31):23847-23851). Since PPT1 and PPT2 reside in lysosomal compartments that are not topologically compatible with cytosolic deacylation reactions and APT1 deacylation activity has only been demonstrated in vitro with limited substrates, enzyme(s) that deacylate proteins in cells remain unclear. Nonetheless, our results with mechanism-based inhibitors suggest that serine hydrolases with active sites similar to that of PPT1 may contribute to the observed thioesterase activity on Lck.

We also assessed the effect of 2-bromopalmitate (2BP), a palmitoyltransferase inhibitor commonly used to block S-acylation (Jennings B C, et al. (2009) 2-Bromopalmitate and 2-(2-hydroxy-5-nitro-benzylidene)-benzo[b]thiophen-3-one inhibit DHHC-mediated palmitoylation in vitro. J Lipid Res 50(2):233-242); Resh M D (2006) Use of analogs and inhibitors to study the functional significance of protein palmitoylation. Methods 40(2):191-197)). Interestingly, 2BP also decreased Lck depalmitoylation rate (FIG. 42). The actual targets of 2BP in cells are unknown and several enzymes have been suggested to interact with 2BP (Coleman R A, Rao P, Fogelsong R J, & Bardes E S (1992) 2-Bromopalmitoyl-CoA and 2-bromopalmitate: promiscuous inhibitors of membrane-bound enzymes. Biochim Biophys Acta 1125(2):203-209). It is possible that 2BP, which harbors a reactive α-bromo-carboxyl functional group poised for nucleophilic attack, might also inhibit putative thioesterases. This raises concerns over the use of 2BP as a specific palmitoyltransferase inhibitor in cells and subsequent interpretation of data using 2BP. Collectively, these experiments demonstrate that such a dual detection method can be used to evaluate effects of chemical inhibitors on palmitate turnover. Development of more specific inhibitors using this assay should facilitate discovery and characterization of cellular factors that control palmitate turnover in cells.

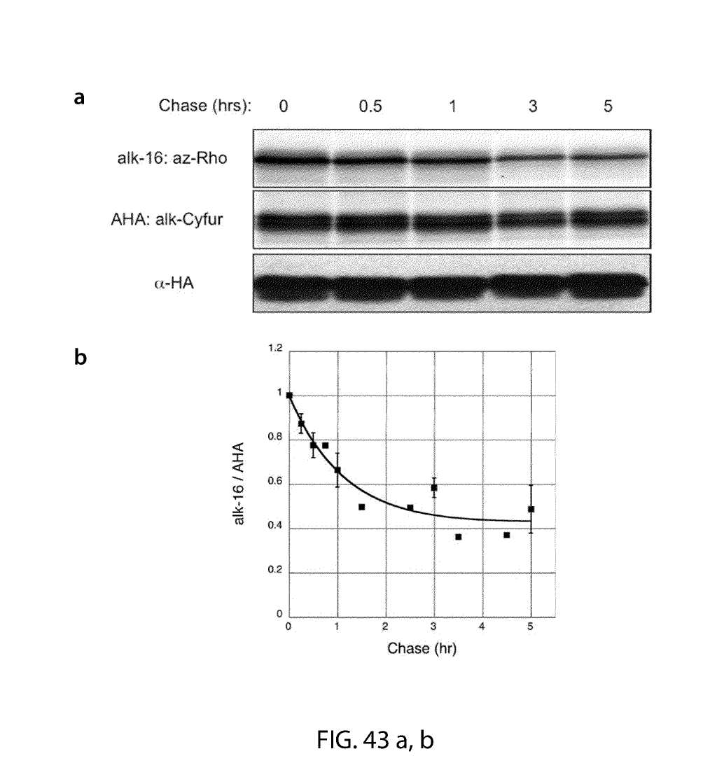

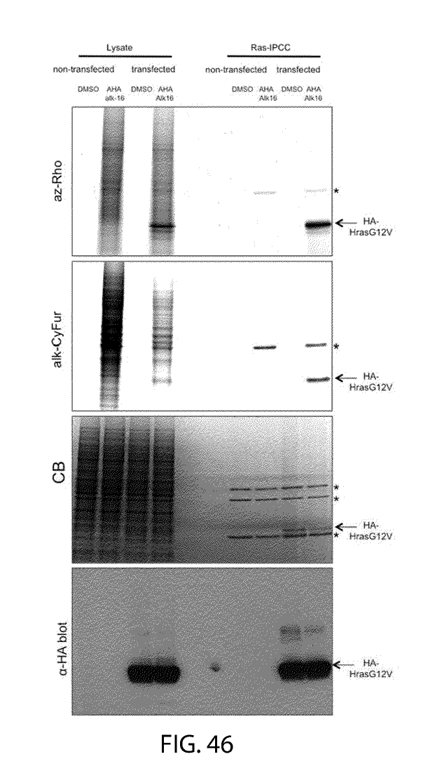

Generality of the tandem imaging method for S-acylated proteins. We expanded the tandem imaging method beyond N-myristoylated proteins by employing more general chemical reporters of protein synthesis. Azidohomoalanine (AHA), an azide-bearing methionine surrogate shown to label newly synthesized proteins with no observed toxicity, is an attractive alternative (Beatty K E, et al. (2006) Fluorescence visualization of newly synthesized proteins in mammalian cells. Angew Chem Int Ed Engl 45(44):7364-7367); (Dieterich D C, Link A J, Graumann J, Tirrell D A, & Schuman E M (2006) Selective identification of newly synthesized proteins in mammalian cells using bioorthogonal noncanonical amino acid tagging (BONCAT). Proc Natl Acad Sci USA 103(25):9482-9487)). We chose to evaluate dual labeling and orthogonal fluorescence detection of H-Ras^{G12V}, which is S-prenylated and S-palmitoylated at the C-terminus and contains four Met residues. The HA-tagged H-Ras^{G12V} construct was transfected into HeLa cells and metabolically labeled with alk-16 and AHA. Immunopurification of HA-tagged H-Ras^{G12V} from HeLa cells followed by sequential CuACC showed incorporation of both chemical reporters (FIG. 46). Subsequent pulse-chase experiments revealed significantly faster chase kinetics for alk-16 than AHA, demonstrating dynamic S-acylation and minimal protein turnover in the time points analyzed (FIG. 43A). The palmitate half-life on H-Ras^{G12V} was estimated to be ˜50 minutes calculated over several experiments (n=5) (FIG. 43B). This is the first report of palmitate turnover rate on H-Ras^{G12V}, which is consistent with more rapid palmitate cycling observed for other GTP-activated oncogenic Hras isoforms relative to non-oncogenic isoforms (Baker T L, Zheng H, Walker J, Coloff J L, & Buss J E (2003) Distinct rates of palmitate turnover on membrane-bound cellular and oncogenic H-ras. J Biol Chem 278(21):19292-19300). Simultaneous measurements of palmitate cycling and protein turnover will crucial for determining whether a protein of interest is indeed dynamically S-acylated. The combined use of alk-16 with AHA potentially allows analysis of palmitate turnover on any S-acylated protein at a fraction of the time and cost required for typical pulse-chase experiments with radioactive analogs.

### Concluding Remarks

S-acylation, unlike other forms of protein lipidation, is reversible. Dissection of the S-acylation/deacylation cycles is required to fully appreciate the biological roles of this dynamic PTM. Given the limitations of conventional pulse-chase experiments involving radioactive analogs, new tools are needed for studies of such dynamic modifications. Integral to our approach is the selective labeling and detection of two different co-/post-translational events on a protein of interest, thereby allowing simultaneous measurement of palmitate and protein turnover rates in the same biological sample. Combining on-bead sequential click chemistry with orthogonal pairs of fatty acid chemical reporters and fluorescent detection tags, we determined turnover rates of Lck-bound palmitate in Jurkat cells upon changes in cellular states or in response to pharmacological perturbations. Use of AHA with alk-16 allows such analyses to be generalized beyond N-myristoylated proteins, which we showed with H-Ras^{G12V}. Besides functional characterization of dynamic S-palmitoylation in distinct cellular states, we envision this strategy to be useful in uncovering cellular factors regulating the S-acylation/deacylation cycle, including putative thioesterases with in vivo deacylating activity. Finally, given its modularity and the wide spectrum of chemical reporters currently available (Sletten E M & Bertozzi C R (2009) Bioorthogonal chemistry: fishing for selectivity in a sea of functionality. Angew Chem Int Ed Engl 48(38):6974-6998), this approach could be readily adapted to study other dynamic protein modifications.

### Materials and Methods

Cell culture growth. Jurkat (human T cell lymphoma) cells were propagated in RMPI 1640 supplemented with 10% fetal bovine serum, 100 U/mL penicillin and 100 μg/mL streptomycin in a humidified CO₂ incubator at 37° C. Cell densities were maintained between 1×10⁵ and 2×10⁶ cells per mL. HeLa cells were cultured in DMEM, supplemented with 10% fetal bovine serum (FBS), 100 U/mL penicillin with 100 μg/mL streptomycin and maintained in a humidified 37° C. incubator with 5% CO₂.

Transfection of N-terminal HA-tagged H-Ras^{G12V}. For transfection studies, HeLa cells were grown in a 10 cm culture plate supplemented with DMEM contains 10% fetal bovine serum in a humidified CO₂ incubator to approximately 90% confluence before transfection with 12-15 μg of DNA using Lipofectamine 2000 (Invitrogen). The N-terminal HA-tagged H-Ras^{G12V} (PCNC10) construct was kindly provided by Dr. Marilyn Resh (Memorial Sloan-Kettering Cancer Center). Cells were transfected about 16 hours prior to metabolic labeling and subsequent chase conditions as described below.

Pulse chase metabolic labeling. Jurkat T cells were labeled with 20 μM az-12 and 20 μM alk-16 in RMPI 1640 supplemented with 2% charcoal-filtered fetal bovine serum, 100 U/mL penicillin and 100 μg/mL streptomycin. Similarly for H-Ras studies, transfected HeLa cells were incubated with 1 mM azidohomoalanine (AHA) and 20 μM alk-16 in methonine-free DMEM (Invitrogen) supplemented with 2% charcoal-filtered fetal bovine serum. The same volume of DMSO was used in the negative controls. After 2 hours incubation, the labeled cells were chased with pre-warmed RMPI 1640 or DMEM containing 200 μM palmitate, 10% fetal bovine serum and/or 100 U/mL penicillin and 100 μg/mL. 100 μM 2-bromopalmitate (2BP) (Fluka) or 20 μM (MAFP) (Sigma) were added to the chase medium to investigate the effects of small molecule inhibitors on palmitate turnover. To determine palmitate turnover upon T cell activation, 100 mM pervanadate, prepared by dissolving sodium orthovanadate with 300 mM H₂O₂, was added to the chase medium for a final pervanadate concentration of 0.1 mM. Samples were taken at various time points during the chase, washed once with PBS and flash frozen in liquid nitrogen prior to storage at −80° C.

Preparation of Cell Lysates. Frozen Jurkat or Hela Cell Pellets were Lysed in Chilled Brij lysis buffer (1% Brij-97, 150 mM NaCl, 50 mM triethanolamine pH 7.4, 10× Roche EDTA-free protease inhibitor cocktail, 10 mM phenylmethysulfonyl fluoride (PMSF)) with vigorous vortexing (3×20 s), placing tubes on ice during intervals to avoid heating of samples. For pervanadate-induced activation studies, 1:50 dilution of phosphatase inhibitor cocktail 2 (Sigma) was added to the lysis buffer. The lysates were spun at 1,000 g for 5 minutes at room temperature to remove cellular debris. Typical lysate concentrations of 4-8 mg/ml were obtained, as quantified using the BCA assay (Pierce).

Immunoprecipitations. Lck and Fyn proteins were immunoprecipitated from 800 μg of Jurkat cell lysates using a mouse anti-Lck (p56^{Lck}) monoclonal (Clone 3A5, Invitrogen) and a rabbit anti-Fyn polyclonal (Upstate) respectively at recommended concentrations. 25 μL of packed Agarose A beads (Roche) was used for each sample. For HA-tagged H-Ras^{G12V} analysis, 15 μL of anti-HA beads (Monoclonal anti-HA agarose conjugate, clone HA-7) was added to 200-300 μg of HeLa cell lysates. After 2 hours incubation on a platform rocker at 4° C., the beads were washed thrice with 1 mL of ice-cold RIPA buffer (1% Nonidet P 40, 1% sodium deoxycholate, 0.1% SDS, 50 mM triethanolamine pH 7.4, 150 mM NaCl) prior to sequential on-bead click chemistry.

Sequential on-bead Cu^{I}-catalyzed azide-alkyne cycloaddition (CuAAC)/click chemistry. The beads were resuspended in 20 μL of PBS and 2.25 μL freshly premixed click chemistry reaction cocktail [az-Rho (100 μM, 10 mM stock solution in DMSO), tris(2-carboxyethyl)phosphine hydrochloride (TCEP) (1 mM, 50 mM freshly prepared stock solution in deionized water), tris[(1-benzyl-1H-1,2,3-triazol-4-yl)methyl]amine (TBTA) (100 μM, 2 mM stock solution in 1:4 DMSO:t-butanol) and CuSO₄.5H₂O (1 mM, 50 mM freshly prepared stock solution in deionized water)] for a total approximate reaction volume of 25 μL for 1 hour at room temperature. The beads were washed thrice with 1 mL of ice-cold RIPA buffer (1% Nonidet P 40, 1% sodium deoxycholate, 0.1% SDS, 50 mM triethanolamine pH 7.4, 150 mM NaCl) and resuspended in 20 μL of SDS buffer (4% SDS, 50 mM triethanolamine pH 7.4, 150 mM NaCl). 2.25 μL of freshly premixed click chemistry reagents (alk-Cyfur in place of az-Rho) were added. After 1 hour at room temperature, the reaction mixture was diluted with 8.7 μL 4× reducing SDS-loading buffer (40% glycerol, 200 mM Tris-HCl pH 6.8, 8% SDS, 0.4% bromophenol blue) and 1.3 μL 2-mercaptoethanol, heated for 5 min at 95° C., and 20 μL was loaded per gel lane for separation by SDS-PAGE (4-20% Bio-Rad Criterion Tris-HCl gel).

In-gel fluorescence scanning. Proteins separated by SDS-PAGE were visualized by first shaking the gel in 40% methanol, 10% acetic acid for at least 1 hour and directly scanning it on a GE healthcare Typhoon 9400 variable mode imager. Rhodamine-associated signal was detected at excitation 532 nm/emission 580 nm while orthogonal detection of Cyfur-associated signal was achieved at excitation 633 nm/emission 670 nm.

Hydroxylamine treatment of gels. After an initial fluorescence scan to determine pretreatment fluorescence, the gel was rinsed with deionized water and incubated with freshly prepared 1 M NH₂OH (pH 7.4) for 2 hours at room temperature on a shaker. The gel was subsequently rinsed with deionized water and incubated with shaking for 2 hours in 40% methanol, 10% acetic acid at room temperature prior to scanning for post-treatment fluorescence.

Western blots. Proteins separated by SDS-PAGE were transferred to nitrocellulose membranes (50 mM Tris, 40 mM glycine, 0.0375% SDS, 20% MeOH in deionized water, Bio-Rad Trans-Blot Semi-Dry Cell, 20 V, 40 min), which were blocked with 10% non-fat milk, 5% BSA, 0.1% Tween-20 in PBS (0.1% PBST) and washed with 0.1% PBST before incubation with appropriate antibodies. Membranes were incubated with a mouse anti-Lck (p56^{Lck}) monoclonal (Clone 3A5, Invitrogen) followed by light chain specific HRP-conjugated affiniPure goat anti-mouse secondary (Jackson Immunoresearch Laboratories) for anti-Lck blots. Likewise, anti-Fyn blots were treated with mouse anti-Fyn monoclonal (S1, Chemicon) followed by goat anti-mouse-HRP conjugated secondary antibody (Upstate). Anti-HA blots were treated with rabbit anti-HA polyclonal (CloneTech) followed by goat anti-rabbit-HRP conjugated secondary antibody (Upstate). Anti-phosphotyrosine blots were blocked with 5% BSA, 0.1% PBST prior to incubation with HRP-conjugated anti-phosphotyrosine mouse monoclonal (PY99, Santa Cruz). Blots were developed using the enhanced chemiluminescent kit (GE Healthcare).

Image processing and calculations. All images were quantified using ImageJ. Taking the ratio of background-corrected alk-16 to az-12 associated fluorescent signal accounted for protein turnover and protein load at each time point of a pulse chase analysis. To allow comparison between pulse chase experiments, alk-16/az-12 values within each dataset were normalized such that alk-16/az-12=1.0 at t=0. Since the data did not form a straight line when plotted on a logarithmic scale, which was observed by others Baker T L, Zheng H, Walker J, Coloff J L, & Buss J E (2003) Distinct rates of palmitate turnover on membrane-bound cellular and oncogenic H-ras. J Biol Chem 278(21):19292-19300, data for each protein or chase condition was fitted to a two-phase exponential decay model using the KaleidaGraph graphing and data analysis software. The equation used was a biphasic exponential decay line m1*exp(−m2*m0)+m3*exp(−m4*m0), which starts at m1+m3 and decays with rate constants m2 and m4. The half-life of protein-bound palmitate (t_{1/2}) was defined as the length time required for the normalized alk-16/az-12 signal to decrease 50% if the decay were to occur solely at the initial rate, which is ln(2)/m2 with m1=0.5.

Chemical synthesis of azidohomoalanine (AHA). All chemicals were obtained either from Sigma-Aldrich, MP Biomedicals, Alfa Aesar, TCI, Fluka or Acros and were used as received unless otherwise noted. The silica gel used in flash column chromatography was Fisher S704 (60-200 Mesh, Chromatographic Grade). Analytical thin layer chromatography (TLC) was conducted on Merck silica gel plates with fluorescent indicator on glass (5-20 μM, 60 Å) with detection by ceric ammonium molybdate, basic KMnO₄ or UV light. The ¹H and ¹³C NMR spectra were obtained on a Bruker AVANCE-600 spectrometer equipped with a cryoprobe. Chemical shifts were reported in μppm values and J values were reported in Hz. MALDI-TOF mass spectra were obtained on an Applied Biosystems Voyager-DE. Fatty acid chemical reporters (az-12, az-15, alk-12 and alk-16) (Charron G, et al. (2009) Robust fluorescent detection of protein fatty-acylation with chemical reporters. J Am Chem Soc 131(13):4967-4975) and clickable fluorescent detection tags (Tsou L K, Zhang M M, & Hang H C (2009) Clickable fluorescent dyes for multimodal bioorthogonal imaging. Org. Biomol. Chem. DOI: 10.1039/b917119n) were synthesized in our laboratory as previously described. Literature procedures were followed for synthesis of the precursors imidazole-1-sulfonyl azide hydrochloride (Goddard-Borger E D & Stick R V (2007) An efficient, inexpensive, and shelf-stable diazotransfer reagent: imidazole-1-sulfonyl azide hydrochloride. Org Lett 9(19):3797-3800).

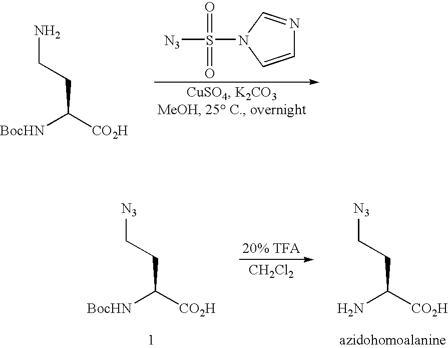

(S)-2-amino-4-azidobutanoic acid (Azidohomoalanine): The diazotransfer reagent, imidazole-1-sulfonyl azide hydrochloride (Tsou L K, Zhang M M, & Hang H C (2009) Clickable fluorescent dyes for multimodal bioorthogonal imaging. Org. Biomol. Chem. DOI: 10.1039/b917119) (1.0 g, 5 mmol, 1.1 equvi.) was added to a stirred suspension of commercially available Boc-Dab-OH (1.0 g, 4.6 mmol, 1 equvi.), potassium carbonate (1.17 g, 8.5 mmol), and copper (II) sulfate pentahydrate (11 mg, 46 μmol, 1 mol %) in methanol (25 mL). Upon completion of the reaction (TLC) after overnight reaction at room temperature, the mixture was concentrated and diluted in 100 mL of ethyl acetate. The organic phase was washed with 1% HCl (100 mL) twice and water (100 mL) once, followed by drying in sodium sulfate. Flash chromatography with 3:1 (hexanes:ethyl acetate) (R_{f}=0.4) furnished compound 1. The identity and purity of compound 1 was checked with MALDI-TOF mass spectrometry and ¹H NMR. The combined organic fractions were further treated with 20% TFA in dry dichloromethane (20 mL) for 4 hours at room temperature. Upon completion of the reaction (TLC), TFA was evaporated at reduced pressure and azeotroped with toluene (5 mL) for three times. Product was redissolved in 10 mL of deionized water and lyophilized to furnish azidohomoalanine as white powder (541 mg, 82% overall yield in two steps). ¹H NMR (600 MHz, D₂O): δ=4.15 (t, 1H, J=6.3 Hz), 3.60 (m, 2H), 2.26-2.12 (m, 2H). ¹³C-NMR (125 MHz, D₂O): δ=173.1, 52.3, 47.7, 29.7; MALDI-TOF: calcd. for C₄H₉N₄O₂ [M+H]⁺145.06, found 145.13. Data were similar to previously reported synthesis of AHA (Link A J, Vink M K, & Tirrell D A (2007) Preparation of the functionalizable methionine surrogate azidohomoalanine via copper-catalyzed diazo transfer. Nat Protoc 2(8):1879-1883).

### Example 11

### Protein Acetylation

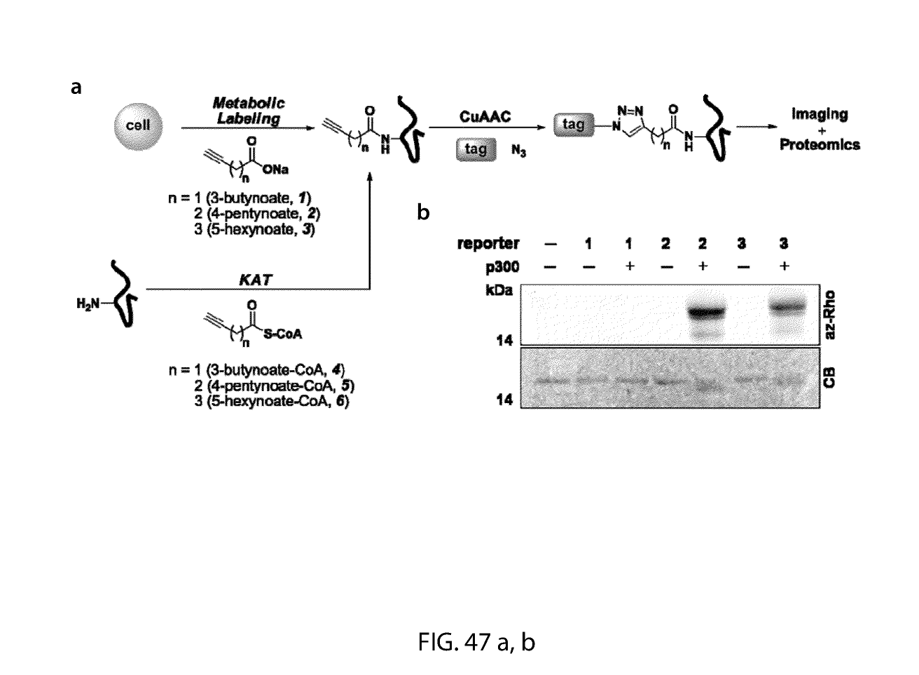

Protein acetylation is a prevalent post-translational modification (PTM) that modulates diverse biological activities in eukaryotes as well as bacterial pathogenesis ((Yang, X. J.; Seto, E. Mol Cell 2008, 31, 449-61); (Mukherjee, S.; Hao, Y.-H.; Orth, K. Trends in Biochemical Sciences 2007, 32, 210-2161,2)). In particular, reversible protein acetylation regulated by lysine acetyltransferases (KATs) and lysine deacetylases (KDACs) plays key roles in controlling gene expression and is misregulated in a variety of diseases (Yang, X. J.; Seto, E. Mol Cell 2008, 31, 449-61). Identifying the protein substrates of specific KATs is crucial for elucidating the function(s) of acetylation (Lin, Y.-y. Cell 2009, 136, 1073). Acetylation is primarily visualized by employing radiolabeled acetate or acetyl-CoA (Brownell, J. E.; Allis, C. D. Proc. Nat. Acad. Sci. U.S.A. 1995, 92, 6364-6368), however, autoradiography exhibits low-sensitivity and is hazardous to handle. Alternatively, bioorthogonal chemical reporters in conjunction with chemoselective ligation methods have afforded new opportunities for sensitive detection of PTMs as well as protein and nucleic acid synthesis (Sletten, E. M.; Bertozzi, C. R. Angew Chem Int Ed Engl 2009, 48, 6974-98). This chemical approach allows the specific installation of bioorthogonal functionalities (azide/alkyne) onto proteins of interest for imaging or proteomics applications. Chloroacetyl-CoA is reported substrate of Gcn5-related KATs in vitro, but does not afford specific detection of acetylated proteins in cells (Yu, M.; de Carvalho, L. P. S.; Sun, G.; Blanchard, J. S. J Am Chem Soc 2006, 128, 15356). Herein, we report alkyne-derivatized chemical reporters that enable rapid detection and identification of acetylated proteins in vitro and in cells via Cu(I)-catalyzed azide-alkyne cycloaddition (CuAAC) with fluorescent or biotinylated tags (FIG. 47A).

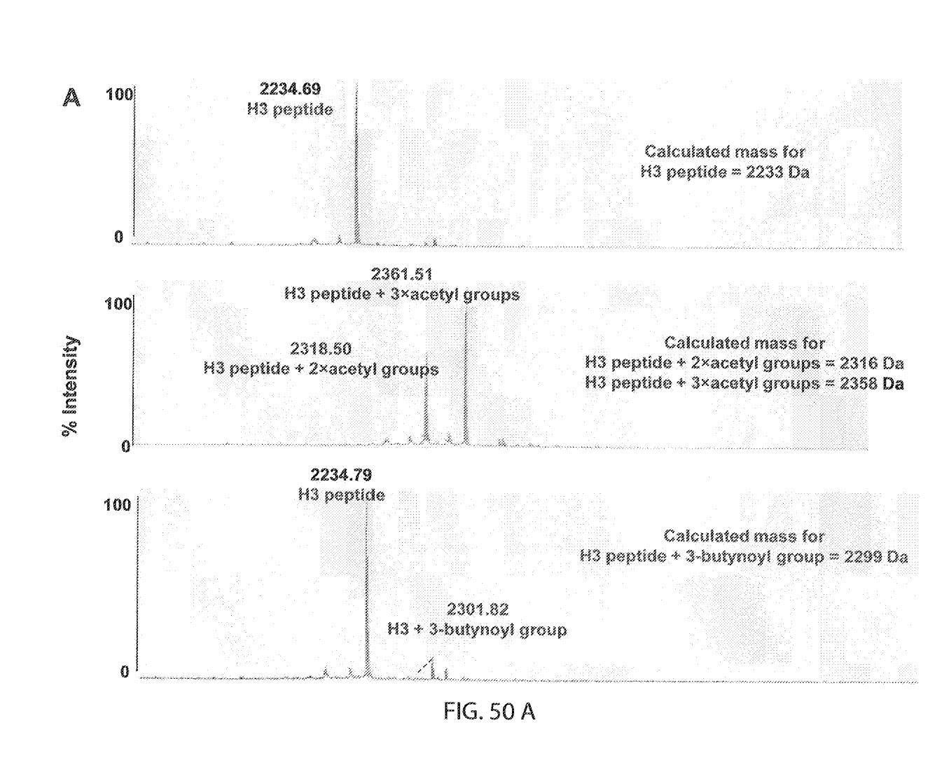

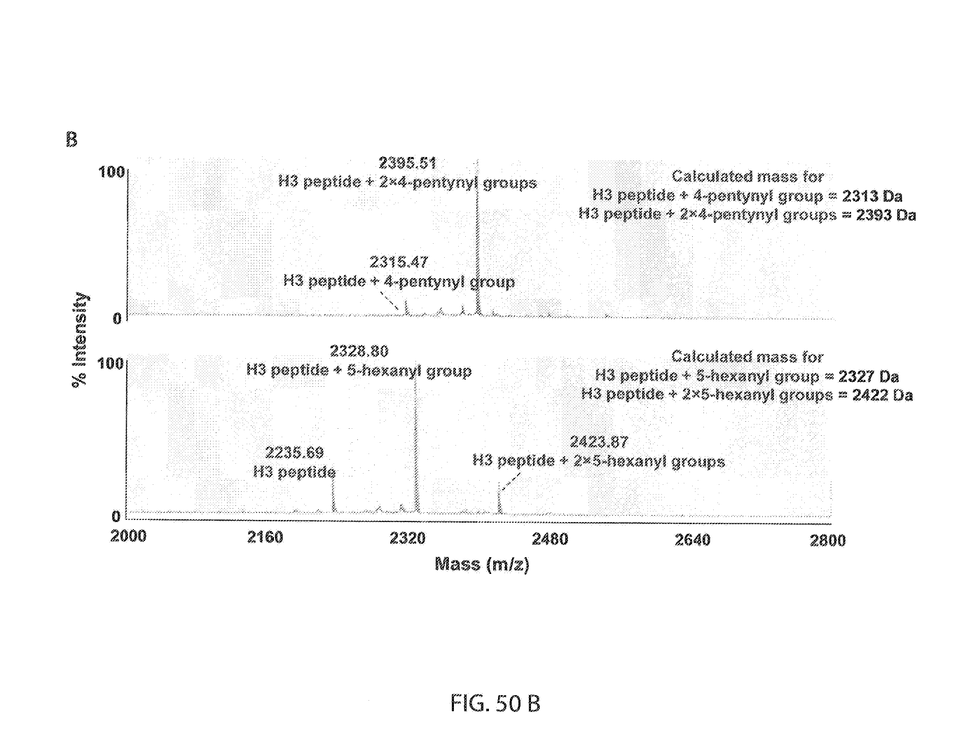

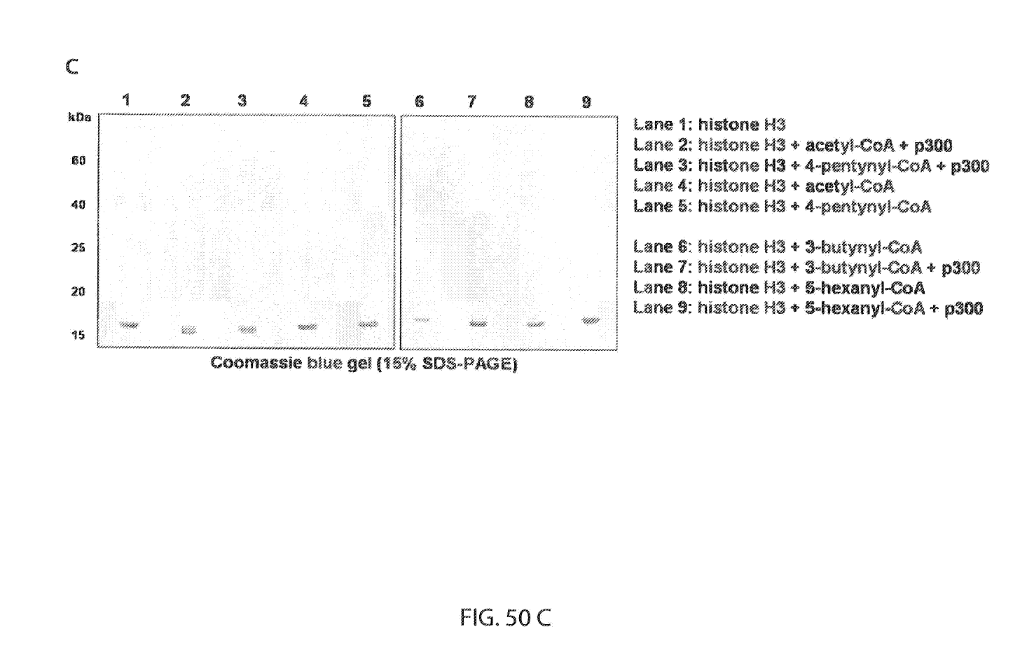

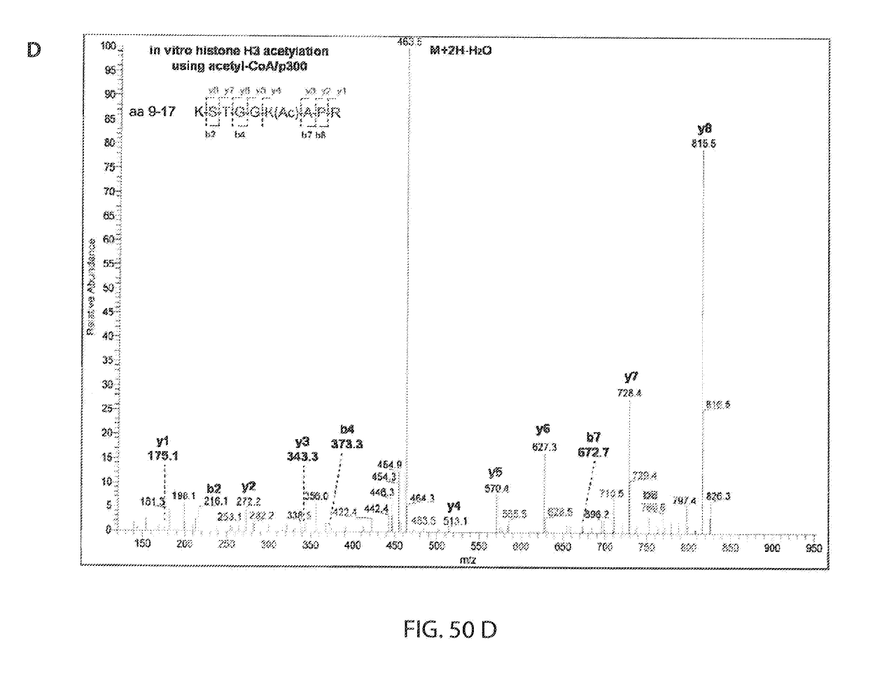

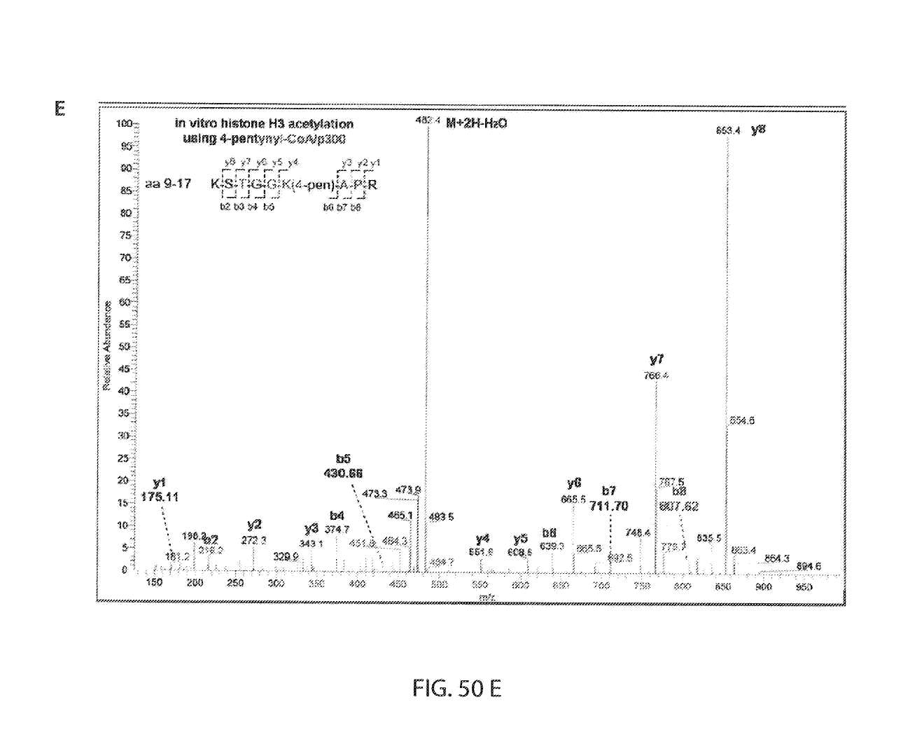

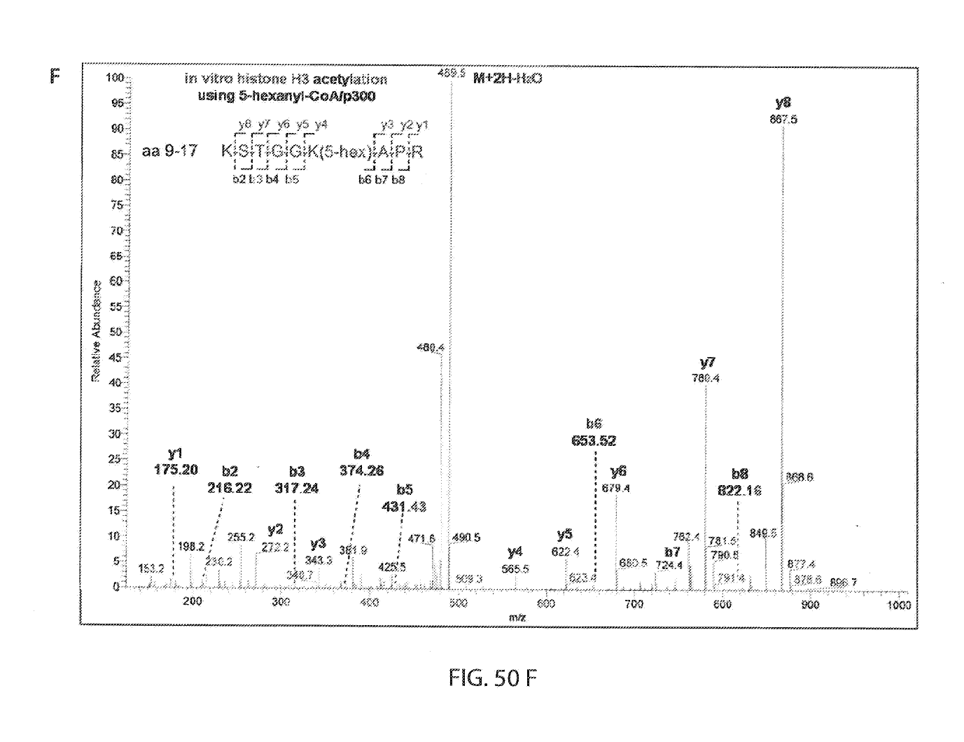

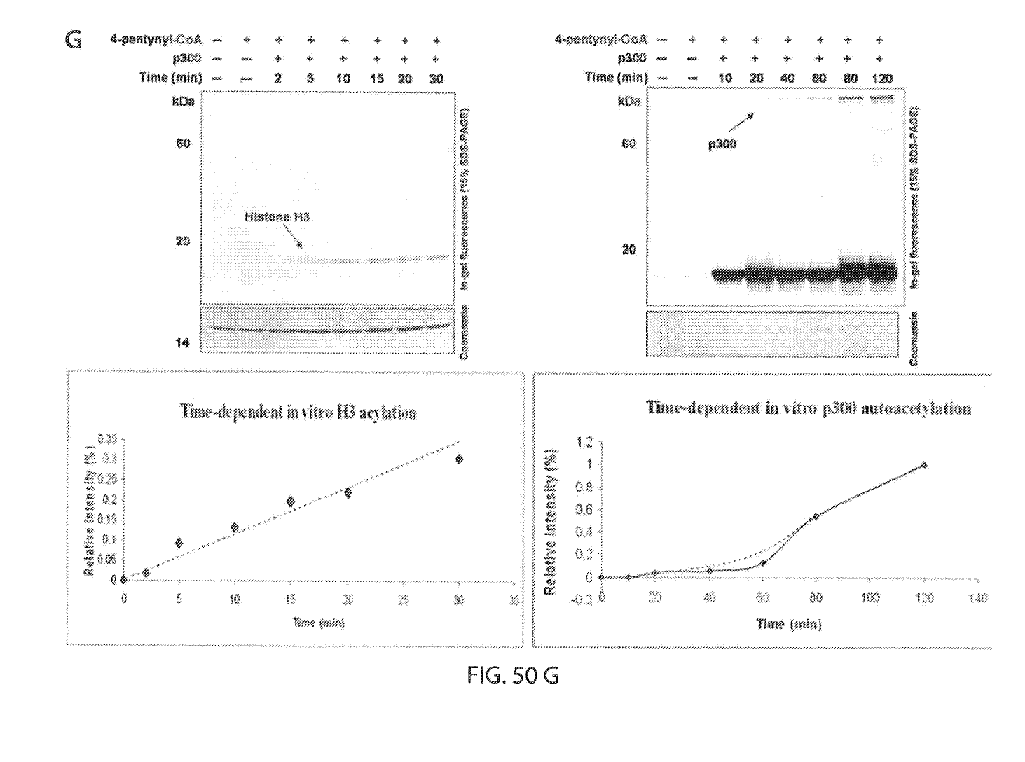

We first investigated whether alkynyl-acetyl-CoA could be utilized by KATs in vitro. Three alkynyl-acetyl-CoA analogs, 3-butynyl-CoA (Analog 1), 4-pentynyl-CoA (Analog 2) and 5-hexynyl-CoA (Analog 3), were synthesized and evaluated as acyl-CoA donors for KAT p300-catalyzed acylation of histone H3 peptide. Mass spectrometry (MS) analysis of these in vitro acylation reactions revealed that amongst three analogs, Analog 2 is readily utilized by p300, while analog 3 was a less efficient acyl-donor substrate (FIG. 50A). Analog 1, however, does not appear to be utilized efficiently by p300 in vitro. To verify that histone H3 was modified by 4-pentynyl-CoA and 5-hexanyl-CoA on lysine residues, in-gel trypsin digestion and tandem MS analysis was performed on control and p300-catalyzed reactions (FIGS. 50B and 50C). Lysine residues of histone H3 were similarly modified by p300 with 4-pentynyl-CoA, 5-hexanyl-CoA and acetyl-CoA, suggesting that alkynyl-acetyl-CoAs can be utilized by p300 without significant perturbations on acceptor substrate specificity of p300 or sites of modification (Table 2). The data presented here demonstrate that alkynyl-acetyl-CoA analogs (2 and 3) can be efficient substrates of KATs, which are consistent with the previous observations of p300 acetyl-donor substrate promiscuity that results in lysine propionylation and butyrylation in vitro and in cells ((Chen, Y.; Sprung, R.; Tang, Y.; Ball, H.; Sangras, B.; Kim, S. C.; Falck, J. R.; Peng, J.; Gu, W.; Zhao, Y. Mol Cell Proteomics 2007, 6, 812-819); (Cheng, Z.; Tang, Y.; Chen, Y.; Kim, S.; Liu, H.; Li, S. S. C.; Gu, W.; Zhao, Y. Mol Cell Proteomics 2009, 8, 45-52)).

TABLE 2

| | | | | | | | |
|---|---|---|---|---|---|---|---|
| | Spectral | | | | | | |
| | Counts | | | | | | |
| Peptide Sequence | of | | | | | | |
| (K: modified | Peptide | | | | | | |
| residue) | H3 | H3 + AcCoA | H3 + AcCoA + p300 | H3 + 2 | H3 + 2 + p300 | H3 + 3 | H3 + 3 + p300 |

| | | | | | | | |
|---|---|---|---|---|---|---|---|
| KQLATK | | | | | | 4 | 10 |
| (18-23) | | | | | | | |
| (SEQ ID NO: 1) | | | | | | | |
| KSAPATGGVK | 28 | 21 | 6 | 23 | 17 | 36 | 38 |
| (27-36) | | | | | | | |
| (SEQ ID NO: 2) | | | | | | | |
| SAPATGGVK | 3 | 2 | | 9 | 7 | 12 | 17 |
| (28-36) | | | | | | | |
| SEQ ID NO: 3 | | | | | | | |
| YRPGTVALR | 1 | 3 | 2 | 2 | 3 | 47 | 57 |
| (41-49) | | | | | | | |
| (SEQ ID NO: 4) | | | | | | | |
| STELLIR | | | | | | 7 | 16 |
| (57-63) | | | | | | | |
| (SEQ ID NO: 5) | | | | | | | |
| KLPFQR | | | 1 | 1 | | | 8 |
| (64-69) | | | | | | | |
| (SEQ ID NO: 6) | | | | | | | |
| EIAQDFK | 25 | 23 | 22 | 27 | 25 | 74 | 89 |
| (73-79) | | | | | | | |
| (SEQ ID NO: 7) | | | | | | | |
| RVTIMPK | | | | | | 1 | |
| (116-122) | | | | | | | |
| (SEQ ID NO: 8) | | | | | | | |
| VTIMPK | 5 | 10 | 1 | 5 | 4 | 3 | 8 |
| (117-122) | | | | | | | |
| (SEQ ID NO: 9) | | | | | | | |
| KSTGGKAPR | | 1 | 8 | | 16 | 2 | 11 |
| (9-17, K14) | | | | | | | |
| (SEQ ID NO: 10) | | | | | | | |
| KSTGGKAPR | | 18 | 60 | | 12 | | 2 |
| (9-17, K9, K14) | | | | | | | |
| (SEQ ID NO: 11) | | | | | | | |
| STGGKAPR | | | | | 9 | | |
| (10-17, K14) | | | | | | | |
| (SEQ ID NO: 12) | | | | | | | |
| KQLATK | | | 2 | | 10 | 2 | 19 |
| (18-23, K18) | | | | | | | |
| (SEQ ID NO: 13) | | | | | | | |
| KQLATKAAR | | | | 1 | 3 | | |
| (18-26, K23) | | | | | | | |
| (SEQ ID NO: 14) | | | | | | | |
| KQLATKAAR | | 4 | 138 | | 18 | | 18 |
| (18-26, K18, K23) | | | | | | | |
| (SEQ ID NO: 15) | | | | | | | |
| QLATKAAR | | | 1 | | | | 1 |
| (19-26, K23) | | | | | | | |
| (SEQ ID NO: 16) | | | | | | | |
| KSAPATGGVK | | 12 | 34 | 3 | 25 | 2 | 12 |
| (27-36, K27) | | | | | | | |
| (SEQ ID NO: 17) | | | | | | | |
| KSAPATGGVK | | | 4 | | | | |
| (27-36, K27, K36) | | | | | | | |
| (SEQ ID NO: 18) | | | | | | | |
| KSAPATGGVKKPHR | | | 34 | | 6 | | |
| (27-40, K27, K36) | | | | | | | |
| (SEQ ID NO: 19) | | | | | | | |
| KSAPATGGVKKPHR | | | 28 | | | | |
| (27-40, K27, K36, | | | | | | | |
| K37) | | | | | | | |
| (SEQ ID NO: 20) | | | | | | | |
| RYQKSTELLIR | | 2 | 5 | | | | |
| (53-63, K56) | | | | | | | |
| (SEQ ID NO: 21) | | | | | | | |
| RVTIMPKDIQLAR | | | 2 | | | | |
| (116-128, K122) | | | | | | | |
| (SEQ ID NO: 22) | | | | | | | |
| VTIMPKDIQLAR | | | 2 | | | | |
| (117-128, K122) | | | | | | | |
| (SEQ ID NO: 23) | | | | | | | |

Table 2. List of spectral counts of peptides acquired from in-gel trypsin digestion of in vitro acetylated and acylated histone H3 (the gel and selected MS/MS spectra were shown in FIG. 50b-50c). Raw tandem mass spectra were searched against the human IPI protein database version 3.56 using SEQUEST search engine (Thermo Scientific). Cysteine carbamidomethylation was searched as fixed modification, while methionine/tryptophan oxidation, asparagines/glutamine deamindation, lysine/serine/threonine/cysteine acetylation, N-terminal acetylation/4-pentynylation/5-hexynylation and lysine 4-pentynylation/5-hexynylation were searched as variable modifications. Each peptide spectrum must meet several selection thresholds including >95% for peptide identification probability, >1.0 for SEQUEST XCorr score, and ±6 ppm for actual minus calculated peptide mass. Those lysine-modified peptides listed in control experiments (H3+AcCoA, H3+2 and H3+3) were derived from p300-independent acetylation/acylation (2: 4-pentynyl-CoA, 3: 5-hexanyl-CoA). For each chosen lysine-modified peptide, the spectral count ratio of p300-catalyzed modification/control must be greater than 2.

Alkynyl-acetyl-CoA analogs were then evaluated for fluorescence detection of acetyltransferase activity by CuAAC with azido-rhodamine (az-Rho) (Charron, G.; Zhang, M. M.; Yount, J. S.; Wilson, J.; Raghavan, A. S.; Shamir, E.; Hang, H. C. J Am Chem Soc 2009, 131, 4967-75). Both Analogs 2 and 3 serve as sensitive reagents for visualizing p300-acylation of histone H3 (FIG. 47B). In the absence of p300 or alkynyl-acetyl-CoA, only minimal fluorescent labeling of histone H3 was observed, demonstrating the specificity of this detection method for monitoring KAT activity. Moreover, the fluorescence intensity of histone H3 acylation and p300-autoacylation were time-dependent, confirming enzyme-catalyzed acylation of substrates (FIG. 50D). These in vitro studies demonstrate that alkynyl-acetyl-CoA analogs together with CuAAC-mediated fluorescence detection facilitates rapid analysis of protein acetylation with picomolar sensitivity within minutes compared to days or weeks required for radioactivity.

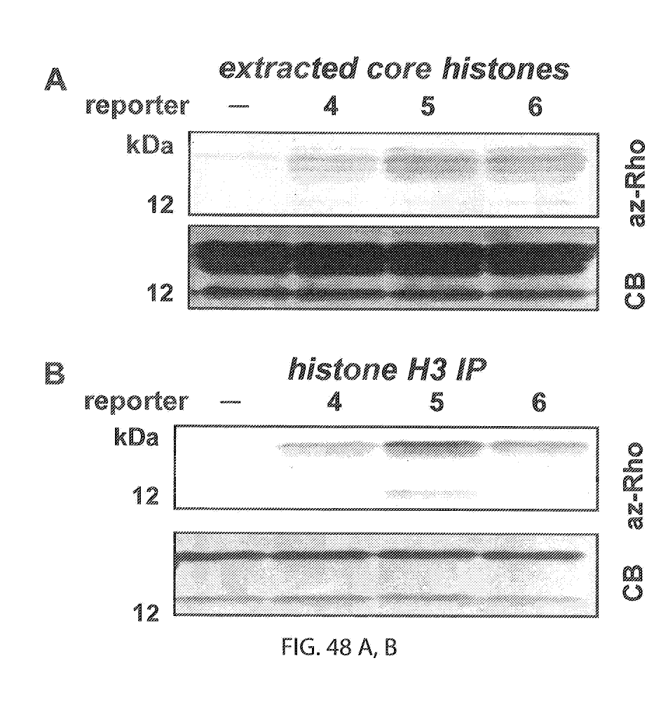

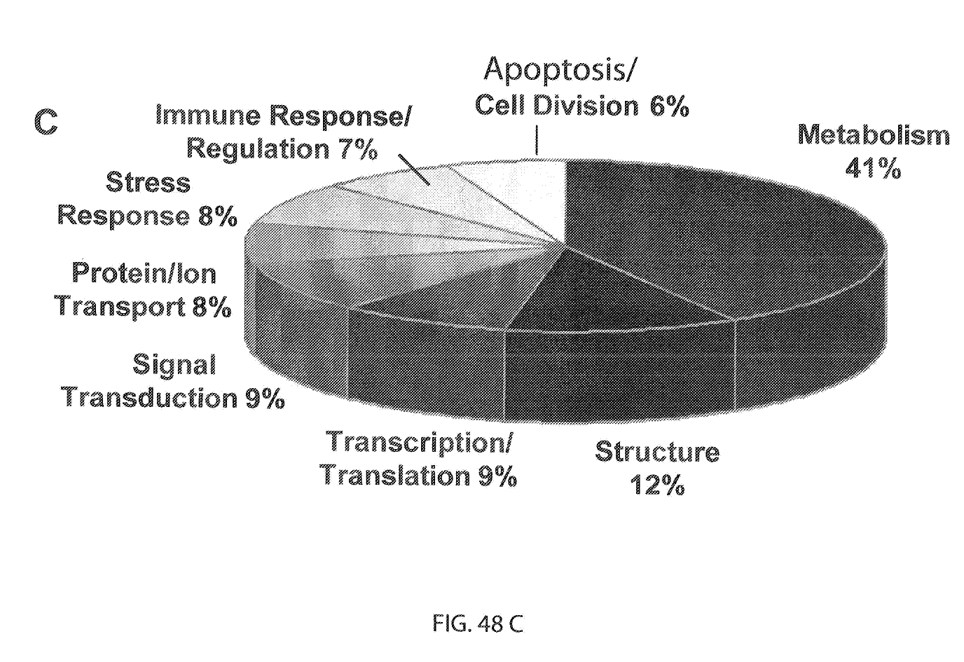

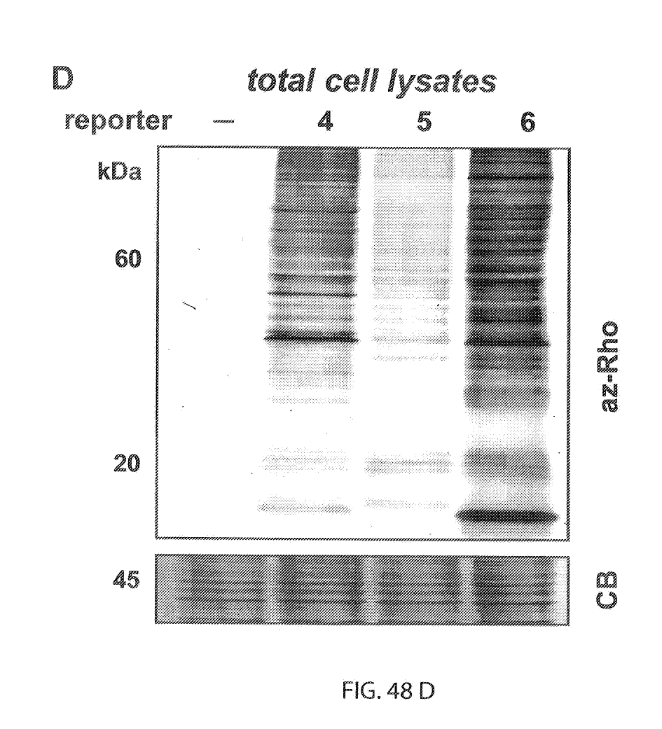

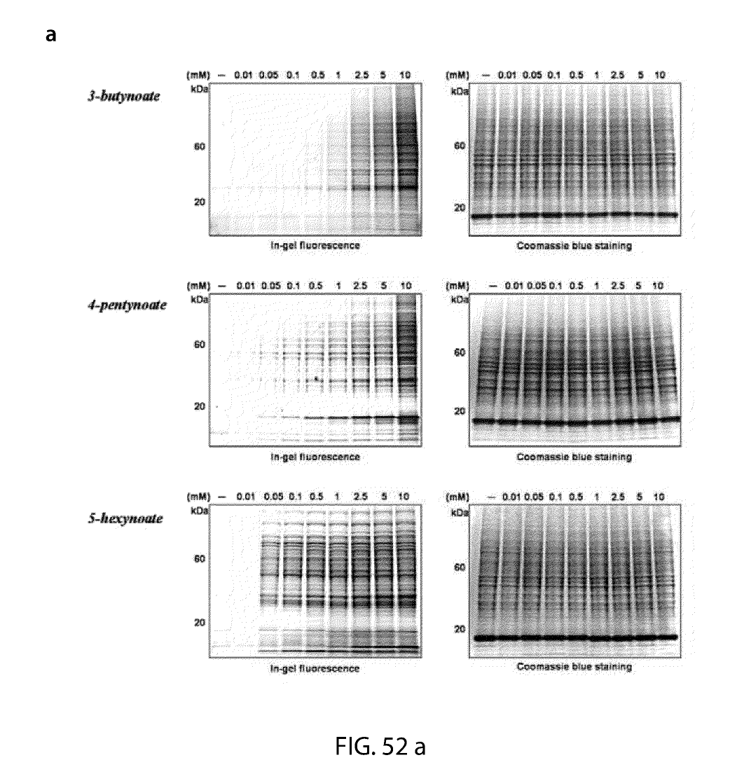

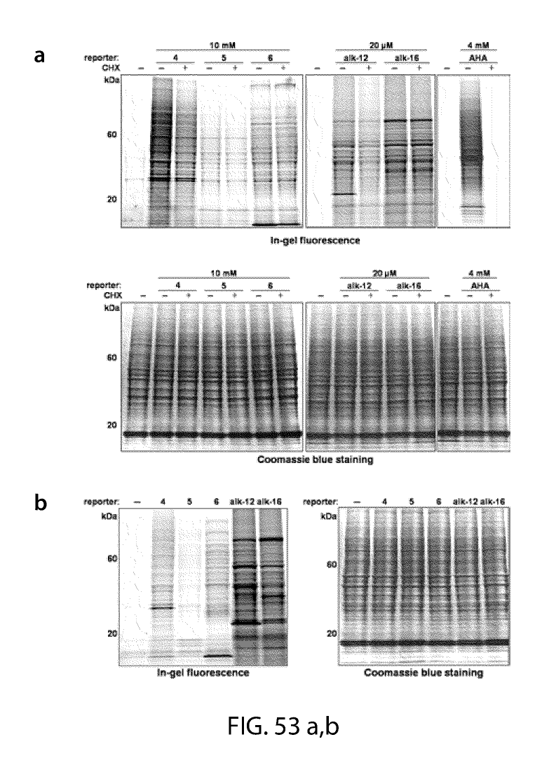

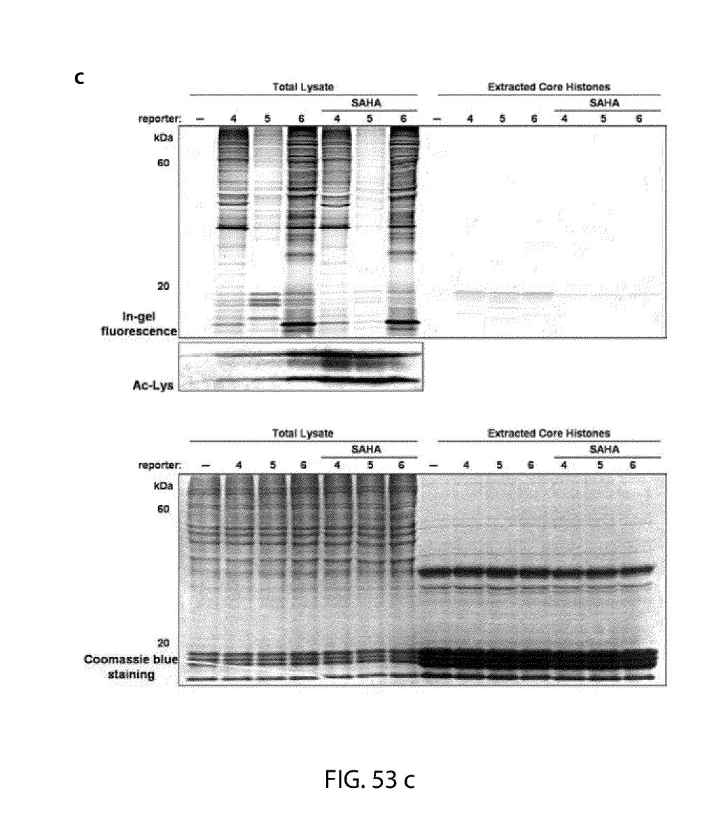

We next investigated whether the alkynyl-acetate analogs can also be utilized by endogenous KATs in cells. Three alkynyl-acetate analogs, 3-butynoate (Analog 4), 4-pentynoate (Analog 5) and 5-hexynoate (Analog 6), were hence prepared as sodium salts and examined for metabolic incorporation in Jurkat T cells via CuAAC-mediated fluorescent detection. The results showed that selective protein labeling was dose- and time-dependent and optimal results were achieved with 2.5-10 mM of alkynyl-acetate analogs and 6-8 hrs of metabolic incorporation (FIGS. 52A and 52B). Selective labeling of the enriched core histones and immunoprecipitated histone H3 from metabolically-labeled Jurkat T cells demonstrated that known lysine-acetylated proteins were specifically labeled by Analogs 4, 5 and 6 (FIGS. 48A and 48B). Profiling the total cell lysates revealed many alkynyl-acetate analog-labeled proteins that varied among the different analogs (FIG. 48C). The majority of proteins labeled by alkynyl-acetate analogs were insensitive to cycloheximide and distinct from proteins targeted by longer chain alkynyl fatty acids (FIGS. 53A and 53B). Protein labeling with alkynyl-acetate analogs was also slightly reduced when coincubated with SAHA (FIG. 53C). These observations suggest that the alkynyl-acetate analogs are primarily installed post-translationally onto proteins in cells, which target distinct proteins compared to fatty acid chemical reporters and are sensitive to KDAC inhibitors.

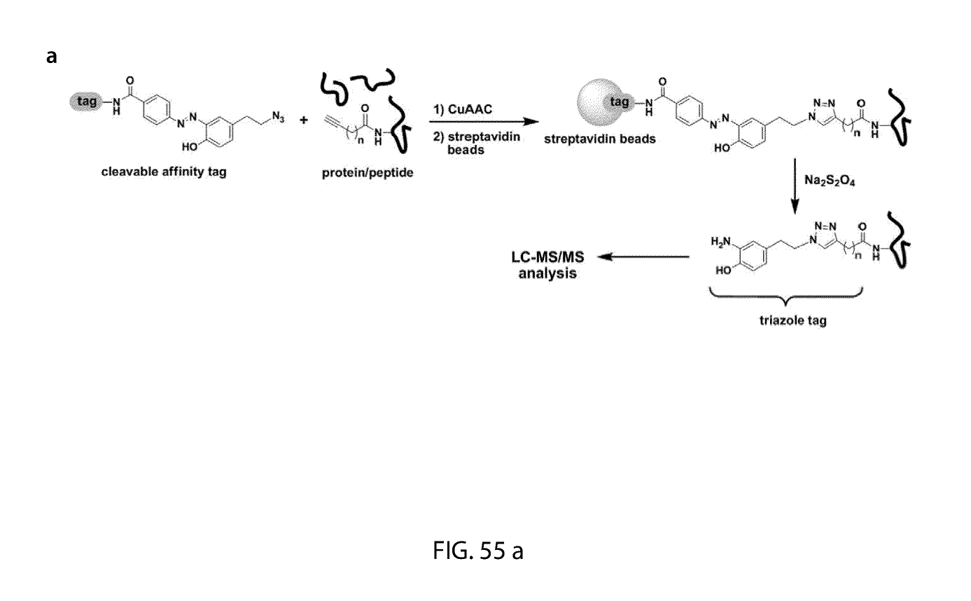

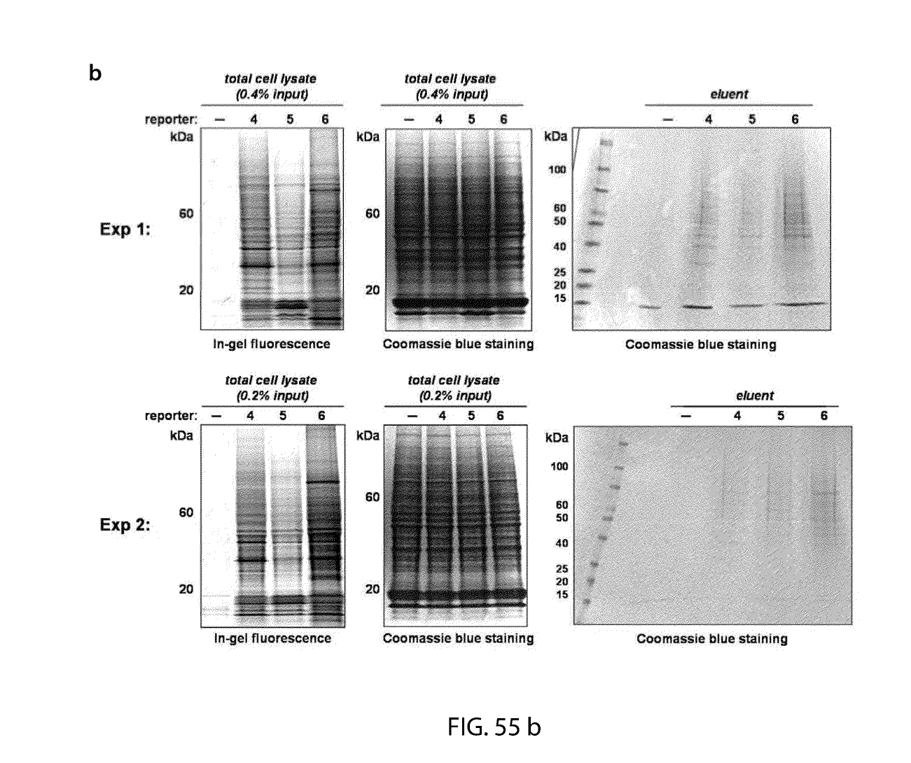

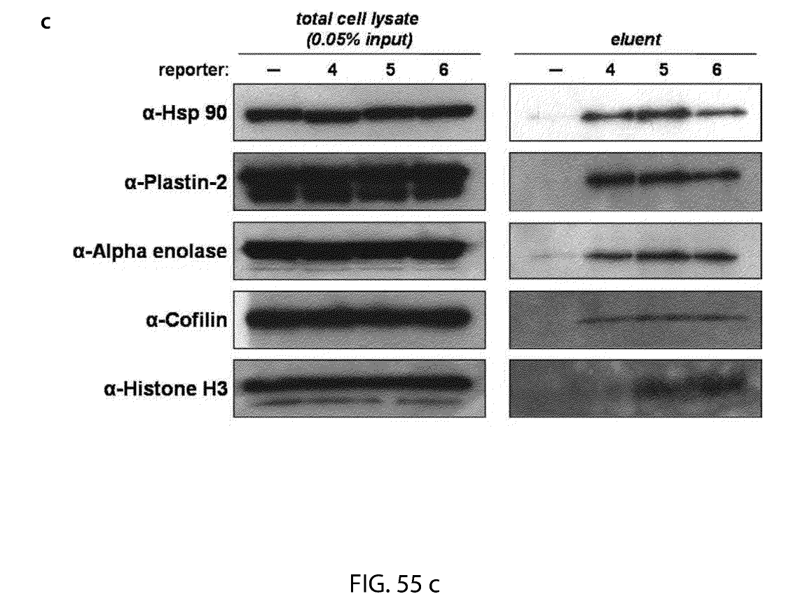

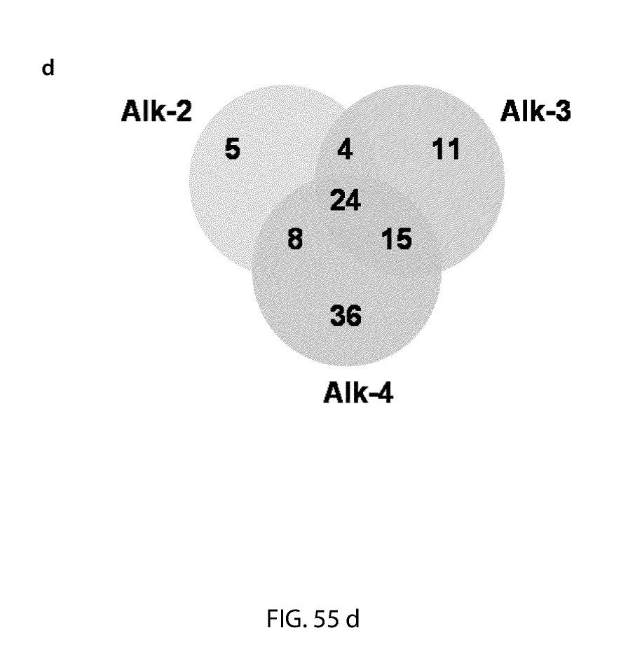

To identify alkynyl-acetate analogs-labeled proteins, total lysates of metabolically labeled Jurkat T cell were subjected to CuAAC with the cleavable azido-diazo-biotin tag followed by affinity-purification on streptavidin beads (FIG. 55). Subsequent treatment of streptavidin beads with sodium dithionite (Na₂S₂O₄) enabled efficient elution of captured biotinylated proteins and gel-based protein identification using the LTQ-Orbitrap mass spectrometery. A survey of the protein hits selectively recovered from alkynyl-acetate analog-labeled cell lysates revealed many known lysine-acetylated proteins (84.5%) as well as many candidate acetylated proteins, indicating the high specificity of these chemical reporters for targeting the lysine acetylome. Based on our proteomics data and in vitro p300-acylation studies, a qualitative comparison among these alkynyl-acetate analogs (1-6) suggests that 4-pentynoate is the optimal chemical reporter for detecting protein acetylation, as it is efficiently utilized by p300 in vitro and primarily targets lysine-acetylated proteins in cells. Although 3-butynoate and 5-hexynoate can also label lysine-acetylated proteins in cells, 3-butynyl-CoA is not efficiently utilized by p300 in vitro and 5-hexynoate may also target cellular fatty-acylated proteins (i.e. transferrin receptor, SNAP-23). Based upon these findings, we focused on 4-pentynoate for additional proteomic studies.

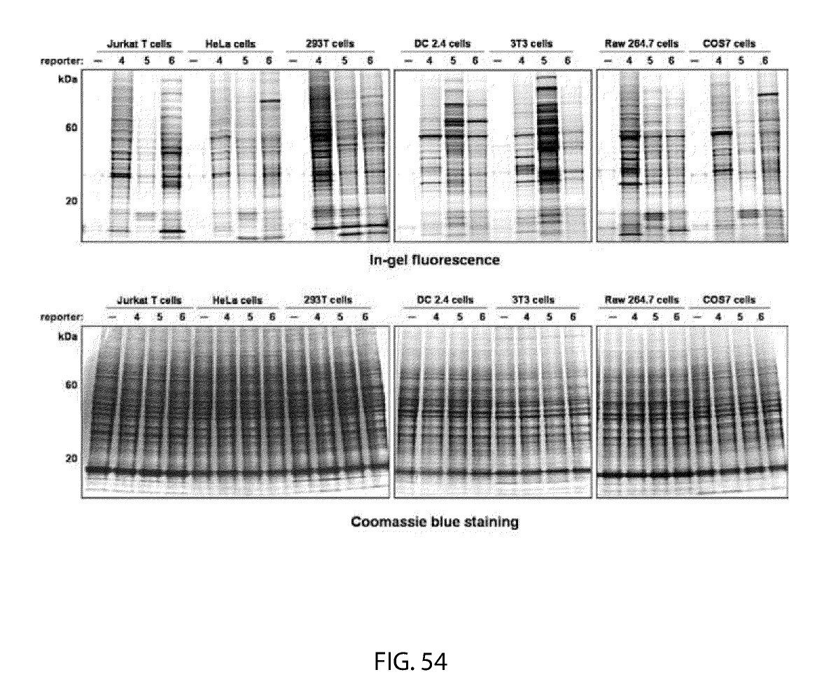

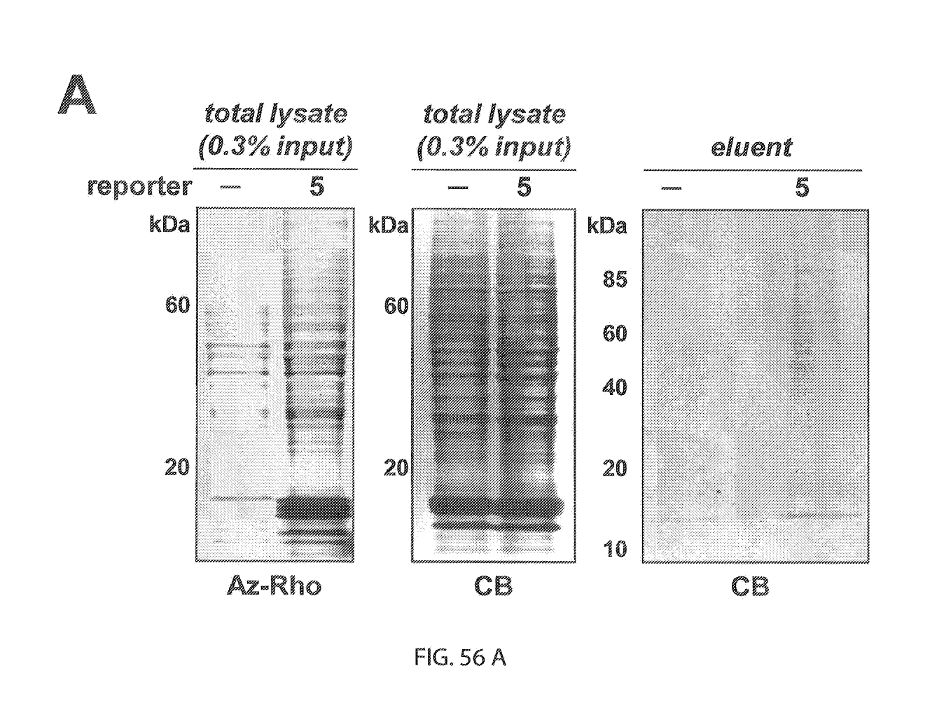

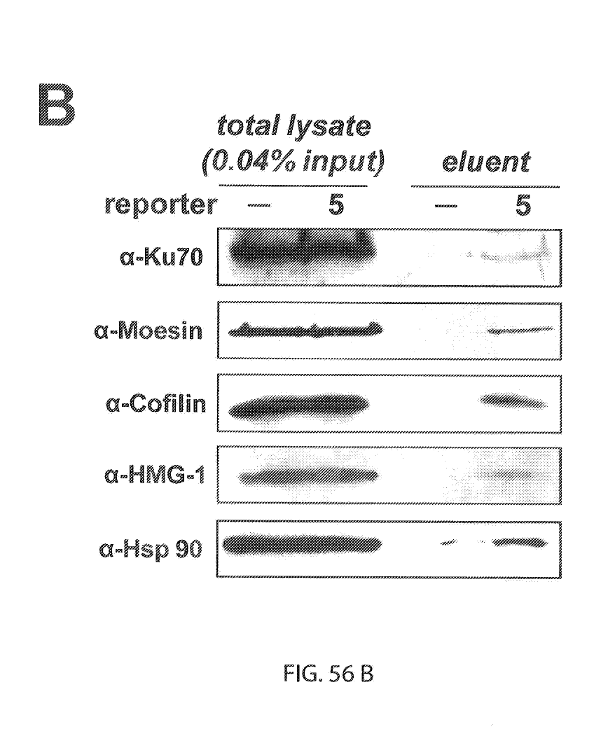

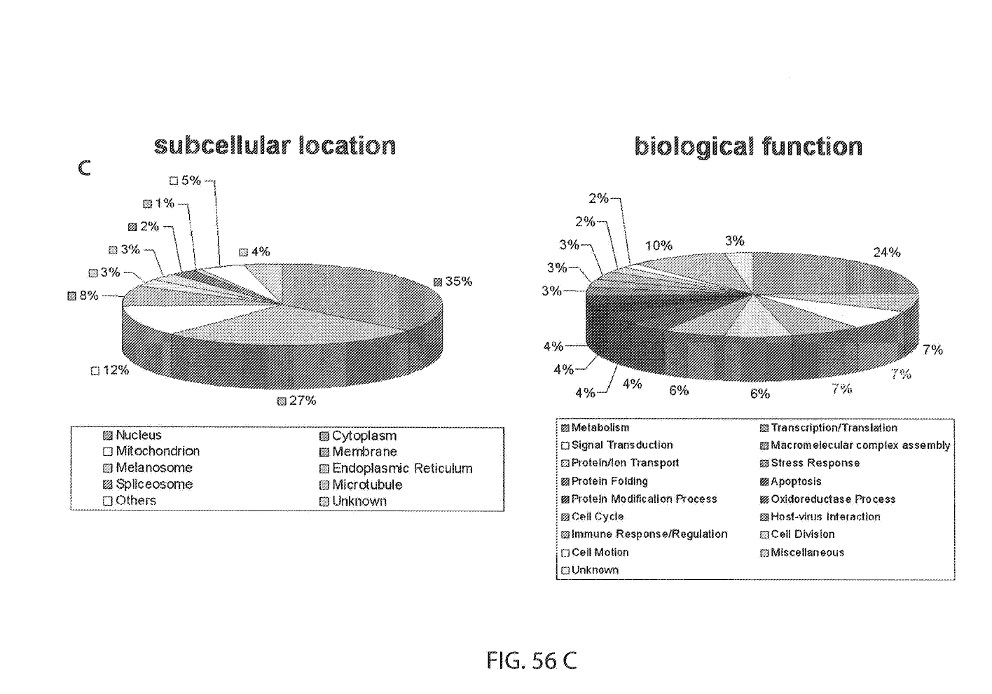

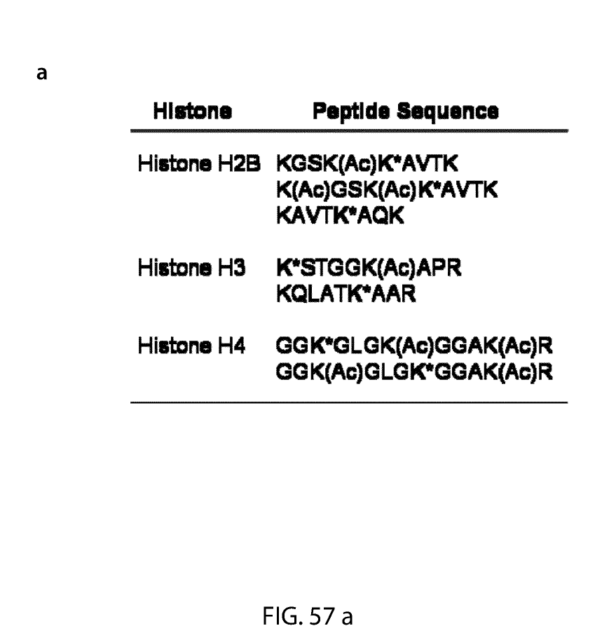

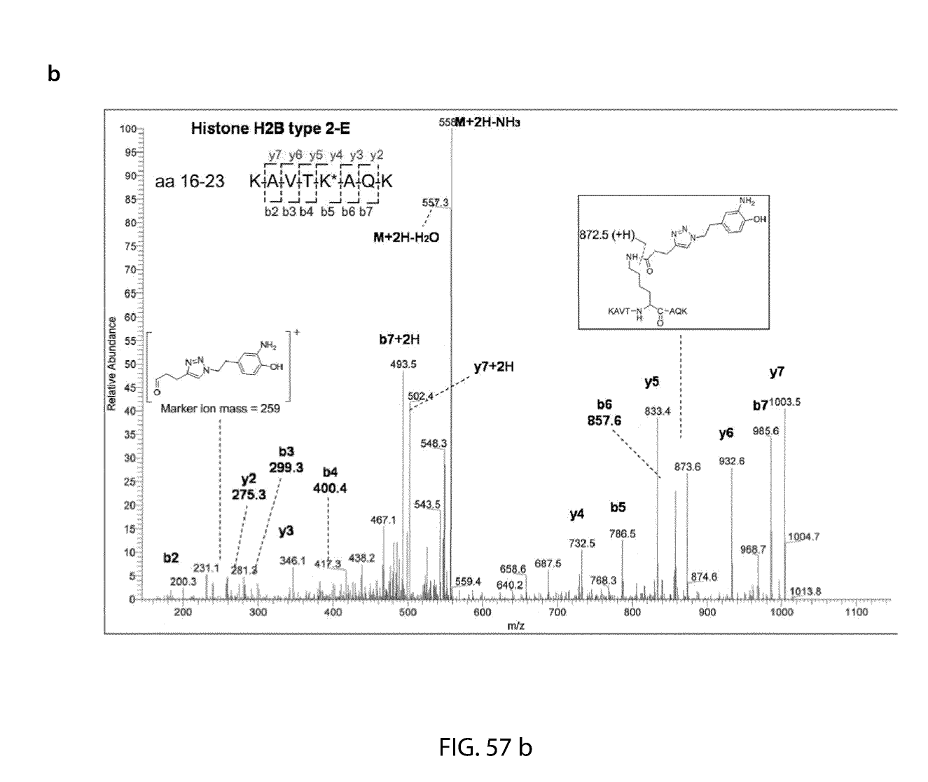

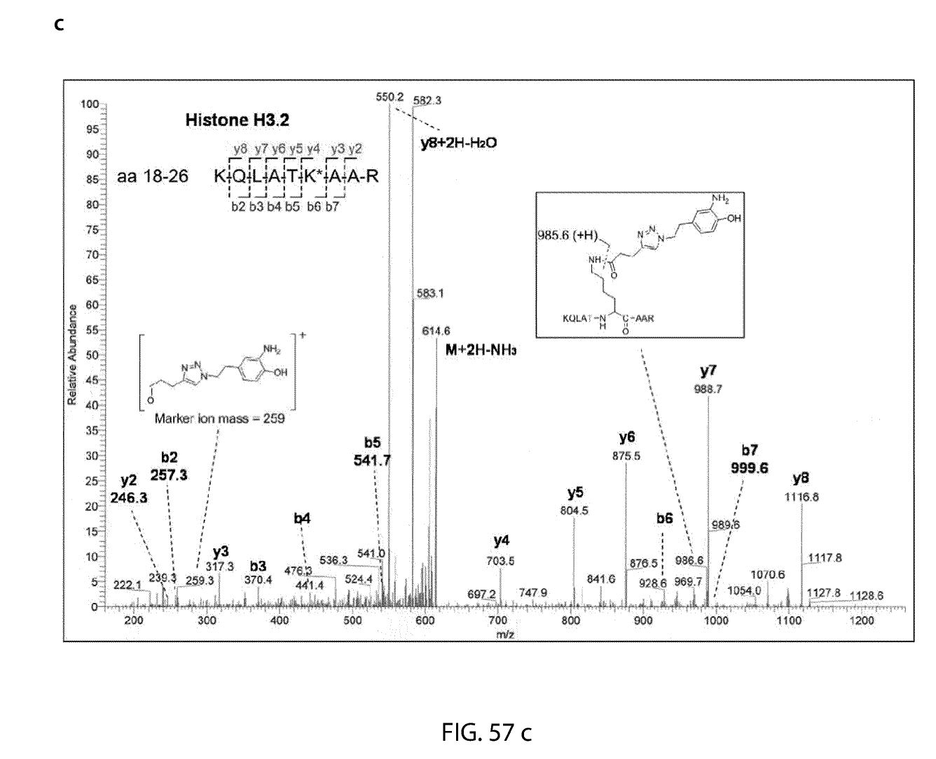

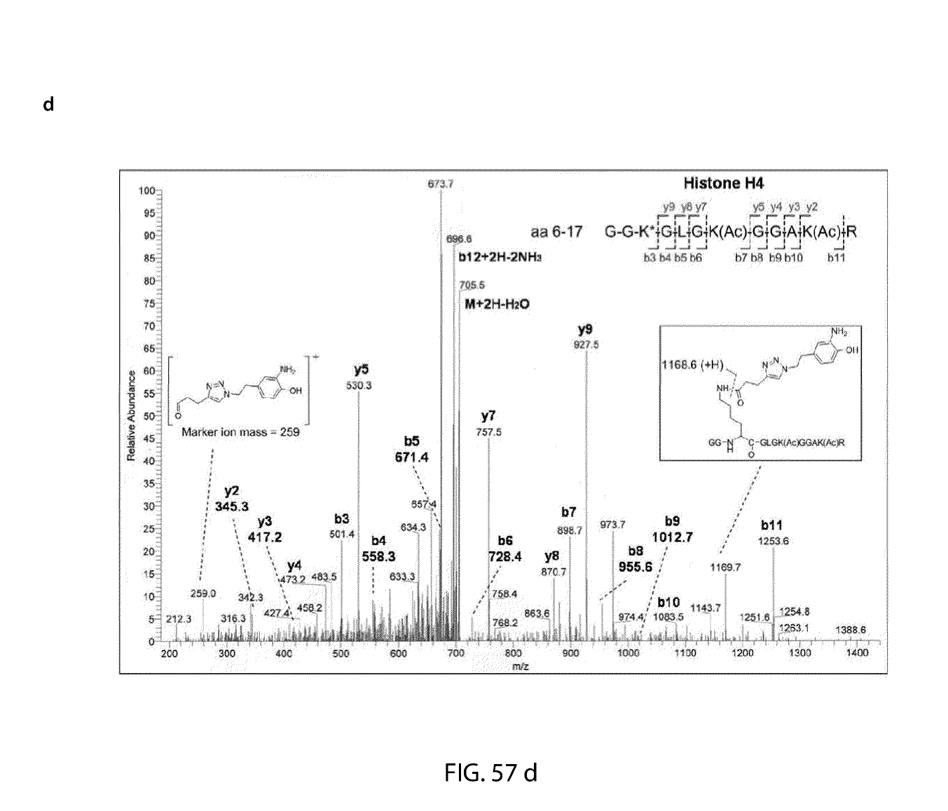

In total, from three independent experiments, we identified approximately 194 4-pentynote-labeled proteins from Jurkat T cells (FIG. 56A, 86% of which were also identified by anti-acetyl-Lys proteomic studies (Choudhary, C.; Kumar, C.; Gnad, F.; Nielsen, M. L.; Rehman, M.; Walther, T. C.; Olsen, J. V.; Mann, M. Science 2009, 325, 834-840). We confirmed the enrichment of acetylated proteins identified by mass spectrometry, including Ku70, moesin, cofilin, coronin-1A, Hsp90, HMG-1 and adenosine deaminase by Western blot analysis of the affinity-enriched protein fractions (FIG. 56A). Our proteomic data suggest that the majority of acetylated proteins reside in the nucleus and cytoplasm and are associated with diverse cellular functions that range from metabolism, signal transduction to gene expression (FIGS. 48D and 56C). To verify that 5 targets lysine residues on proteins in cells, in-solution trypsin digestion was performed with 4-pentynoate-labeled Jurkat T cell lysates to specifically enrich 4-pentynoate-modified peptides. The CuAAC-biotinylated peptides were captured by streptavidin beads and eluted with Na₂S₂O₄ for tandem MS sequencing. MS/MS analysis of peptides demonstrated that 4-pentynoate is metabolically incorporated into known sites of lysine acetylation on histone H₂B, H3 and H4 (FIG. 57). The characteristic marker ion (mass=259) corresponding to the fragmentation peak of the modified lysine residue (4-pentynoate+CuAAC/Na2S2O4 cleavage adduct) was clearly observed in all MS/MS spectra of histone H₂B, H3 and H4. These experiments collectively demonstrate that alkynyl-acetate analogs such as 4-pentynyl-CoA and 4-pentynoate function as efficient bioorthogonal chemical reporters for protein acetylation in vitro and in cells, respectively. Notably, metabolic labeling of various mammalian cell lines with alkynyl-acetate analogs revealed distinct and analog-specific patterns of acetylomes in diverse cell types, which highlights the generality and utility of these bioorthogonal chemical reporters for protein acetylation detection (FIG. 54).

Unraveling the functions of protein acetylation remains a challenging task. The bioorthogonal chemical reporters presented here provide readily accessible non-radioactive reagents for fluorescence profiling and large-scale analysis of protein acetylomes. Moreover, alkynyl-acetyl-CoA analogs enable rapid and sensitive detection of KAT activities that should be useful for assigning protein substrates in protein mixtures. This chemical approach provides complementary experimental tools to anti-acetyl-Lys antibodies 12, MS/MS11 and bioinformatic methods, (Basu, A.; Rose, K. L.; Zhang, J.; Beavis, R. C.; Ueberheide, B.; Garcia, B. A.; Chait, B.; Zhao, Y.; Hunt, D. F.; Segal, E.; Allis, C. D.; Hake, S. B. Proc. Nat. Acad. Sci. U.S.A. 2009, 106, 13785-13790). The incorporation of quantitative proteomic methods and bump-hole strategies, (Dephoure, N.; Howson, R. W.; Blethrow, J. D.; Shokat, K. M.; O'Shea, E. K. Proc. Nat. Acad. Sci. U.S.A. 2005, 102, 17940-17945) in the future should expand the utility of these chemical tools and facilitate the functional analysis of protein acetylation in physiology and disease.

### General Methods and Materials:

Unless otherwise noted, all the chemical reagents were purchased from either Sigma-Aldrich or Fisher Scientific. ¹H and ¹³C NMR spectra were recorded on Bruker DPX-400 or Bruker AVANCE-600 instrument. Chemical shifts are reported in δ ppm, and J values are reported in Hz. MALDI-TOF mass spectra were acquired on Applied Biosystems Voyager-DE mass spectrometer. HPLC was conducted on Agilent 1100 series HPLC system with HPLC-grade acetonitrile (CH₃CN) and ultrapure water from Milli-Q Advantage A10 Purification System. In-gel fluorescence scanning was performed using Amersham Biosciences Typhoon 9400 variable mode imager (excitation 532 nm, 580 nm filter, 30 nm band-pass). All contrast/brightness adjustments on the images were applied to the whole gels and blots. All the image adjustments were performed in Photoshop.

Histone H3 peptide (aa 2-21 (L21Y): ARTKQTARKSTGGKAPRKQY (SEQ ID NO:24), >90% purity based on HPLC analysis) was purchased from the Proteomics Resource Center at the Rockefeller University. Recombinant histone H3 (human or Xenopus laevis) was purchased from Millipore. RPMI media 1640 and Dulbecco's modified Eagle media (DMEM) were purchased from Gibco. EDTA-free protease inhibitor was purchased from Roche Applied Science. Pre-stained protein ladder was purchased form Invitrogen. Pre-cast polyacrylamide gels (4-20% or 15% Criterion Tris-HCl gels) were purchased from Bio-Rad Laboratories. Primary antibodies (anti-histone H3, anti-Hsp90, anti-α Enolase, anti-Cofilin, anti-L-plastin, anti-moesin, anti-HMG-1, anti-Ku70, anti-HMG-1, anti-ADA and anti-acetylated lysine) were purchased either from Santa Cruz Biotechnology or Millipore. Secondary antibodies were purchased from Jackson ImmunoResearch Laboratories. Mass spectrometry grade trypsin was purchased from Promega.

### Synthesis of Acetyl-CoA Analogs

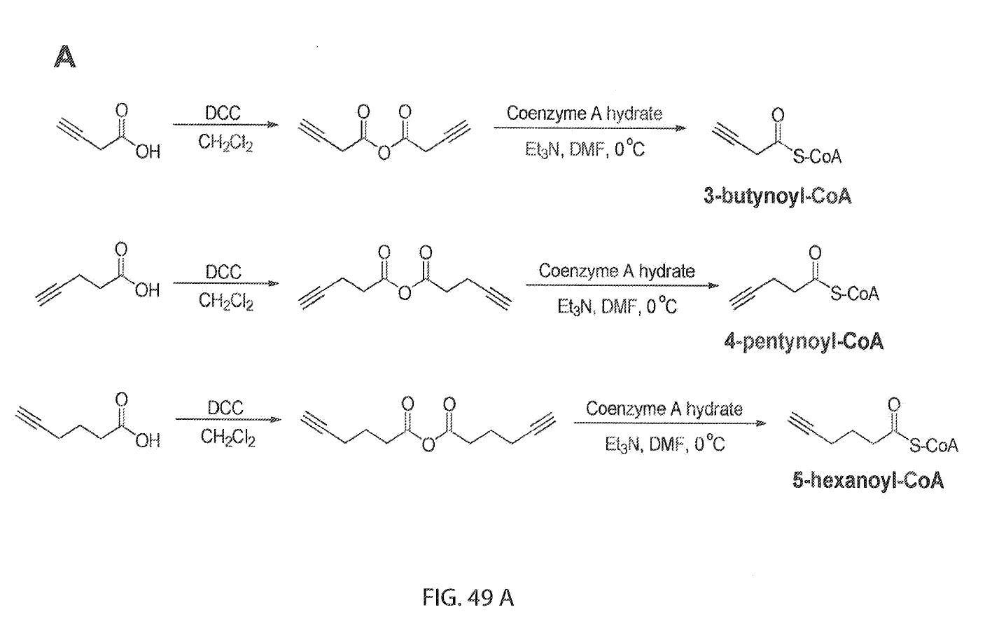

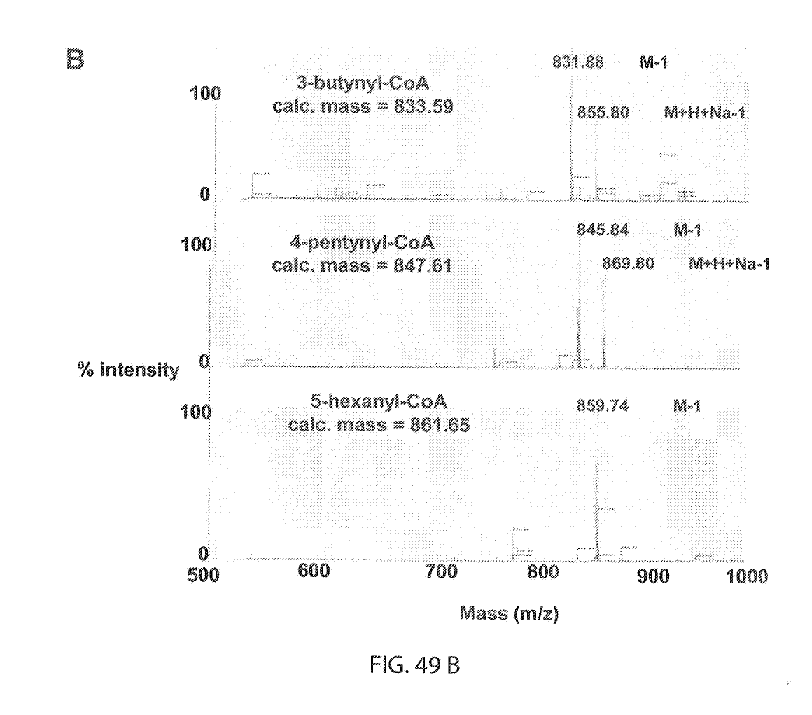

2 mmol of 3-butynoic acid (made from Jones oxidation of 3-butyn-1-ol⁴), 4-pentynoic acid (Fluka), or 5-hexynoic acid (Aldrich) was dissolved in 10 mL of anhydrous dichloromethane. To this solution was added 1 mmol of N,N′-dicyclohexylcarbodiimide (DCC) under argon, and the reaction was allowed to proceed at room temperature for 4 hrs. The anhydride products can be observed on TLC plate by using ethyl acetate/hexane (1/1) as the developing solvent system. The reaction mixture was then concentrated down to dryness, redissolved in anhydrous dimethylformamide (DMF) under argon and cooled to 4° C. in ice-bath. To this reaction mixture was added 0.2 equivalent of coenzyme A.hydrate (Sigma, Cat. No: C4282) and 0.6 equivalent of triethylamine (Et₃N). The reaction was kept in ice bath and stirred for 30 mins. Upon the reaction was complete (as determined by LC/MS), it was neutralized to pH 7 by dropwise addition of 0.1 N aqueous hydrochloride and then concentrated to dryness under high vacuum. The dried crude material was then redissolved in H₂O/CH₃CN (1/1) and filtered through the syringe filter devise (Millipore, Cat. No. SLCR013NL, 0.45 μm, hydrophilic PTFE, 13 mm). The filtrate was subjected to HPLC purification with the elution method set as 5% CH₃CN/95% H₂O to 50% CH₃CN/50% H₂O over 30 min. The alkynyl-acetyl-CoA analogs typically elute at ˜21 min under this condition. The reaction products were confirmed by MALDI-TOF mass spectrometry and all the mass spectra were acquired in negative mode as shown in FIG. 49b. MALDI-TOF MS data of alkynyl-acetyl-CoA analogs: 3-butynyl-CoA: calcd for C₂₄H₃₈N₇O₁₇P₃S ([M−H]⁻) 832.11, found 831.88 4-pentynyl-CoA: calcd for C₂₆H₃₉N₇O₁₇P₃S ([M−H]⁻) 846.13, found 845.84. 5-hexanyl-CoA: calcd for C₂₇H₄₁N₇O₁₇P₃S ([M−H]⁻) 860.14, found 859.74.

### Preparation of Sodium 3-Butynoate, Sodium 4-Pentynoate and Sodium 5-Hexynoate

3-Butynoic acid, 4-pentynoic acid or 5-hexynoic acid (6 mmol) was dissolved in 20 mL ddH₂O. Aqueous NaOH solution (6 mmol, ˜0.18N) was added dropwise to reaction mixture. The reaction mixture was then filtered through 0.45 μm membrane, frozen in liquid nitrogen and lyophilized to dryness. All the sodium forms of alkynyl-acetate analogs were appeared as white powders. To prepare accurate stock solutions of sodium 3-butynoate, 4-pentynoate and 5-hexynoate, 10 mg of each were dissolved in 600 μL D₂O. 1.5 μL of anhydrous CH₃CN was added to each sample as an internal standard for peak integration. After gently vortexing and short spin, 400 μL of the well-mixed solution was transferred into NMR tube for ¹H-NMR analysis. As shown in FIG. 51, To normalize the concentration of each acetate analog added to the cells, we calculated the ratio of the peak integration value of the terminal alkyne proton relative to CH₃CN proton in each spectrum. Based on these ratios, we adjusted the concentration of acetate analogs to prepare 1M stock solutions.

### Characterization of Alkynyl-Acetate Analogs

To calibrate the chemical shifts of each alkynyl-acetate analog, MeOH (0.5 μL) was added to serve as a NMR calibration standard in D₂O. The characteristic peaks of MeOH in D₂O are 62.61 (CH₃) in ¹H-NMR and 649.50 (CH₃) in ¹³C NMR.

3-butynoate: ¹H NMR (D₂O, 600 MHz) δ 3.15 (d, 2H, J=2.7 Hz), 2.46 (t, 1H, J=2.6 Hz); ¹³C NMR (D₂O, 150 MHz) δ 197.76, 101.88, 93.21; HMRS (ESI-TOF) calcd for C₄H₄NaO₂ ([M+H]⁺) 107.0109, found 107.0416.

4-pentynoate: ¹HNMR (D₂O, 600 MHz) δ 2.36-2.43 (m, 4H), 2.34 (t, 1H, J=2.3 Hz); ¹³C NMR (D₂O, 150 MHz) δ 194.33, 98.49, 82.72, 28.34; HMRS (ESI-TOF) calcd for C₅H₆NaO₂ ([M+H]⁺) 121.0265, found 121.0184.

5-hexynoate: ¹H NMR (D₂O, 600 MHz) δ 2.35 (t, 1H, J=2.7 Hz), 2.27 (t, 2H, J=7.4 Hz), 2.21 (td, 2H, J=7.2, 2.6 Hz), 1.75 (pentet, 2H, J=7.3 Hz); ¹³C NMR (D₂O, 150 MHz) δ 195.88, 98.56, 82.53, 37.79, 30.50; HMRS (ESI-TOF) calcd for C₆H₈NaO₂ ([M+H]⁺) 135.0422, found 135.0321.

Preparation of Recombinant p300

Flag-tagged human p300 was prepared as previously described by baculoviral infection of SF9 cells (Woojin An and Robert G. Roeder, Journal of Biological Chemistry 278 (3), 1504 (2003)). Cells were lysed in lysis buffer (20 mM Tris, pH 7.5, 500 mM NaCl, 4 mM MgCl₂, 0.4 mM EDTA, 20% glycerol, 2 mM DTT, and supplemented with protease inhibitors). Clarified cell lysates were diluted to 300 mM NaCl, and NP-40 was added to 0.1% final concentration. Flag M2 agarose resin (Sigma) was added to the lysate and incubated for 4 hours at 4° C. Resin was washed extensively with BC buffer (20 mM Tris, pH 7.9, 20% glycerol) containing 300 mM KCl, and p300 protein was eluted by 0.5 mg/ml Flag peptide in BC buffer containing 100 mM KCl. Protein concentration and purity were estimated by comparison to BSA standards on SDS gels stained with Gelcode Blue stain (Pierce).

MALDI-TOF Analysis of p300-Catalyzed Acylation of Histone H3 Peptide In Vitro

In vitro acylation reactions were carried out based on the reported procedure⁶ with some modifications. 10 μL reaction solution containing 25 μmol H3 peptide, 20 μM acetyl-CoA or alkynyl-acetyl-CoA, 50 ng of p300, 50 mM, pH 7.9 Tris buffer and 10% glycerol was incubated for 2 hrs at 30° C. The peptide products were then extracted with ZipTip (Millipore) and eluted with 1.5 μL of 50% CH₃CN (with 0.1% TFA). Eluent (1 μL) mixed with the ionization matrix (1 μL), α-cyano-4-hydroxycinnamic acid, was spotted onto MALDI plates and subjected to MS analysis. The experimental results were shown in FIG. 50A.

In Vitro Acylation of Histone H3 Protein by p300 and Mapping Protein Modification Sites

5 μL reaction solution containing histone H3 (0.3-1.7 μg), 160 μM alkynyl-acetyl-CoA analogs or alkynyl-acetate analogs, 100 ng of p300, 50 mM, pH 7.9 Tris buffer and 10% glycerol was incubated for 2 hrs at 30° C. Following the in vitro reaction, proteins were separated on 15% SDS-PAGE and stained with coomassie brilliant blue R-250 staining solution (Bio-Rad). The gel slices containing histone H3 products were excised from each lane, washed with 50 mM ammonium bicarbonate (ABC) twice, destained with 50 mM ABC/CH₃CN (50/50) twice and dehydrated with CH₃CN. After drying the gel slices in a SpeedVac, gel slices were rehydrated with trypsin solution (2 μg of trypsin for each vial/gel slice) and incubated in 37° C. water bath for 18 hrs. Trypsin-digested peptides in solution were collected, dried in SpeedVac, resuspended in H₂O (with 0.1% TFA) and submitted samples to nano-HPLC/MS/MS analysis (Thermo LTQ-Orbitrap in the Proteomic Resource Center at Rockefeller University).

LC-MS analysis was performed with a Dionex 3000 nano-HPLC coupled to an LTQ-Orbitrap ion trap mass spectrometer (ThermoFisher). Peptides were pressure loaded onto a home made 75 μm diameter, 15 cm C₁₈ reverse phase column and separated with a gradient running from 95% buffer A (HPLC water with 0.1% formic acid) and 5% buffer B (HPLC grade acetonitrile with 0.1% formic acid) to 55% B over 30 min, next ramping to 95% B over 10 min and holding 95% B for 10 min. One full MS scan (300-2000 MW) was followed by 3 data dependent scans of the nth most intense ions with dynamic exclusion enabled. The spray voltage was set to 1.94 kV and the flow rate through the column was set to 0.25 μL/min.

Raw tandem mass spectra were searched against the human IPI protein database version 3.56 using SEQUEST search engine (Thermo Scientific). Cysteine carbamido-methylation was searched as fixed modification, while methionine/tryptophan oxidation, asparagines/glutamine deamindation, lysine/serine/threonine/cysteine acetylation, N-terminal acetylation/3-butynylation/4-pentynylation/5-hexynylation and lysine 3-butynylation/4-pentynylation/5-hexynylation were searched as variable modifications. Peptide tolerance was set as 10.0 ppm, fragment ion tolerance was set as 1.0 AMU and trypsin was set as the digestion enzyme. The resulting searching files were then analyzed and visualized by Scaffold 2 proteome software.

Each identified protein must contain at least 2 unique peptides that are exclusively assigned to this protein with a minimum protein identification probability of 99%. Each peptide spectrum must meet several selection thresholds including >95% for peptide identification probability, >1.0 for SEQUEST XCorr score, and ±6 ppm for actual minus calculated peptide mass.

The experimental results were shown in FIG. 50B-C, and Table 2.

In-Gel Fluorescent Detection of p300-Catalyzed In Vitro Acylation

Following 2 hrs incubation at 30° C., 15 μL reaction was stopped by adding 10 μL of 4% SDS buffer (4% SDS, 150 mM NaCl, 50 mM triethanolamine, pH 7.4). CuAAC reaction was carried out by adding 2 μL of freshly-premixed “click chemistry cocktail” (100 μM az-Rho, 1 mM TCEP, 100 μM TBTA and 1 mM CuSO₄) to the acylation reaction¹. CuAAC reactions were allowed to proceed at room temperature for 1 hr, and proteins were subsequently separated on 15% SDS-PAGE. The gels were soaked in destaining solution (40% MeOH, 10% AcOH, 50% H₂O) for 1-2 hrs to remove the non-covalently bound az-Rho dye. After washing with dd H₂O, the gel was subjected to in-gel fluorescence scanning. The experimental results were shown in FIG. 47B and FIG. 50D.

### Cell Culture

Jurkat T cells were cultured in tissue culture flasks in RPMI media supplemented with 10% fetal bovine serum (FBS), penicillin (100 units/mL) and streptomycin (0.1 mg/mL). RAW264.7 macrophages, NIH3T3 fibroblasts, HeLa cells, 293T cells, COS-7 cells and DC2.4 cells were cultured in petri dishes in DMEM media supplemented with 10% FBS, penicillin (100 units/mL) and streptomycin (0.1 mg/mL). Cells were incubated in a 5% CO₂ humidified incubator at 37° C.

### Metabolic Labeling and Preparation of Total Cell Lysates

Jurkat T cells (20×10⁶) were cultured in 4 mL of RPMI medium 1640 supplemented with 2% FBS, 1% penicillin and streptomycin and labeled with bioorthogonal chemical reporters (1 M stock solutions) at concentrations described. At the indicated time points, cells were harvested and washed twice with PBS. Trypan blue exclusion was used to determine the cell viability during metabolic labeling. Bioorthogonal chemical reporters did not appear to influence cell viability. To prepare the total cell lysates, cell pellets were resuspended in 50 μL of lysis buffer (7 mM PMSF, 10×EDTA-free protease inhibitors and 800 μM MgCl₂, 0.05% SDS, 10 mM triethanol-amine, pH 7.4) followed by coincubating with 0.74 of benzonase nuclease (Sigma) for 20 min. The cell suspensions were then fully lysed by adding 150 μL of 4% SDS buffer (4% SDS, 150 mM NaCl, 50 mM triethanolamine, pH 7.4) with subsequent vigorous vortexing. Insoluble cell debris was removed by centrifugation at 20000 g for 10 min. The supernatant was collected to yield total cell lysate. Protein concentration was determined by BCA assay.

### CuAAC Reactions and In-Gel Fluorescence Scanning

For a typical profile of mammalian acetylome, 50 μg of total cell lysates was reacted with freshly pre-mixed “click chemistry cocktail” (100 μM az-Rho, 1 mM TCEP, 100 μM TBTA and 1 mM CuSO₄). The final protein concentration was typically 1 mg/mL in 4% SDS buffer. The reaction was allowed to stand at room temperature for 1 hr. Proteins were then precipitated by CHCl₃-MeOH precipitation method and washed thrice with chilled MeOH (−20° C.). Dried protein pellets were resuspended in 4% SDS buffer and then separated by SDS-PAGE. The gel was subsequently subjected to in-gel fluorescence scanning to acquire the image.

### Acid-Extraction of Core Histones

The method for acid extraction of core histones was based on the reported protocol with some modifications. To Jurkat T cells (10×10⁶ cells) was added 1 mL of ice-cold hypotonic lysis buffer (10 mM TEA, pH 7.4, 1 mM KCl, 1.5 mM MgCl₂, 1 mM PMSF, 10 mM SAHA) supplemented with 1×EDTA-free protease inhibitor cocktail. The resuspended cells were homogenized by an ice-cold tight-fitting dounce homogenizer and lysed by three cycles of freeze thaw lysis. Intact nuclei were pelleted by spinning at 10000 g for 10 min at 4° C. The supernatant was discarded and nuclear pellet was washed twice with ice-cold hypotonic buffer. The nuclear pellet was then resuspended in 0.4N H₂SO₄ and agitated overnight on rotator at 4° C. The nuclear debris was pelleted by spinning at 16000 g for 10 min at 4° C. The supernatant containing extracted core histones were collected and then precipitated with MeOH (5 volume) at −80° C. overnight. Precipitated histone proteins were spun down at 16000 g for 10 min at 4° C. and wash twice with 500 uL of ice-cold MeOH. Protein pellets were air-dried at room temperature and then resuspended in dd H₂O. The histone protein concentration was determined by BCA assay.

### Histone H3 Immunoprecipitation

Core histones (50 μg) extracted from Jurkat T cells described above were co-incubated with anti-histone H3 antibody (1 μg, Santa Cruz Biotechnology, C-16), 25 uL protein-G agarose bead slurry (Roche) at 4° C. on end-over-end rotator for 2 hr. The beads were spun down (10,000 g, 30 sec) and washed trice with ice-cold modified RIPA lysis buffer (1% Triton X-100, 1% sodium deoxycholate, 0.1% SDS, 150 mM NaCl, 10 mM Tris, pH 7.4 in ddH₂O). The beads were then resuspended in 20 uL of 4% SDS lysis buffer and incubated with 2.5 uL of freshly-premixed “click chemistry cocktail” for 1 hr at room temperature. 5 uL of 4×LDS loading buffer and 1 uL β-mercaptoethanol were added to each reaction. The mixtures were heated at 95° C. for 5 min and spun at 1000 g for 30 sec. 25 uL of the reaction supernatant was directly loaded onto 15% Tris-HCl gel and separated by SDS-PAGE. The gel was then destained for 2 hr at room temperature and scanned by 9400 Typhoon imager.

### Immunoblotting

Proteins separated by SDS-PAGE were transferred onto PVDF membrane. After blocking with 5-10% milk in PBST (PBS with 1% Tween-20), the membrane was washed trice with PBST and then coincubated with anti-histone H3 antibody (Millipore) or anti-Ac-Lys antibody (Millipore). The following procedures were based on the protocols provided by Millipore.

### Proteomic Analysis of Alkynyl Acetate Analogs-Labeled Proteins

3-butynoate-, 4-pentynoate- or 5-hexynoate-labeled total cell lyates (15-25 mg) were diluted into 4% SDS buffer (the final protein concentration=1 mg/mL) and reacted with freshly pre-mixed click chemistry reagents (100 μM azido-diazo-biotin tag (J. Wilson, Yang, Y.-Y., Raghavan, A., Charron, G. & Hang, H. C., submitted (2009), 1 mM TCEP, 100 μM TBTA and 1 mM CuSO₄) for 2 hrs at room temperature (FIG. 55). Proteins were then precipitated by MeOH (5 volume) at −20° C. overnight. Precipitated proteins were centrifuged at 5,200 g for 30 min at 4° C. and washed thrice with ice-cold MeOH. To capture the biotinylated proteins by streptavidin beads, the air-dried protein pellet was resuspended in 2-3 mL of 4% SDS buffer containing 10 mM EDTA, subsequently, the protein suspension was diluted into NP-40 lysis buffer (1% NP-40, 150 mM NaCl, 50 mM Tris, pH 8.0) to reduce SDS concentration down to 1%. Pre-washed streptavidin beads were then incubated with this protein solution at room temperature for 1 hr on end-over-end rotator. The captured proteins were sequentially washed trice with modified RIPA lysis buffer (1% Triton X-100, 1% sodium deoxycholate, 0.1% SDS, 150 mM NaCl, 10 mM Tris, pH 7.4), 1× GIBCO's PBS (+0.2% SDS), PBS and ammonium bicarbonate (ABC). For cysteine reduction and alkylation, incubating the captured proteins with freshly-made 2 mM dithiothreitol in 8M urea for 10 min followed by alkylation for another 30 min with freshly prepared iodoacetamide (the final concentration is 6 mM). Wash the captured proteins with ABC buffer trice. To release the proteins from the streptavidin beads, the beads were resuspended in ABC buffer and transferred to a dolphin tube. Pellet the beads, and elute the captured proteins by incubating the beads with elution solution (0.01% SDS, 25 mM sodium dithionite, 50 mM ABC buffer) for 1 hr at room temperature. Spin down the beads and collect the eluent. Repeat the same elution procedure for 1-2 times. Concentrate the eluent by using the microcon centrifugal filter device (10 kDa NMWL). The concentrated eluent was then dried in SpeedVac. Resuspend the dried pellets in 1×LDS/5% β-mercaptoethanol. 60% volume of this resuspended solution was loaded onto SDS-PAGE for in-gel trypsin digestion, while the remaining sample was loaded onto another SDS-PAGE for validation of protein candidates by Western blot.

The resultant enriched 3-butynoate-, 4-pentynoate- or 5-hexynoate-labeled proteome was visualized by coomassie blue staining. The gel images were shown in FIG. 55B and FIG. 56A. Each lane was sliced into 8 or 12 fractions. Each excised gel slice was placed in microcentrifuge tube. The gel slices were further cut into more pieces, washed with 50 mM ammonium bicarbonate (ABC) twice, destained with 50 mM ABC/acetonitrile (50/50) twice, and then dehydrated in 100% acetonitrile. After drying the gel pieces in a Speed Vac, gel pieces were rehydrated with trypsin solution (2 μg of trypsin for each vial/gel slice) and incubated in 37° C. water bath for 18 hrs. The eluted trypsin-digested peptides were then collected and dried in SpeedVac. Resuspended the dried eluents in H₂O (with 0.1% TFA) and submitted the samples to nano-HPLC/MS/MS analysis (Thermo LTQ-Orbitrap in the Proteomic Resource Center at Rockefeller University).

### Mapping the Modification Sites of 4-Pentynoate-Metabolically Labeled Proteins

For modification site-mapping experiments, 20 mg of total Jurkat T cell lysates was subjected to CuAAC reaction with azido-diazo-biotin tag, followed by MeOH precipitation for overnight at −20° C. The precipitated proteins were pelleted, washed and air-dried. Resuspend the protein pellet in 8M urea. To reduce and alkylate cysteine residues, the proteins were first treated with 2 mM dithiothreitol for 30 min, and then treated with 6 mM iodoacetamide for 30 min. To remove the reducing and alkylating reagents, the proteins were precipitated in MeOH at −20° C. for overnight, afterwards, the protein pellet was washed thrice with ice-cold MeOH. Resuspend the air-dried protein pellet in freshly-prepared 8M urea. Upon the majority of protein was solubulized, the concentration of urea was reduced to 1.5 M by diluting into 50 mM ABC buffer. Solubilized proteins were then subjected to trypsin digestion (1:50 w/w) in the presence of 20 mM methylamine at 37° C. for overnight. The extent of trypsin digestion was determined by MALDI-TOF and SDS-PAGE. To enrich the biotinylated peptides, the trypsin-digested peptides were incubated with pre-washed streptavidin beads for 2 hr at room temperature on end-over-end rotator. Wash the beads sequentially with 1.5 M Urea, 1× DIBCO's PBS (+0.2% SDS), 1×PBS and ammonium bicarbonate (ABC). To elute the bound peptides, the beads were transferred to dolphin tubes and then incubated with the elution buffer (0.01% SDS, 25 mM sodium dithionite, 50 mM ABC buffer) for 1 hr at room temperature. Pellet the beads and collect the eluent. Repeat the cleavage step once. Dry the eluent in SpeedVac, and then resuspend the dried peptides in dd H₂O (+0.1% TFA). The peptides were cleaned up by C8 cartridge (Waters), eluted with 70% CH₃CN+20% H₂O+0.1% TFA and dried in SpeedVac. The details of analysis of raw tandem mass spectra and selection criterion for candidate proteins have been described, however, the mass of 258.1117, corresponding to the triazole tag (FIG. 55A), was included here as a variable lysine modification in order to search for 4-pentynoate-modification sites. In addition, to investigate the labeling specificity of 4-pentynoate in cells, we searched for other amino acid residues that can be potentially modified by 4-pentynoate, including N-terminal amine, serine, threonine and cysteine. Moreover, to elucidate whether the chain length of 4-pentynoate could possibly be altered in cellular biosynthetic machineries, we searched for different chain-lengths of triazole tag as variable modifications on lysine residues (the set carbon number for acyl-moiety of triazole tag ranges from 1 to 18). The experimental results were shown in FIG. 57.

In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated.

As various modifications could be made in the constructions and methods herein described and illustrated without departing from the scope of the invention, it is intended that all matter contained in the foregoing description or shown in the accompanying drawings shall be interpreted as illustrative rather than limiting. The breadth and scope of the present invention should not be limited by any of the above-described exemplary embodiments, but should be defined only in accordance with the following claims appended hereto and their equivalents.

What is claimed is:

1. A method for detecting one or more acylated protein(s) produced by a cell, the method comprising the steps of:
(a) obtaining an protein lysate from a cell provided with one or more chemical reporter(s);
(b) labeling one or more protein(s) in said protein lysate with one or more detection tag(s); and
(c) detecting one or more acylated protein(s) labelled with said detection tag(s), thereby detecting one or more acylated protein(s) produced by a cell.

2. The method of claim 1, wherein in said detection of one or more acylated protein(s) comprises quantitative detection of said one or more acylated protein(s).

3. The method of claim 1, wherein said cell is provided with said one or more chemical reporter(s) by incubating said cell with said one or more chemical reporter(s).

4. The method of claim 1, wherein said cell is provided with one or more chemical reporter(s) in step (a) by in vivo administration of one or more of said chemical reporter(s) to a non-human organism.

5. The method of claim 1, further comprising the step of separating one or more detection tag labeled protein(s) from step (b) before detection in step (c).

6. The method of claim 1, wherein said one or more acylated protein(s) is an acetylated protein.

7. The method of claim 6, wherein said chemical reporter(s) is/are:
i) of the formula R—(CH₂)ₙ—CO—S—CoA, wherein R is an alkynyl or an azido group and n=2 or 3; or,
ii) of the formula R—(CH₂)ₙ—COOH, wherein R is an alkynyl group or an azido group, and wherein n=2 or 3, or
iii) a corresponding cationic salt of the chemical reporter of the formula R—(CH₂)ₙ—COOH, wherein R is an alkynyl group or an azido group, and wherein n=2 or 3.

8. A method for detecting one or more acylated protein(s) produced by a cell, the method comprising the steps of:
(a) obtaining a protein lysate comprising one or more protein(s) acylated by one or more chemical reporter(s) from a cell provided with one or more chemical reporter(s);
(b) labeling acylated protein(s) in said protein lysate of (a) with one or more detection tag(s) attached to an affinity purification tag by a cleavable linkage;
(c) capturing one or more acylated protein(s) linked to said affinity purification tag in step (b) on a solid support comprising an agent that binds said affinity purification tag;
(d) releasing from said solid support of (c) one or more acylated protein(s) labelled with said detection tag by cleaving said cleavable linkage of said detection tag to said affinity purification tag; and
(e) detecting one or more said acylated protein(s) released in step (d), thereby detecting one or more acylated protein(s) produced by a cell.

9. The method of claim 8, wherein said detection tag further comprises a detectable label that remains linked to said detection tag attached to said acylated protein, following cleavage of said cleavable linkage to said affinity purification tag in step (d).

10. The method of claim 9, wherein said detection tag comprises a compound of the formula (A): 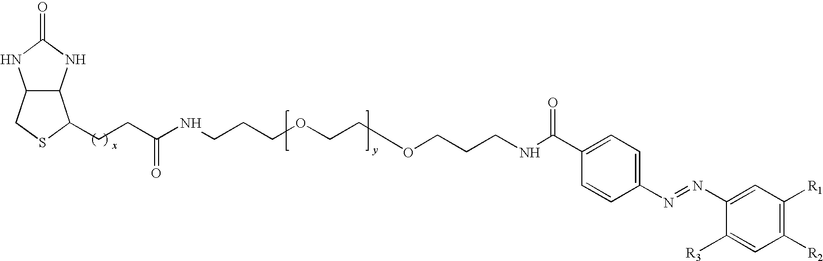
wherein:
x is an integer 1 to 5 inclusive;
y is an integer 1 to 10 inclusive;
R₁ is hydrogen, —(CH₂)z-N═N═N, or —(CH₂)z-NH—CO—(CH₂)z-C≡CH, wherein z is an integer between 2 to 5 inclusive;
R₂ is —H, —O—(CH₂)z-N═N═N, or —O—(CH₂)z-C≡CH, wherein z is an integer between 2 to 5 inclusive;
and R₃ is H or OH.

11. The method of claim 9, wherein said detection tag attached to an affinity purification tag by a cleavable linkage comprises (II) (E)-4-((5-(2-azidoethyl)-2-hydroxyphenyl)diazenyl)-N-(15-oxo-19-(2-oxohexahydro-1H thieno[3,4-d]imidazol-4-yl)-4,7,10-trioxa-14-azanonadecyl)benzamide or (III) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-2-azido-ethyl)-phenylazo]-benzamide.

12. The method of claim 9, wherein said detection tag attached to an affinity purification tag by a cleavable linkage comprises (I) (E)-4-((5-(2-hept-6-ynamidoethyl)-2-hydroxyphenyl)diazenyl)-N-(15-oxo-19-(2-oxohexa hydro-1H-thieno[3,4-a]imidazol-4-yl)-4,7,10-trioxa-14-azanonadecyl)benzamide or (IV) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-buta-4-ynl)-phenylazo]-benzamide.

13. The method of claim 9, wherein said detectable label is a fluorophore selected from the group consisting of a 2-dicyanomethylene-3-cyano-2,5-dihydrofuran fluorophore, rhodamine,

14. A kit comprising:
(a) one or more chemical reporter(s);
(b) one or more detection tag(s) attached to an affinity purification tag by a cleavable linkage; and
(c) containers for said chemical reporter(s) and said detection tag(s).

15. The kit of claim 14, wherein said one or more detection tag(s) further comprise(s) a detectable label that remains linked to said detection tag attached to said acylated protein, following cleavage of said cleavable linkage to said affinity purification tag in step (d).

16. The kit of claim 14, wherein said detection tag comprises a compound of the formula (A): 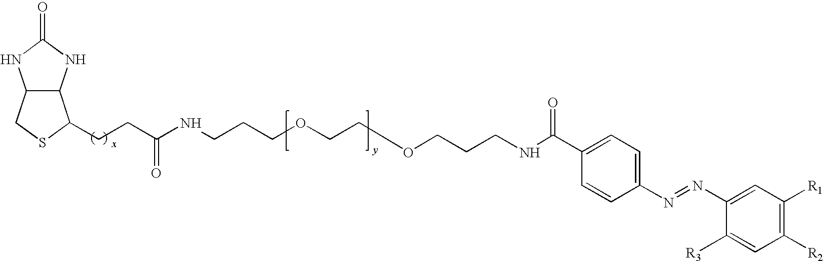
wherein:
x is an integer 1 to 5 inclusive;
y is an integer 1 to 10 inclusive;
R₁ is hydrogen, —(CH₂)z-N═N═N, or —(CH₂)z-NH—CO—(CH₂)z-C≡CH, wherein z is an integer between 2 to 5 inclusive;
R₂ is —H, —O—(CH₂)z-N═N═N, or —O—(CH₂)z-C≡CH, wherein z is an integer between 2 to 5 inclusive;
and R₃ is H or OH.

17. The kit of claim 14, wherein said detection tag attached to an affinity purification tag by a cleavable linkage comprises (II) (E)-4-((5-(2-azidoethyl)-2-hydroxyphenyl)diazenyl)-N-(15-oxo-19-(2-oxohexahydro-1H thieno[3,4-d]imidazol-4-yl)-4,7,10-trioxa-14-azanonadecyl)benzamide or (III) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-2-azido-ethyl)-phenylazo]-benzamide.

18. The kit claim 14, wherein said detection tag attached to an affinity purification tag by a cleavable linkage comprises (I) (E)-4-((5-(2-hept-6-ynamidoethyl)-2-hydroxyphenyl)diazenyl)-N-(15-oxo-19-(2-oxohexa hydro-1H-thieno[3,4-a]imidazol-4-yl)-4,7,10-trioxa-14-azanonadecyl)benzamide or (IV) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-buta-4-ynl)-phenylazo]-benzamide.

19. A compound of the formula (A): 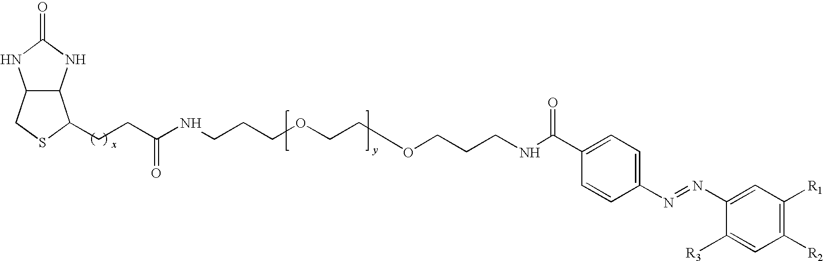
wherein:
x is an integer 1 to 5 inclusive;
y is an integer 1 to 10 inclusive;
R₁ is hydrogen, —(CH₂)z-N═N═N, or —(CH₂)z-NH—CO—(CH₂)z-C≡CH, wherein z is an integer between 2 to 5 inclusive;
R₂ is —H, —O—(CH₂)z-N═N═N, or —O—(CH₂)z-C≡CH, wherein z is an integer between 2 to 5 inclusive;
and R₃ is H or OH.

20. The compound of claim 19, wherein R₁ is —H, —(CH₂)₂—N═N═N, or —(CH₂)₂—NH—CO—(CH₂)₄—C≡CH, R₂ is —H, —O—(CH₂)₂—N═N═N, or —O—(CH₂)₂—C≡CH, and R₃ is H or OH.

21. The compound of claim 19, wherein said compound is selected from the group consisting of:
(I) (E)-4-((5-(2-hept-6-ynamidoethyl)-2-hydroxyphenyl)diazenyl)-N-(15-oxo-19-(2-oxohexa hydro-1H-thieno[3,4-a]imidazol-4-yl)-4,7,10-trioxa-14-azanonadecyl)benzamide;
(II) (E)-4-((5-(2-azidoethyl)-2-hydroxyphenyl)diazenyl)-N-(15-oxo-19-(2-oxohexahydro-1H thieno[3,4-d]imidazol-4-yl)-4,7,10-trioxa-14-azanonadecyl)benzamide;
(III) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-2-azido-ethyl)-phenylazo]-benzamide; and
(IV) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-buta-4-ynl)-phenylazo]-benzamide.

22. The compound of claim 21, wherein said compound is (III) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-2-azido-ethyl)-phenylazo]-benzamide.

23. The compound of claim 21, wherein said compound is (IV) N-(15-oxo-19-((3aS,4S,6aR)-2-oxohexahydro-1H-thieno[3,4-d]imidazol-4-yl)-4,7,10-trioxa-14-azanonadecyl)-4-[4-(oxa-buta-4-ynl)-phenylazo]-benzamide.

24. The compound of claim 21, wherein said compound is (I) (E)-4-((5-(2-hept-6-ynamidoethyl)-2-hydroxyphenyl)diazenyl)-N-(15-oxo-19-(2-oxohexa hydro-1H-thieno[3,4-a]imidazol-4-yl)-4,7,10-trioxa-14-azanonadecyl)benzamide.

25. The compound of claim 21, wherein said compound is (II) (E)-4-((5-(2-azidoethyl)-2-hydroxyphenyl)diazenyl)-N-(15-oxo-19-(2-oxohexahydro-1H thieno[3,4-d]imidazol-4-yl)-4,7,10-trioxa-14-azanonadecyl)benzamide.
